(19) United States
(12) Patent Application Publication
(10) Pub. No.: US 2016/0237449 A1
(43) Pub. Date: Aug. 18, 2016

(54) TRANSGENIC PLANTS FOR NITROGEN FIXATION

(71) Applicant: UNIVERSITY OF BREMEN, Bremen (DE)

(72) Inventors: Barbara Reinhold-Hurek, Bremen (DE); Thomas Hurek, Bremen (DE); Liwei Hu, Zhengzhou, Henan (CN); Qi Wang, Hangzhou, Zhejiang (CN); Haiyuan Yang, Nanjing, Jiiangsu (CN)

(73) Assignee: University of Bremen, Bremen (DE)

(21) Appl. No.: 15/021,928

(22) PCT Filed: Sep. 15, 2014
(86) PCT No.: PCT/EP2014/069624

§ 371 Date: Mar. 14, 2016

(30) Foreign Application Priority Data
Sep. 13, 2013 (EP) 13184351.8

(51) Int. Cl. C12N 15/82; C12N 9/02; G01N 33/84; C12Q 1/68

(52) U.S. Cl. CPC C12N 15/8261; C12N 15/8271; C12Q 1/6895; C12N 9/0095; G01N 33/84; C12Q 2600/13; C12Q 2600/158; C12Y 118/06001; G01N 2333/415; G01N 2458/15; G01N 2560/00

(57) ABSTRACT

Provided are genetic material and nucleic acid sequences useful in increasing yield, biomass, growth rate, vigor, nitrogen use efficiency and/or abiotic stress tolerance, preferably tolerance to nutrient deficiency of a plant. Specifically, the improvement of nitrogen fixation properties in cultivated plants is described.

### FIELD OF THE INVENTION

Generally, the present invention relates to genetic material and nucleic acid sequences useful of increasing yield, biomass, growth rate, vigor, nitrogen use efficiency and/or abiotic stress tolerance, preferably tolerance to nutrient deficiency of a plant. Specifically, the present invention relates to the improvement of nitrogen fixation properties in cultivated plants. The present invention is concerned with transgenic plants, methods of their manufacture and uses of such plants. Also, the present invention relates to methods useful in the manufacture and assessment of such plants. Specifically, the present invention pertains to the field of nitrogen fixation and allows imparting nitrogen fixation properties or enhancing such properties in a plant.

### BACKGROUND OF THE INVENTION

Plant nutrition is essential to the growth and development of plants and therefore also for the quantity and quality of plant products. A huge amount of fertilizers is used worldwide to support plant growth and development. One of the nutrients of major importance is nitrogen, which is usually the rate-limiting element in plant growth. Nitrogen is part of numerous important compounds found in living cells, like amino acids, proteins, nucleic acids and chlorophyll. Because of the high nitrogen requirements of crop plants, nitrogen fertilization is a major worldwide agricultural investment, with approximately 80 million metric tonnes of nitrogen fertilizers applied annually. Nitrogen fertilizers are predominantly made from natural gas by a high energy demanding process. Apart from requiring approximately 1% of the world's total energy supply (Smith B. E., Science 2002 (297), 1654-1655), production of artificial nitrogen fertilizers e.g. by the Haber-Bosch-Mechanism also uses up non-renewable fossil fuels. Nitrogen fertiliser is thus perhaps one of the most energy-intensive inputs to agricultural production worldwide. One third of nitrogen fertilizer is lost by emission of greenhouse gases and leaching, causing adverse environmental impacts. For example, N₂O is a potent greenhouse gas with a relative global warming potential 300 times higher than that of CO₂. Roughly 10% of the human induced greenhouse effect was attributed to N₂O produced predominantly by agricultural management practices but also by industrial processes (Crutzen et al., Chem. Phys. Discuss. 2007 (7), 11191-11205).

Because of the high costs of nitrogen fertilizer and the adverse effect of their use, it is desirable to develop strategies to reduce the consumption of artificial nitrogen fertilizer without impairing yield and plant health.

Traditionally, an alternative to the use of artificial nitrogen fertilizers is three-field crop rotation which uses leguminous plants as green manure. However, it is not the plants themselves which are able to biologically fix atmospheric nitrogen (N₂, also shortened to N in further description); only procaryotic microorganisms comprise nitrogenase, the enzyme complex required for biological nitrogen fixation. Leguminous plants use such nitrogen fixing microorganisms as endosymbionts, particularly microorganisms of genus Rhizobium, e.g., Rh. meliloti. Other nitrogen fixing microorganisms are actinomycetes of genus Franckia, which produce root tubercles for example in plants of genera Hippophae, Ceanothus and Casuarina, particularly Casuarina equisetifolia. Further nitrogen fixing microorganisms are found in cyanobacteria, for example of genus Anabaena like Anabaena azollae, and Nostoc.

Unfortunately, cooperation of plants and microorganisms for biological nitrogen fixation is a complex process requiring coordination of metabolism of both the plant and the microorganism. The relationship between plant and microorganism is on the brink of parasitism, as there is a constant danger that microorganisms could just consume their host instead of merely imparting beneficial nitrogen fixation to the host. Thus, a profit of plants from biological nitrogen fixation does not occur in high amounts in connection with most important agricultural plants, particularly those of family Poaceae, further in particular of subfamily Ehrhartoidae, Pooideae and Panicoideae, Chloridoideae, even more in particular of the tribe Oryzeae, Triticeae, Paniceae, and Andropogoneae, and even more in particular of genus Oryza, particularly O. sativa and O. glaberrima (cultivated rice), genus Hordeum, particularly H. vulgare (barley), genus Secale, particularly S. cereale (rye), genus Triticum, particularly T. aestivum (bread wheat) and T. monococcum, genus Triticosecale (triticale), genus Saccharum, particularly S. officinarum, S. robustum, S. sinense, S. barberi, S. edule and S. spontaneum (sugar cane), genus Eleusine, particularly E. coracana, genus Sorghum, particularly S. bicolor (Sorghum), genus Pennisetum, P. glaucum (millet) and genus Zea, particularly Z. mays (maize).

Unfortunately, the use of green manure is not always possible, for example in the cultivation of rice. Also, green manure cannot be planted together with other crops, thereby decreasing the annual crop yield per hectare. Furthermore, it has been found to be impossible so far to transfer nitrogen fixing organisms to species which are not already adapted to co-cultivation with such microorganisms. Either the respective plants will suffer from infections by such microorganisms, or they will not sustain growth of the microorganisms.

It was therefore an object of the present invention to provide means for reducing the need for artificial nitrogen fertilizers in agriculture, in the cultivation of rice, barley, rye, wheat, triticale, sugar cane, sorghum and maize, most particularly in rice.

### SUMMARY OF THE INVENTION

The present invention generally relates to methods of increasing yield, biomass, growth rate, tolerance to low nutrient soil, vigor and/or nitrogen use efficiency of a plant, comprising introducing isolated genetic material of Oryza longistaminata to a target plant or plant cell. For this aim, the present invention concerns methods for producing a transgenic plant cell and a transgenic plant, comprising the step of introducing genetic material of Oryza longistaminata into a plant cell of a different species to obtain a plant cell or plant, respectively, thereby providing a habitat for nitrogen fixing microorganisms, or thereby modifying or increasing the biological nitrogen fixation of a community comprising the plant cell, or plant respectively, and nitrogen fixing microorganisms. The isolated material may be introduced by several means into the target plants or plant cells, as defined in detail further below, wherein the target plants or plant cells are selected from the family of Poaceae. The present invention also extends towards polynucleotides and vectors comprising the isolated genetic material and cultivated plants, plant cells or seeds thereof in which said genetic material has been introduced. The present invention further relates to the use of said genetic material for alteration of the association of the root of a plant with a nitrogen fixing microorganism, reducing nitrogen fertilization demand of the plant and/or improving nitrogen sustainability of cereals.

In detail the present invention relates to:

[1] A method of increasing yield, biomass, growth rate, vigor, nitrogen gain derived from biological nitrogen fixation, nitrogen use efficiency, abiotic stress tolerance and/or preferably of increasing tolerance to nutrient deficiency of a plant, comprising introducing isolated genetic material of Oryza longistaminata to a target plant or plant cell.

[2] The method of [1], wherein the isolated genetic material is selected from the group consisting of the nucleic acid sequences set forth in Table 1 or nucleic acid sequences encoding a polypeptide at least 60% identical to an amino acid sequence encoded by the nucleic acid sequences set forth in Table 1.

[3] The method of [1] or [2], wherein the target plant or plant cell is selected from the group of plants or plant cells, respectively, of
family Poaceae,
subfamily Ehrhartoidae, Pooideae, Panicoideae or Chloridoideae,
tribum Oryzeae, Triticeae, Paniceae or Andropogoneae,
genus Oryza, Hordeum, Secale, Triticum, Triticosecale, Saccharum, Eleusine, Sorghum, Pennisetum or Zea; and
Oryza sativa, Oryza glaberrima (cultivated rice), Hordeum vulgare (barley), Secale cereale (rye), Triticum aestivum (bread wheat), Triticum monococcum, Saccharum officinarum, Saccharum robustum, Saccharum sinense, Saccharum barberi, Saccharum edule, Saccharum spontaneum (sugar cane), Eleusine coracana, Sorghum bicolor (Sorghum), Pennisetum glaucum (millet), Zea mays (maize), or hybrids thereof.

[4] The method of any one of [1] to [3], wherein the genetic material is introduced by amphiploidization, single chromosome addition/substitution, centric translocation and/or homologous recombination.

[5] The method according to any one of [1] to [4], wherein the genetic material of Oryza longistaminata is introduced to the target plant or plant cell by
wide hybridization,
chemical methods,
microinjection,
electroporation,
particle acceleration and/or particle bombardment,
one or more viral vectors or bacterial vectors, preferably Agrobacterium,
receptor-mediated mechanisms,
injection of genetic material into a reproductive organ of the target plant,
protoplast transformation, and/or
injection into an immature embryo of the target plant.

[6] The method according to any one of [1] to [5], further comprising the steps of

(i) growing a root and/or shoot of the plant or plant cell obtained by the method of any one of [1] to [5] inoculated with a nitrogen fixing microorganism or not inoculated in soil;

(ii) determining expression levels of nitrogenase mRNA in the plant roots;

(iii) selecting a plant having higher root-associated expression levels of nitrogenase mRNA than the parental cultivated plant; and/or

(iv) determining the level of plant nitrogen gained by nitrogen fixation by isotopic, 15N-based methods.

[7] The method according to any one of [1] to [6], wherein the nitrogen fixing microorganism is a bacterium, preferably of family Rhodocyclaceae.

[8] The method to any one of [1] to [7], wherein the introduction of the isolated genetic material to the target plant or plant cell imparts, increases or modifies root association of the plant with a nitrogen fixing microorganism, and/or reduces nitrogen fertilization demand of the plant or the plant cell.

[9] A polynucleotide comprising the isolated genetic material of [1] or [2].

[10] A vector comprising the polynucleotide of [9].

[11] The vector of [10], wherein the nucleic acid sequences as defined in [2] are operably linked to heterologous control sequences capable of directing transcription and preferably expression of the nucleic acid sequences in a host cell, preferably plant cell.

[12] A cultivated plant, plant cell or seed thereof comprising the genetic material as defined in [1] or [2], the polynucleotide of [9], the vector of [10] or [11] and/or obtainable by a method according to any of [1] to [8], preferably exogenously expressing the genetic material.

[13] Use of genetic material of Oryza longistaminata for imparting, increasing or modifying root association of a plant with a nitrogen fixing microorganism, reducing nitrogen fertilization demand of a plant, and/or improving nitrogen sustainability of cereals.

Further embodiments of the present invention will be apparent from the description, the figures and Examples that follow.

### BRIEF DESCRIPTION OF THE DRAWINGS

FIG. 1: Results of polymerase chain reaction (PCR) analysis, showing the detection of nitrogen fixing bacteria in root but not in shoot samples from Oryza longistaminata. A typical result obtained from 5 plants is shown. Plants were grown in a growth chamber in soil from an unfertilized fallow of a rice field in the Carmague, France. Genomic DNA of Azoarcus sp. BH72 is used for spiking of plant samples.

FIG. 2: Levels of nifH transcript fragments in roots of wild and cultivated species of rice. Asian cultivated rice (Oryza sativa) roots showed significantly lower transcript levels than wild rice roots (O. longistaminata). In interspecific hybids (F1) high transcript levels were retained (multiple t tests with multiple comparison post test; *, P<0.05). Values are means with SD from at least 3 replicates. All values from the rice cultivars were compared to those from wild rice. NifH transcripts were quantified by real-time RT-PCR. The actin gene was used to normalize the quantification of nifH expression.

FIG. 3: Increasing the capacity for BNF in Asian cultivated rice by wide hybridization with wild rice. Incorporation of ¹⁵N₂ tracer into re-grown shoots of a wild rice species Oryza longistaminata, F1-hybrids, Asian cultivated rice (Oryza sativa) and backcrosses with Oryza sativa. Plants were grown in a gas-tight phytotron chamber and sampled after the 2nd consecutive cut, after growth under a headspace of 0.41±0.15 atom % ¹⁵N excess. In contrast to the O. sativa cultivar and the backcross tested, two different F1-hybrids retained high N2-gain potential similar to wild rice (one-way ANOVA and Bonferroni's multiple comparison post test *, P<0.05).

### DETAILED DESCRIPTION OF THE INVENTION

Basic plant biochemistry and physiology have provided the means of better understanding of crop production systems in relation to Nitrogen (N) supply. The interactions between carbon dioxide (CO₂) and nitrate (NO₃⁻) assimilation and their dynamics are of key importance for crop production. An adequate supply of NO₃⁻, its assimilation to amino acids (for which photosynthesized carbon compounds are required) and their availability for protein synthesis, are essential for metabolism. An adequate supply of NO₃⁻ stimulates leaf growth and photosynthesis, the former via cell growth and division, the latter by larger contents of components of the light reactions, and those of CO₂ assimilation and related processes. With adequate N, at full genetic potential of a plant, more C-assimilation per unit N would increase biomass. As indicated above, nitrogen fertilization is broadly used in the agriculture achieve adequate N supply, with adverse effects as recited above as well. An alternative would be represented, e.g., by increasing the N assimilation or utilization in a plant cell or a plant, which in turn would exhibit improved nitrogen contents, altered amino acid or protein compositions, vigorous growth characteristics, increased vegetative yields or better seed yields and qualities.

On the above background, the present invention generally relates to a method of increasing yield, biomass, growth rate, vigor, nitrogen use efficiency (NUE) and/or abiotic stress tolerance, preferably tolerance to nutrient deficiency of a plant, comprising introducing isolated genetic material of Oryza longistaminata to a target plant or plant cell.

A way for identification of such plants, plant cells and plant lines engineered to possess such improved agronomic characteristics may lay, e.g., in examination of any of following parameters: 1) the rate of growth, measured in, terms of rate of increase in fresh or dry weight; 2) vegetative yield of the mature plant, in terms of fresh or dry weight; 3) the seed or fruit yield; 4) the seed or fruit weight; 5) the total nitrogen content of the plant; 6) the total nitrogen content of the fruit or seed; 7) the free amino acid content of the plant; 8) the free amino acid content of the fruit or seed; 9) the total protein content of the plant; and 10) the total protein content of the fruit or seed. The procedures and methods for examining these parameters are well known to those skilled in the art.

As used herein the term “increasing” refers to at least about 1%, 2%, 3%, 4%, 5%, preferably at least about 10%, 15%, 20%, more preferred at least about 25%, 30%, 35%, 40%, 45% and most preferred at least about 50%, 60%, 70%, or 80% increase in at least parameter of a plant as compared to a native plant [i.e., a plant not modified by the introduction of the genetic material of the invention or equivalent biomolecules, i.e. polynucleotides or polypeptides obtainable by the expression of the genetic material, e.g., a non-transformed plant of the same species which is grown under the same growth conditions).

As indicated above, within the method of the According to the present invention, a desired plant is one that exhibits improvement over the control plant (i.e., progenitor plant) by increase of one or more of the aforementioned parameters.

Generally speaking, the present invention concerns generation of a transgenic plant or transgenic plant cell by introducing genetic material of Oryza longistaminata, which has a high ability to use nitrogen efficiently, to a target plant or plant cell. The introduction of said genetic material reduces the plant's or plant cell's demand for nitrogen fertilization preferably by imparting, increasing or modifying root association of a plant with a nitrogen fixing microorganism and/or increases yield, biomass, growth rate, vigor, nitrogen use efficiency, abiotic stress tolerance, and/or preferably tolerance to nutrient deficiency of the plant or plant cell. In addition or alternatively, in one embodiment, the genetic material is preferably of the strain O. longistaminata Xa21, also known as O. longistaminata IRGC 110404 or CRRI 12156-1.

In one embodiment, the present invention relates to the method as defined hereinabove, wherein the isolated genetic material is selected from the group consisting of the nucleic acid sequences set forth in Table 1 below or nucleic acid sequences encoding a polypeptide at least 60% identical, preferably 65, 70 or 75% identical, more preferably 80% identical, still more preferably 90% identical, and particularly preferred at least 91%, 92%, 93%, 94%, 95%, 96%, 97%, 98% or 99% identical to an amino acid sequence encoded by the nucleic acid sequences set forth in Table 1.

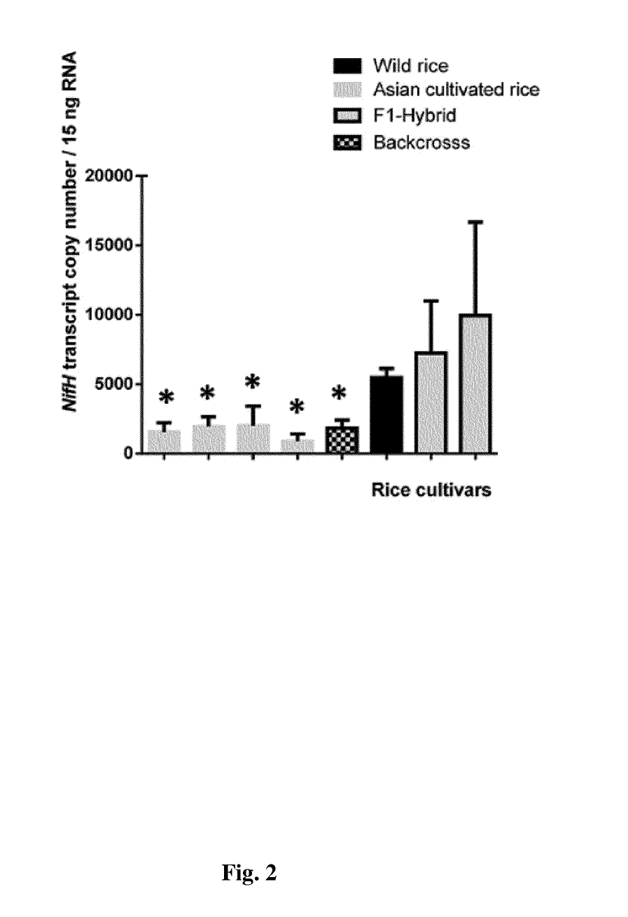

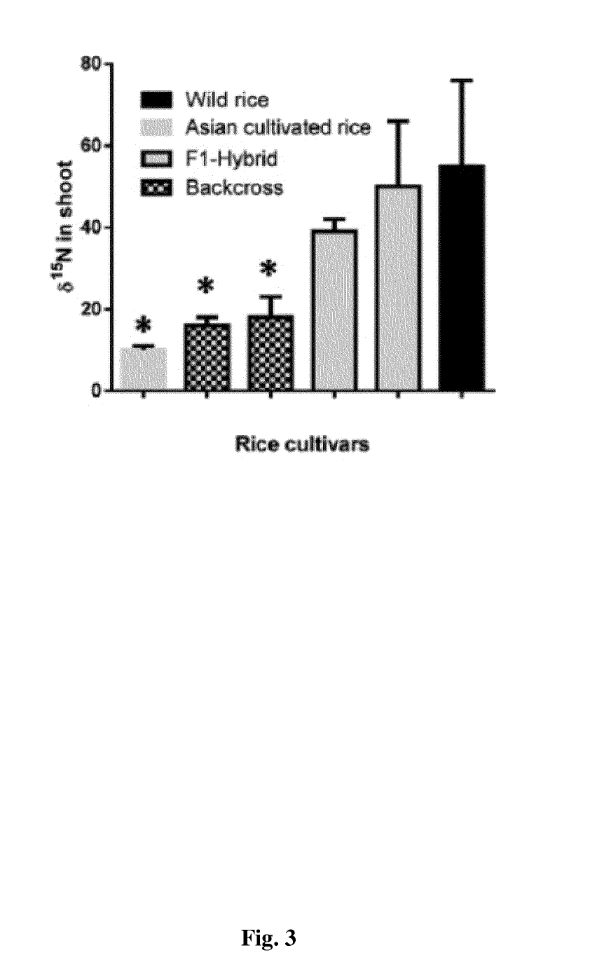

In a particularly preferred embodiment of the present invention one or more of the nucleic acid sequences set forth in Table 1 or polynucleotides encoding substantially the same or equivalent polypeptides are introduced into the target plant. As shown in FIGS. 2 and 3 the introduction of one or more of these sequences by techniques such as wide hybridization leads to an increased NifH transcript copy number in the roots and increased incorporation of ¹⁵N₂ tracer into re-grown shoots indicating higher nitrogen fixation in the modified plants (F1-Hybrid in FIGS. 2 and 3)

According to some embodiments of the invention the isolated genetic material comprises a nucleic acid sequence which is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 93%, at least about 94%, at least about 95%, at least about 96%, at least about 97%, at least about 98%, at least about 99%, e.g., 100% identical to the nucleic acid sequences as indicated in Table 1 and selected from the group consisting of the sequences indicated in Table 1.

Naturally, the plant to be modified is not of species Oryza longistaminata. In one preferred embodiment, the plant to be modified is a cultivated plant and/or comprises at least one, two, three, four, five, six, seven, eight, nine, ten or more of the sequences set forth in Table 1. In one embodiment, the subject plant comprises one or more chromosomes or part(s) thereof of the wild type species, preferably comprising one or more of the sequences indicated in Table 1.

In one embodiment, the present invention relates to the method as defined hereinabove, wherein the target plant or plant cell is selected from the group of plants or plant cells, respectively, of
family Poaceae,
preferably of subfamily Ehrhartoidae, Pooideae, Panicoideae or Chloridoideae,
more preferred of tribum Oryzeae, Triticeae, Paniceae or Andropogoneae,
still more preferred of genus Oryza, Hordeum, Secale, Triticum, Triticosecale, Saccharum, Eleusine, Sorghum, Pennisetum or Zea; and
in particular preferred of Oryza sativa, Oryza glaberrima (cultivated rice), Hordeum vulgare (barley), Secale cereale (rye), Triticum aestivum (bread wheat), Triticum monococcum, Saccharum officinarum, Saccharum robustum, Saccharum sinense, Saccharum barberi, Saccharum edule, Saccharum spontaneum (sugar cane), Eleusine coracana, Sorghum bicolor (Sorghum), Pennisetum glaucum (millet), Zea mays (maize), and hybrids thereof, preferably having at least one parent of such species, genus, tribe, subfamily or family. Preferably, the target plant or plant cell is selected from Oryza sativa, Oryza glaberrima or hybrids thereof.

The genetic material as defined above may be introduced in several forms and by many methods known in the art into the target cells or plants. In one embodiment, the present invention relates to the method as defined hereinabove, wherein the genetic material is introduced by amphiploidization, single chromosome addition/substitution, centric translocation and/or homologous recombination. These techniques are well known in the art; see, e.g., Cox et al., in Critical Reviews in Plant Sciences 21 (2002), 59-91 and references cited therein. Furthermore, methods of constructing and transfecting of artificial chromosomes (chromosome addition) are known in the art and reviewed or described, e.g., in Houben A., The Plant Cell January 20 (2008), 8-10 and Phan et al. Transgenic Res. 16 (2007), 341-51. Chromosome substitution is described for example in Wan et al., Theor Appl Genet 110 (2004), 71-79; and Yano M, Curr Opin Plant Biol 4 (2001), 130-135.

After a suitable construct comprising the genetic material as defined hereinabove has been made, transgenic plants of interest can be generated using transformation methods well known in the art. The construct according to the present invention can be in general a polynucleotide comprising or essentially consisting of the genetic material as defined hereinabove, for example, a plasmid, a bacmid, a phagemid, a cosmid, a phage, a virus or a chromosome. An exogenous nucleic acid molecule can be introduced into a monocot plant for ectopic expression using a variety of transformation methodologies including Agrobacterium-mediated transformation (Hiei et al., The Plant Journal 6 (1994), 271-282) and direct gene transfer methods such as electroporation and microprojectile-mediated transformation (see, generally, Wang et al. (eds), Transformation of Plants and Soil Microorganisms, Cambridge, UK: University Press, 1995, which is incorporated herein by reference).

Transformation methods based upon the soil bacterium, Agrobacterium tumefaciens, are particularly useful for introducing an exogenous nucleic acid molecule into a seed plant. The wild-type form of Agrobacterium contains a Ti (tumor-inducing) plasmid that directs production of tumorigenic crown gall growth on host plants. Transfer of the tumor-inducing T-DNA region of the Ti plasmid to a plant genome requires the Ti plasmid-encoded virulence genes as well as T-DNA borders, which are a set of direct DNA repeats that delineate the region to be transferred. An Agrobacterium-based vector is a modified form of a Ti plasmid, in which the tumor inducing functions are replaced by the nucleic acid sequence of interest to be introduced into the plain host.

Agrobacterium-mediated transformation generally employs cointegrate vectors or, preferably, binary vector systems, in which the components of the Ti plasmid are divided between a helper vector, which resides permanently in the Agrobacterium host and carries the virulence genes, and a shuttle vector, which contains the particular genetic material as defined hereinabove, e.g., an isolated gene of interest bounded by T-DNA sequences. A variety of binary vectors is well known in the art and are commercially available, for example, from Clontech (Palo Alto, Calif.). Methods of co-culturing Agrobacterium with cultured plant cells or wounded tissue such as root explants, hypocotyledons, stem pieces or tubers, for example, also are well known in the art (Glick and Thompson, Methods in Plant Molecular Biology and Biotechnology. CRC Press, Boca Raton, Fla., pp 179-20519, 1993). Wounded cells within the plant tissue that have been infected by Agrobacterium can develop organs de novo when cultured under the appropriate conditions; the resulting transgenic shoots eventually give rise to transgenic plants that comprise the genetic material as defined hereinabove and/or ectopically express, e.g., a polynucleotide and/or polypeptide/protein from there within. Agrobacterium also can be used for transformation of whole seed as described in Bechtold et al., C. R. Acad. Sci. Paris. Life Sci. 316 (1993), 1194-1199, (which is incorporated herein by reference). Agrobacterium-mediated transformation is useful for producing a variety of transgenic seed plants (Wang et al., supra, 1995). Methods Mol Biol. 2012; 847:51-7. doi: 10.1007/978-1-61779-558-9_5.

A high-efficiency Agrobacterium-mediated transformation system of several rice Oryza sativa L. elite japonica and many indica varieties is described by Ozawa in Methods Mol. Biol. 847 (2012), 51-57.

Microprojectile-mediated transformation also can be used to produce a transgenic plant that comprises and/or ectopically expresses the genetic material as defined hereinabove. This method, first described by Klein et al. (Nature 327 (1987), 70-73), which is incorporated herein by reference), relies on microprojectiles such as gold or tungsten that are coated with the desired nucleic acid molecule by precipitation with calcium chloride, spermidine or PEG. The microprojectile particles are accelerated at high speed into a plant tissue using a device such as the BIOLISTIC PD-1000 (Biorad, Hercules, Calif.).

Microprojectile-mediated delivery, also known as “particle acceleration” and “particle bombardment” is especially useful to transform plants that are difficult to transform or regenerate using other methods. Microprojectile-mediated transformation has been used, for example, to generate a variety of transgenic plant species, including cotton, tobacco, maize, hybrid poplar and papaya (see Glick and Thompson, supra, 1993) as well as cereal crops such as wheat, oat, barley, sorghum and rice (Duan et al., Nature Biotech. 14:494-498, 1996; Shimamoto, Curr. Opin. Biotech. 5:158-162, 1994; each of which is incorporated herein by reference). In view of the above, the skilled artisan will recognize that Agrobacterium-mediated or microprojectile-mediated transformation, as disclosed herein, or other methods known in the art can be used to produce a transgenic seed plant of the invention.

Alternative gene transfer and transformation methods useful in the invention include, but are not limited to liposomes (Caboche M., Physiol. Plant. 79 (1990), 173-176); electroporation (Riggs et al. (1986) Proc. Natl. Acad. Sci. USA 83:5602-5606); chemical-mediated methods (Zatloukal, et al., Ann. N.Y. Acad. Sci., 660 (1992), 136-153); calcium phosphate co-precipitation techniques; micro- or macroinjection (Crossway et al. (1986) Biotechniques 4:320-334); direct DNA transformation, and may involve Ti plasmids, Ri plasmids, or plant virus vectors; receptor-mediated mechanisms (Curiel et al., Hum. Gen. Ther., 3(2):147-154, 1992; Wagner, et al., Proc. Natl. Acad. Sci. USA, 89(13): 6099-6103, 1992) injection of genetic material into a reproductive organ of the target plant (Zhou, et al., Methods in Enzymology, 101: 433, 1983; Hess, Intern Rev. Cytol., 107: 367, 1987; Luo, et al., Plant Mol. Biol. Reporter, 6: 165, 1988; Pena, et al., Nature, 325: 274, 1987); protoplast transformation (U.S. Pat. No. 5,508,184); injection into an immature embryo of the target plant (Neuhaus, et al., Theor. Appl. Genet., 75 (1987), 30-36) and wide hybridization (Kumari et al., Annuals of the Sri Lanka Department of Agriculture 7 (2005), 157-164). These and further plant transformation methods are known to the person skilled in the art, the above references are exemplary only. Such methods are described in detail also in the patent application US 2010/0275332 A1, in the section “Transformation”, paragraphs [0129-0145] on pages 16-17, the disclosure content of which is incorporated herein by reference in its entirety.

Accordingly, in one embodiment the present invention concerns the method as described hereinabove, wherein the genetic material of Oryza longistaminata is introduced to the target plant or plant cell by
wide hybridization,
chemical methods,
microinjection,
electroporation,
particle acceleration and/or particle bombardment,
one or more viral vectors or bacterial vectors, preferably Agrobacterium,
receptor-mediated mechanisms,
injection of genetic material into a reproductive organ of the target plant,
cytoplast fusion
protoplast transformation, and/or
injection into an immature embryo of the target plant.

Methods using either a form of direct gene transfer or Agrobacterium-mediated transfer usually, but not necessarily, are undertaken with a selectable marker which may provide resistance to an antibiotic (e.g., kanamycin, hygromycin or methotrexate) or a herbicide (e.g., phosphinothricin). The choice of selectable marker for plant transformation is not, however, critical to the invention.

For certain plant species, different antibiotic or herbicide selection markers may be preferred. Selection markers used routinely in transformation include the nptII gene which confers resistance to kanamycin and related antibiotics (Messing & Vierra, Gene 19: 259-268 (1982); Bevan et al., Nature 304:184-187 (1983)), the bar gene which confers resistance to the herbicide phosphinothricin (White et al., Nucl Acids Res 18: 1062 (1990), Spencer et al., Theor Appl Genet 79: 625-631(1990)), the hph gene which confers resistance to the antibiotic hygromycin (Blochinger & Diggelmann, Mol Cell Biol 4: 2929-2931), and the dhfr gene, which confers resistance to methotrexate (Bourouis et al., EMBO J. 2: 1099-1104 (1983)).

The genetic material as defined hereinabove may be isolated and/or introduced in several forms into the plants and/or plant cells, for example in the form of a polynucleotide. The polynucleotide of the present invention may comprise or essentially consist of the genetic material as defined hereinabove. It may be, e.g., DNA, cDNA, RNA or synthetically produced DNA or RNA or a recombinantly produced chimeric nucleic acid molecule comprising any of those polynucleotides either alone or in combination. Therefore, in one embodiment the present invention relates to a polynucleotide comprising the isolated genetic material of the present invention or at least one nucleic acid sequence as defined hereinabove, i.e. selected from the group consisting of the nucleic acid sequences set forth in Table 1 below or nucleic acid sequences encoding a polypeptide at least 60% identical, preferably 65, 70 or 75% identical, more preferably 80% identical, still more preferably 90% identical, and particularly preferred at least 91%, 92%, 93%, 94%, 95%, 96%, 97%, 98% or 99% identical to an amino acid sequence encoded by the nucleic acid sequences set forth in Table 1.

Furthermore, in a preferred embodiment the present invention also relates to a vector comprising the polynucleotide as defined supra. Preferably, the polynucleotide of the invention is operatively linked to expression control sequences allowing expression in prokaryotic or eukaryotic cells. Accordingly, in one embodiment the vector as defined herein above is provided, wherein the nucleic acid sequences set forth in Table 1 or nucleic acid sequences encoding a polypeptide at least 60% identical to an amino acid sequence encoded by the nucleic acid sequences set forth in Table 1 are operably linked to heterologous control sequences capable of directing transcription and preferably expression of the nucleic acid sequences in a host cell, preferably plant cell.

Expression of said polynucleotide comprises transcription of the polynucleotide into a translatable mRNA. Regulatory elements ensuring expression in eukaryotic cells, preferably mammalian cells, are well known to those skilled in the art. They usually comprise regulatory sequences ensuring initiation of transcription and optionally poly-A signals ensuring termination of transcription and stabilization of the transcript. Additional regulatory elements may include transcriptional as well as translational enhancers, and/or naturally associated or heterologous promoter regions.

For expression in plants, suitable promoters must be chosen for both the receptor expression cassettes and the target expression cassette. Unless specifically noted, the promoters discussed below may be used to direct expression in plants of either the receptor polypeptides or the target polypeptide. These promoters include, but are not limited to, constitutive, inducible, temporally regulated, developmentally regulated, chemically regulated, tissue-preferred and tissue-specific promoters.

Preferred constitutive promoters include but are not limited to the CaMV 35S and 19S promoters (U.S. Pat. No. 5,352,605). Additionally preferred promoters include but are not limited to one of several of the actin genes, which are known to be expressed in most cell types. The promoter described by McElroy et al., Mol. Gen. Genet. 231 (1991), 150-160, can be easily incorporated into the receptor expression cassettes of the present invention and are particularly suitable for use in monocotyledonous hosts. Yet another preferred constitutive promoter is derived from ubiquitin, which is another gene product known to accumulate in many cell types. The ubiquitin promoter has been cloned from several species for use in transgenic plants (e.g. sunflower—Binet et al., Plant Science 79 (1991), 87-94; maize—Christensen et al., Plant Molec. Biol. 12 (1989), 619-632). The maize ubiquitin promoter has been developed in transgenic monocot systems and its sequence and vectors constructed for transformation of monocotyledonous plants are disclosed in patent publication EP 0 342 926. The ubiquitin promoter is suitable for use in the present invention in transgenic plants, especially monocotyledons. Further useful promoters are the U2 and U5 snRNA promoters from maize (Brown et al., Nucleic Acids Res. 17: 8991 (1989)) and the promoter from alcohol dehydrogenase (Dennis et al., Nucleic Acids Res. 12: 3983 (1984)).

Tissue-specific or tissue-preferential promoters useful in the present invention in plants, particularly maize, are those which direct expression in root, pith, leaf or pollen. Such promoters are disclosed in U.S. Pat. No. 5,625,136, incorporated herein by reference in its entirety. Also useful are promoters which confer seed-specific expression, such as those disclosed by Schernthaner et al, EMBO J., 7 (1988) 1249-1255; anther-specific promoters ant32 and ant43D disclosed in U.S. Pat. No. 5,477,002, incorporated herein by reference in its entirety; anther (tapetal) specific promoter B6 (Huffman et al., J. Cell. Biochem. 17B: Abstract #D209 (1993)); pistil-specific promoters such as a modified S13 promoter (Dzelkalns et al., Plant Cell 5 (1993), 855).

Also useful in the present invention are chemically-induced promoters. Particular promoters in this category useful for directing the expression of the receptor polypeptides or target polypeptide in plants are disclosed, for example, in U.S. Pat. No. 5,614,395, incorporated herein by reference in its entirety.

The 5′ regulatory region of either the receptor expression cassette or the target expression cassette may also include other enhancing sequences. Numerous sequences have been found to enhance gene expression in transgenic plants. For example, a number of non-translated leader sequences derived from viruses are known to enhance expression. Specifically, leader sequences from Tobacco Mosaic Virus (TMV, the “Ω-sequence”), Maize Chlorotic Mottle Virus (MCMV), and Alfalfa Mosaic Virus (AMV) have been shown to be effective in enhancing expression (e.g. Gallie et al., Nucl. Acids Res., 15 (1987), 8693-8711; Skuzeski et al. Plant Molec. Biol., 15 (1990) 65-79).

Various intron sequences have been shown to enhance expression when added to the 5′ regulatory region, particularly in monocotyledonous cells. For example, the introns of the maize Adh1 gene have been found to significantly enhance the expression of the wild-type gene under its cognate promoter when introduced into maize cells (Callis et al., Genes Develop., 1 (1987), 1183-1200).

In addition to promoters, a variety of 3′ transcriptional terminators are also available for use in the present invention. Transcriptional terminators are responsible for the termination of transcription and correct mRNA polyadenylation. Appropriate transcriptional terminators and those which are known to function in plants include the CaMV 35S terminator, the tml terminator, the nopaline synthase terminator, the pea rbcS E9 terminator and others known in the art. These can be used in both monocotyledons and dicotyledons.

In addition to incorporating one or more of the aforementioned elements into the 5′ regulatory region of a target expression cassette, other elements peculiar to the target expression cassette may also be incorporated. Such elements include but are not limited to a minimal promoter. By minimal promoter it is intended that the basal promoter elements are inactive or nearly so without upstream activation. Such a promoter has low background activity in plants when there is no transactivator present or when enhancer or response element binding sites are absent. One minimal promoter that is particularly useful for target genes in plants is the Bz1 minimal promoter which is obtained from the bronzel gene of maize.

Another advantage of the quantification method of the present invention is that it can be used to select plants having a low demand of artificial nitrogen fertilization. Accordingly, in one embodiment the present invention also relates to the method of the present invention as defined herein above further comprising the steps of
growing root(s) and/or shoot(s) of the plant or the plant cell inoculated with a nitrogen fixing microorganism or not inoculated in soil;
determining expression levels of nitrogenase mRNA in the plant root(s);
selecting a plant having higher root-associated expression levels of nitrogenase mRNA than the parental cultivated plant; and/or
determining the level of plant nitrogen gained by nitrogen fixation by isotopic, ¹⁵N-based methods.

As indicated above, it is not the plants themselves which are able to biologically fix atmospheric nitrogen (N2, also shortened to N in further description); only procaryotic microorganisms comprise nitrogenase, the enzyme complex required for biological nitrogen fixation. Therefore, it is envisaged to modify and preferably to increase the root association of the plant, by the method of the present invention wherein the genetic material as defined hereinabove has been introduced with such a biosymbiontic organism.

Without wishing to be bound by theory, experiments performed within the scope of the present invention indicated that Oryza longistaminata is particularly able to support root-associated microorganisms which are capable of biological nitrogen fixation due to which O. longistaminata shows a high ability to use nitrogen efficiently (NUE). The inventors have also found that it is possible to transfer genetic material from O. longistaminata to other species, preferably to O. sativa, to impart, enhance or otherwise modify the ability of such plant of other species to sustain root-associated nitrogen fixing microorganisms, and to use the nitrogen provided by such root-associated microorganisms. This finding is particularly surprising as it had become apparent that it is not possible to transfer nitrogen fixing microorganisms from leguminous plants to those of other species and maintain them in a stable association which provides biologically fixed nitrogen to the other species. Furthermore, it was surprisingly found that a stable association of nitrogen fixing microorganisms to plants could be established by merely transferring genetic material of O. longistaminata to the other species without requiring genetic modification of the nitrogen fixing microorganisms themselves.

According to the present invention, root association of a plant with nitrogen fixing microorganisms means that one or more species of nitrogen fixing microorganisms colonizes the interior of roots or root nodules or other tissue of the plant such that after inoculation of a plant with said microorganism(s) and growing of said plant for at least 1 month without further addition of said microorganism(s), viable cells of said microorganism(s) can still be detected in the plant tissue. Preferably, the nitrogen fixing microorganism(s) are endophytic.

Accordingly, in one embodiment the present invention concerns the method of the present invention as defined hereinabove, wherein the introduction of the isolated genetic material to the target plant or plant cell imparts, increases or modifies root association of the plant with at least one nitrogen fixing microorganism, and/or reduces nitrogen fertilization demand of the plant or the plant cell. In this respect, in one embodiment the nitrogen fixing microorganism(s) is/are diazotroph(s), preferably a (diazotrophic) bacterium, more preferably of family Rhodocyclaceae, still more preferred of genus Azoarcus, Azospira, Azovibrio or Azonexus and most preferably of genus Azoarcus. Preferably such a microorganism is further characterized by harboring genes for nitrogen fixation known in the art such as ninHDK, anfHDK, and/or vnfHDK.

O. longistaminata belongs to the group of Oryza sativa-like plants, and is traditionally identified by its AA genome type having 24 chromosomes (cf. Liakat Ali et al., Rice 2010 (3): 218-234). The species Oryza longistaminata and particularly the strain Xa21 had previously only gained attention due its resistance to bacterial blight and bacterial leaf streak and other pests and pathogens. It was the more surprising that this well analyzed species comprises a hitherto overlooked but important physiological property, i.e. sustaining a stable root-association with nitrogen fixing microorganisms, and that this property could be transferred to plants of other species. In particular, the inventors have found that O. longistaminata has a high proven input of biologically fixed nitrogen into plant biomass of (extrapolated) 80 kg N per ha per year even without inoculation with nitrogen fixing microorganisms. Furthermore, the inventors found that F1 generation of plants obtained by wide hybridization of O. sativa×O. longistaminata can gain similar amounts of fixed nitrogen as O. longistaminata, while O. sativa cultivars showed drastically lower nitrogen input as measured by ¹⁵N uptake.

Furthermore, in one embodiment the method of the present invention preferably comprises the step of crossbreeding a plant obtained according to any of the previous claims with a wild type plant of the target plant's species. This is preferably achieved by backcrossing of a plant of the F1 generation of O. longistaminata×the target species with a wild type of the target species. Preferably, even in F2 generations a stable association of nitrogen fixing microorganisms and plant roots of the F2 generation can be maintained. This is of particular advantage since F1 offspring of wide hybridization is sterile and may comprise traits of O. longistaminata which are not required for the desired transgenic plant or plant cell.

Furthermore, the inventors observed that it is difficult to quantify biological nitrogen fixation in plants. To screen the results of nitrogen transfer according to the present invention, preferably by wide hybridization, it is therefore advantageous to provide a method for rapid analysis of a biochemical trait correlating with nitrogen fixation.

In one embodiment thus, the present invention also relates to a method for quantification of nitrogenase gene expression in plant roots, comprising the steps of

(i) extracting mRNA of plant shoot roots comprising nitrogen fixing microorganisms,

(ii) quantification of nitrogenase mRNA by normalization

a) on plant ribosomal RNA levels, preferably of actin levels,

b) on in vitro amplified and quantified nitrogenase mRNA levels.

It is a particular advantage of the method of the present invention that it allows to quantify expression of nitrogenase in one day, whereas quantification by ¹⁵N uptake requires cultivation of plants for approximately two months. The analysis method of the present invention therefore allows a rapid characterization of offspring obtained by a transfer method of the present invention according to the expected nitrogen fixation and uptake rate of said offspring.

Furthermore, in one embodiment the present invention also relates to a cultivated plant, plant cell or seed thereof comprising the genetic material, the polynucleotide, the vector as defined hereinabove and/or obtainable by a method as described herein above, preferably exogenously expressing the genetic material.

The present invention also concerns a use of genetic material of Oryza longistaminata for imparting, increasing or modifying root association of a plant with a nitrogen fixing microorganism, reducing nitrogen fertilization demand of a plant, and/or improving nitrogen sustainability of cereals.

Acquisition of expression levels of nitrogenase mRNA is preferably performed by RT-PCR with nifH-specific primers and quantified using quantitative, real-time RT-PCR. RNA extraction and primer sets were described previously (T. Hurek, L. L. Handley, B. Reinhold-Hurek, Y. Piché, MPMI 15, 233 (2002), and Burbano, C. S., Y. Liu, K. L. Rösner, V. M. Reis, J. Caballero-Mellado, B. Reinhold-Hurek, T. Hurek. Environ. Microbiol. Rep. 3, 383 (2011) both documents are incorporated herein by reference for the purpose of describing determination of nifH transcript levels.

### DEFINITIONS AND EMBODIMENTS

It is to be noted that the term “a” or “an” entity refers to one or more of that entity; for example, “a plant cell,” is understood to represent one or more plant cells. As such, the terms “a” (or “an”), “one or more,” and “at least one” can be used interchangeably herein.

A cultivar and a cultivated plant, respectively, is a systematic group of cultivated plants that is clearly distinct, uniform, and stable in its characteristics and which, when propagated by appropriate means, retains these characteristics. A cultivar-group is a group of properly named cultivars, based on one or more criteria. A cultivated plant is a plant whose origin or selection is primarily due to the intentional activities of mankind Such a plant may arise either by deliberate or, in cultivation, accidental hybridization, or by selection from existing cultivated stock, or may be a selection from minor variants within a wild population and maintained as a recognizable entity solely by deliberate and continuous propagation; see, e.g., Trehane et al., Int. code of nomenclature of cultivated plants, Regnum Veg. 133 (1995), 1-175.

Thus, while Oryza longistaminata may be considered a wild type, non-cultivated plant rice Oryza sativa and elite rice varieties such japonica are considered cultivated plants. As mentioned before, the plant to be modified in accordance with the present invention is a cultivated plant.

### Polypeptides:

As used herein, the term “polypeptide” is intended to encompass a singular “polypeptide” as well as plural “polypeptides”, and refers to a molecule composed of monomers (amino acids) linearly linked by amide bonds (also known as peptide bonds). The term “polypeptide” refers to any chain or chains of two or more amino acids, and does not refer to a specific length of the product. Thus, “peptides,” “dipeptides,” “tripeptides, “oligopeptides,” “protein,” “amino acid chain,” or any other term used to refer to a chain or chains of two or more amino acids, are included within the definition of “polypeptide,” and the term “polypeptide” may be used instead of, or interchangeably with any of these terms.

The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for purposed of the invention, as are native or recombinant polypeptides which have been separated, fractionated, or partially or substantially purified by any suitable technique.

“Recombinant peptides, polypeptides or proteins” refer to peptides, polypeptides or proteins produced by recombinant DNA techniques, i.e. produced from cells, microbial or plant cells, transformed by an exogenous recombinant DNA expression construct encoding the fusion protein including the desired peptide. Proteins or peptides expressed in most bacterial cultures will typically be free of glycan. Proteins or polypeptides expressed in yeast may have a glycosylation pattern different from that expressed in mammalian cells.

Also included as polypeptides of the present invention are fragments, derivatives, analogs and variants of the foregoing polypeptides and any combination thereof. The terms “fragment,” “variant,” “derivative” and “analog” include peptides and polypeptides having an amino acid sequence sufficiently similar to the amino acid sequence of the natural peptide. The term “sufficiently similar” means a first amino acid sequence that contains a sufficient or minimum number of identical or equivalent amino acid residues relative to a second amino acid sequence such that the first and second amino acid sequences have a common structural domain and/or common functional activity. For example, amino acid sequences that comprise a common structural domain that is at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100%, identical are defined herein as sufficiently similar. Preferably, variants will be sufficiently similar to the amino acid sequence of the preferred polypeptides of the present invention. Such variants generally retain the functional activity of the polypeptides of the present invention. Variants include peptides that differ in amino acid sequence from the native and wt polypeptide, respectively, by way of one or more amino acid deletion(s), addition(s), and/or substitution(s). These may be naturally occurring variants as well as artificially designed ones.

Determination of Similarity and/or Identity of Molecules

“Similarity” between two polypeptides is determined by comparing the amino acid sequence of one polypeptide to the sequence of a second polypeptide. An amino acid of one polypeptide is similar to the corresponding amino acid of a second polypeptide if it is identical or a conservative amino acid substitution. Conservative substitutions include those described in Dayhoff, M. O., ed., The Atlas of Protein Sequence and Structure 5, National Biomedical Research Foundation, Washington, D.C. (1978), and in Argos, EMBO J. 8 (1989), 779-785. For example, amino acids belonging to one of the following groups represent conservative changes or substitutions: -Ala, Pro, Gly, Gln, Asn, Ser, Thr; -Cys, Ser, Tyr, Thr; -Val, Ile, Leu, Met, Ala, Phe; -Lys, Arg, His; -Phe, Tyr, Trp, His; and -Asp, Glu. For nucleic acids, such as RNA and DNA, similarity or identity between two molecules is mostly determined just by distinguishing between matches, where the two residues are the same, and mismatches between both sequences.

The determination of percent identity or similarity between two sequences is preferably accomplished using the mathematical algorithm of Karlin and Altschul (1993) Proc. Natl. Acad. Sci USA 90: 5873-5877. Such an algorithm is incorporated into the BLASTn and BLASTp programs of Altschul et al. (1990) J. Mol. Biol. 215: 403-410 available at NCBI (http://www.ncbi.nlm.nih.gov/blast/Blast.cge).

The determination of percent identity or similarity is performed with the standard parameters of the BLASTn and BLASTp programs.

BLAST polynucleotide searches are performed with the BLASTn program. For the general parameters, the “Max Target Sequences” box may be set to 100, the “Short queries” box may be ticked, the “Expect threshold” box may be set to 10 and the “Word Size” box may be set to 28. For the scoring parameters the “Match/mismatch Scores” may be set to 1, −2 and the “Gap Costs” box may be set to linear. For the Filters and Masking parameters, the “Low complexity regions” box may not be ticked, the “Species-specific repeats” box may not be ticked, the “Mask for lookup table only” box may be ticked, and the “Mask lower case letters” box may not be ticked.

BLAST protein searches are performed with the BLASTp program. For the general parameters, the “Max Target Sequences” box may be set to 100, the “Short queries” box may be ticked, the “Expect threshold” box may be set to 10 and the “Word Size” box may be set to “3”. For the scoring parameters the “Matrix” box may be set to “BLOSUM62”, the “Gap Costs” Box may be set to “Existence: 11 Extension: 1”, the “Compositional adjustments” box may be set to “Conditional compositional score matrix adjustment”. For the Filters and Masking parameters the “Low complexity regions” box may not be ticked, the “Mask for lookup table only” box may not be ticked and the “Mask lower case letters” box may not be ticked.

### Polynucleotides:

The term “polynucleotide” is intended to encompass a singular nucleic acid as well as plural nucleic acids, and refers to an isolated nucleic acid molecule or construct, e.g., messenger RNA (mRNA) or plasmid DNA (pDNA). A polynucleotide may comprise a conventional phosphodiester bond or a non-conventional bond (e.g., an amide bond, such as found in peptide nucleic acids (PNA)). The term “nucleic acid” refers to any one or more nucleic acid segments, e.g., DNA or RNA fragments, present in a polynucleotide. By “isolated” nucleic acid or polynucleotide is intended a nucleic acid molecule, DNA or RNA, which has been removed from its native environment. For example, a recombinant polynucleotide encoding an polypeptide contained in a vector is considered isolated for the purposes of the present invention. Further examples of an isolated polynucleotide include recombinant polynucleotides maintained in heterologous host cells or purified (partially or substantially) polynucleotides in solution. Isolated RNA molecules include in vivo or in vitro RNA transcripts of polynucleotides of the present invention. Isolated polynucleotides or nucleic acids according to the present invention further include such molecules produced synthetically. In addition, polynucleotide or a nucleic acid may be or may include a regulatory element such as a promoter, ribosome binding site, or a transcription terminator.

As used herein, a “coding region” is a portion of nucleic acid which consists of codons translated into amino acids. Although a “stop codon” (TAG, TGA, or TAA) is not translated into an amino acid, it may be considered to be part of a coding region, but any flanking sequences, for example promoters, ribosome binding sites, transcriptional terminators, introns, and the like, are not part of a coding region. Two or more coding regions of the present invention can be present in a single polynucleotide construct, e.g., on a single vector, or in separate polynucleotide constructs, e.g., on separate (different) vectors. Furthermore, any vector may contain a single coding region, or may comprise two or more coding regions, e.g., a single vector may separately encode an immunoglobulin heavy chain variable region and an immunoglobulin light chain variable region. In addition, a vector, polynucleotide, or nucleic acid of the invention may encode heterologous coding regions, either fused or not fused to a nucleic acid encoding, e.g., a gene from the isolated genetic material as defined hereinabove, or a fragment, variant, or derivative thereof. Heterologous coding regions include without limitation specialized elements or motifs, such as a secretory signal peptide or a heterologous functional domain.

In certain embodiments, the polynucleotide or nucleic acid is DNA. In the case of DNA, a polynucleotide comprising a nucleic acid which encodes a polypeptide normally may include a promoter and/or other transcription or translation control elements operably associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” or “operably linked” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein.

### Hybridization Processes

The hybridization and amphiploidization processes are facilitated when the selected species are dioecious or self-incompatible, have a short juvenile phase, and are amenable to efficient procedures for cytogenetic and embryological analyses of root tips and ovules, respectively. The use of male sterile lines or emasculation procedures are required if the plants are not dioecious or self-incompatible. Hybrids are produced between sexual varieties or lines that display appropriate degrees of divergence in photoperiod responses and female developmental schedules.

Intraspecific hybrids are made using standard techniques as taught plant breeding texts, e.g. Poehlman, Breeding Field Crops (1987). The successful production of interspecific or intergeneric hybrids may require hormone treatments to the florets and embryo rescue procedures as taught in recent references involving wide hybridization, e.g. Z. W. Liu et al., Hybrids and Backcross Progenies between Wheat (Triticum aestivum L.) and Apomict Australian Wheatgrass [Elymus rectisetus (Nees in Lehm.) A. Love & Connor]: Karyotypic and Genomic Analyses, 89 Theor. Appl. Genet. 599-605 (1994) (incorporated herein by reference). Hybrids are verified by their intermediate phenotype

### Amphiploidization Processes

The chromosome numbers of hybrids are doubled using standard colchicine techniques, e.g. J. Torabinejad et al., Morphology and Genome Analyses of Interspecific Hybrids of Elymus scabrus, Genome 29 (1987), 150-155 (incorporated herein by reference). Alternatively, recently developed tissue culture techniques may be used, as described, e.g., in Leblanc et al., Chromosome Doubling in Tripsacum: the Production of Artificial, Sexual Tetraploid Plants, Plant Breed. 114 (1995), 226-30 (incorporated herein by reference); Salon & Earle, Determination of Mode of Reproduction of Synthetic Tetraploids of Eastern Gamagrass, Agron. Abs. Pg 114 (1994) (incorporated herein by reference); Cohen & Yao, In Vitro Chromosome Doubling of Nine Zantedeschia Cultivars, Plant Cell, Tiss. Org. Cult. 47 (1996), 43-49 (incorporated herein by reference); and Chalak & Legave, Oryzalin Combined with Adventitious Regeneration for an Efficient Chromosome Doubling of Trihaploid Kiwifruit, Plant Cell Rep. 16 (1996), 97-100 (incorporated herein by reference). Partially amphiploid 2n+n Bnr hybrids are often produced in low frequencies (0.5% to 3%) when interspecific F1s are backcrossed, e.g. Z. W. Liu et al., Theor. Appl. Genet. 89 (1994), 599-605, and this frequency may be much higher if tendencies for apomixes (unreduced egg formation) exist in the hybrids as taught in O. Leblanc et al., Reproductive Behavior in Maize Tripsacum Polyhaploid Plants: Implications for the Transfer of Apomixis into Maize, J. Hered. 87 (1996), 108-111 (incorporated herein by reference). Thus, a preferred method for doubling chromosomes of intraspecific and interspecific hybrids is to use one or more of the colchicine (or other known spindle inhibitor chemical) treatment methods listed above. Likewise, a preferred method for doubling chromosomes of interspecific hybrids involves backcrossing to one of the sexual parents and counting chromosomes in root tips to determine partial amphiploidy (usually triploidy).

This is followed by backcrossing to the other parent to obtain a full amphiploid, or to the same parent to obtain a partial amphiploid (three genomes from one parent and one genome from the other). Amphiploidization may precede or follow hybridization.

As used herein the phrase “plant yield” refers to the amount (e.g., as determined by weight or size) or quantity (numbers) of tissues or organs produced per plant or per grow season. Hence increased yield could affect the economic benefit one can obtain from the plant in a certain growing area and/or growing time.

It should be noted that a plant yield can be affected by various parameters including, but not limited to, plant biomass; plant vigor; growth rate; seed yield; seed or grain quantity; seed or grain quality; oil yield; content of oil, starch and/or protein in harvested organs (e.g., seeds or vegetative parts of the plant); number of flowers (florets) per panicle (expressed as a ratio of number of filled seeds over number of primary panicles); harvest index; number of plants grown per area; number and size of harvested organs per plant and per area; number of plants per growing area (density); number of harvested organs in field; total leaf area; carbon assimilation and carbon partitioning (the distribution/allocation of carbon within the plant); resistance to shade; number of harvestable organs (e.g. seeds), seeds per pod, weight per seed; and modified architecture [such as increase stalk diameter, thickness or improvement of physical properties (e.g. elasticity)]. As indicated above, in one embodiment the method of the present invention leads to an increase of at least one of the group of parameters selected from the above list, comprising yield, biomass, growth rate, vigor, nitrogen use efficiency and/or abiotic stress tolerance, preferably tolerance to nutrient deficiency of a plant.

As used herein the phrase “seed yield” refers to the number or weight of the seeds per plant, seeds per pod, or per growing area or to the weight of a single seed, or to the oil extracted per seed. Hence seed yield can be affected by seed dimensions (e.g., length, width, perimeter, area and/or volume), number of (filled) seeds and seed filling rate and by seed oil content. Hence increase seed yield per plant could affect the economic benefit one can obtain from the plant m a certain growing area and/or growing time; and increase seed yield per growing area could be achieved by increasing seed yield per plant, and/or by increasing number of plants grown on the same given area. Additionally or alternatively to the above indicated thus, in one embodiment the method of the present invention leads to an increase of at least one of the group of parameters concerning seed or grain selected from the above list comprising yield and/or quality of a plant's seed or grain, preferably number or weight of the seeds/grains per plant, seeds/grains per pod, or per growing area or the weight of a single seed/grain, or amount and/or quality of the oil extracted per seed/grain.

The term “seed” (also referred to as “grain” or “kernel”) as used herein refers to small embryonic plant enclosed in a covering called the seed coat (usually with some stored food), the product of the ripened ovule of gymnosperm and angiosperm plants which occurs after fertilization and some growth within the mother plant.

As used herein the phrase “plant biomass” refers to the amount (e.g., measured in grams of air-dry tissue) of a tissue produced from the plant in a growing season, which could also determine or affect the plant yield or the yield per growing area. An increase in plant biomass can be in the whole plant or in parts thereof such as aboveground (harvestable) parts, vegetative biomass, roots and seeds.

As used herein the phrase “growth rate” refers to the increase m plant organ/tissue size per time (can be measured in cm² per day).

As used herein the phrase “plant vigor” refers to the amount (measured by weight) of tissue produced by the plant in a given time. Hence increased vigor could determine or affect the plant yield or the yield per growing time or growing area. In addition, early vigor (seed and/or seedling) results in improved field stand.

Improving early vigor is an important objective of modern breeding programs in both temperate and tropical rice cultivars. Long roots are important for proper soil anchorage in water-seeded rice. Where rice is sown directly into flooded fields, and where plants must emerge rapidly through water, longer shoots are associated with vigor. Where drill-seeding is practiced, longer mesocotyls and coleoptiles are important for good seedling emergence. The ability to engineer early vigor into plants would be of great importance in agriculture. For example, poor early vigor has been a limitation to the introduction of maize (Zea mays L.) hybrids based on Com Belt germplasm in the European Atlantic.

As used herein, the phrase “non-stress conditions” refers to the growth conditions (e.g., water, temperature, light-dark cycles, humidity, salt concentration, fertilizer concentration in soil, nutrient supply such as nitrogen, phosphorous and/or potassium), that do not significantly go beyond the everyday climatic and other abiotic conditions that plants may encounter, and which allow optimal growth, metabolism, reproduction and/or viability of a plant at any stage in its life cycle (e.g., in a crop plant from seed to a mature plant and back to seed again). The person skilled in the art is aware of normal soil conditions and climatic conditions for a given plant in a given geographic location. It should be noted that while the non-stress conditions may include some mild variations from the optimal conditions (which vary from one type/species of a plant to another), such variations do not cause the plant to cease growing without the capacity to resume growth.

The phrase “abiotic stress” as used herein refers to any adverse effect on metabolism, growth, reproduction and/or viability of a plant. Accordingly, abiotic stress can be induced by suboptimal environmental growth conditions such as, for example, salinity, water deprivation, flooding, freezing, low or high temperature, heavy metal toxicity, anaerobiosis, nutrient deficiency, atmospheric pollution or UV irradiation.

The phrase “abiotic stress tolerance” as used herein refers to the ability of a plant to endure an abiotic stress without suffering a substantial alteration in metabolism, growth, productivity and/or viability.

As used herein the phrase “fertilizer use efficiency” refers to the metabolic process(es) which lead to an increase in the plant's yield, biomass, vigor, and growth rate per fertilizer unit applied. The metabolic process can be the uptake, spread, absorbent, accumulation, relocation (within the plant) and use of one or more of the minerals and organic moieties absorbed by the plant, such as nitrogen, phosphates and/or potassium, preferably referring to nitrogen however.

As used herein the phrase “nitrogen use efficiency (NUE)” refers to the metabolic process(es) which lead to an increase in the plant's yield, biomass, vigor, abiotic stress tolerance, growth rate per nitrogen unit applied, nitrogen gain derived from biological nitrogen fixation and/or preferably to tolerance to nutrient deficiency of a plant. The metabolic process can be uptake/increased nitrogen gain derived from biological nitrogen fixation, spread, absorbent, accumulation, relocation (within the plant) and/or use of nitrogen absorbed by the plant.

As used herein the phrase “nitrogen-limiting conditions” refers to growth conditions which include a level (e.g., concentration) of nitrogen (e.g., ammonium or nitrate) applied which is below the level needed for normal plant metabolism, growth, reproduction and/or viability.

Improved plant NUE is translated in the field into either harvesting similar quantities of yield, while implementing less fertilizers, or increased yields gained by implementing the same levels of fertilizers. Thus, improved NUE has a direct effect on plant yield in the field. Thus, the polynucleotides and polypeptides of some embodiments of the invention positively affect plant yield, seed yield, and/or plant biomass. In addition, the benefit of improved plant NUE will certainly improve crop quality and biochemical constituents of the seed such as protein yield and oil yield.

It should be noted that improved abiotic stress tolerance will confer plants with improved vigor also under non-stress conditions, resulting in crops having improved biomass and/or yield e.g., elongated fibers for the cotton industry, higher oil content.

The term “isolated” refers to an entity such as a polynucleotide, polypeptide, gene etc. at least partially separated from the natural environment e.g., from a plant cell.

These and other embodiments are disclosed and encompassed by the description and examples of the present invention. Further literature concerning any one of the materials, methods, uses and compounds to be employed in accordance with the present invention may be retrieved from public libraries and databases, using for example electronic devices. For example the public database “Medline” may be utilized, which is hosted by the National Center for Biotechnology Information and/or the National Library of Medicine at the National Institutes of Health. Further databases and web addresses, such as those of the European Bioinformatics Institute (EBI), which is part of the European Molecular Biology Laboratory (EMBL) are known to the person skilled in the art and can also be obtained using internet search engines. An overview of patent information in biotechnology and a survey of relevant sources of patent information useful for retrospective searching and for current awareness is given in Berks, TIBTECH 12 (1994), 352-364.

The above disclosure generally describes the present invention. Unless otherwise stated, a term as used herein is given the definition as provided in the Oxford Dictionary of Biochemistry and Molecular Biology, Oxford University Press, 1997, revised 2000 and reprinted 2003, ISBN 0 19 850673 2. Several documents are cited throughout the text of this specification. Full bibliographic citations may be found at the end of the specification immediately preceding the claims. The contents of all cited references (including literature references, issued patents, published patent applications as cited throughout this application and manufacturer's specifications, instructions, etc.) are hereby expressly incorporated by reference; however, there is no admission that any document cited is indeed prior art as to the present invention.

A more complete understanding can be obtained by reference to the following specific examples which are provided herein for purposes of illustration only and are not intended to limit the scope of the invention.

### EXAMPLES

The Examples and Figures which follow further illustrate the invention, but should not be construed to limit the scope of the invention in any way.

The practice of the present invention will employ, unless otherwise indicated, conventional techniques of cell biology, cell culture, molecular biology, transgenic biology, microbiology, and recombinant DNA, which are within the skill of the art.

Methods in molecular genetics and genetic engineering are described generally in the current editions of Molecular Cloning: A Laboratory Manual, (Sambrook et al., (1989) Molecular Cloning: A Laboratory Manual, 2nd ed., Cold Spring Harbor Laboratory Press); DNA Cloning, Volumes I and II (Glover ed., 1985); Oligonucleotide Synthesis (Gait ed., 1984); Nucleic Acid Hybridization (Hames and Higgins eds. 1984); Transcription And Translation (Hames and Higgins eds. 1984).

Detailed methods how to assess yield, biomass, growth rate, vigor, nitrogen use efficiency and abiotic stress tolerance and in particular tolerance to nutrient, i.e. nitrogen deficiency of a plant, in particular cereals; methods of producing genetically engineered plants including appropriate vectors and transformation protocols; determining expression levels of nitrogenase mRNA in plant shoot roots and association root association of the plant with a nitrogen fixing microorganism, and/or reduction of nitrogen fertilization demand of a plant are known to the person skilled in the art and are described in, e.g., international applications WO2010/049897 and WO2013/066805, the disclosure content of which is incorporated herein by reference.

### Example 1

### Gene Mining

The following description summarizes the process how putative plant candidate genes involved in high nitrogen fixation potential in O. longistaminata have been defined.

Abbreviations: BNF—biological nitrogen fixation; ESTs—expressed sequence tags; nifH—one of the structural genes encoding nitrogenase, the bacterial key enzyme for nitrogen fixation

### First RNA Analyses:

As no genome sequence of Orya longistaminata is currently available for gene mining, deep sequencing of the root transcriptome of O. longistaminata was carried out on plants adapted to high BNF rates in soil without N-fertilizer. In total, early next-generation sequencing (pyrosequencing) revealed 60,155 ESTs which could be aligned to japonica or indica rice genome sequences, out of which remarkably 9.993 were genes that had not yet been detected to be expressed in Oryza yet. Approx. 11,000 ESTs were potential novel sequence tags found only in our O. longistaminata transcript collection.

However, they might partially also originate from other organisms as the soil system was not sterile. These sequence data were a valuable resource for the identification of genes involved in supporting high nitrogen fixation rates and for development of an O. longistaminata microarray for transcriptome analysis and gene mining, the data have been published (Yang et al., BMC Genomics. 11 (2010), 705).

### Narrowing Down the Number of Candidate Genes

Studies on Differential Gene Expression Related to High BNF Capacity in O. longistaminata.

Previous experiments have shown that the population of diazotrophs in O. longistaminata roots is drastically changed shortly after application of nitrogen fertilizer, and also expression of bacterial nitrogenase genes (nifH) ceases rapidly. Therefore, genes were identified herein of O. longistaminata that are differentially expressed under external N-depleted conditions of high nitrogen fixation rates in comparison to N-fertilized conditions lacking BNF.

Pot experiments were carried out with a single dose of nitrogen fertilizer as ammonium nitrate (corresponding to 100 kg N/ha), and plants were adapted to these conditions for several weeks. Expression of bacterial nifH genes was below the detection level in these fertilized plants. From roots of these plants and plants supported by nitrogen fixation, RNAs were extracted, and an SSH—(Suppression subtractive hybridization) was constructed using the PCR-Select cDNA Subtraction Kit (Clontech). From 1358 clones sequenced by Sanger sequencing, 412 different ESTs were detected that were putatively up-regulated under conditions of BNF. This approach provided novel sequence resources and putative candidate genes for support of high nitrogen fixation potential, and for microarray development.

### Studies on Differential Gene Expression by Microarray Analysis:

In a similar biological set-up as above, O. longistaminata plants were set up in soil that were adapted to BNF or to nitrogen fertilization, respectively. RNAs from these roots were used to compare the expression profile. For transcriptome microarrays, a microarray designed for O. sativa transcriptome analysis (Agilent) has been upgraded with additional genes detected in the SSH approach. Application of this chip showed that 55 of the latter genes were found to be upregulated during BNF as expected.

As a second array, probes have been developed for the ESTs detected in the O. longistaminata roots. In this 40K-Array (Agilent), approx. 770 genes/ESTs were differentially regulated under BNF conditions versus N-fertilized conditions. Out of those, 375 ESTs were up-regulated with a factor of at least 2-fold. They were considered as promising candidate genes.

### Genome Analysis:

However, ESTs from Next Generation Sequencing (NGS) were relatively short, which makes microarray construction and annotation of complete genes difficult, as well as bioinformatic analysis. Particularly, many of the ESTs may comprise different regions of the same target gene. To obtain a larger amount of full-length sequences, which are especially important for novel or low-identity ESTs, a draft genome sequence has been generated from the here described O. longistaminata accession. Custom sequencing was done by next-generation sequencing. First a non-saturating low-cost approach has been chosen including the combination of two plates of 454-pyrosequencing (one containing 8 kb libraries), and one channel of paired-end Illumina HighSeq sequencing. Mapping of the ESTs from NGS to the draft genome thus allowed to reduce the list of candidate genes again to the genes indicated in Table 1, below.

TABLE 1
Genes identified within the experiments of the present invention as involved
in high nitrogen fixation potential in O. longistaminata. Indicated are the
predicted coding sequences (CDS) and predicted amino acid/protein
sequences. Annot. = Annotation of homologues/similar Genes in O. sativa.
Sequences 4, 61 and 66, marked with a star (*) in the Gene no. column were
stronger expressed in the F1 hybrids than in the O. sativa. parent plant.

| Gene | | | Predicted protein |
|---|---|---|---|
| no. | Annot. | Predicted CDS | (amino acid) sequence |
| 1 | Xa21_Os04g0450100 | ATGGGGAGCCTTGGCGGAGAGGAGGGGGCGAGGCCGGCGGAGGGGGCGCGGAGGCCGAGGTTCCTGTG | MGSLGGEEGARPAEGARRPRFLC |
| | | CCTGCACGGGTTCCGGACGAGCGGGGAGATCATGCGGAAGCAGGTGGTGGGGAAGTGGCCCGCCGACG | LHGFRTSGEIMRKQVVGKWPADV |
| | | TCACGGCGCGCCTCGACCTCGTCTTCGCCGACGCGCCGTTCCCCGCCGAGGGGAAGTCCGACGTCGAG | TARLDLVFADAPFPAEGKSDVEG |
| | | GGCATCTTCGACCCGCCCTACTACGAGTGGTTCCAGTTCGACAAGAATTTTACAGAGTACAGGAATTT | IFDPPYYEWFQFDKNFTEYRNFD |
| | | CGACGAATGCCTGAACTACATCGAGGAATTGATGATTAAAGAAGGGCCTTTTGATGGACTGATGGGTT | ECLNYIEELMIKEGPFDGLMGFS |
| | | TCTCTCAGGGCTCGATTCTATCTGGCGCCCTTCCGGGCCTCCAAGAACAAGGGTTGGCCCTGACTAGA | QGSILSGALPGLQEQGLALTRVP |
| | | GTTCCTAAGATCAAGTACCTCATAATCATAGGAGGCGCCAAGTTTCAGTCGCCGACGGTTGCCGAGAA | KIKYLIIIGGAKFQSPTVAEKAY |
| | | GGCGTACGCCAATAACATCAAATGCCCCTCGGTTCACTTCCTCGGTGACACTGACTTTCTGAAAACCC | ANNIKCPSVHFLGDTDFLKTHGE |
| | | ATGGCGAAAAGCTTATAGAATCATATGTGGATCCATTCATCATTCGTCATCCAAAGGGCCACACTGTT | KLIESYVDPFIIRHPKGHTVPRL |
| | | CCAAGGCTTGATGAGAAGAGTCTGGAAATCATGCTCCATTTCCTCGACAAGATTGAGAAGGAGACGGC | DEKSLEIMLHFLDKIEKETALEH |
| | | GCTCGAGCATTCATCCACTGATGTTGACGAAAAGGAAATGTGCTTGTGA | SSTDVDEKEMCL* |
| | | SEQ ID NO: 1 | SEQ ID NO: 2 |
| 2 | Xa21_Os03g0678800 | ATGGACGACATGGCCAAGCTAGCTCAGATGCCGCGCGCTCCTCTGCTCGTCGTCCTGATGCTGATGCT | MDDMAKLAQMPRAPLLVVLMLML |
| | | GCTGGGTGTCGGCGCGGCGGCCGTGCCGGAGTACAGGGAGGCGCCGCACTTCACCAACTCGGCGGCAG | LGVGAAAVPEYREAPHFTNSAAA |
| | | CGCGGTGCCCGCCGCCGCTGCCGGCGACGGACGCGGACGCGGCGTGCTCGCCGCACGCGGCGGTGCAC | RCPPPLPATDADAACSPHAAVHV |
| | | GTGGCCATGACGCTGGACGCGCCGTACCTGAGGGGGACCATGGCCGCGGTGCTCTCCGTCCTCCGCCA | AMTLDAPYLRGTMAAVLSVLRHA |
| | | CGCCTCCTGCCCGGAGTCCGTCCACTTCCACTTCCTCGCCTCCTCCTCCTCGTCGCCGGAGGCGGCGG | SCPESVHFHFLASSSSSPEAAAA |
| | | CGGCGGCGCGGGAGCTGAGGGACACCGTGCGGGCGTCGTTCCCGTCGCTGGCGTTCCGGGTGTACCCG | ARELRDTVRASFPSLAFRVYPFD |
| | | TTCGACGAGTCCCGCGTCGCGGGGCTCATCTCCACCTCCATCCGCGGCGCGCTCGACCGCCCGCTCAA | ESRVAGLISTSIRGALDRPLNYA |
| | | CTACGCCCGCTCCTACCTCGCCACCACGCTCCCCGCCTGCGTCCGCCGCGTCGTCTACCTCGACTCCG | RSYLATTLPACVRRVVYLDSDVV |
| | | ACGTCGTGGTCACCGACGACATCGCCGCGCTCGCCGCGACGCCGCTGCCCGGGGAGACCGCCGTGGCG | VTDDIAALAATPLPGETAVAAPE |
| | | GCGCCCGAGTACTGCGGCGCCAACTTCACCGCCTACTTCACGCCGGGGTTCTGGGCGTCGCGGGCGCT | YCGANFTAYFTPGFWASRALSES |
| | | GTCGGAGTCCGCGTTCGCGGGGCGGCGCGCGTGCTACTTCAACACGGGGGTGATGGTGCTGGACCTGC | AFAGRRACYFNTGVMVLDLPRWR |
| | | CCCGGTGGCGGCGCGCGGGGTACACGGCGCAGATCGAGGAGTGGATGGAGCTGCAGAGGCGGGTGCGC | RAGYTAQIEEWMELQRRVRIYEL |
| | | ATCTACGAGCTGGGCTCGCTGCCGCCGTTCCTCCTCGTGTTCGCCGGCCGCATCGCCGCCGTCGACCA | GSLPPFLLVFAGRIAAVDHRWNQ |
| | | CCGCTGGAACCAGCACGGCCTCGGCGGCGACAACTACCGGGGGCTCTGCCGCGGCCTGCACGCCGGCG | HGLGGDNYRGLCRGLHAGAVSLL |
| | | CCGTCAGCCTCCTGCACTGGAGCGGCAAGGGGAAGCCATGGGACAGGCTCGACGCCGGCAAGCCGTGC | HWSGKGKPWDRLDAGKPCPLDAV |
| | | CCGCTCGACGCCGTCTGGGCCAAGTACGACCTGCTCCGCCCGGCGGCGGCGATCGAGACCTCGTGA | WAKYDLLRPAAAIETS* |
| | | SEQ ID NO: 3 | SEQ ID NO: 4 |
| 3 | Xa21_OsJ_21238 | ATGTCGTCTGCTGCCATTGCCATTAGCCCGATGGTGGAAGAGTTTGCTCTCCTGCCCATCTGCTTCGA | MSSAAIAISPMVEEFALLPICFD |
| | | CGGCAGCCGGTCGCCCCACTGTTTGTCCGGGTCACAGCTGCAGGATTCGATTATCATCTTTCTTGCTG | GSRSPHCLSGSQLQDSIIIFLAV |
| | | TGCCTGGTGCCCCACCGATGCCGATGAGTGTGCTCGGCTCGGAGTCCATCGCCTCGGTCAAGCTGCGC | PGAPPMPMSVLGSESIASVKLRI |
| | | ATTCAGCGGTTCAAGGGGTTCGTGGTTAACAAGCAGAGGCTTGTGCTTGATGGGCACGAGCTGGCAAG | QRFKGFVVNKQRLVLDGHELARN |
| | | GAACAACTGCCATGTGAAGGATTACGGGCTTGCCGATGGAAATGTTCTGCACCTTGTTATCCGCTTGG | NCHVKDYGLADGNVLHLVIRLAD |
| | | CTGATCTTCGCTTGATTAACATCGAGACTACCAGTGGAAAGAAGTTTCAGTTCCAGGTGGACCAGAGC | LRLINIETTSGKKFQFQVDQSRN |
| | | CGCAATGTCAAGTATCTCAAGAGTAAGCTGGCAGTGGAGGGAGATGAGGACCTTGGTGAGGATCACAA | VKYLKSKLAVEGDEDLGEDHKLE |
| | | GCTTGAATGTGATGGCAAGGAGCTTGAGGACCACCAGCTGATTGCTGATATTAGCAAGAAGGATGATG | CDGKELEDHQLIADISKKDDAVI |
| | | CTGTGATCCATTTGTTCATCCGCAAGCCAGCAAAGTTACGGACACAGCAAGTTGATAAAGACACGGTG | HLFIRKPAKLRTQQVDKDTVVTV |
| | | GTTACAGTTGTCACTCCGCAGGAGAAAGAGAACCTTCAAAATGAAGCTCATGCTGTGAACCCTGCTAA | VTPQEKENLQNEAHAVNPAKPAG |
| | | ACCAGCTGGTGCTAGGCCTGCTCTTGTTGAACCAATTATCGTTAACCACAAGGTGAAGCTATCACTTG | ARPALVEPIIVNHKVKLSLEVMR |
| | | AAGTGATGAGAATGATTAGCTCAGCCATAGCTGGTCTTGAGAATGGATACTTGCCAGTGATGTCAGCT | MISSAIAGLENGYLPVMSAEGSG |
| | | GAAGGTTCAGGGGGTGTTTACTTCATGCAAGATGCATCAGGAGAGAAGAATATTGCAGTGTTTAAGCC | GVYFMQDASGEKNIAVFKPRDEE |
| | | CAGAGATGAGGAACCTATGGCTAAAAACAACCCAAGAGGGCTTCCAGTGTCGACTGATGGAGAAGGCA | PMAKNNPRGLPVSTDGEGMKRGT |
| | | TGAAGAGGGGGACACTTGTAGGGGAAGGTGCATTTAGGGAAGTTGCTGCTTATATTCTTGACCATCCA | LVGEGAFREVAAYILDHPIGDHE |
| | | ATTGGTGATCATGAATCTGAAGAACGTATCGGGTTCTCTGGTGTGCCTCCCACAGCATTGGTCCGGAG | SEERIGFSGVPPTALVRSLHRGK |
| | | CTTACACAGGGGGAAGAGCTTCAAGATTGGATCACTGCAGATGTTCATTCAGAACAATGGAAGTTGTG | SFKIGSLQMFIQNNGSCEDMGPR |
| | | AGGATATGGGTCCCCGAGCATTTCCAGTGAAGGAGGTCCACAAAATAGCAGTGTTAGATCTTAGGTTA | AFPVKEVHKIAVLDLRLANADRH |
| | | GCAAACGCTGATCGTCATGCAGGGAACATTTTAGTGTGCAAGGATGAAGAAGGTGGCAATTACAAGTT | AGNILVCKDEEGGNYKLVPIDHG |
| | | GGTTCCAATTGATCATGGTTACTGCTTGCCAGAGAAGTTTGAAGATTGTACCTTTGAGTGGCTGTACT | YCLPEKFEDCTFEWLYWPQAREP |
| | | GGCCCCAGGCTCGTGAACCATTCAGTGATGAAACCATTGCATACATCAAATCACTAGACGCTGAAGAG | FSDETIAYIKSLDAEEDIKLLKF |
| | | GACATCAAGCTTCTCAAGTTCCATGGGTGGGAGCTGTCTGCAAGATGTGCTCGTGTCCTGCGCATCAG | HGWELSARCARVLRISTMLLKKG |
| | | CACCATGCTTTTAAAGAAAGGTGCAGCACGAGGCCTCACACCGTATGACATCGGGCGTATACTGTGCA | AARGLTPYDIGRILCRETVNRDS |
| | | GGGAAACTGTGAATAGAGACTCTGAGATTGAGGACATCGTCCAGGAAGCCGAGGACCATGTTCTCCCA | EIEDIVQEAEDHVLPGSSEVIFL |
| | | GGGTCAAGTGAAGTAATCTTCTTAGAAACCGTATCTGAAATCATAGACCGTCACCTCGACAAGAAGTT | ETVSEIIDRHLDKKFA* |
| | | CGCTTAG SEQ ID NO: 5 | SEQ ID NO: 6 |
| 4* | Xa21_Os03g0764100 | ATGTCGAGCGCGTCGTCCATGGAAGCGCTCCACGCCGCGGTGCTCAAGGAGGAGCAGCAGCAGCACGA | MSSASSMEALHAAVLKEEQQQHE |
| | | GGTGGAGGAGGCGACGGTCGTGACGAGCAGCAGCGCCACGAGCGGGGAGGAGGGCGGACACCTGCCGC | VEEATVVTSSSATSGEEGGHLPQ |
| | | AGGGGTGGGCGAAGCGGAAGCGGTCGCGCCGCCAGCGATCGGAGGAGGAGAACCTCGCGCTCTGCCTC | GWAKRKRSRRQRSEEENLALCLL |
| | | CTCATGCTCGCCCGCGGCGGCCACCACCGCGTCCAAGCGCCGCCTCCGCTCTCGGCTTCGGCGCCCCC | MLARGGHHRVQAPPPLSASAPPP |
| | | GCCGGCAGGTGCGGAGTTCAAGTGCTCCGTCTGCGGCAAGTCCTTCAGCTCCTACCAGGCGCTCGGCG | AGAEFKCSVCGKSFSSYQALGGH |
| | | GCCACAAGACGAGCCACCGGGTCAAGCTGCCGACTCCGCCCGCAGCTCCCGTCTTGGCTCCCGCCCCC | KTSHRVKLPTPPAAPVLAPAPVA |
| | | GTCGCCGCCTTGCTGCCTTCCGCCGAGGACCGCGAGCCAGCCACGTCATCCACCGCCGCGTCCTCCGA | ALLPSAEDREPATSSTAASSDGA |
| | | CGGCGCGACCAACAGAGTCCACAGGTGTTCCATCTGCCAGAAGGAGTTCCCCACCGGGCAGGCGCTCG | TNRVHRCSICQKEFPTGQALGGH |
| | | GCGGGCACAAGAGGAAGCACTACGACGGTGGCGTAGGCGCCGGCGCCGGCGCATCTTCAACCGAGCTC | KRKHYDGGVGAGAGASSTELLAT |
| | | CTGGCCACGGTGGCCGCCGAGTCCGAGGTGGGCAGCTCCGGCAACGGCCACTCCGCCACCCGGGCGTT | VAAESEVGSSGNGHSATRAFDLN |
| | | CGACCTCAACCTCCCGGCCGTGCCGGAGTTCGTGTGGCGGCCGTGCTCCAAGGGCAAGAAGATGTGGG | LPAVPEFVWRPCSKGKKMWDDEE |
| | | ACGACGAGGAGGAGGTCCAGAGCCCCCTCGCCTTCAAGAAGCCGCGCCTTCTCACCGCGTAA | EVQSPLAFKKPRLLTA* |
| | | SEQ ID NO: 7 | SEQ ID NO: 8 |
| 5 | Xa21_OsJ_26543 | ATGGGGCAGGCTAGGGCTCACCTCCTAGCCTCCCTGTGGGCTTTCTACCTCATCCTAGCAATCTCCAT | MGQARAHLLASLWAFYLILAISM |
| | | GGTCACTGGCGACCTCACCAATGACCTCGACATACTGTGGGGAAACTCAAAGGTGTTCTACGACAGCT | VTGDLTNDLDILWGNSKVFYDSS |
| | | CCGGCAAACAGACTATCTCACTGACTCTTGACCGCTGGACGACCTCCGCGTTCCGGTCGAAGAGCACA | GKQTISLTLDRWTTSAFRSKSTY |
| | | TACCTCTTCAGTAGGATCGACATGGACATCAAGCTCGTTGCTGGAGACTCAGCTGGAACCGTGACTAC | LFSRIDMDIKLVAGDSAGTVTTL |
| | | TCTTTATATGATAACAGAGGGCCTATGGCAATTCCACGACGAGATTGACCTTGAGTTCTTGGGGAACT | YMITEGLWQFHDEIDLEFLGNST |
| | | CCACTGGCGAGCCCTACACCCTGCACACTAACCTGTACGCCAGAGGGAAAGGTGGCAGAGAGAAGCGG | GEPYTLHTNLYARGKGGREKRYK |
| | | TACAAGCTCTGGTTTGATCCCACTGCAGACTTCCATACCTACACCATCATCTGGAACCAAAGAAATAT | LWFDPTADFHTYTIIWNQRNILI |
| | | CCTAATCCTTGTTGATGACAAGCTGATCCGGCAGATCAAGAACAATTTGATGTACAGTGTCCCGTACC | LVDDKLIRQIKNNLMYSVPYPTY |
| | | CAACCTATCAGCCAATGAGGGTGTATGGCAGCATCTGGAATGCCGATGACTGGGCAACAATGGGAGGG | QPMRVYGSIWNADDWATMGGRVK |
| | | CGGGTGAAGACTGACTGGTCGCAGGCGCCATTCACAGCATACTTCCGGAACTACAGGGCTATCGCTTG | TDWSQAPFTAYFRNYRAIACPPQ |
| | | CCCCCCACAACAGAGCTCCCCACTCTGCGGCCAGAGCTCTGGAAATTGGTTCAACCAGGAGCTGGATG | QSSPLCGQSSGNWFNQELDVTRK |
| | | TGACACGGAAACAGCAACTGCAGGAGGTGGATGCCAACTACAAGATCTACGATTACTGCACTGACACC | QQLQEVDANYKIYDYCTDTKRFK |
| | | AAGAGGTTCAAAGATAATCTCCCCAAAGAGTGTACGATAAACTAG | DNLPKECTIN* |
| | | SEQ ID NO: 9 | SEQ ID NO: 10 |
| 6 | Xa21_Os01g0186900 | ATGAGCTCGCTGACGGCGGCGCAGTTCCGGTCGCTGTTTGAGATGGCCGGAGGGGAGGATATCTCGTT | MSSLTAAQFRSLFEMAGGEDISF |
| | | TCTGTTTGGTGATGATGAGGCGGCGGCGGTGGCGGCGCCGGTGGAGTTGCAGGGGAAGAGGGGGTGGG | LFGDDEAAAVAAPVELQGKRGWE |
| | | AGGAGGTGGATCAAGGGGAGGGGTCTGGGGCGGTGGCGGCGAAGAGGCAGCGGTCGCCGACGAGCTCG | EVDQGEGSGAVAAKRQRSPTSSR |
| | | AGGGAGAATAGTAGCGGGAGTAATAATGAGGGTGGGCAGGAGGAGGTGTCTGAGGCGGCGGCGGCCAT | ENSSGSNNEGGQEEVSEAAAAMA |
| | | GGCGGCGGCGGTGGGGAGGGGTGGGGGGAGGAGGCTGTGGGTGAAGGAGAGGGACTCGGAGTGGTGGG | AAVGRGGGRRLWVKERDSEWWDT |
| | | ACACGGTGAGTAGCCCGGATTACCCGGACTCGGAGTTCAGGAAGGCGTTCCGCATGTCCAAGGCCACG | VSSPDYPDSEFRKAFRMSKATFE |
| | | TTCGAGGTGGTCTGCGACGAGCTCGCCGCCGCCGTCGCCAAGGAGGACACCATGCTCCGCGCCGCCAT | VVCDELAAAVAKEDTMLRAAIPV |
| | | CCCGGTGCGCAAGCGCGTCGCCGTCTGCGTCTGGCGCCTCGCCACGGGGGAGCCCCTGAGGCTGGTGT | RKRVAVCVWRLATGEPLRLVSKR |
| | | CCAAGCGCTTCGGCCTCGGGATCTCCACCTGCCACAAGCTCGTGCTCGAGGTGTGCGCCGCGCTCAAG | FGLGISTCHKLVLEVCAALKAMV |
| | | GCCATGGTGATGCCCAAGGTGGTGCGCTGGCCCGAGGCCGGCGACGCGGCGGCCATCGCCGCCCACTT | MPKVVRWPEAGDAAAIAAHFEAI |
| | | CGAGGCCATCTCCGGGATCTCCGGCGTCGTCGGCGCGATCTACACCACCCACATCCCCATCATCGCGC | SGISGVVGAIYTTHIPIIAPKSN |
| | | CCAAGTCCAACGTCGCCTCCTACTACAACCGCCGCCACACGGAGCGCAACCAGAAGACCTCCTACTCC | VASYYNRRHTERNQKTSYSMTVQ |
| | | ATGACCGTCCAGTGCGTCGTCGACTCCACCGGCGCCTTCACCGACGTCTGCATCGGCTGGCCGGGGTC | CVVDSTGAFTDVCIGWPGSNSDE |
| | | CAACTCCGACGAGGAGGTGCTCGAGAAGTCGGCGCTGTACCTGCACCGCGGCGTGCCGGGGCTGATCC | EVLEKSALYLHRGVPGLIQGQWV |
| | | AGGGCCAGTGGGTGGTCGGCGGCGGGAGCTTCCCGCTCATGGACTGGATGCTCGTGCCCTACACCCAC | VGGGSFPLMDWMLVPYTHQNLTW |
| | | CAGAACCTGACATGGGCGCAGCACATGCTCAACGAGAAGGTCGCCGCCGTGCGCGGCGTGGCGCGCGA | AQHMLNEKVAAVRGVARDAFERL |
| | | CGCGTTCGAGCGCCTCAAGCGCCGGTGGGGCTGCCTCCAGAAGCGCACCGAGGTGAAGCTGCTCGACC | KRRWGCLQKRTEVKLLDLPTVLG |
| | | TCCCCACCGTGCTCGGCGCCTGCTGCGTCCTCCACAACATCTGCGAGCGCAGCGGCGACGCCGTCGAC | ACCVLHNICERSGDAVDADDCTF |
| | | GCCGACGACTGCACCTTCGACCTCTTCGACGACGACATGGTCGCCGAGAACGCCGTCCGCTCCACCGC | DLFDDDMVAENAVRSTAAAQARD |
| | | CGCCGCGCAGGCCCGCGACGCCATCGCCCACAACCTCCTCCACAGCGGCGCCGGCGCATCCTTCTTCT | AIAHNLLHSGAGASFF* |
| | | GA SEQ ID NO: 11 | SEQ ID NO: 12 |
| 7 | Xa21_Os01g0802700 | ATGATTACGGGTTCGGAGGTGTACCAGGTGGTGGAGGCGATGGCGCCGCTGTACACGGCGGCGGCGCT | MITGSEVYQVVEAMAPLYTAAAL |
| | | GGGGTACGGGTCGGTGCGGTGGCTGAAGGCGTTCTCCAACGAGCAGTGCGCCGGGATCAACCACTTCG | GYGSVRWLKAFSNEQCAGINHFV |
| | | TGGCGCTCTACGCCGTGCCGGTGCTCATCTTCGACATGGTGTCCACCAACGACGTGTACAAGATGAAC | ALYAVPVLIFDMVSTNDVYKMNG |
| | | GGCCGCCTCATCGCCGCCGACACGCTGCAGAAGGCCGTGCTGCTGCTGGGCCTCATGGCGTGGGCGCT | RLIAADTLQKAVLLLGLMAWALW |
| | | CTGGGAGAGGTCGCGCGCGCGCCGCGCCGGGGCGAAGGCGGTGTCGTCGCCGCTGCAGTGGGTCATCA | ERSRARRAGAKAVSSPLQWVITC |
| | | CCTGCTTCTCCGTCGCGTCGCTGCCCAACACCATCATCATGGGCGTGCCGCTCCTCAACGGCATGTAC | FSVASLPNTIIMGVPLLNGMYGP |
| | | GGGCCCGTGTCCAAGGACCTCATGAAGCAGATCGTCGTCATGCAGTTCTGCATCTGGTACAACGTCAT | VSKDLMKQIVVMQFCIWYNVIIF |
| | | CATCTTCCTCTACGAGTACATGGCGGCGCGTAGATCGGCCTCGGCGCCGCCGCCGGCGTCGTCGGAGG | LYEYMAARRSASAPPPASSEGSA |
| | | GCAGCGCCAAGATCAGCCCTTCGTCGCCGGTGAAAGCTGCGGCGGCGGCGGCGGCGGACGCGAACGGC | KISPSSPVKAAAAAAADANGNAV |
| | | AATGCTGTCGCGGCCGACCGGCCGCAAGAAGTGGCGGTGAACATCGAAATCACGGAGATGGCGGCGTC | AADRPQEVAVNIEITEMAASTAR |
| | | CACGGCACGAGACGGCGTGTCCGGCGAGACGACGGCGGCCGCCAAGGAGGTGAGCGCCGGTGAAGTTG | DGVSGETTAAAKEVSAGEVAPVE |
| | | CTCCGGTGGAAGAAGAGGAGGCCTCTGCGCCAGCGCCGTCGATGAAGCACGTCATCTGGATGGCGGTG | EEEASAPAPSMKHVIWMAVKKLL |
| | | AAGAAGCTGCTACAGATTCCGAATACCTATGCAAGCTTCCTTGGCCTCATCTGGTCTCTAATCGCATT | QIPNTYASFLGLIWSLIAFKCGF |
| | | CAAGTGTGGATTCTCGATGCCAAAAATCGTCGAGGACTCTCTGTTCACCATTCGTACCACCGCTGTTG | SMPKIVEDSLFTIRTTAVGLSMF |
| | | GCCTAAGCATGTTTTCTTCAGGGACGTTCATAGCGCGGCAGTCGCGGTTCGTGCCGTGCGGATACAAG | SSGTFIARQSRFVPCGYKIASFS |
| | | ATCGCGTCGTTCTCCATGGTCATCAAGTTTCTGATAGGCCCGGTTGTGATGCTGTTCGCCTCGCTCGT | MVIKFLIGPVVMLFASLVIGMHG |
| | | CATCGGCATGCACGGCACACTTCTGCACATCGCTGTTGTGCAGGCGGCTCTCCCCCTGGCAGTGACAT | TLLHIAVVQAALPLAVTSFVYAE |
| | | CATTTGTGTACGCTGAAGAGTACAAGGTCCACGCAGACATCATGAGCACAGGGGTTATTCTTGGGATA | EYKVHADIMSTGVILGIFISLPV |
| | | TTTATATCACTTCCTGTGACAATTGTTTACTATATTCTGTTGGGGCTGTGA | TIVYYILLGL* |
| | | SEQ ID NO: 13 | SEQ ID NO: 14 |
| 8 | Xa21_OJ1759_F09.33 | ATGGCGAAGGCGAGCATGGTGGTGCCTGAGCAGGTGGGCGCGGCGGCGGCGGCGCAGGTGGGGTGCCC | MAKASMVVPEQVGAAAAAQVGCP |
| | (Os09g0369400) | CTGTCCGGGCACGACGCTGTTCCCGTACCCGCCGCCGCGCGCCGGGATCGCCGTGCGGCGCAAGTGCC | CPGTTLFPYPPPRAGIAVRRKCL |
| | | TGCAGGCGGCGCAGCAGCTGGAGCTCGGCGCCGGGCTGCGCGGCGGCTGGGTGGAGTCCATGCGGGCG | QAAQQLELGAGLRGGWVESMRAS |
| | | TCGTCGCCCACCCACGCCAAGGCCGCCGCCGCCCTCGCCGCCGGCGTCGACGAGGAGCACGCCGCCTG | SPTHAKAAAALAAGVDEEHAAWM |
| | | GATGGTCCGTTTCCGTTCACCGATTGATCGATGTTCGTCGCGTTCTTGGCGCGCGCGCGCTGACACTG | VRFRSPIDRCSSRSWRARADTDM |
| | | ACATGAACCGTGCATTTCCGTTCGTCTTTGTGCAGGCGAGGCACCCGTCGGCGCTGGGCGAGTTCGAG | NRAFPFVFVQARHPSALGEFEKV |
| | | AAGGTGGTGGCGGCGTCGAAGGGGAAGCAGATCGTCATGTTCCTCGACTACGACGGCACCCTCTCCCC | VAASKGKQIVMFLDYDGTLSPIV |
| | | CATCGTCGACGACCCCGACGCCGCCTTCATGAGCGAGACGATGCGGATGGCCGTGCGCAGCGTGGCGA | DDPDAAFMSETMRMAVRSVAKHF |
| | | AGCACTTCCCGACGGCGATCGTGAGCGGGCGGTGCCGCGACAAGGTGTTCGAGTTCGTCAAGCTCGCC | PTAIVSGRCRDKVFEFVKLAELY |
| | | GAGCTGTACTACGCGGGGAGCCACGGCATGGACATCAAGGGCCCCGCCTCCCGCCACGCCGCCGTCAA | YAGSHGMDIKGPASRHAAVKSPP |
| | | GTCTCCTCCCCACAACAAGGGCGTCCTCTTCCAGCCGGCCAGCGAGTTCCTCCCCATGATCGAGCAGG | HNKGVLFQPASEFLPMIEQVHDR |
| | | TGCACGACCGCCTCGAGCAGGCGACCCGCTCCATCCCGGGCGCCAAGGTCGAGAACAACAAGTTCTGC | LEQATRSIPGAKVENNKFCVSVH |
| | | GTCTCCGTCCACTTCCGGTGCGTCGACGAGAAGAGTTGGGGGGCGTTGGCGGAGACGGTGAGGAGGGT | FRCVDEKSWGALAETVRRVVREF |
| | | GGTGAGGGAGTTCCCGCGGCTGCGGCTGAGCCAGGGGAGGATGGTGTTCGAGGTGCGGCCGACCATCA | PRLRLSQGRMVFEVRPTIKWDKG |
| | | AGTGGGACAAGGGCAAGGCCCTCGAGTTCCTCCTCGACTCGCTCGGTTTCGCCGACTGCAGCGACGTG | KALEFLLDSLGFADCSDVLPVYI |
| | | CTGCCGGTCTACATCGGCGACGACCGCACGGACGAGGACGCGTTCAAGGGCCTGGGGATCCTGGTGTC | GDDRTDEDAFKGLGILVSKHPKE |
| | | GAAGCACCCCAAGGAGACGACCGCCTCCTTCTCCCTCCAGGAGCCCGCCGAGGTGATGGAGTTCTTGC | TTASFSLQEPAEVMEFLLRLVEW |
| | | TGCGGCTCGTGGAGTGGAATCGCCTGTCCAGGACACGGTTGAGGCTGTAA | NRLSRTRLRL* |
| | | SEQ ID NO: 15 | SEQ ID NO: 16 |
| 9 | Xa21_OsI_24487 | ATGCCTCACTCCTCCTACCTCGCCCTCGCCCTCGCCGTTGCGGCGGTTCTTGCTATGGCCGCCGTCGA | MPHSSYLALALAVAAVLAMAAVE |
| | | GGTGTCCGGCCTCGGCTTCGACCTCCACCACCGCTACTCCCCCATCGTGCAGCGGTGGGCGGAGGAGC | VSGLGFDLHHRYSPIVQRWAEER |
| | | GTGGCCACGCCGGCGTGTCCTGGCCTGCCGGCGCCGAGGTGATCGGCTCGCCCGAGTACTACTCCGCG | GHAGVSWPAGAEVIGSPEYYSAL |
| | | CTGTCCCGACACGACCACGCCCTCTTCGCGCGACGCGGGCTCGCCCAGGGCGACGGCCTCGTCACCTT | SRHDHALFARRGLAQGDGLVTFA |
| | | CGCCGACGGCAACATCACCCTGCGGCTCGACGGATCGCTGCACTACGCGGAGGTGGCGGTGGGCACGC | DGNITLRLDGSLHYAEVAVGTPN |
| | | CAAACACGACGTTCCTGGTGGCGCTCGACACCGGCAGCGACCTCTTCTGGGTGCCCTGCGACTGCAAG | TTFLVALDTGSDLFWVPCDCKQC |
| | | CAGTGCGCTCCCCTCGGCAACCTCACGGCGGTGGACGGCGGCGGCGGCCCGGAGCTCAGGCAGTACAG | APLGNLTAVDGGGGPELRQYSPS |
| | | CCCGAGCAAGTCGTCGACGAGCAAGACGGTCACCTGCGCCAGCAACCTCTGCGACCAGCCCAACGCCT | KSSTSKTVTCASNLCDQPNACAT |
| | | GCGCCACGGCCACCAGCAGCTGCCCCTACGCCGTCCGCTACGCCATGGCCAACACCTCCTCCTCCGGC | ATSSCPYAVRYAMANTSSSGELV |
| | | GAGCTCGTCGAGGACGTGCTCTACCTCACCAGGGAGAAGGGCGCCGCCGCCGCCGCCGGAGCCGCCGT | EDVLYLTREKGAAAAAGAAVKTP |
| | | GAAAACTCCCGTGGTGTTCGGGTGCGGGCAGGTGCAGACGGGCTCGTTCCTCGACGGCGCCGCCGCCG | VVFGCGQVQTGSFLDGAAADGLM |
| | | ACGGCCTCATGGGTCTCGGCATGGAGAAGGTGTCCGTGCCCAGCATCCTCGCCAGCACCGGCGTCGTC | GLGMEKVSVPSILASTGVVKSNS |
| | | AAGTCGAACAGCTTCTCCATGTGCTTCAGCAAAGACGGCCTCGGCCGGATCAACTTCGGCGACACCGG | FSMCFSKDGLGRINFGDTGSADQ |
| | | CAGCGCCGACCAGTCCGAGACGCCATTCATCGTCAAATCCACACACTCGTACTACAACATCAGCATCA | SETPFIVKSTHSYYNISITSMSV |
| | | CGTCGATGAGCGTCGGGGACAAGAACTTGCCGTTGGGGTTCTACGCCATTGCTGACTCCGGCACGTCC | GDKNLPLGFYAIADSGTSFTYLN |
| | | TTCACCTACCTCAACGACCCGGCCTACACGGCCTACACCACCAATTTCAACGCGCAGATCAGCGAGAG | DPAYTAYTTNFNAQISERRANFS |
| | | GAGGGCCAACTTCAGCGGCAGTACTCGTTCTGGTCCATTTCCTTTCGAGTACTGCTACTCGTTGAGTC | GSTRSGPFPFEYCYSLSPDQTTV |
| | | CTGATCAAACAACGGTGGAACTGCCTATTGTGAGCCTGACGACCAATGGAGGAGCCGTGTTCCCGGTG | ELPIVSLTTNGGAVFPVTRPVYP |
| | | ACCAGGCCTGTTTACCCCATCGCGGTTCAGATGACAAATGGAAAGATTCGTGTCATTGGTTACTGCCT | IAVQMTNGKIRVIGYCLAVIKSD |
| | | GGCTGTCATCAAGAGCGATCTCCCCATCGACATCATTGGCCAGAACTTCATGACCGGGCTCAAGGTCG | LPIDIIGQNFMTGLKVVFNREKS |
| | | TCTTCAACCGTGAGAAATCCGTCCTGGGCTGGCAAAAGTTCGACTGTTACAAGGACGAGAAGATGACA | VLGWQKFDCYKDEKMTDDGSGVG |
| | | GACGACGGGTCAGGCGTCGGGAGCCCGAGCCCATCGCCGGGGCCGACCACCCACGTCTTCCCGCAGCC | SPSPSPGPTTHVFPQPQESDSPA |
| | | GCAGGAGAGCGACTCGCCGGCGGGCAGAACGCCCATCCCCGGCGCGGCGCCGGTGCCGCGGTCGAGCA | GRTPIPGAAPVPRSSSAAAGGRA |
| | | GCGCGGCAGCTGGCGGCCGTGCAGGCGTCCGGCTCCTGTCGTCCATGGCGCTCATGCTGCTGGCTGCT | GVRLLSSMALMLLAAATVVF* |
| | | GCTACAGTAGTTTTCTGA SEQ ID NO: 17 | SEQ ID NO: 18 |
| 10 | Xa21_Os04g0119500 | ATGTCGGCGTTGCTCGATCTGCTCATGGGGGTCTTCCGCCAGCCGACCCTCGGCGGCGTCGCCCTCGA | MSALLDLLMGVFRQPTLGGVALE |
| | | GCTCGCCTCCCTCGCCGCGCCGCTCTGGCTCGCCGCCCTCGTCGGCCTACTCATCGGCTGGGCGTGGC | LASLAAPLWLAALVGLLIGWAWR |
| | | GCCCGCGCTGGGCCCTCGCCGTCGTCGGGGAGGCGCCGCCGGAGACCCCCGCCCTTCCGCCACCGCGC | PRWALAVVGEAPPETPALPPPRA |
| | | GCCGCCGCCGAGGACTCCTGCCGCAACAACGAGCTTGTACCCGTCGTTCCTAGAACTGCGGTTGCCGC | AAEDSCRNNELVPVVPRTAVAAA |
| | | CGCCACGCCAGTGGAGGATGAGGGATTGGCCGTCGACACCGGAGATTTGATGCACCTCCGCCGCCTTG | TPVEDEGLAVDTGDLMHLRRLVE |
| | | TCGAGGAGAAGGACGGCGGGCCGGCGTGGATTCACATGATGGACAAGACGCTGCCCACGATGCGCTAC | EKDGGPAWIHMMDKTLPTMRYQA |
| | | CAAGCATGGAGGAGAGATCCTGAGGGTGGCCCGCCTCAATACCGCACTAGCACTATCTTTGAGGATGC | WRRDPEGGPPQYRTSTIFEDASP |
| | | ATCGCCAGAAGTTGTCAGGGATTTCTTCTGGGATGATGAGTTCCGGATTAAGAACACTTGGGACGACA | EVVRDFFWDDEFRIKNTWDDMLL |
| | | TGCTTCTTCAGCACGAGACGTTGGAGGAGTGCACAAAGACTGGAACGATGGTTCTCCGGTGGGTCAGG | QHETLEECTKTGTMVLRWVRKFP |
| | | AAGTTCCCGTTCTTCTGCAGTGACAAGGAGTACATTATCGGTCGCAGGATATGGGCATCTGGAAAGAC | FFCSDKEYIIGRRIWASGKTYYC |
| | | GTATTACTGTGTAACAAAGGGTGTGCCTCGCCCCTCTGTTCCAAGGTGCAACAAACCTCGTCGTGTGG | VTKGVPRPSVPRCNKPRRVDVYY |
| | | ACGTGTACTACTCTAGTTGGTGCATCCGTCCAGTCGAAGCAAGAAACGGTGATGGCTCAATGACCGCA | SSWCIRPVEARNGDGSMTACEVL |
| | | TGTGAGGTGCTCCTGTTCCATCATGAGGAAATGGGGATTCCAAGGGAAATTGCAAAGCTCGGTGTTCG | LFHHEEMGIPREIAKLGVRQGMW |
| | | GCAGGGGATGTGGGGCTGCGTCAAAAGGATTGAGCCTGGCTTGAGGGCATACCAAATCGCAAGGACTG | GCVKRIEPGLRAYQIARTAGEPL |
| | | CTGGCGAGCCCCTTTCAAAATGCGCTGCCATGGCACATGTCAACACGAAGGTTGATCCTGATGAGCTC | SKCAAMAHVNTKVDPDELITAED |
| | | ATCACTGCAGAGGACAAGACCGAGGCCAGTTCAACCAACAACGCTGAAGCTGAGAAACCAAAGCACTG | KTEASSTNNAEAEKPKHWTGNIP |
| | | GACAGGCAATATACCCAAAGTCATCCTTGTAGGTGGCGCAGTTGCCCTGGCATGCACGTTTGACCATG | KVILVGGAVALACTFDHGLLTKA |
| | | GACTGTTAACAAAGGCTCTCATATTTGGTACGGCGAGGAGGTTTGCAGGACCAGGAAGGAGATGA | LIFGTARRFAGPGRR* |
| | | SEQ ID NO: 19 | SEQ ID NO: 20 |
| 11 | Xa21_Os10g0455100 | ATGGCAGGCACTAAGCTTGCAGCTCTTGGGTTTATTGTCCTCTTGAGCATTGGATTAGCTAGTGCTGT | MAGTKLAALGFIVLLSIGLASAV |
| | | AAGGGTAGAAAGATACTCAAATGCCCAAGGTTCGGGCACAGGAAGTGGAGAGGGCACAGGAAGCGTGA | RVERYSNAQGSGTGSGEGTGSVN |
| | | ACGGTGCGGGAGCTGGGTTGGGAAGTGGGACTGGGTCTGGTAGCAGTGGTTACAATGGTGCCCATGCA | GAGAGLGSGTGSGSSGYNGAHAS |
| | | AGTGGTGGAGGAGGAGGCGGAGGCGGTGGTTACAGCCAATATGGTGGGTCAGGGTCTGGTAGTGGATC | GGGGGGGGGYSQYGGSGSGSGSG |
| | | TGGTTCGGGCTCAGGCTCTAGCCAGACCAGTCAAAATGGGTATTATGGTTATGGTGGATCATCAAGTG | SGSGSSQTSQNGYYGYGGSSSAG |
| | | CTGGCGGTAGCGGTGCTGGTGCTGGAGCTGGACAAGCTGGTGGTTACTGGCCATCCTACGGTCATGGG | GSGAGAGAGQAGGYWPSYGHGSG |
| | | TCTGGTAGTGGTACCGGCTCAGGCTCTAGTGAGGCTACTAATACCTGGTATGGACAGTATGCAAATGC | SGTGSGSSEATNTWYGQYANANA |
| | | AAATGCTGGTGGCAACGGTGGTGGTAACGGTCAGGGCCAATATGGTGGCAGCGGTGCTGGTGTAGGTT | GGNGGGNGQGQYGGSGAGVGSGS |
| | | CTGGATCTGGGTTCGGTGATGCGAACCCTTAA SEQ ID NO: 21 | GFGDANP* SEQ ID NO: 22 |
| 12 | Xa21_P0512G09.2-1 | ATGGCCGGAGGGGAGGATATCTCGTTTCTGTTTGGTGATGATGAGGCGGCGGCGGTGGCGGCGCCGGT | MAGGEDISFLFGDDEAAAVAAPV |
| | | GGAGTTGCAGGGGAAGAGGGGGTGGGAGGAGGTGGATCAAGGGGAGGGGTCTGGGGCGGTGGCGGCGA | ELQGKRGWEEVDQGEGSGAVAAK |
| | | AGAGGCAGCGGTCGCCGACGAGCTCGAGGGAGAATAGTAGCGGGAGTAATAATGAGGGTGGGCAGGAG | RQRSPTSSRENSSGSNNEGGQEE |
| | | GAGGTGTCTGAGGCGGCGGCGGCCATGGCGGCGGCGGTGGGGAGGGGTGGGGGGAGGAGGCTGTGGGT | VSEAAAAMAAAVGRGGGRRLWVK |
| | | GAAGGAGAGGGACTCGGAGTGGTGGGACACGGTGAGTAGCCCGGATTACCCGGACTCGGAGTTCAGGA | ERDSEWWDTVSSPDYPDSEFRKA |
| | | AGGCGTTCCGCATGTCCAAGGCCACGTTCGAGGTGGTCTGCGACGAGCTCGCCGCCGCCGTCGCCAAG | FRMSKATFEVVCDELAAAVAKED |
| | | GAGGACACCATGCTCCGCGCCGCCATCCCGGTGCGCAAGCGCGTCGCCGTCTGCGTCTGGCGCCTCGC | TMLRAAIPVRKRVAVCVWRLATG |
| | | CACGGGGGAGCCCCTGAGGCTGGTGTCCAAGCGCTTCGGCCTCGGGATCTCCACCTGCCACAAGCTCG | EPLRLVSKRFGLGISTCHKLVLE |
| | | TGCTCGAGGTGTGCGCCGCGCTCAAGGCCATGGTGATGCCCAAGGTGGTGCGCTGGCCCGAGGCCGGC | VCAALKAMVMPKVVRWPEAGDAA |
| | | GACGCGGCGGCCATCGCCGCCCACTTCGAGGCCATCTCCGGGATCTCCGGCGTCGTCGGCGCGATCTA | AIAAHFEAISGISGVVGAIYTTH |
| | | CACCACCCACATCCCCATCATCGCGCCCAAGTCCAACGTCGCCTCCTACTACAACCGCCGCCACACGG | IPIIAPKSNVASYYNRRHTERNQ |
| | | AGCGCAACCAGAAGACCTCCTACTCCATGACCGTCCAGTGCGTCGTCGACTCCACCGGCGCCTTCACC | KTSYSMTVQCVVDSTGAFTDVCI |
| | | GACGTCTGCATCGGCTGGCCGGGGTCCAACTCCGACGAGGAGGTGCTCGAGAAGTCGGCGCTGTACCT | GWPGSNSDEEVLEKSALYLHRGV |
| | | GCACCGCGGCGTGCCGGGGCTGATCCAGGGCCAGTGGGTGGTCGGCGGCGGGAGCTTCCCGCTCATGG | PGLIQGQWVVGGGSFPLMDWMLV |
| | | ACTGGATGCTCGTGCCCTACACCCACCAGAACCTGACATGGGCGCAGCACATGCTCAACGAGAAGGTC | PYTHQNLTWAQHMLNEKVAAVRG |
| | | GCCGCCGTGCGCGGCGTGGCGCGCGACGCGTTCGAGCGCCTCAAGCGCCGGTGGGGCTGCCTCCAGAA | VARDAFERLKRRWGCLQKRTEVK |
| | | GCGCACCGAGGTGAAGCTGCTCGACCTCCCCACCGTGCTCGGCGCCTGCTGCGTCCTCCACAACATCT | LLDLPTVLGACCVLHNICERSGD |
| | | GCGAGCGCAGCGGCGACGCCGTCGACGCCGACGACTGCACCTTCGACCTCTTCGACGACGACATGGTC | AVDADDCTFDLFDDDMVAENAVR |
| | | GCCGAGAACGCCGTCCGCTCCACCGCCGCCGCGCAGGCCCGCGACGCCATCGCCCACAACCTCCTCCA | STAAAQARDAIAHNLLHSGAGAS |
| | | CAGCGGCGCCGGCGCATCCTTCTTCTGA | FF* |
| | | SEQ ID NO: 23 | SEQ ID NO: 24 |
| 13 | Xa21_OsI_03946 | ATGGCGTCGCTCACCACCACCGCCGCCGCGTCCTCCCCGGCCGCGCTGCCGGCGGCCTCGACCACCGC | MASLTTTAAASSPAALPAASTTA |
| | | CCCGGCGTCGTCCGTGTCCCCGGCCGCCGCGGCCGCCAAGCGGCCGCACCTCGCCGGGGACGACGCGC | PASSVSPAAAAAKRPHLAGDDAP |
| | | CGTGGCGGGCGGCGACCGGCGCCGCGGGGATCCGCCCCGTCCCGCGCATCCACCACGCCCCCGTCCTC | WRAATGAAGIRPVPRIHHAPVLR |
| | | CGCGTCGCGCAGGACGATTATTCCGCCTACGCCCTCGCCGTCATGAAGCATCCGGATCCGATAGGGGA | VAQDDYSAYALAVMKHPDPIGEG |
| | | GGGGTTGGCCATGGAGGCTATGGTGGAGGCGGCCGGGCCGGAGTGCATCGTGCCCGGGCAGCAGGCGC | LAMEAMVEAAGPECIVPGQQAPL |
| | | CGCTGCGGCTGCTGGGCCTCAAGGTTTGGCCCCTTGATATAGAGATGAAGTTCCTGGAGCCATTTGGA | RLLGLKVWPLDIEMKFLEPFGRE |
| | | CGGGAACTACATTCCATGAAAAAGTTCATGGACAAGTCATGCAGCGTTATGGACTCATCTATGTCAAA | LHSMKKFMDKSCSVMDSSMSNK* |
| | | TAAGTAG SEQ ID NO: 25 | SEQ ID NO: 26 |
| 14 | Xa21_Os03g0241900 | ATGGCCGCCAAGCAGAGCCTCTACCCGGAGGTGAACCAGTCCCACCCCGACCTCAACACCGCCTTCCT | MAAKQSLYPEVNQSHPDLNTAFL |
| | | CGCCAACCCCAACCGCGCCGCCACCGCCAGCCCCGGCGGCTCGCTCTACCCCTCCGTCGACCCGCAGC | ANPNRAATASPGGSLYPSVDPQQ |
| | | AGCTCGCCGAGAACCTCTTCCCCGACGCCGCCGATGACGCCGCGCCGCCGCCTCCCACCACCGAGGAG | LAENLFPDAADDAAPPPPTTEEA |
| | | GCCCTCGTCGCGGTTCCGGGCGCGCAGCTCCACCTCGTCGACCCCGACCGCAGCATGGACCTCGGTGC | LVAVPGAQLHLVDPDRSMDLGAG |
| | | CGGCACGCTCTCCGTCGTCCGCCTCCGCCAGGGGGACCACTCCGTCGCCGTCCTCGCGAGGCTCGTCC | TLSVVRLRQGDHSVAVLARLVPE |
| | | CGGAGAAGCGCAGCCAGCGCCGCGGCGGCCTCTTCGGCTTCTTGAGCGGCGGCGGGAAGGCCGGCGAC | KRSQRRGGLFGFLSGGGKAGDGA |
| | | GGCGCCGCGCAGGAGCCCGTGCAGTGGCCGCTCACCCGCGACGTCGCCGCCGTCAAGCTCGACACCGC | AQEPVQWPLTRDVAAVKLDTAHY |
| | | GCACTACTTCTTCTCGCTCCACGTCCCGCACACAGATCACGAGGATGACGACGCCGAGGAGGCCGAGG | FFSLHVPHTDHEDDDAEEAEAEK |
| | | CGGAGAAGGACGCGGACGGGGAGGCGGCGCTGAGCTACGGGCTCACGGTCGCCAGCAAGGGGCAGGAG | DADGEAALSYGLTVASKGQEAVL |
| | | GCGGTCCTGGCCCAGCTGGACAAGGTACTGGAGGAGTACACCACCTTCTCGGTGAAGCAAGTGGAACC | AQLDKVLEEYTTFSVKQVEPAAK |
| | | CGCGGCGAAGGAGAAGTCGGGGGTGATGGACACGAAGGCGGTGACGGAGATCACGCCGGAGGAGGCCG | EKSGVMDTKAVTEITPEEAVGDK |
| | | TTGGGGACAAGAAGGAGGTGGTGGAGGAGCAATCGGCGGCGTTCTGGACGACGATCGCGCCGAACGTT | KEVVEEQSAAFWTTIAPNVDDYS |
| | | GACGACTACAGCTCGTCGGTGGCGAGGCTGATCGCGCGAGGCTCGGGGCAGCTGGTGAGGGGCATCAT | SSVARLIARGSGQLVRGIIWCGD |
| | | CTGGTGCGGGGACATCACAGCGGAGGGCCTCCGGTGTGGGGAGGCGGTGATGAAGAAGAGCGTGGGGC | ITAEGLRCGEAVMKKSVGPSGKP |
| | | CGAGTGGGAAGCCGTCACAGGTGAAGCCGAGCACTATCAGGAGGATGAAGAGGGCTAGAAGGGTTACG | SQVKPSTIRRMKRARRVTKMSNR |
| | | AAGATGTCCAACAGGGTAGCAAATTCAATCCTCTCTGGTGTTCTAAAGGTCTCTGGGTTTGTTACCAG | VANSILSGVLKVSGFVTSTVLNS |
| | | CACTGTGCTGAACTCGAAACCTGCCCAGAAATTCTTTAAGTTGATGCCGGGCGAAGTTATTCTTGCTT | KPAQKFFKLMPGEVILASLDGFG |
| | | CACTCGATGGATTTGGGAAAGTATGGGATGCTGTAGAGGTGTCTGGAAAGAATGTGATGCGGACATCG | KVWDAVEVSGKNVMRTSSVVTTS |
| | | TCGGTTGTGACAACTTCTGTTGTAACACATAGATACGGAGATCAAGCTGGACAGGTCACACAGGATTA | VVTHRYGDQAGQVTQDYLHATGN |
| | | CCTTCACGCCACTGGAAACGCTCTTGGAGTCGCATGGGCTGTCTTCAAAATTCGGAAAGCTCTCGACC | ALGVAWAVFKIRKALDPKGNLKK |
| | | CAAAGGGCAATCTGAAGAAGTCGTCTCTGGCGAGCGCTGCAGCCCATGCTGTAGCTAAAGAATCGATA | SSLASAAAHAVAKESISRQKRK* |
| | | TCTCGGCAAAAAAGGAAGTGA SEQ ID NO: 27 | SEQ ID NO: 28 |
| 15 | Xa21_Os05g0498400 | ATGTCTGATCTCGACGTTCAGCTTCCATCTGCCTTCGATCCGTTTGCTGAGGCAAATGCTGAGGACTC | MSDLDVQLPSAFDPFAEANAEDS |
| | | CGGCGCTGGCCCTGGAGCAAAGGATTATGTGCATGTGCGCATCCAGCAGCGCAACGGCAGAAAGAGTC | GAGPGAKDYVHVRIQQRNGRKSL |
| | | TAACTACTGTTCAGGGCTTAAAGAAGGAGTACAGTTACAACAAGATCCTCAAGGATCTGAAAAAGGAG | TTVQGLKKEYSYNKILKDLKKEF |
| | | TTCTGCTGCAATGGTACTGTAGTCCAAGATCCGGAGCTTGGCCAGGTCATTCAGCTTCAAGGTGATCA | CCNGTVVQDPELGQVIQLQGDQR |
| | | GCGTAAGAATGTTGCCACGTTCCTAGTTCAGGCTGGGATTGCTAAGAAGGACAACATCAAGATTCACG | KNVATFLVQAGIAKKDNIKIHGF |
| | | GTTTCTAA SEQ ID NO: 29 | * SEQ ID NO: 30 |
| 16 | Xa21_Os05g0585500 | ATGGGCGCCCGCGCCTCTCGCCACCGCCAATCCCCCGACCAATCCCAATCCCAATCCCAATCCCCATC | MGARASRHRQSPDQSQSQSQSPS |
| | | CCCACACCACAAGCACCACCACCACCACCAAACCACCCGGGCCCCCAAGCCCAAGCCCAAGCCCCAGC | PHHKHHHHHQTTRAPKPKPKPQP |
| | | CGCCGCCGCCGCCGCAGCAGCAGCCGCGGTCTCAGCCTCCGCCGCCGCCGCCGCGGCAGCAGCTCCAG | PPPPQQQPRSQPPPPPPRQQLQP |
| | | CCCCAGCAGGCGGCGGCGGAGGATGGGGTGGGGCGGGTGCTGGGGCGGCCCATGGAGGACGTCCGGGC | QQAAAEDGVGRVLGRPMEDVRAT |
| | | GACCTACACCTTCGGGCGGGAGCTGGGGCGGGGGCAGTTCGGGGTGACCTACCTCGCCACCCACAAGC | YTFGRELGRGQFGVTYLATHKPT |
| | | CCACCGGCCGACGCTACGCCTGCAAGTCCATCGCCGCCCGCAAGCTCGCCCGCCCCGACGACCTCGAC | GRRYACKSIAARKLARPDDLDDV |
| | | GACGTCCGCCGCGAGGTCCACATCATGCACCACCTCACCGGCCACCGCAACATCGTCGAGCTGCGCGG | RREVHIMHHLTGHRNIVELRGAY |
| | | CGCCTACGAGGACCGCCACTCCGTCAACCTCGTCATGGAGCTCTGCGAGGGCGGCGAGCTCTTCGACC | EDRHSVNLVMELCEGGELFDRII |
| | | GCATCATCGCCAGGGGCCACTACTCCGAGCGCGCCGCCGCCGCCCTCTGCAGGGAGATCGTCTCCGTC | ARGHYSERAAAALCREIVSVVHS |
| | | GTCCACAGCTGCCACTCCATGGGGGTCATGCACAGGGACCTCAAGCCCGAGAACTTCCTCTTCCTCAA | CHSMGVMHRDLKPENFLFLNKRE |
| | | CAAGCGCGAGGACTCCCCGCTCAAAGCCACCGATTTTGGCCTCTCCGTCTTCTTCAAGCCCGGTGAGC | DSPLKATDFGLSVFFKPGEQFRD |
| | | AGTTCAGAGATCTTGTTGGAAGTGCATATTATGTGGCTCCTGAAGTCCTCAAACGACGCTATGGAGCT | LVGSAYYVAPEVLKRRYGAEADI |
| | | GAGGCAGACATATGGAGTGCTGGAGTTATCCTTTACATCCTTCTATCAGGCGTTCCTCCGTTCTGGGC | WSAGVILYILLSGVPPFWAENED |
| | | AGAAAACGAGGACGGTATATTTGATGCTGTTCTGCAAGGTCATATCGACTTCTCATCTGAACCATGGC | GIFDAVLQGHIDFSSEPWPSISS |
| | | CTTCTATATCTAGTGGCGCAAAAGACTTGGTCAAGCGGATGCTTCGGCAGGACCCAAAGGAGCGGTTA | GAKDLVKRMLRQDPKERLTAAEI |
| | | ACTGCTGCTGAAATTTTGAACCACCCATGGATTAGAGAGGATGGAGAGGCCCCAGATAAACCACTTGA | LNHPWIREDGEAPDKPLDITVIS |
| | | TATTACAGTGATCAGTAGAATGAAGCAGTTCAGAGCAATGAACAAACTTAAGAAGGTTGCCTTGAAGG | RMKQFRAMNKLKKVALKVVAENL |
| | | TCGTTGCAGAGAACTTGTCAGAGGAAGAGATTGTGGGTTTAAAGGAAATGTTCAAATCTTTAGATACT | SEEEIVGLKEMFKSLDTDNSGTI |
| | | GATAACAGTGGGACAATAACTCTTGAAGAACTAAGAGCTGGTCTACCAAAGCTTGGCACTAAAATTTC | TLEELRAGLPKLGTKISESELRQ |
| | | GGAATCAGAATTAAGGCAGTTGATGGAAGCGGCTGATGTTGATGGAAATGGGTCCATTGATTATGTTG | LMEAADVDGNGSIDYVEFISATM |
| | | AATTTATATCAGCAACGATGCACATGAATAGATTAGAGAAGGAAGATCACATATATAAAGCATTTGAA | HMNRLEKEDHIYKAFEYFDKDHS |
| | | TATTTTGACAAGGACCACAGCGGGTTCATAACAGTCGATGAATTGGAAGAAGCTCTGACGAAGTACGA | GFITVDELEEALTKYDMGDEATI |
| | | CATGGGTGATGAAGCGACAATTAAAGAAATAATTGCTGAAGTGGATACAGACCATGATGGAAGAATTA | KEIIAEVDTDHDGRINYQEFVAM |
| | | ACTACCAGGAGTTTGTTGCCATGATGAAGAACAACAGCCCTGAGATTGTTCCAAATCGACGGCGGATG | MKNNSPEIVPNRRRMF* |
| | | TTTTAA SEQ ID NO: 31 | SEQ ID NO: 32 |
| 17 | Xa21_Os07g0680600 | ATGGCAATCTCAGACGCCAGAGTGGTTCGCCATGTGAGGTGTCCCAAATGCTTCAGTGTTCTTCAGGA | MAISDARVVRHVRCPKCFSVLQE |
| | | GCCCACCGGCGCGCCGGTGTACCAATGCGGTGGATGCGGCACTACCCTCCGAGCGAAAAACCGCACCG | PTGAPVYQCGGCGTTLRAKNRTG |
| | | GCAACTCACAGGAGGTGATCAGTGCTCCCTCCTCTTTGGGGAGTGGCCTGCCTCCTCACAGCAAGCAC | NSQEVISAPSSLGSGLPPHSKHL |
| | | CTGGGCTCCAGTGATGTTGCAAGCACCAGTGGATCCACTCCTGAAGCTCAAATCAGCGGTGGGCAGCA | GSSDVASTSGSTPEAQISGGQQG |
| | | AGGAGCTGACATGACAAGCCGACGCGAGACCGATGATCTCGTGTCGGCGAGGAACAATGCTCCTGAGC | ADMTSRRETDDLVSARNNAPERV |
| | | GAGTGGTGCCAGCTGAGGAGGAGGAAGAGCATGTTCAGAGTATTAGTCAGCTAGCTGTTGGCAATTCT | VPAEEEEEHVQSISQLAVGNSED |
| | | GAAGATCTCACAAGAGGAGATGCAGCTACTGCAGCTGATGCACAGTGCAGTGACAGAGCATCTGAAGG | LTRGDAATAADAQCSDRASEGKV |
| | | CAAGGTGCAATTTTCAGAGAGAAGAGAAGACAGTAACACCAATCTACAAGATGTTCAGCGAAGCAGAG | QFSERREDSNTNLQDVQRSRDDE |
| | | ATGACGAATCTGATGCTGAAGGCAAGAAAAGTTCAGAGGAAACATCCCAATCTCCGCGCCACGATGTC | SDAEGKKSSEETSQSPRHDVVEL |
| | | GTCGAGCTTCCACCAAGTTCAGCTCAAACCCCAGACTCACAACCTGCACCGGCCGTGTTGAGAAGGGA | PPSSAQTPDSQPAPAVLRREDDP |
| | | GGATGATCCGGCGACGAGTCCTCCTCATGGCCATGCGCGTCGATCACCAGAGAACTTGGCGCCGCTTC | ATSPPHGHARRSPENLAPLQKRI |
| | | AGAAGAGGATCCTGAAGACAGTGGACAACCTGAAAGACGACCTCTCTGAACTTTTCAGTAAATCTCCT | LKTVDNLKDDLSELFSKSPELNK |
| | | GAACTGAACAAGCCAAGGACGCACGCGCGCCCTCCTCGTCTTCCCAGGCAAGAAGGCTACGCTCCTCG | PRTHARPPRLPRQEGYAPRDAAM |
| | | CGACGCGGCCATGGCGGCGGCGGCGAGCATCCAAGCCATCAGAGCGCGTCACGCAGCTGTCCACCGTC | AAAASIQAIRARHAAVHRPGYIA |
| | | CCGGCTACATAGCTCGTGCAGGCAAGCCTGGTCAGCTCGCTGCTCCACCTCCACCACGTGGTTTGCCG | RAGKPGQLAAPPPPRGLPSRRYR |
| | | TCGCGGCGCTACCGCCGGTGCAGGGCTGATCACCCGTGCTGCCACGACGCGCGCCATGGGCCGTCGTG | RCRADHPCCHDARHGPSCHHGCC |
| | | CCATCACGGTTGCTGCCCCCCTCACCATGGCAAGCAAGCGTGCACCAGCTGCCGGGGACAACATTGCT | PPHHGKQACTSCRGQHCCRPRTQ |
| | | GCCGGCCAAGAACGCAAGAACCTCCTGCGCCGCGGCGGCCGGCGGCGGCGGCGGCAAAGGAGGTCGTC | EPPAPRRPAAAAAKEVVKRRAPP |
| | | AAGCGGCGGGCGCCACCCAGGAACCATTGCCGGCCGGTGCTCAAGGGCGCGCCGTTCATCATCTGCTC | RNHCRPVLKGAPFIICSSCFKLV |
| | | CAGCTGCTTCAAGCTGGTCCAGGTGCCCGCCGACTTCGCCGTCTCGACCAAGACGGTGCGCAAGCTGC | QVPADFAVSTKTVRKLRCGSCSA |
| | | GTTGCGGCTCATGCTCCGCCGTCCTCTGCTACTCCTACCGGGACCCCGACAGGAAGAAGCACGGCGAT | VLCYSYRDPDRKKHGDQYSADGS |
| | | CAGTACAGCGCCGACGGCTCACCGGCGGCGCCGCGCGGCCACGGGCGGCGAGGCGACAAGTTCGCGTT | PAAPRGHGRRGDKFAFLDDFGHV |
| | | CCTGGACGACTTCGGCCACGTCGACGTGAGCTACTCCACGGAGGACGAGCAGCCGCTGCACGTGTCGA | DVSYSTEDEQPLHVSRNSSFNTV |
| | | GGAACTCGTCGTTCAACACCGTCGACGAGATGGCGGCGGCGGCGACGGCGCCGCCGCAGCAGCAGCAT | DEMAAAATAPPQQQHGSSLHRLM |
| | | GGCTCCAGCCTGCACCGGCTGATGGGGTACGGCTCGGCGAGCGACCTGCTGTTCCGGCAGCACTCGCC | GYGSASDLLFRQHSPDLYESFSE |
| | | TGACCTGTACGAGAGCTTCTCCGAGAGGACGACGCCGGAGGCCGCCGCGCTGTACGACCGGAAAGGGA | RTTPEAAALYDRKGKGVCVDLDD |
| | | AAGGCGTCTGCGTCGACCTCGACGACGACGGCGGCGACGATGACTCAGACGAGGATTGCAGCGGCGCG | DGGDDDSDEDCSGALKRSRLRGS |
| | | CTGAAGAGGTCGAGGCTGAGAGGGTCCGGTTGGCCATTGCCGGGGATACTCAACAGCAAGGGGACGCC | GWPLPGILNSKGTPGMGAIRIKS |
| | | TGGAATGGGAGCAATCAGGATCAAGTCATAG SEQ ID NO: 33 | * SEQ ID NO: 34 |
| 18 | Xa21_protkinase | ATGGAATTAGTAGAAGTGGAGTGGCACGGGGAAGACATAAGCAGGCAATTAAGAGTGCGCCGCCTGAA | MELVEVEWHGEDISRQLRVRRLN |
| | | TGCTGGCTTTCTAAGCATCGACTGCGGCCTAGAGGACACCAGCGGCTACAAGGAAGACAAGACTGGCA | AGFLSIDCGLEDTSGYKEDKTGI |
| | | TCATCTACGTCTCCGACGGCTCCTACGTCGATGCCGGGGAGAACCACCGTGTGTCGGCGGAGGAAGAG | IYVSDGSYVDAGENHRVSAEEEN |
| | | AACGCGAAATTGCCACGTCCGTACGCGACGCTCCGGAGCTTCCCTTCCGGCATGCGGAATTGCTACAC | AKLPRPYATLRSFPSGMRNCYTL |
| | | GCTGCCCGCGGACGCTGGGGCCAAGTACCTTGTCCGGGTGGTGTCCTTCTACGGCAACTACGACGGGA | PADAGAKYLVRVVSFYGNYDGKN |
| | | AGAACAGCTCATCGTCGTTGCAGTTCGACCTGCACCTCGGGACAAACTACTGGGCCACTGTCCGCGCT | SSSSLQFDLHLGTNYWATVRADG |
| | | GACGGCAACAACGTGTACGAGGCGCTGTTCGTGGCCTGGGCGAGCTGGGTGCCGGTGTGTCTCGTGAA | NNVYEALFVAWASWVPVCLVNTG |
| | | CACCGGCCGTGGCACGCCGTTCGTGTCCGCGGTGGAGCTGAGGCAGCTCAGCGGCGAGCTCTACCCGG | RGTPFVSAVELRQLSGELYPGLN |
| | | GCCTCAATGCCAGCCAGTCCTTGTCCATGTATGGACGGCGCAACATGGGTTCAAACATTTCATATACA | ASQSLSMYGRRNMGSNISYTRYP |
| | | CGTTACCCCCACGATCGATATGACCGGTACTGGTGGCAGGTATGGTCTGATCCAATGTGGAAGAACAT | HDRYDRYWWQVWSDPMWKNISTP |
| | | TTCCACTCCATCAACCATCGAACAGAGCTCCATCTTCGCTTTGCCCCTCCCCGTTATCCAGACCGCAG | STIEQSSIFALPLPVIQTAVETV |
| | | TTGAGACGGTCAGCAATAAGACAATGCTCAACATTACATGGCAAGCGGAAACACCCCGAGGCTTTTTG | SNKTMLNITWQAETPRGFLVFLH |
| | | GTTTTCTTGCACTTCGCCGACTTCCAGAACAGTCAATTCCGACAGTTCAAGGTCGCTTTTAACAATGA | FADFQNSQFRQFKVAFNNDYRDN |
| | | CTATCGTGATAACTATAGCCCACCATACATGACCACAGATGCCTTGTTTGGCTTGACAAGGGCCACCA | YSPPYMTTDALFGLTRATSGMYS |
| | | GTGGCATGTACAGCATCAGTATTGGAGCCACCGATGCATCTAAGCTGCCGCCGATGCTAAACGCGATT | ISIGATDASKLPPMLNAIEAYTL |
| | | GAGGCATACACTCTCATGTCCCGTGACAACCCAATAACGTTCCCAAACGATTTTGACACTATCATGGC | MSRDNPITFPNDFDTIMAIKHEY |
| | | AATTAAACATGAATACGGGATAAAGAAGAATTGGATGGGTGATCCATGTTTCCCAACACAATTTGCAT | GIKKNWMGDPCFPTQFAWDGVKC |
| | | GGGATGGCGTGAAATGCAACAATGCAAGTGGTAACAATACGAGGATAATATATTTGGATCTCTCGAAC | NNASGNNTRIIYLDLSNNNLHGP |
| | | AACAACTTGCATGGGCCGATATCTAATAACTTTACATTGCTCACGGCACTCAAGTACCTGAATTTGTC | ISNNFTLLTALKYLNLSGNQLNA |
| | | GGGCAACCAGCTGAATGCACCCATTCCTAAGTCCCTCTGTGAAAACAATAATACAGGATCATTGATTC | PIPKSLCENNNTGSLILSFDSDR |
| | | TCAGTTTCGATTCTGACCGAGAAACGTGCACAATAATACCGCCTCCAACAATAAAAAAATTGAACAAA | ETCTIIPPPTIKKLNKAILAISV |
| | | GCAATCTTAGCTATTTCGGTAGTGGTTCCTGTGATGGCCATTGCTATTCTTGTTCTCGCTTATTTGAT | VVPVMAIAILVLAYLIWRQKRKP |
| | | CTGGAGACAGAAAAGAAAACCAAACATATCAATAGATGATACTCCGAGAGAACGACAAGCTGAAAATG | NISIDDTPRERQAENVPASRKNH |
| | | TTCCAGCAAGCAGAAAAAACCATGGGAATGCCTTGCTAATGGTTGAGAATCGCCAATTCACATACAAG | GNALLMVENRQFTYKELEKLTNK |
| | | GAGCTTGAGAAGTTAACTAATAAGTTTGAACGCTTCATTGGACAAGGAGGTTTTGGACTCGTGTACTA | FERFIGQGGFGLVYYGLIEDGIE |
| | | CGGACTTATAGAAGATGGTATTGAGGTTGCTGTCAAGATACGTTCTGAGTCATCGTGTCATGGACTCG | VAVKIRSESSCHGLDEFFAEVQS |
| | | ATGAATTTTTCGCTGAGGTTCAAAGTTTGACAAAGGTGCATCACAGAAATCTAGTTTCCTTGGTTGGT | LTKVHHRNLVSLVGYCWEKNHLA |
| | | TACTGCTGGGAGAAGAATCATTTAGCACTAGTTTATGAGTACATGGCTCGAGGCACTCTTTGTGATCA | LVYEYMARGTLCDHLRGNHGFSE |
| | | TCTGAGAGGTAATCATGGTTTTAGTGAAACCTTGAATTGGAAAACACGAGTCCGAGTTTTGGTTGAAG | TLNWKTRVRVLVEAAQGLDYLHK |
| | | CTGCACAAGGACTGGATTATCTGCATAAAGGTTGTAGTCCGCCAATAATTCACGGAGATGTGAAGACG | GCSPPIIHGDVKTSNILLDQNLQ |
| | | AGTAACATTCTGTTGGATCAAAATTTACAAGCTAAAATTGCAGATTTTGGTCTTTCCAAGACATATCT | AKIADFGLSKTYLSDTQTHISVN |
| | | TAGCGACACACAGACGCACATATCAGTCAATCCAGCTGGGTCAGCTGGTTATATGGACCCTGGGTACT | PAGSAGYMDPGYFHTGRLTESCD |
| | | TTCACACAGGCAGACTCACGGAGAGTTGTGATGTTTATAGTTTTGGAATTGTTCTGTTAGAGATAGCC | VYSFGIVLLEIATGESPILPGQD |
| | | ACCGGCGAGTCTCCCATACTACCAGGTCAAGACCACATTATTCAGCGTGTGAATAAGAAGATTGCTAC | HIIQRVNKKIATGNISLVADARL |
| | | CGGTAATATCAGCTTGGTTGCTGATGCACGACTCAGAGGTGCCTATGATATCAGCTCAATGTGGAAGG | RGAYDISSMWKVVDIGLLCTTDT |
| | | TGGTGGACATCGGACTGTTATGCACTACTGACACTGGTGCAAAAAGGCCGACAATGGCTGCTGTGGTT | GAKRPTMAAVVVRLNESLALEEA |
| | | GTGCGACTCAATGAGAGCCTTGCATTGGAGGAAGCTCATTGCGAGAGTGGCTTCAGGGGAAGCACAGG | HCESGFRGSTGTSCDTTVSTSTF |
| | | CACATCGTGTGATACCACAGTTTCAACATCTACGTTTGGGCCTTCAGCAAGATGA | GPSAR* |
| | | SEQ ID NO: 35 | SEQ ID NO: 36 |
| 19 | Xa21_Os07g0190000 | ATGGCGCTCCAGGCATCGTCGTCGCCGTCCTTGTTCCGCGCGATCCCGACGAACACCAACGCTTCTTG | MALQASSSPSLFRAIPTNTNASC |
| | | CCGGCGAAAGTTTCAGGTCAGGGCGAGCGCGGCGGCGGCGGCGGCGAACGGCGGCGGGGATGGGAAGG | RRKFQVRASAAAAAANGGGDGKV |
| | | TGATGATGAGGAAGGAGGCGGCGTCGGGCGCGTGGAAGATCGACTACTCCGGCGAGAAGCCGGCGACG | MMRKEAASGAWKIDYSGEKPATP |
| | | CCTCTGCTTGACACGGTGAACTACCCGGTCCACATGAAGAACCTCTCGACGCCGGAGCTGGAGCAGCT | LLDTVNYPVHMKNLSTPELEQLA |
| | | GGCGGCAGAGCTCCGGGCGGAGATCGTGCACACGGTGTCCAAGACGGGGGGCCACCTGAGCTCCAGCC | AELRAEIVHTVSKTGGHLSSSLG |
| | | TGGGCGTCGTCGAGCTCGCCGTGGCGCTGCACCATGTGTTCGACACGCCGGAGGACAAGATCATCTGG | VVELAVALHHVFDTPEDKIIWDV |
| | | GACGTCGGGCACCAGGCGTACCCGCACAAGATCCTGACGGGGCGGCGTTCGCGGATGCACACCATCCG | GHQAYPHKILTGRRSRMHTIRQT |
| | | GCAGACCTCCGGGCTCGCCGGGTTCCCGAAGCGCGACGAGAGCGCGCACGACGCGTTCGGCGCGGGCC | SGLAGFPKRDESAHDAFGAGHSS |
| | | ACAGCTCGACGAGCATCTCGGCGGCGCTCGGGATGGCAGTGGCGAGGGACCTCCTCGGGAAGAAGAAC | TSISAALGMAVARDLLGKKNHVI |
| | | CACGTCATCTCGGTGATCGGAGACGGCGCCATGACCGCCGGCCAGGCGTACGAGGCGATGAACAACTC | SVIGDGAMTAGQAYEAMNNSGYL |
| | | GGGCTACCTCGACTCCAACATGATCGTGGTGCTCAACGACAACAAGCAGGTGTCCCTCCCGACGGCGA | DSNMIVVLNDNKQVSLPTATLDG |
| | | CGCTCGACGGCCCCGCCACGCCCGTCGGCGCGCTGAGCAAGGCGCTGACGAAGCTCCAGTCGAGCACC | PATPVGALSKALTKLQSSTKLRR |
| | | AAGCTCCGCCGCCTCCGCGAGGCTGCGAAGACGGTGACGAAGCAGATCGGCGGGCAGGCGCACGAGGT | LREAAKTVTKQIGGQAHEVAAKV |
| | | GGCGGCGAAGGTGGACGAGTACGCGCGCGGCATGGTGAGCGCGTCGGGCTCGACGCTGTTCGAGGAGC | DEYARGMVSASGSTLFEELGLYY |
| | | TCGGGCTCTACTACATCGGCCCCGTCGACGGCCACAACGTCGACGACCTCGTCGCCATCTTCAACAAG | IGPVDGHNVDDLVAIFNKVKSMP |
| | | GTGAAGTCGATGCCGGCGCCGGGGCCGGTGCTCGTCCACATCGTGACGGAGAAGGGGAAGGGCTACCC | APGPVLVHIVTEKGKGYPPAEAA |
| | | GCCGGCGGAGGCCGCCGCCGACCGGATGCACGGCGTGGTCAAGTTCGACCCGACGACGGGGAAGCAGT | ADRMHGVVKFDPTTGKQFKSKCS |
| | | TCAAGTCCAAGTGTTCGACGCTGTCCTACACCCAGTACTTCGCCGAGGCGTTGATCCGGGAGGCCGAG | TLSYTQYFAEALIREAEADDKVV |
| | | GCCGACGACAAGGTGGTCGGCATCCACGCCGCCATGGGCGGCGGCACGGGGCTCAACTACTTCCACAA | GIHAAMGGGTGLNYFHKRFPERC |
| | | GAGGTTCCCGGAGCGGTGCTTCGACGTGGGGATCGCGGAGCAGCACGCCGTGACGTTCGCCGCGGGGC | FDVGIAEQHAVTFAAGLAAEGLK |
| | | TCGCCGCCGAGGGGCTCAAGCCATTCTGCGCCATCTACTCGTCGTTCCTGCAGCGCGGGTACGACCAG | PFCAIYSSFLQRGYDQVVHDVDL |
| | | GTGGTGCACGACGTCGACCTGCAGCGGCTGCCGGTGAGGTTCGCCATGGACAGGGCGGGGCTCGTCGG | QRLPVRFAMDRAGLVGADGPTHC |
| | | CGCCGACGGGCCCACGCACTGCGGCGCGTTCGACGTCGCCTACATGGCGTGCCTCCCGAACATGGTCG | GAFDVAYMACLPNMVVMAPADEA |
| | | TCATGGCGCCCGCCGACGAGGCCGAGCTCATGCACATGGTCGCCACCGCCGCCGCCATCGACGACCGC | ELMHMVATAAAIDDRPSCFRFPR |
| | | CCTAGCTGCTTCCGCTTCCCCCGCGGCAATGGCATTGGCGCCGTCCTCCCTCCCAACCACAAGGGCAC | GNGIGAVLPPNHKGTPLEVGKGR |
| | | ACCTCTCGAGGTCGGGAAGGGGAGGGTGCTCGTAGGAGGGAACAGGGTGGCGCTGCTAGGGTACGGCA | VLVGGNRVALLGYGTMVQACMKA |
| | | CGATGGTGCAGGCATGCATGAAGGCGGCAGAGGCGCTGAAGGAGCACGGCATCTACGTCACCGTCGCC | AEALKEHGIYVTVADARFCKPLD |
| | | GACGCGCGTTTCTGCAAGCCGCTCGACACAGGGCTTATCCGGGAGCTCGCCGCCGAGCACGAGGTCCT | TGLIRELAAEHEVLVTVEEGSIG |
| | | CGTCACTGTGGAGGAGGGCTCCATCGGAGGGTTCGGTTCCCACGTCGCCCACTACCTCAGCCTCAGCG | GFGSHVAHYLSLSGLLDGPLKLR |
| | | GCCTCCTTGATGGCCCCCTCAAGTTGAGGTCCATGTTCTTGCCAGACAGGTACATAGACCATGGTGCA | SMFLPDRYIDHGAPVDQLEEAGL |
| | | CCAGTAGACCAGCTTGAGGAGGCAGGATTGACACCAAGGCACATTGCTGCCACTGTGCTGTCCTTGCT | TPRHIAATVLSLLGRPLEALQLS |
| | | TGGGAGGCCATTGGAGGCACTTCAGCTCAGCTGA SEQ ID NO: 37 | * SEQ ID NO: 38 |
| 20 | Xa21_Os03g0109400 | ATGGCTGCGGCAGTGGCAATGCGAGGGAGTAGCAGTGATGGAGGTGGCTATGATAAGGTTTCCGGGAT | MAAAVAMRGSSSDGGGYDKVSGM |
| | | GGACTCCGGTAAATATGTGCGCTACACGCCTGAGCAGGTGGAGGCGCTTGAGCGGGTGTACGCCGATT | DSGKYVRYTPEQVEALERVYADC |
| | | GCCCCAAGCCAACCTCCTCCCGCAGGCAGCAACTGCTGCGTGAGTGCCCCATACTTGCTAACATCGAG | PKPTSSRRQQLLRECPILANIEP |
| | | CCGAAGCAGATCAAGGTCTGGTTCCAGAACAGAAGGTGCCGGGATAAGCAGCGGAAGGAGTCTTCACG | KQIKVWFQNRRCRDKQRKESSRL |
| | | GCTTCAGGCTGTCAACAGGAAATTGACGGCAATGAACAAGCTTCTTATGGAAGAGAATGAACGACTCC | QAVNRKLTAMNKLLMEENERLQK |
| | | AGAAGCAGGTCTCCCAATTGGTTCATGAGAATGCCCACATGCGACAGCAGCTGCAGAATACTCCGCTG | QVSQLVHENAHMRQQLQNTPLAN |
| | | GCAAATGATACAAGCTGTGAATCAAATGTGACTACCCCTCAAAACCCTTTAAGGGATGCAAGTAACCC | DTSCESNVTTPQNPLRDASNPSG |
| | | CTCTGGGCTCCTTTCAATTGCAGAGGAGACCTTGACAGAGTTCCTCTCAAAGGCTACTGGTACAGCTA | LLSIAEETLTEFLSKATGTAIDW |
| | | TTGATTGGGTCCAGATGCCTGGGATGAAGCCTGGTCCGGATTCGGTTGGTATTGTGGCCATTTCACAT | VQMPGMKPGPDSVGIVAISHGCR |
| | | GGTTGCCGTGGTGTTGCTGCCCGTGCCTGTGGTTTGGTGAACCTAGAACCAACAAAAGTGGTAGAGAT | GVAARACGLVNLEPTKVVEILKD |
| | | ATTGAAAGATCGTCCATCTTGGTTCCGTGATTGTCGAAACCTGGAAGTCTTTACAATGATTCCAGCAG | RPSWFRDCRNLEVFTMIPAGNGG |
| | | GAAATGGAGGAACGGTTGAACTTGTCTACACACAGTTGTATGCTCCAACAACTTTAGTTCCTGCACGA | TVELVYTQLYAPTTLVPARDFWT |
| | | GATTTTTGGACGATACGGTACACAACCACAATGGAAGATGGCAGTCTTGTGGTCTGTGAGAGATCTTT | IRYTTTMEDGSLVVCERSLSGSG |
| | | AAGTGGTTCAGGGGGCGGTCCAAGTGCTGCCTCTGCTCAGCAATATGTGAGAGCGGAAATGCTTCCAA | GGPSAASAQQYVRAEMLPSGYLV |
| | | GTGGATACCTGGTTCGCCCATGTGAAGGTGGGGGATCAATTGTGCACATAGTGGACCATCTGGATCTT | RPCEGGGSIVHIVDHLDLEAWSV |
| | | GAGGCATGGAGTGTTCCTGAGGTGCTTCGGCCACTCTATGAATCTTCAAGGGTAGTCGCTCAGAAAAT | PEVLRPLYESSRVVAQKMTTAAL |
| | | GACTACTGCGGCACTCCGGCACATCAGACAAATTGCTCAAGAAACAAGTGGGGAAGTGGTGTATGCCT | RHIRQIAQETSGEVVYALGRQPA |
| | | TGGGGAGGCAACCAGCAGTGCTACGGACTTTTAGTCAAAGGCTGAGCAGAGGCTTTAACGATGCCATT | VLRTFSQRLSRGFNDAISGFNDD |
| | | AGTGGTTTCAATGATGATGGGTGGTCTATAATGGGCGGAGACGGTGTTGAAGATGTAGTTATTGCTTG | GWSIMGGDGVEDVVIACNSTKKI |
| | | CAACTCAACTAAGAAAATTAGGAGTAGCAGCAATGCTGGCATCGCCTTTGGAGCCCCCGGAGGTATTA | RSSSNAGIAFGAPGGIICAKASM |
| | | TATGTGCTAAGGCATCAATGTTACTGCAGAGTGTTCCTCCAGCAGTACTGGTTCGATTTCTGAGGGAG | LLQSVPPAVLVRFLREHRSEWAD |
| | | CATAGATCTGAATGGGCCGATTACAATATTGATGCATATTTGGCTTCAACTCTGAAAACAAGTGCATG | YNIDAYLASTLKTSACSLPGLRP |
| | | TTCACTTCCTGGGTTGCGACCCATGAGATTTTCTGGGAGCCAAATCATCATTCCACTTGCTCACACAG | MRFSGSQIIIPLAHTVENEEILE |
| | | TCGAGAATGAGGAGATTCTTGAAGTTGTTCGCCTTGAGGGTCAACCTCTTACTCATGATGAAGCTCTT | VVRLEGQPLTHDEALLSRDIHLL |
| | | CTTTCAAGGGATATCCACCTGCTTCAGCTCTGCACTGGAATAGACGAGAAGTCTGTGGGATCCTCCTT | QLCTGIDEKSVGSSFQLVFAPID |
| | | TCAGCTTGTCTTTGCACCGATTGATGATTTCCCAGATGAAACTCCATTGATTTCTTCTGGCTTCCGTG | DFPDETPLISSGFRVIPLDMKTD |
| | | TTATACCACTTGATATGAAAACAGATGGTGCATCCTCTGGTAGGACATTAGATTTGGCATCTAGTCTT | GASSGRTLDLASSLEVGSATAQA |
| | | GAAGTAGGTTCAGCAACAGCTCAAGCCTCCGGAGATGCATCTGCAGATGATTGTAACTTGCGATCTGT | SGDASADDCNLRSVLTIAFQFPY |
| | | TCTGACGATCGCTTTTCAATTCCCTTATGAGTTGCATCTCCAAGACAGTGTTGCAGCTATGGCTCGCC | ELHLQDSVAAMARQYVRSIVSAV |
| | | AATATGTCCGTAGCATTGTTTCTGCTGTGCAAAGAGTGTCAATGGCTATCTCTCCCTCTCAAACTGGT | QRVSMAISPSQTGLNAGQRIISG |
| | | CTAAATGCCGGACAGAGGATAATCTCTGGTTTCCCTGAAGCAGCAACCCTTGCTCGATGGGTTTGCCA | FPEAATLARWVCQSYHYHLGVEL |
| | | GAGCTACCATTACCATCTAGGGGTAGAGTTACTTAGTCAATCAGATGGAGATGCAGAACAATTGTTGA | LSQSDGDAEQLLKMLWHYQDAIL |
| | | AGATGCTATGGCATTACCAAGATGCTATTTTGTGCTGCTCATTCAAGGAAAAACCGGTGTTTACATTT | CCSFKEKPVFTFANKAGLDMLET |
| | | GCCAACAAAGCAGGACTGGACATGCTAGAAACTTCCCTTGTCGCCTTACAGGACCTCACATTGGACAG | SLVALQDLTLDRIFDEPGKEALF |
| | | GATCTTTGATGAGCCTGGAAAAGAAGCATTGTTCTCAAACATTCCCAAATTGATGGAACAGGGCCATG | SNIPKLMEQGHVYLPSGVCMSGM |
| | | TCTACCTGCCATCAGGCGTGTGCATGTCAGGAATGGGTCGGCATGTTTCTTTCGATCAGGCCGTGGCT | GRHVSFDQAVAWKVLAEDSNVHC |
| | | TGGAAAGTGCTTGCCGAGGATAGTAATGTCCACTGCCTGGCCTTCTGTTTCGTCAACTGGTCCTTTGT | LAFCFVNWSFV* |
| | | GTGA SEQ ID NO: 39 | SEQ ID NO: 40 |
| 21 | Xa21_Os01g0184100 | ATGGAGAGCGCCATGTTCGGGCTGGAGACGCCGCTGATGACGGCGCTGCAGCACCTGCTCGACATCCC | MESAMFGLETPLMTALQHLLDIP |
| | | CGACGGCGAGGGCGGCGCCGCCGGGAAGCAGGGCGCGACCGGTGGCCCCACGCGTGCGTACGTGCGCG | DGEGGAAGKQGATGGPTRAYVRD |
| | | ACGCGCGGGCCATGGCGGCGACCCCCGCCGACGTGAAGGATCTCCCCGGCGCGTACGCGTTCGTGGTG | ARAMAATPADVKDLPGAYAFVVD |
| | | GACATGCCTGGGCTCAAGTCCTCCGACATCAAGGTGCAGGTGGAGGAGGAGAGGGTGCTGGTGATCAG | MPGLKSSDIKVQVEEERVLVISG |
| | | CGGCGAGCGGCGGCGCGGCGGCGGGGAGGAGGAGAAGGAGGAGTCGTGCAAGTACCTGCGGATGGAGC | ERRRGGGEEEKEESCKYLRMERR |
| | | GGCGGATGGGCAAGTTCATGCGCAAGTTCGTGCTCCCCGACAACGCCGACGTCGACAAGATCTCCGCC | MGKFMRKFVLPDNADVDKISAVC |
| | | GTGTGCCAGGACGGCGTGCTCACCGTCACCGTCGAGAAGCTCCCGCCGCCGGAGCCCAAGAAGCCCAA | QDGVLTVTVEKLPPPEPKKPKTI |
| | | GACCATCGAGGTCAAGGTCGCGTGA | EVKVA* |
| | | SEQ ID NO: 41 | SEQ ID NO: 42 |
| 22 | Xa21_Os11g0703900 | ATGGCGGGCAAGGGCGAGGGTCCGGCCATCGGCATCGACCTGGGCACGACCTACTCGTGCGTGGGCGT | MAGKGEGPAIGIDLGTTYSCVGV |
| | | GTGGCAGCATGACCGCGTGGAGATCATCGCCAACGACCAGGGCAACCGCACCACCCCCTCCTACGTCG | WQHDRVEIIANDQGNRTTPSYVG |
| | | GCTTCACCGACTCCGAGAGGCTCATCGGAGATGCTGCCAAGAACCAGGTCGCCATGAACCCCATCAAC | FTDSERLIGDAAKNQVAMNPINT |
| | | ACCGTCTTCGATGCCAAGCGTCTCATTGGCAGGAGGTTTAGCGATGCTTCTGTTCAGAGTGACATTAA | VFDAKRLIGRRFSDASVQSDIKL |
| | | GCTCTGGCCCTTCAAGGTGATTGCTGGACCTGGTGACAAGCCTATGATTGTTGTCCAGTACAAGGGTG | WPFKVIAGPGDKPMIVVQYKGEE |
| | | AGGAGAAGCAGTTTGCTGCTGAAGAGATCTCCTCCATGGTCCTCATCAAGATGCGTGAGATTGCTGAG | KQFAAEEISSMVLIKMREIAEAY |
| | | GCCTACCTTGGCACCACCATCAAGAATGCCGTTGTCACTGTTCCTGCCTACTTCAATGACTCCCAGAG | LGTTIKNAVVTVPAYFNDSQRQA |
| | | GCAGGCCACCAAGGATGCTGGAGTGATTGCTGGTCTCAATGTCATGCGTATCATCAACGAGCCGACTG | TKDAGVIAGLNVMRIINEPTAAA |
| | | CTGCAGCTATTGCCTATGGTCTTGACAAGAAGGCCACCAGCGTTGGTGAGAAGAATGTCCTCATCTTT | IAYGLDKKATSVGEKNVLIFDLG |
| | | GACCTTGGAGGTGGTACCTTTGATGTTTCCCTCCTTACCATTGAGGAGGGTATCTTTGAGGTCAAGGC | GGTFDVSLLTIEEGIFEVKATAG |
| | | CACAGCTGGTGACACCCATCTTGGTGGTGAAGATTTTGACAACCGCATGGTCAACCACTTTGTGCAAG | DTHLGGEDFDNRMVNHFVQEFKR |
| | | AATTCAAGAGGAAGAACAAGAAGGATATCACTGGCAACCCCAGGGCTCTCAGGAGGTTGAGGACAGCT | KNKKDITGNPRALRRLRTACERA |
| | | TGTGAGAGGGCGAAGAGGACACTGTCCTCCACTGCCCAGACCACCATTGAGATCGATTCCCTGTATGA | KRTLSSTAQTTIEIDSLYEGIDF |
| | | GGGCATTGACTTCTACTCAACCATCACCCGTGCCAGGTTTGAGGAGCTCAACATGGATCTCTTCAGGA | YSTITRARFEELNMDLFRKCMEP |
| | | AGTGTATGGAGCCTGTGGAGAAGTGCCTCAGGGATGCTAAGATGGACAAGAGCTCTGTTCATGATGTT | VEKCLRDAKMDKSSVHDVVLVGG |
| | | GTCCTTGTTGGTGGCTCCACTAGGATCCCGAGGGTGCAGCAGCTCCTGCAGGATTTCTTCAACGGCAA | STRIPRVQQLLQDFFNGKELCKN |
| | | GGAGCTTTGCAAGAACATCAACCCAGATGAGGCTGTTGCTTATGGTGCTGCTGTCCAGGCTGCCATCT | INPDEAVAYGAAVQAAILSGEGN |
| | | TGAGTGGTGAGGGCAACGAGAAGGTCCAGGACCTCCTCCTGTTGGATGTCACCCCTCTCTCTCTCGGT | EKVQDLLLLDVTPLSLGLETAGG |
| | | TTGGAGACTGCTGGTGGTGTCATGACCGTCTTGATCCCAAGGAACACCACCATTCCCACCAAGAAGGA | VMTVLIPRNTTIPTKKEQVFSTY |
| | | GCAGGTCTTCTCCACCTACTCTGACAACCAGCCTGGTGTGCTCATCCAGGTTTATGAGGGTGAGAGGA | SDNQPGVLIQVYEGERTRTRDNN |
| | | CCAGGACACGTGACAACAACCTGCTGGGCAAGTTTGAGCTCTCTGGAATCCCTCCTGCTCCCAGGGGT | LLGKFELSGIPPAPRGVPQITVC |
| | | GTTCCACAGATCACTGTTTGCTTCGACATTGATGCCAATGGTATCCTGAACGTGTCTGCTGAGGACAA | FDIDANGILNVSAEDKTTGQKNK |
| | | GACCACCGGGCAGAAGAACAAGATCACCATCACCAACGACAAGGGCAGGCTCAGCAAGGAGGAGATTG | ITITNDKGRLSKEEIEKMVQEAE |
| | | AGAAGATGGTCCAGGAGGCCGAGAAGTACAAGTCAGAGGATGAGGAGCACAAGAAGAAGGTGGAGTCC | KYKSEDEEHKKKVESKNALENYA |
| | | AAGAACGCGCTGGAGAACTACGCCTACAACATGCGCAACACCATCAAGGACGAGAAGATCGCCTCGAA | YNMRNTIKDEKIASKLPAADKKK |
| | | GCTACCGGCAGCGGACAAGAAGAAGATCGAGGATGCCATCGACCAGGCCATCCAGTGGCTGGACGGCA | IEDAIDQAIQWLDGNQLAEADEF |
| | | ACCAGCTCGCTGAGGCTGATGAGTTCGATGACAAGATGAAGGAGCTGGAGGGCATCTGCAACCCCATC | DDKMKELEGICNPIIAKMYQGAG |
| | | ATCGCCAAGATGTACCAGGGCGCTGGCGCGGACATGGCCGGCGGCATGGACGAGGACGATGCTCCCCC | ADMAGGMDEDDAPPAGGSGAGPK |
| | | GGCTGGCGGCAGCGGTGCTGGCCCCAAGATCGAGGAGGTCGACTAA | IEEVD* |
| | | SEQ ID NO: 43 | SEQ ID NO: 44 |
| 23 | Xa21_OsI_34361 | ATGGCAGCGGCGGCGGCGAGGTACGACGTGGAGAAGGGAGGGAGGAAGAGGGAGGGGGAGGAGGAGAG | MAAAAARYDVEKGGRKREGEEER |
| | | GTACGGTTCGCCGGCGGCGGTGGCGCAGTACCCGCAGCGGGAGGGGGAGAGGGAGTGGGTGCCGTGGC | YGSPAAVAQYPQREGEREWVPWL |
| | | TCGTGCCGGCCATCCTCGTCGCCAACGTCGTCGTCTTCGCCGTCGCCATGTACGTCAACAACTGCCCC | VPAILVANVVVFAVAMYVNNCPS |
| | | AGCTCCCACGCCTCTCGCGGCCGCCGAGGCGGCGCGTGCGTCGCCGGATTCCTCCGCCGCTTCTCCTT | SHASRGRRGGACVAGFLRRFSFQ |
| | | CCAGCCTCTCTCCGAGAACCCGCTCCTCGGCCCCTCCTCCGCCACATTGCAGAAGATGGGAGCACTGG | PLSENPLLGPSSATLQKMGALVW |
| | | TATGGGACAAGGTTGTTCATGAGCATCAGGGATGGAGGCTAGTGACCTGCATCTGGCTGCACGCTGGT | DKVVHEHQGWRLVTCIWLHAGVV |
| | | GTTGTTCATCTGCTTGCCAACATGCTGAGCCTCGTGCTCATCGGACTCAGGCTCGAGCAGCAGTTTGG | HLLANMLSLVLIGLRLEQQFGYM |
| | | ATACATGAGAATTGGCATCATCTACCTTGTTTCTGGCATTGGAGGCAGCGTTCTTTCATCTCTGTTCA | RIGIIYLVSGIGGSVLSSLFIRN |
| | | TCAGAAACAGCATCTCCGTCGGTGCTTCTGGCGCTCTCTTTGGTCTTCTTGGAGCCATGCTGTCAGAG | SISVGASGALFGLLGAMLSELFT |
| | | CTTTTCACCAACTGGACCATATACACGAACAAGGCTGCAGCTCTAGTGACGCTCTTGATCGTCATCGC | NWTIYTNKAAALVTLLIVIAINL |
| | | GATCAACCTCGCCATCGGCATCCTCCCTCACGTCGACAACTTCGCCCACATCGGAGGGTTCTTGACTG | AIGILPHVDNFAHIGGFLTGFLL |
| | | GGTTCCTCCTCGGATTCATCTTCCTGATGCGCCCACACTACGGATGGATGCAGCGATATGTCCTGCCT | GFIFLMRPHYGWMQRYVLPSSVK |
| | | TCCTCTGTCAAGTACACCTCCAAGAAGTACCTGGCTTATCAATGGATCTTGCTGGCCATGGCCTCGGT | YTSKKYLAYQWILLAMASVLAVI |
| | | CCTAGCTGTCATCGGATTCGCCGTTGGGCTGTCGATGCTCTTCAGAGGGGTGAATGCTAATGAACGCT | GFAVGLSMLFRGVNANERCHWCH |
| | | GCCATTGGTGCCACTACCTGAGCTGCATTCCTACCTCGAGATGGACCTGCGGGAACTGA | YLSCIPTSRWTCGN* |
| | | SEQ ID NO: 45 | SEQ ID NO: 46 |
| 24 | Xa21_OsI_09185 | ATGGCCGACGGGGGAGAGAAGTGCCGGGACGCGGCCGGCGAGGGCGGCGGCGGCGGCGACCTGTACGC | MADGGEKCRDAAGEGGGGGDLYA |
| | | CGTGCTCGGGCTCAAGAAGGAGTGCTCCGACGCCGACCTCAAGCTCGCGTACCGGAAGCTCGCCATGA | VLGLKKECSDADLKLAYRKLAMR |
| | | GATGGCATCCGGACAAATGCTCATCCTCCAGCAGTGCAAAGCACATGGAGGAAGCCAAGGAGAAGTTC | WHPDKCSSSSSAKHMEEAKEKFQ |
| | | CAGGAGATCCAGGGCGCCTATTCCGTCCTCTCAGACTCAAACAAGCGGTTCCTCTACGACGTGGGGGT | EIQGAYSVLSDSNKRFLYDVGVY |
| | | ATATGATGATGACAACAATGACGATGACAACCTGCAGGGGATGGGGGACTTCATTGGTGAGATGGCCC | DDDNNDDDNLQGMGDFIGEMAQM |
| | | AGATGATGAGCCAGGCACGGCCAACGAGGCAGGAGAGCTTTGAAGAACTGCAGCAGCTATTCGTAGAC | MSQARPTRQESFEELQQLFVDMF |
| | | ATGTTCCAAGCTGATCTTGATTCGGGTTTCTGCAATGGACCCTCAAAGTGCTACCATACCCAGGCCCA | QADLDSGFCNGPSKCYHTQAQSQ |
| | | AAGCCAGACTCGAACATCCTCAACCTCCCCTTCGACGTCACCGTCTCCACCGCCTCCAGTAGCTACTG | TRTSSTSPSTSPSPPPPVATEAE |
| | | AGGCAGAATCGCCATCATGTAATGGTATTAATAAGCGTGGTTCATCAGCAATGGACTCTGGGAAGCCT | SPSCNGINKRGSSAMDSGKPPRA |
| | | CCAAGAGCCAGCGAAGTCGGTGCTGGACAGAGTCAATCAGGGTTTTGTTTCGGGCAGAAGAGTGATGC | SEVGAGQSQSGFCFGQKSDAKQA |
| | | TAAACAAGCGGCGAAGACGCGAAGCGGGAACACGGCCAGCCGGAGGAGGAACGGCCGGAAGCAGAAGG | AKTRSGNTASRRRNGRKQKVSSK |
| | | TGTCGTCGAAGCACGACGTCTCGTCTGAGGACGAGATGCCAGGTCACCCTTACACTGTTAAAGCTAAA | HDVSSEDEMPGHPYTVKAKN* |
| | | AACTAA SEQ ID NO: 47 | SEQ ID NO: 48 |
| 25 | Xa21_Os06g0682900 | ATGGCGATGCGGCGGTGGAGAGAGGATGCGGTGGCGCTGTCGCTGCGCGGGTGCGGGTTCGGCGACGG | MAMRRWREDAVALSLRGCGFGDG |
| | | GGACGACGACCGGCCGGAGAAGCCGCGGCGGTACGGGGTGACGGAGATGAGGAGCCCCTTGTACGCCT | DDDRPEKPRRYGVTEMRSPLYAF |
| | | TCCGCCCCGCCCACCACGCGCTCCAGGAAATATTGGATTCCCTAGGTCCTTTTGTTGATGGTTTGAAG | RPAHHALQEILDSLGPFVDGLKF |
| | | TTTACTGGTGGATCTCATAGTTTGATGGGAAAAGAACTGGTTAGAGAGATTACCGATTTAGCACACAA | TGGSHSLMGKELVREITDLAHKH |
| | | GCATGACATATATGTGAGTACAGGAGATTGGGCAGAGCATCTTCTGCATCAAGGACCTTCTTTTTTCA | DIYVSTGDWAEHLLHQGPSFFKQ |
| | | AGCAATATGTCGAGGAGTGCAAGGCATTAGGATTTGACACCATCGAGCTGAATGCTGGATCTCTCAAG | YVEECKALGFDTIELNAGSLKLP |
| | | CTCCCGGAAGAAGCTCTCCTGAGATTAGTCCGCCTCATCAAGACTAGTGGTTTGCAAGCAAAGCCCCT | EEALLRLVRLIKTSGLQAKPLFS |
| | | GTTTTCAGTGAAGTTTGACAGTTCTGATATTCCTCCATCAGGAGATAGGGCATTTGGGGCATACATAG | VKFDSSDIPPSGDRAFGAYIVPV |
| | | TTCCAGTGAAGCAGAATTCAGAAAGAGTTGAGGATGTTGACCTGTTGATAAGGAGAGCCGAGAGGTGC | KQNSERVEDVDLLIRRAERCLEA |
| | | TTGGAAGCAGGGGCAGACATGATCATGATCGATGCCGATGACATTTGCCAGCGCGCCGACTCTCTACG | GADMIMIDADDICQRADSLRADI |
| | | GGCAGACATCGTCGCCAAGATCGTAGGCCGGCTAGGGCTCGAGAAGACCATGTTCGAGGCTTCCAACC | VAKIVGRLGLEKTMFEASNPNTS |
| | | CCAACACCTCCGAGTGGTTCGTCAGAAGATACGGGCCACGGGTGAATCTGTTCGTGGATCACTCCGAC | EWFVRRYGPRVNLFVDHSDVMNL |
| | | GTGATGAACCTGGAGCGTCTCCGAGGCTTCAATATGCGTGGAGTCTGCAACTCTCCCTTGTTCGGAAT | ERLRGFNMRGVCNSPLFGIGSPF |
| | | CGGCTCGCCCTTCTTCCTGATGTGA SEQ ID NO: 49 | FLM* SEQ ID NO: 50 |
| 26 | Xa21_Os01g0134700 | ATGGCGGCGAAGCGGCTTCACGACGGCTACGGACAGGAGGACCAGCCCGACGACAAGCGTGTGCGCCG | MAAKRLHDGYGQEDQPDDKRVRR |
| | | CATGCCGTCCTTCTCGACGGTGATCCGTGAGGCCTTGATGGTGAAGCAGATGCAGACTCTGTTCGTCG | MPSFSTVIREALMVKQMQTLFVA |
| | | CACTGGAGCCACTTCTCCGTAGAGTGGTGCAGGAGGAGCTCCAGGCAGGTCTCGTGCGCAGCCCGCGT | LEPLLRRVVQEELQAGLVRSPRY |
| | | TACATCGAGAGGATGTCGCCTGAGACGCCGCCGGCTCAGCCGCCGATGTGGAAGCTGGCGTTCCGGTT | IERMSPETPPAQPPMWKLAFRFK |
| | | CAAGCCGCAGCTCCCGATCTTCACCGGGAGCAAGATCGAGGACGTGAACGGCAACCCACTGGAGATCA | PQLPIFTGSKIEDVNGNPLEIIL |
| | | TCCTCATCGACGTCGACACCGGCGCGCCGGCGACGATCTCGCAGCCTCTGCGCGTCGAGGTCGTCCCG | IDVDTGAPATISQPLRVEVVPVL |
| | | GTCCTGGGCGACTTCCCGCCGGACGACCGCGAGCACTGGACGGCGGAGGAGTTCCAGCAGAGGGGCAT | GDFPPDDREHWTAEEFQQRGIVK |
| | | CGTGAAGGAGCGCTCCGGCAAGCGCCCGCTCCTCACCGGCGACGTCAGCCTCACCATGCGCGACGGCT | ERSGKRPLLTGDVSLTMRDGCVA |
| | | GCGTCGCCGTGAACGAGCTCCAGTTCACGGACAACTCCTCCTGGGTTCGCTGCCGCAGGTTCCGCATC | VNELQFTDNSSWVRCRRFRIGVR |
| | | GGCGTCCGCGTCGTGCCGGGCAGCTACGACGGCCCAAGAATCGGCGAGGCCATGACCGAGCCCTTCGT | VVPGSYDGPRIGEAMTEPFVVRD |
| | | CGTCAGAGACCACCGCGGCGAACTGTACAGGAAGCACTACCCTCCCGTTCTTGGCGACGATGTCTGGA | HRGELYRKHYPPVLGDDVWRLEK |
| | | GGCTTGAGAAGATTGGCAAGGAGGGTGCCTTCCACAGGAAGCTGACGCAGCACAACGTCAGGAACGTG | IGKEGAFHRKLTQHNVRNVQEFL |
| | | CAGGAGTTCTTGCGGCTGCTCACGGTTAAGCCGGACGAGCTGCGCGCTATCATGGGCGACGGCATGAC | RLLTVKPDELRAIMGDGMTDRMW |
| | | CGATCGCATGTGGGAGGTGACGACGAGCCACGCCAAGAAGTGCGTCCCCGGCGACAAGGTGTACATGT | EVTTSHAKKCVPGDKVYMYSTQH |
| | | ACAGCACGCAGCACGGCACCGTCTACGTCAACTCCATCTTCGAGCTCGTGAAGGTCGAGCTCGCCGGC | GTVYVNSIFELVKVELAGVEYQL |
| | | GTTGAGTACCAGCTGCACCAGCTGAACAGGGGTCAGAGGGCGTTCGTGCATCAGCTGCTGCTGGCGGC | HQLNRGQRAFVHQLLLAAYEQRN |
| | | GTACGAGCAGCGAAACAACCTCCAAGAGGCCGACGCCATGGCGCTCCACTGCAACGACGTTCCTCTGC | NLQEADAMALHCNDVPLLQNAAE |
| | | TGCAGAATGCAGCGGAGATAACTATCCCGGCGCTTGGAGACACGCAGCTGTGGATTCAGAACAGTCTC | ITIPALGDTQLWIQNSLNSQEID |
| | | AACAGCCAAGAAATCGACTTCCAGGTCGACGAGATCCCCCAAGCTAACTTTGCGCTTCAGTGGACTGG | FQVDEIPQANFALQWTGQMYNIS |
| | | CCAAATGTACAACATTTCAGGCTAA SEQ ID NO: 51 | G* SEQ ID NO: 52 |
| 27 | Xa21_Os01g0135700 | ATGTCGAACACCACCGAGACGAAGATGCCGCAGCAGCAGCAGCAGGTTGAGAAGCCGACGGCGTTGGC | MSNTTETKMPQQQQQVEKPTALA |
| | | GCCGGCGGACGCGGAGATCGAGAGGGTCTTCACCCGCTTCGACGCGGACGGGGACGGCAGGATCTCCC | PADAEIERVFTRFDADGDGRISP |
| | | CCTCGGAGCTCGCCGCGGTGACGCGCGCCATCGCGCCGCCGCCCAGCGAGTCGGCCGGCGGCCGCGAG | SELAAVTRAIAPPPSESAGGREV |
| | | GTGGCCGCCATGATGGACGAGCTCGACACCGACCGCGACGGCTTCGTCGACCTCGGCGAGTTCGCGGC | AAMMDELDTDRDGFVDLGEFAAF |
| | | GTTCCACGGCCGCGGGCGCGGGGACGCCGAGCACGAGGCCGAGCTGCGCGCCGCGTTCGACGTGTACG | HGRGRGDAEHEAELRAAFDVYDV |
| | | ACGTCGACGGCGACGGCCGCATCACCGCGGCCGAGCTCGGCAAGGTCCTCGCCCGGATCGGCGAGGGC | DGDGRITAAELGKVLARIGEGCS |
| | | TGCAGCGCCGAGGAGTGCGAGAGGATGATCGCCTCCGTCGACGTCGATGGCGATGGCTGCGTCGGGTT | AEECERMIASVDVDGDGCVGFEE |
| | | CGAGGAGTTCAAGAAGATGATGTCCCGTGATGCCGCTGCAACCGGCGGCGCTGACAAGGCCAAGACGG | FKKMMSRDAAATGGADKAKTE* |
| | | AGTAA SEQ ID NO: 53 | SEQ ID NO: 54 |
| 28 | Xa21_Os01g0534800 | ATGGATTTCGAGCTGCGCAAGGCGCGGGAGAAGCTGGAGCGGGAGCAGCGGGAGCGCGTGCAGCACGC | MDFELRKAREKLEREQRERVQHA |
| | | CAAGGCCAAGGCCGAACGCGAGCGCCGCGCCAAGGCCGAGGCCGCGCGCCGCCGCGACGCGCTCGAGG | KAKAERERRAKAEAARRRDALEA |
| | | CGTCCCACCGCGAGCGCCGCCTCGACGCCGCGCGCGCGCAGGAAGAGGCTCAGCAAAAGATGGAAGAG | SHRERRLDAARAQEEAQQKMEEV |
| | | GTGATGCAACTTGGAAAGGGGATTTCTTTCTCGCACATGTTTGAAGCTCTTCGATATGATGGTCCTGG | MQLGKGISFSHMFEALRYDGPGD |
| | | GGACAAGATCAAGTTGCCACCATCGTCTTTTAAGGAATTGTCTGATGAAGGAGCACTGGACAAAGGTC | KIKLPPSSFKELSDEGALDKGPM |
| | | CCATGTACTTCAGGTTGTCAAAGGTTAGAGACAGTGTTCCGGGTGCCCCTCAGGATCATGATGCCGAT | YFRLSKVRDSVPGAPQDHDADEA |
| | | GAGGCAACATGTTGTGGTGTTCTTGAGTTCACTGCAAGGGAAGGCTCCGCAGAACTCACTCCACATGT | TCCGVLEFTAREGSAELTPHVWN |
| | | GTGGAACAATCTGTTCCGGGGTGATAGCCCAGATGTTCCTCTGATTGAGGTTAGGTATGTCAGTTTAC | NLFRGDSPDVPLIEVRYVSLPKG |
| | | CCAAAGGGACATATGCGAAGCTGAAGCCAGTAGGAGTGGGGTTCTCAGACCTTCCTAACCATAGGGCT | TYAKLKPVGVGFSDLPNHRAVLE |
| | | GTCCTTGAAACGGCACTCCGCAATCATGCAACACTATCTGAAAATGATTTTGTTGTGGTGAATTATGG | TALRNHATLSENDFVVVNYGQLQ |
| | | GCAACTGCAATACAAGTTGAAGGTTCTTGAATTAAAACCTGCATCAAGTGTTTCTGTCCTGGAGACAG | YKLKVLELKPASSVSVLETDVEV |
| | | ATGTGGAAGTTGACATTGAAGGACCAGATTCAGTTTTGGACAACGTAGAGAATCAACATGTGCTTGTG | DIEGPDSVLDNVENQHVLVPLET |
| | | CCACTTGAGACTGGAAAGGTTGAATCTGGAGTTGTGGAAGAAGGAAAGTTTAGGTACTACAAATTTCT | GKVESGVVEEGKFRYYKFLVDEG |
| | | TGTTGATGAAGGTATGGGTGAGAAAGTAGTTTCTAGGCATGCAAATATTGAGGTTAAGATAGAGACAG | MGEKVVSRHANIEVKIETDTSGG |
| | | ATACAAGTGGTGGTGACACTGATATTTATGTATCAAGGCATCCTTTAGTTTTCCCAACTCAGCACCGT | DTDIYVSRHPLVFPTQHRHEWSS |
| | | CACGAATGGTCTTCTCATGAAATGGGATCTAAGGTCCTCATACTGAAACCAAGGGATGCCAGTCTCGC | HEMGSKVLILKPRDASLASGTYS |
| | | CAGTGGTACCTACAGTATTGGAGTTTATGGTTTCAAGGGAACCACTAAATACCAACTTTCTGTGGCTA | IGVYGFKGTTKYQLSVAIKDVLN |
| | | TTAAAGATGTTCTTAATGGCCAAAGGATTGGTGAGCAGGCTAGTGCCTCATCTAGTGTTGATGTTGAT | GQRIGEQASASSSVDVDSVVCKN |
| | | TCAGTGGTGTGCAAGAACTGCAAACGCTATATATCCAGCCGAACTTCACTACTTCATGAAGCATACTG | CKRYISSRTSLLHEAYCVRHNVV |
| | | TGTGAGACACAATGTTGTTTGCATGCATGATGGGTGTGGTGTTGTTCTTCGAAAGGAGGAAGCAACAG | CMHDGCGVVLRKEEATDHVHCNK |
| | | ATCACGTGCATTGCAACAAGTGTGGGCAAGCTTTCCAGCAGAGAGAAATGGAGAAGCACATGAAAGTT | CGQAFQQREMEKHMKVFHEPLQC |
| | | TTCCATGAGCCACTACAATGCCCTTGCGGGGTGGTCCTTGAAAAGGAAGATATGGTTCAACACCAATC | PCGVVLEKEDMVQHQSSTCPLRL |
| | | CTCAACCTGCCCTTTGCGCTTGATTGTGTGCCGATTTTGTGGTGACACTGTTCAAGCTGGTGGAGAAC | IVCRFCGDTVQAGGEPLDARDRL |
| | | CACTTGATGCTCGGGATCGGCTTCGCAACATGTGTGAGCACGAGAGTATCTGTGGATCCAGGACTGCA | RNMCEHESICGSRTAPCDSCGRS |
| | | CCATGTGATTCTTGTGGACGGTCAGTCATGCTGAAGGACATGGACATTCATGTGATTGCTGTGCATCA | VMLKDMDIHVIAVHQKS* |
| | | GAAGAGCTAA SEQ ID NO: 55 | SEQ ID NO: 56 |
| 29 | Xa21_Os01g0570800 | ATGACTCTGCGGCCACTCAACACAGAGAGAAGCTTCCTCCTCTCCTCCCCGAAGCCACACAGCCCCAG | MTLRPLNTERSFLLSSPKPHSPR |
| | | GGATGCCTGCTCGCCGCCGGTGAGGTCGCCCTCCTCCACCAGGCTCCTCGCCTGCAGGAAGCTCCCCT | DACSPPVRSPSSTRLLACRKLPS |
| | | CCTCCTCCACCAACAGCAAGCCCATGGCCACCGGCGCCGGCGTCCTCGAGAGGTCTCTCAGCTTCAAG | SSTNSKPMATGAGVLERSLSFKN |
| | | AACTGGGAGCCCACCGCGGCGGAGGTCGCCGCGCCGCCGCCGCACGATGAGGCGGCGAGCCGGTGCAT | WEPTAAEVAAPPPHDEAASRCIN |
| | | CAACGGCGCGCGGCCGGGGATCCTGCTGCTGCAGCAGAGCCCCAAGGCCAAGCAGGGCGACGCCGCCG | GARPGILLLQQSPKAKQGDAAAT |
| | | CGACGTCGCCGGCGCAGGCCGCGCTGATCGAGTTCATCTCGCCGAAGCCGCGGTCGGAGCTGGACCAG | SPAQAALIEFISPKPRSELDQAA |
| | | GCGGCGACCAAGGTGCAGAAGCTGTTCAAGGGGCACCGCACCCGGCGGAACCTCGCCGACTGCGCCAT | TKVQKLFKGHRTRRNLADCAIVV |
| | | TGTCGTGGAGGAGCTGTGGTGGAAGGCCTACGACTCCGCCTGCCTCAACATCAAGTCCATCTCCTTCT | EELWWKAYDSACLNIKSISFFDE |
| | | TCGACGAGGCCAAGCAGGAGACCGCGGCGTCGCGCTGGTCCAGGGCCGGCAAGCGCATCGCCAAGGTC | AKQETAASRWSRAGKRIAKVGKG |
| | | GGCAAGGGCCTCTCCAAGAACGAGAAAGCCCAGAAGCTCGCGCTCCAGCACTGGCTCGAAGCTATCGA | LSKNEKAQKLALQHWLEAIDPRH |
| | | CCCGCGCCACCGCTACGGCCACAACTTGCATCTGTACTACGACATCTGGTCTGCGAGTTCGAGCACTG | RYGHNLHLYYDIWSASSSTEPFF |
| | | AGCCATTCTTTTACTGGCTGGATGTAGGAGCTGGGAGAGACATGCATCACCAAAAGTGCCCGAGAAGC | YWLDVGAGRDMHHQKCPRSKLYS |
| | | AAGCTTTATTCACAGCTGATCATGTACCTTGGACCAAACGAGAGGGAGGCTTTTGAAGTTGTTGTGGA | QLIMYLGPNEREAFEVVVEGGKL |
| | | GGGTGGGAAGCTGATGTACAGGAAGAGTGGAGTTCTTGTGAACACCACTGAAGATTCCAAGTGGATAT | MYRKSGVLVNTTEDSKWIFVLST |
| | | TTGTGTTGAGCACAACAAGATCATTATATGTTGGACAGAAGAAGAAGGGTAAATTTCAGCACTCAAGT | TRSLYVGQKKKGKFQHSSFLAGA |
| | | TTCCTAGCTGGGGCAGCTACAACGGCTGCTGGAAGATTGGTTGCCAAAGATGGTGTTCTTCAGGCGAT | ATTAAGRLVAKDGVLQAIWPYSG |
| | | ATGGCCATACAGCGGTCACTACCTCCCAACAGAAGAGAACTTCAGAGAGTTCATCAGCTTCTTGGAGG | HYLPTEENFREFISFLEENSVDL |
| | | AGAACAGTGTTGATCTTGCCGATGTCAAGAGATGTTCGGTCGACGACGATGAATTCCCGTCATTCAAG | ADVKRCSVDDDEFPSFKKTEEKP |
| | | AAGACAGAGGAGAAACCAGAGGAAGCTGAGAAGCCTACTGAACCCACACATGATGAGATCATGGACAG | EEAEKPTEPTHDEIMDSSQIELP |
| | | CTCACAGATTGAGCTGCCTGAGGTAGACATAGTGAAGGAAGCGGTTGTCGAAAACAGCGAGGACACCG | EVDIVKEAVVENSEDTEVAPIMA |
| | | AGGTGGCACCGATCATGGCCAGCCGCCCATCCTTCAAATGGGCAACCGCGAATGGCGCTCGCATTGGC | SRPSFKWATANGARIGCVRDYPA |
| | | TGTGTCCGGGACTACCCGGCTGATCTCCAGAGCATGGCCCTTGAGCATGTCAATCTTTCACCAAGAGT | DLQSMALEHVNLSPRVVPSPTTN |
| | | GGTGCCCTCTCCTACTACAAACAGGCTGCCGATACCGTCTCCTCGGCCAAGCCCGAAGATCAGGCTGT | RLPIPSPRPSPKIRLSPRLHYMG |
| | | CACCTAGGCTGCACTACATGGGTCTCCCGACGCCTACCGGTTGCAAGCTCCCGATCCCGAGCCCGGAG | LPTPTGCKLPIPSPEIRRSPRDQ |
| | | ATCAGGAGGTCACCAAGGGATCAGTTCATGGGGTTTCAGACACCGTCAGTGTCTCTCACACTCCCAAA | FMGFQTPSVSLTLPKLGK* |
| | | GCTTGGGAAGTGA SEQ ID NO: 57 | SEQ ID NO: 58 |
| 30 | Xa21_Os01g0597600 | ATGGCAGATCAGAAGGTGATTCTCGCCGAGCCGCTGCTTCCGGGGAAGGAAGCTGACTTCGCCGACGA | MADQKVILAEPLLPGKEADFADD |
| | | CGACGACGTGGAGGCGCAGCTCACTTCGTACCACACCGGCGCCTCCTTCTCCAGGACGTGCCTCAACC | DDVEAQLTSYHTGASFSRTCLNL |
| | | TCACCAATGCCGTCTCCGGCATCGGGGTGCTGTCCATGCCGTACGCGGTGTCGCAGGGCGGGTGGCTC | TNAVSGIGVLSMPYAVSQGGWLS |
| | | AGCCTGCTGCTCTTCGTCCTCGTCGGCGCCGTGTGCTACTACACCGGCACGCTCATCGAGCGCTGCAT | LLLFVLVGAVCYYTGTLIERCMR |
| | | GCGCGCCGACGGCTCCATCGCCAGCTACCCGGACATCGGCCAGTACGCCTTCGGCGCCACCGGCAGGA | ADGSIASYPDIGQYAFGATGRRA |
| | | GAGCCGTCGCATTCTTCATGTACGTCGAGCTCTACCTCGTCGCCATAAGCTTCCTCGTCCTCGAGGGC | VAFFMYVELYLVAISFLVLEGDN |
| | | GACAATCTCGACAAGCTCTTCCCCGGCGCAACCATGGAGATTCTTGGCTACCAGCTCCACGGCAAGCA | LDKLFPGATMEILGYQLHGKQLF |
| | | GCTATTCATTGTACTCGCCGCCGCCGTCATACTGCCCACTACCTGGCTCAAGAATCTTGGCATGCTCG | IVLAAAVILPTTWLKNLGMLAYV |
| | | CTTACGTGTCAGCGGCCGGGCTCATCGCCTCGGTGGCCTTGACGGCGTCACTGATCTGGGCCGGTGTC | SAAGLIASVALTASLIWAGVAET |
| | | GCCGAGACCGGGTTCCACCGGAACAGCAACACGCTCAACCTCGCCGGAATCCCCACCTCCCTCGGTCT | GFHRNSNTLNLAGIPTSLGLYFV |
| | | CTACTTCGTCTGCTTCACCGGCCACGCCGTCTTCCCGACGATATACTCCTCCATGAAGAACAGCAAAC | CFTGHAVFPTIYSSMKNSKHFSK |
| | | ACTTCTCCAAAGTTCTCTTGATCTCGTCGGTGCTGTGCAGCCTCAACTACGGGCTCACGGCGGTGCTG | VLLISSVLCSLNYGLTAVLGYMI |
| | | GGCTACATGATCTACGGTGACGATGTGCAGTCGCAGGTGACGCTGAACCTGCCCTCCGGCAAGCTCTA | YGDDVQSQVTLNLPSGKLYTKIA |
| | | CACCAAGATCGCCATCGTGATGACGCTGGTGAACCCGTTGGCCAAGTACGCGCTGCTCGTGGCGCCGA | IVMTLVNPLAKYALLVAPITAAV |
| | | TCACGGCGGCCGTCGAGGAGAGGCTGTCTCTGACGCGGGGCAGCGCGCCGGCGAGGGTGGCCATCAGC | EERLSLTRGSAPARVAISTAILA |
| | | ACCGCCATCCTCGCCAGCACGGTGGTGGTGGCGTCGACGGTGCCCTTCTTCGGCTACCTCATGTCATT | STVVVASTVPFFGYLMSFIGSFL |
| | | CATCGGCTCGTTCCTCAGCGTCATGGCGACCGTGCTGTTCCCTTGCCTCTGCTACCTCAAGATCTACA | SVMATVLFPCLCYLKIYKADGIH |
| | | AGGCCGATGGCATCCACCGCACGGAGATGGTGGCCATCGCCGGAATTCTGCTGCTAGGAGTGTTCGTC | RTEMVAIAGILLLGVFVAVTGTY |
| | | GCCGTCACCGGCACGTACACTTCTTTGCTGCAGATTATAGCCACTTTCTGA | TSLLQIIATF* |
| | | SEQ ID NO: 59 | SEQ ID NO: 60 |
| 31 | Xa21_Os01g0612900 | ATGGCATTCGCCGGGGACGCGCGGATCGTCGTCGCCGTGGCGGCGGTGGCGGCGTTCGTCCTCGTCGG | MAFAGDARIVVAVAAVAAFVLVG |
| | | CGTGGCGGCGGAGGGGAAGGGGGAGGGGGGAGGAGGAGGGGTGGGGGTGTGCTTCGAGAGGATATTCA | VAAEGKGEGGGGGVGVCFERIFS |
| | | GCTTCGGGGACTCGCTGACGGACACCGGCAACTTCCTGCTCTCCGTGCCGGAGGACTTCCCGGACCCC | FGDSLTDTGNFLLSVPEDFPDPA |
| | | GCCCGCAGCCTCCCCTACGGCCAGACCTTCTTCGGCCGCCCCTCCGGCCGCTACTCCGACGGCCGCAA | RSLPYGQTFFGRPSGRYSDGRNL |
| | | CCTCCTCGACTTCTTCGGTATCATCCCCCTCCTCCTCTTCATTTCATTTTTCTTCAGATTTTTTTTCT | LDFFGIIPLLLFISFFFRFFFSH |
| | | CTCATCCTGCCATCCGGGACCGGGACCCCCTTCGGCTTCCCTGTTTGTTTTGTTTATTCTGTTGGTGT | PAIRDRDPLRLPCLFCLFCWCRV |
| | | CGGGTCCCCCTCTGGTAA SEQ ID NO: 61 | PLW* SEQ ID NO: 62 |
| 32 | Xa21_OsI_02945 | ATGCCTGCATTAAGGATGAATAAAATGTTAAGCACCGACTATTTAGGGACGCAGGAGCTTCATTCATT | MPALRMNKMLSTDYLGTQELHSF |
| | (Os01g0629400) | TTGCAAGACCACCGAAATATTGGAAGACTCACAGTCTCAAGAGATTGCACTCGATAGAACAGCGGTGG | CKTTEILEDSQSQEIALDRTAVG |
| | | GGTCAACACTTCTAAGTCATCAAAATGTTTGCTCTACTTCTGAAGTATCAGGGGGAAACTTTGGAATC | STLLSHQNVCSTSEVSGGNFGIA |
| | | GCTGAAGTCAGTTTTTTACAGGATGAATATGATGCAGAAACCACCGGTGTACTGCCACCATCATTTTT | EVSFLQDEYDAETTGVLPPSFLS |
| | | GTCCTGTGGCTCTAGATCTATGCTCCCTATATCAGTACCATCATCATCATCATCTAGTCTGGAGACTG | CGSRSMLPISVPSSSSSSLETVL |
| | | TCCTCTTATCTGACTCAACGTACAGCGATCTTCAGGTAAAAGAGACGAACCACAATTCTACAGCAATG | LSDSTYSDLQVKETNHNSTAMDE |
| | | GATGAAAACAATGAGTTCCTTCAGCTGATTCTTAGTAGCAATGATGAAGGATACAATGCTGGAAGTGA | NNEFLQLILSSNDEGYNAGSEFQ |
| | | ATTCCAAGTTTGGGATGTTCTGGACTTCTACTTCTCAGAAAGCTTTTCAGCTGTGCAGTTTGATAGCT | VWDVLDFYFSESFSAVQFDSLMG |
| | | TGATGGGTTTTACAAATGATGTTAGTTCTTCCCATCACGATTGTATGAATCTTGTTGACATGGTAGAG | FTNDVSSSHHDCMNLVDMVERPV |
| | | CGGCCTGTTGCGCTCCTGTCCCTAAATGATACCGAGGAGCAAAATAACTCTACTGATGAGTTCCCTGA | ALLSLNDTEEQNNSTDEFPDDTS |
| | | TGATACATCATCATACCTTCAAATGAAACCCTCAGATTCCGAAACTGAAAGTAATTATGCCTCTCGAG | SYLQMKPSDSETESNYASRDVAV |
| | | ATGTTGCGGTAACTGAATATGTTGATGAAAAGCCACTTTCTAGAGGCCTGCCTGATTTAATGGATGTC | TEYVDEKPLSRGLPDLMDVDSPG |
| | | GACTCACCCGGTCGTCTATCAAAATCAGCAAGATCAAAACAAATCACTCTTGTTCTGGATTTGGACGA | RLSKSARSKQITLVLDLDETLVH |
| | | GACTCTTGTACATTCAACATTGGATCATTGTGACAATGTTGATTTCACCCTTCAAGTTTTCTTTAATA | STLDHCDNVDFTLQVFFNMKNHT |
| | | TGAAGAACCATACAGTTTATGTTAGGCAAAGACCTCATCTGAAGATGTTTCTTGAGAAGGTAGCTCAG | VYVRQRPHLKMFLEKVAQMFELV |
| | | ATGTTTGAGCTTGTCATTTTCACAGCTAGCCAGAGAATTTATGCCGAGCAACTAATAGATAGACTCGA | IFTASQRIYAEQLIDRLDPDGRL |
| | | CCCTGATGGGAGGTTGATTTCACACCGTATTTATCGTGAGTCATGTATATTCTCTGAGGGTTGCTATA | ISHRIYRESCIFSEGCYTKDLTI |
| | | CAAAGGATCTGACAATTCTTGGAGTTGACCTGGCAAAAGTTGTAATAGTTGACAATACTCCACAGGTT | LGVDLAKVVIVDNTPQVFQLQVD |
| | | TTCCAGTTGCAAGTGGACAATGGTATACCAATAAAGAGCTGGTTTGATGACCCCTCAGACCAGGAATT | NGIPIKSWFDDPSDQELVELLPF |
| | | GGTCGAGTTACTCCCATTCCTGGAGACACTTCTTGGTGTGGAGGACGTTAGGCCAATAATCTCGAAGA | LETLLGVEDVRPIISKTFHHTPE |
| | | CATTCCACCATACACCTGAGCAGAATTAG SEQ ID NO: 63 | QN* SEQ ID NO: 64 |
| 33 | Xa21_Os01g0631800 | ATGGCTGCTGGCGTGGGCAAGGTGGGGGAGTGGATAAGGAGGCGGATGATGCCGCGGAGGAGCAAGAG | MAAGVGKVGEWIRRRMMPRRSKS |
| | | CAAGGCGGCCGGCCCGAGCCGGAGCCGGAGCGACGGCGACGGCGACGGCGGCGAGGCCTTGCCGTTGC | KAAGPSRSRSDGDGDGGEALPLP |
| | | CGGCGCCACCGCAGAGGAAGCTCCGGGCGCGGGCGTTGCCGGCGGCGTTGCGGTGGCGGCCGCGGGGG | APPQRKLRARALPAALRWRPRGR |
| | | CGCGTGCTGGCGGTGCTGTACGAGAAGGTGGTGTACCACCTGCTGTGGCTGGTGGAGTCCGTCGTCGT | VLAVLYEKVVYHLLWLVESVVVV |
| | | CGTCGCCAGGCTCTGCTTCTTCGTCATGCGCTTCGGCCTCAAGCAGCTCTGA | ARLCFFVMRFGLKQL* |
| | | SEQ ID NO: 65 | SEQ ID NO: 66 |
| 34 | Xa21_Os01g0717000 | ATGGTGGCGATCGAGAACCAGAGCCAGGGCCAGCGGGCGGCGGAGGCGGCGGCGCAGCCACGGATCCC | MVAIENQSQGQRAAEAAAQPRIP |
| | | CAGGGAGGCGCGGCGGCTACTGCACGAGATGGCGGCGTCGTGGGCGGACGTTGCCGACTGCCGGGCGC | REARRLLHEMAASWADVADCRAL |
| | | TGCAGGTGATTCCGCTCAAGGGCGCCATGACCAACGAGGTGTACCAGGTGCGCTGGCTCAACGGCGCC | QVIPLKGAMTNEVYQVRWLNGAP |
| | | CCCGCCCCCGCAACCACCGCCGCCGCCGAAGGCGGCGAGGCGGAGGCGGAGGCGGCCGCAAGGGAGAG | APATTAAAEGGEAEAEAAARERE |
| | | GGAGGTGAGGAAGGTCCTCGTGCGGATATACGGCGACGGCGTGGAGCTCTTCTTCGACCGCGAGGACG | VRKVLVRIYGDGVELFFDREDEV |
| | | AGGTGCGCACCTTCGAGTGCATGTCTCGCCATGGCCAGGGCCCCCGCCTCCTCGGCCGCTTCCCCAAC | RTFECMSRHGQGPRLLGRFPNGR |
| | | GGCCGCGTCGAGGAGTTCATCCACGCAAGGACACTATCTGCAGCAGACCTTCGTGATCCTGAAATTTC | VEEFIHARTLSAADLRDPEISAL |
| | | AGCTCTGGTGGCGTCAAAACTGAGGGAATTCCACAACCTTGACATGCCTGGCCCGAAATCTGTACTCA | VASKLREFHNLDMPGPKSVLIWD |
| | | TTTGGGACCGACTAAAAAACTGGCTCAAAACTGCAAGGAACCTGTGCTCATCTGATGAATCCAAGAAA | RLKNWLKTARNLCSSDESKKFRL |
| | | TTTCGGCTGGGTAGCCTGGAGAACGAGATTGCTGCATTGGAGAAAGAATTTTCAGGGGATTACCACGG | GSLENEIAALEKEFSGDYHGIGF |
| | | GATTGGTTTTTGCCACAACGATCTTCAGTATGGCAACATCATGATTGATGAAGACACCAACATGTTGA | CHNDLQYGNIMIDEDTNMLTIID |
| | | CTATCATCGATTATGAGTATGCCAGCTTCAATCCAGTTGCGTATGACATCGCTAATCATTTCTGCGAG | YEYASFNPVAYDIANHFCEMAAD |
| | | ATGGCAGCAGACTATCATTCAGAGAAGCCACATGTACTGGACTACAGTAAATATCCAGACACTGATGA | YHSEKPHVLDYSKYPDTDEQERF |
| | | GCAGGAGCGTTTTGTGAAGACGTACCTAAGCAATTCTGTAAGTGAAGAACCTGATACGGAAGAGGTGG | VKTYLSNSVSEEPDTEEVENLLQ |
| | | AGAACCTTCTCCAAAGCATTGAGAAATACACGCTCGCCAGTCATCTAGTTTGGGGCCTCTGGGGAATA | SIEKYTLASHLVWGLWGIISDHV |
| | | ATATCGGATCATGTCAATGATATTGACTTCGACTACAAGGAGTATGCAAGGCAGAGATTCGAGCAGTA | NDIDFDYKEYARQRFEQYWQKKQ |
| | | TTGGCAGAAGAAGCAAGCACTTTTAACTTCATGA SEQ ID NO: 67 | ALLTS* SEQ ID NO: 68 |
| 35 | Xa21_Os01g0797600 | ATGGCGCCCAGAGCAGCTACGGTGGAGAAGGTTGCTGTGGCGCCACCCACCGGGCTTGGTCTTGGCGT | MAPRAATVEKVAVAPPTGLGLGV |
| | | CGGCGGAGGTGTCGGAGCCGGGGGTCCTCACTACAGGGGCGTCCGCAAGCGCCCGTGGGGGCGTTACG | GGGVGAGGPHYRGVRKRPWGRYA |
| | | CCGCGGAGATCCGTGACCCGGCCAAGAAGAGCCGGGTGTGGCTCGGTACCTACGACACGGCAGAGGAG | AEIRDPAKKSRVWLGTYDTAEEA |
| | | GCCGCCCGCGCCTACGACGCCGCCGCTCGAGAGTTCCGGGGTGCCAAGGCAAAAACGAACTTTCCGTT | ARAYDAAAREFRGAKAKTNFPFA |
| | | TGCATCACAGTCGATGGTCGACTGTGGCGGCAGCCCCAGCAGCAATAGCACGGTAGACACCGGTGGCG | SQSMVDCGGSPSSNSTVDTGGGG |
| | | GCGGGGTTCAGACGCCTATGCAGGCCATGCCTCTGCCGCCTACTCTGGACTTGGATTTGTTCCACCGC | VQTPMQAMPLPPTLDLDLFHRAA |
| | | GCGGCTGCTGTGACTGCAGTCGCCGGCACCGGCGTTCGCTTTCCTTTCAGAGGATATCCCGTTGCACG | AVTAVAGTGVRFPFRGYPVARPA |
| | | TCCAGCAACGCATCCTTACTTTTTCTATGAGCAGGCTGCAGCGGCTGCCGCAGCAGCTGCGGCTGGAT | THPYFFYEQAAAAAAAAAAGYRV |
| | | ACCGTGTGATGAAGCTTGCACCGCCGGTCACCGTGGCGGCGGTTGCACAAAGTGACTCCGACTCCTCG | MKLAPPVTVAAVAQSDSDSSSVV |
| | | TCGGTGGTTGATCTCGCGCCGTCACCTCCAGCGGTTACGGCGAACAAGGCGGCAGCTTTCGATCTGGA | DLAPSPPAVTANKAAAFDLDLNR |
| | | TCTGAACCGGCCGCCGCCGGTAGAGAACTAG | PPPVEN* |
| | | SEQ ID NO: 69 | SEQ ID NO: 70 |
| 36 | Xa21_OsJ_04094 | ATGGCCGCGGCGGCGGCGCAGTCCGTGGCGTCGGCTTCGTCGTCGTCCAGCCCCGTCCTGTCGGCCGC | MAAAAAQSVASASSSSSPVLSAA |
| | | GCACCGCCGCCGGCTCAACGACGTCGAGCGAGACGCGTTCGACTACGGCGGCCCGTGCGACGTCGACG | HRRRLNDVERDAFDYGGPCDVDV |
| | | TCGATCATGATGACGACGACGGCGGCGGCGGAGTGCGCCGCGGGCACGGCGCCGGCGTGGCGGGTGTC | DHDDDDGGGGVRRGHGAGVAGVR |
| | | AGGGCGCTGTTCTCGTCGGCCAGGAGGAGCAAGAGGGCGTCCGTGATTATCGATCAGGCATGGCTGCG | ALFSSARRSKRASVIIDQAWLRN |
| | | GAACGTGGTCGCGTGCCTGCTCGGCCTCACCGTCGTCGCCGGGCTCGTGCTGAGCTCGCACCGAGTGA | VVACLLGLTVVAGLVLSSHRVSG |
| | | GCGGCGCCGGCGGCGGGAGGCTCGTGCAGAGGATGGACCTCGGTGACGGCGAAGTGATGGGCTGGACG | AGGGRLVQRMDLGDGEVMGWTEE |
| | | GAGGAGAACTTGACGGCTGTTGCTCGCCAGTCACCTGACACGCCGATGAAGATATGGATGACGCCAGA | NLTAVARQSPDTPMKIWMTPDSE |
| | | TAGTGAAGGTTATGGCAAATGCATTGAACGCCCAAAGAAACATGACAGAATGAACAGTGCAACAGCTG | GYGKCIERPKKHDRMNSATAGYI |
| | | GTTATATAATTGTTAATGCCAATGGTGGATTGAACCAGATGAGACTGGGAATAAGCGACATGGTTGCA | IVNANGGLNQMRLGISDMVAVAK |
| | | GTTGCTAAGCTCATGAATGCAACACTAGTAATCCCTACTCTGGATCACAAATCATTTTGGACTGATCC | LMNATLVIPTLDHKSFWTDPSDF |
| | | AAGTGATTTCAAAGATATATTTGATGTGGAACACTTCAAGAAAACTTTAGAGGGTGACATTTCGATTG | KDIFDVEHFKKTLEGDISIVDSL |
| | | TGGATTCCTTGCCACTAGCTTATAAAGGATTGAAACTTTATATGAGAGCACCCACTTCTTGGGCCAAG | PLAYKGLKLYMRAPTSWAKASYY |
| | | GCTTCTTACTACAGAGCTTTCTCAAGGACACTGAAGAAGGCCAAAGTTGTGAAATTCACACATACAGA | RAFSRTLKKAKVVKFTHTDSRIV |
| | | TTCACGAATTGTTAACAATGGTCTCCCCCCATCCATCCAACGACTCCGTTGTCGGGCAAATTATGAGG | NNGLPPSIQRLRCRANYEALRFH |
| | | CACTAAGATTCCACAAAGAAATTGAAGAGCTGAGTACTGCTCTAGTTGATAGACTTAGGAATGGATCC | KEIEELSTALVDRLRNGSNHYIA |
| | | AATCATTATATCGCACTTCACTTAAGGTATGAGAAGGACATGCTCTCATTTACTGGTTGCAGCCACAA | LHLRYEKDMLSFTGCSHNLTHKE |
| | | CCTAACACATAAAGAGGCAGATGAGCTGAGAGAAATGAGACTCAACGTGCGGCACTGGAAAGAGAAGG | ADELREMRLNVRHWKEKEINSRE |
| | | AAATCAACAGTAGGGAGAGGAGACTCCAAGGACGCTGCCCTATGACTCCTCGTGAAGTAGCTCTTTTC | RRLQGRCPMTPREVALFLKAMGY |
| | | CTCAAAGCTATGGGCTATCCTTCCAGCACAAAGATCTATATTGTAGCAGGTGAAATATATGGTGGACA | PSSTKIYIVAGEIYGGHSMDSLK |
| | | TAGCATGGATTCATTAAAGGCTGAATATCCAAACATATATACACATTATAGTCTGGCAACGGTTGATG | AEYPNIYTHYSLATVDELEPFKL |
| | | AGTTAGAGCCTTTCAAGCTATACCAAAACAGATTAGCTGCCTTGGACTATAATGTAGCCGTCCAAAGT | YQNRLAALDYNVAVQSDVFVYTY |
| | | GATGTTTTTGTGTATACGTATGATGGAAATATGGCTAAAGCAGTACAGGGTCACAGAAGATTTGAAGG | DGNMAKAVQGHRRFEGFQKTINP |
| | | ATTCCAAAAAACCATCAACCCAGACAGGCAGAAGCTTGTGGGACTCATTGACAAGCTTGATGAAGGTA | DRQKLVGLIDKLDEGTLTWNEFQ |
| | | CACTAACATGGAATGAGTTCCAAAGTGAAGTGAAGATACATCATGAAAACAGACTTGGGGGTCCATAT | SEVKIHHENRLGGPYQRLSGRSP |
| | | CAGAGGTTATCTGGTCGAAGTCCAAGGCAAGAGGAGTACTTCTACGCAAATCCCCTTCCAGGGTGCTT | RQEEYFYANPLPGCLCKRMQRIK |
| | | GTGCAAAAGAATGCAAAGGATTAAATGA SEQ ID NO: 71 | * SEQ ID NO: 72 |
| 37 | Xa21_Os01g0934100 | ATGGCGCAGAGGACGCTGGAGCTGACGCTGATCTCGGCCAAGGATCTCAAGGACGTGAACCTCCTCTC | MAQRTLELTLISAKDLKDVNLLS |
| | | CAAGATGGAGGTCTACGCCGTCGTCTCGCTCTCCGGCGACCCGCGCTCGCGGCAGCGGATCGCCACCG | KMEVYAVVSLSGDPRSRQRIATD |
| | | ACCGCGCCGGCGGCCGCAACCCGGCGTGGAACGCCTCCCCGCTCCGGTTCACCGTCCCGGCATCCGGC | RAGGRNPAWNASPLRFTVPASGA |
| | | GCCGGGTCCCTCCACGTCCTCCTCCGCGCCGAGCGCGCCCTCGGCGACCGCGACGTCGGCGAGGTCCA | GSLHVLLRAERALGDRDVGEVHI |
| | | CATCCCTCTCTCGGAGCTCCTCTCCAGCGCCCCCGACGGCGCCGTCCCCGCCAAGTTCGTCTCCTACC | PLSELLSSAPDGAVPAKFVSYQV |
| | | AGGTCCGCAAGATCTCCTCCGGGAAGCCACAGGGCGTCCTCAATTTCTCCTACAAGATCGGCGAGGTA | RKISSGKPQGVLNFSYKIGEVTQ |
| | | ACCCAATCCGGCTCCTACCCTGGTGCCTCTCCTCCGGTCGCGTACGGCCAATCTCCACCGGCGCCAGC | SGSYPGASPPVAYGQSPPAPAYP |
| | | CTACCCGCCCTCCGCCGCCGCCGCCGCCGCGGCCTACCCGCCCCAATCCACCTACCCACCACCGACCG | PSAAAAAAAYPPQSTYPPPTAYP |
| | | CCTACCCAACGGCAGCGAAGGCCGATGGGTCAGCCGCCGCGGCCTACCCACCCCAGTCCGCCTACCCA | TAAKADGSAAAAYPPQSAYPPPG |
| | | CCTCCGGGTAAGGGAAACGAGCCATCGACGGCTTACCCACCTCCCGCCGGCTACCCGCCGGCGACCGG | KGNEPSTAYPPPAGYPPATGSSK |
| | | AAGTAGTAAGCCGGCGAAGGCGGGTGAGCCGGTGACGGCCTACCCAGCCGCCGCGGGACCGAGCACCG | PAKAGEPVTAYPAAAGPSTAAPP |
| | | CCGCGCCACCGCCGCAGTACGGCTACGGATACCCCGCCCAGCCGCCGCCGCCGCAAGCCGGCTACGGC | PQYGYGYPAQPPPPQAGYGYPPP |
| | | TACCCGCCCCCGCCGCCGCAAGCCGGGTACGGCGGCGGCTACGGCTACCCGCCGCAGGCTGGGTACGG | PPQAGYGGGYGYPPQAGYGGYQQ |
| | | CGGCTACCAGCAGCAGGCCGTGAAGCCGGCGAAGAAGAACAACTTCGGGATGGGGCTCGGAGCCGGGC | QAVKPAKKNNFGMGLGAGLLGGA |
| | | TGCTCGGAGGCGCCCTCGGTGGCCTCCTCATCGGAGACGCCATCTCCGACGCGTCGGCGTATGACGCC | LGGLLIGDAISDASAYDAGYDAG |
| | | GGCTACGACGCCGGGTTCGACGACGGCGGTGGCTTCGATTTCTAG | FDDGGGFDF* |
| | | SEQ ID NO: 73 | SEQ ID NO: 74 |
| 38 | Xa21_OsJ_05190 | ATGATGAGGAAGCAATACTCTTTCACTCTTTGCCTGATGCTCCTGCTTGTGGCTGCTCTGCTTTGCAC | MMRKQYSFTLCLMLLLVAALLCT |
| | (Os02g0123800) | CATGAACACCATCTCCGTCGAAGCAGGAAGGAGTGGTTACAATTCAGTGAATCCCGATGGAAGGAGTG | MNTISVEAGRSGYNSVNPDGRSG |
| | | GCTACAATTCAGTCAATCCCGATGGAAGGAGTGGTTACAATTCAGTCAATCCGAATGGGAGGGGTGGC | YNSVNPDGRSGYNSVNPNGRGGY |
| | | TACAATTCGGTCAATCCCAATGACGGCGGTGGCAGCCCATAG | NSVNPNDGGGSP* |
| | | SEQ ID NO: 75 | SEQ ID NO: 76 |
| 39 | Xa21_Os02g0125300 | ATGGACGCCTTCTACTCGACCTCGTCGGCGTACGGCGCGGCGGCGAGCGGCTGGGGCTACGACTCGCT | MDAFYSTSSAYGAAASGWGYDSL |
| | | GAAGAACTTCCGCCAGATCTCCCCCGCCGTCCAGTCCCACCTCAAGCTCGTTTACCTGACACTATGCG | KNFRQISPAVQSHLKLVYLTLCV |
| | | TCGCGCTGGCTGCGTCGGCGGTGGGCGCATACCTGCACGTCGCCTTGAACATCGGCGGGATGTTGACC | ALAASAVGAYLHVALNIGGMLTM |
| | | ATGCTCGGGTGCGTGGGGAGCATCGCCTGGTTGTTCTCGGTGCCTGTCTATGAGGAGAGGAAGAGGTT | LGCVGSIAWLFSVPVYEERKRFG |
| | | TGGGATTCTCTTGGCCGCTGCCCTGCTGGAAGGGGCTTCAGTTGGGCCTCTGATCAAGCTTGCTGTAG | ILLAAALLEGASVGPLIKLAVDF |
| | | ACTTTGACTCAAGCATTCTCGTAACAGCATTTGTTGGAACTGCCATTGCGTTTGGGTGCTTCACTTGC | DSSILVTAFVGTAIAFGCFTCAA |
| | | GCTGCCATTGTTGCCAAGCGTAGGGAGTACCTCTACCTTGGTGGTTTGCTCTCTTCTGGCCTCTCCAT | IVAKRREYLYLGGLLSSGLSILL |
| | | CCTGCTCTGGCTGCAGTTTGCCGCATCCATCTTTGGCCACTCCACCGGCAGCTTCATGTTTGAGGTTT | WLQFAASIFGHSTGSFMFEVYFG |
| | | ACTTTGGCCTGTTGATCTTCCTGGGGTACATGGTGTATGACACGCAGGAGATCATCGAGAGGGCTCAC | LLIFLGYMVYDTQEIIERAHHGD |
| | | CACGGTGACATGGACTACATCAAGCACGCACTCACCCTCTTCACTGACTTCGTGGCCGTCCTTGTCCG | MDYIKHALTLFTDFVAVLVRILV |
| | | GATCCTCGTCATCATGCTCAAGAACGCGTCTGACAAGTCGGAGGAGAAGAAGAGGAAGAAGAGGTCTT | IMLKNASDKSEEKKRKKRS* |
| | | GA SEQ ID NO: 77 | SEQ ID NO: 78 |
| 40 | Xa21_Os02g0134300 | ATGGGCAAGAGGAAGTCCAGAGCAAAACCACCTCCCAAGAAGAGAATGGACAAGCTTGATACTGTCTT | MGKRKSRAKPPPKKRMDKLDTVF |
| | | TTGCTGTCCATTCTGCAACCACGGAAGTAGTGTTGAGTGTCGCATTGATCTAAAGAACTTAATCGGCG | CCPFCNHGSSVECRIDLKNLIGE |
| | | AGGCCTCTTGCAGAATTTGTTTGGAAACTTTTAGCACAGCTGTTAATGCTCTGACTGAACCCATCGAC | ASCRICLETFSTAVNALTEPIDI |
| | | ATATACAGCGAATGGATCGACGAATGCGAGCGCGTGAACAACCCTGAGGAGGACAACGCCAACCACTA | YSEWIDECERVNNPEEDNANHYE |
| | | TGAAGATGACAATGCCAGCCGCTACCGCTACGACGAAGAAGAATAG | DDNASRYRYDEEE* |
| | | SEQ ID NO: 79 | SEQ ID NO: 80 |
| 41 | Xa21_OSJNBa0023I17.12 | ATGGGTTGTTTTTGTATATTTGGCAAGAGAAGGGCAACCCGACAGCAAAGTTCTCAGCATAATGATGA | MGCFCIFGKRRATRQQSSQHNDD |
| | | CCCCTCTGGTGATACGAATATAACAAAATTCACTTACAAGGAGCTTTCAAGGGTTACTGAAAATTTTA | PSGDTNITKFTYKELSRVTENFS |
| | | GCCCGTCTAATAAGATTGGTGAGGGAGGTTTTGGATCTGTGTATAAGGGAATGCTAAGGAATGGAAAG | PSNKIGEGGFGSVYKGMLRNGKL |
| | | CTTGTTGCCGTCAAGGTGCTATCTTTAGAGTCAAGACAGGGAGCCAAGGAATTTCTGAACGAACTCAT | VAVKVLSLESRQGAKEFLNELMA |
| | | GGCAATTTCAAACGTATCTCATGAAAATCTTGTCAAGCTTTATGGCTATTGTGTGGAAGGAAACCAAA | ISNVSHENLVKLYGYCVEGNQRI |
| | | GAATCCTTGTCTACAATTATCTTGAAAATAATAGCCTTGCACAAACACTTTTGGGTTATGGCCACAGC | LVYNYLENNSLAQTLLGYGHSNS |
| | | AACAGCAACATCCAGTTCAACTGGGCAACTCGAGTAAATATTTGTGTTGGTATCGCCCGTGGACTAAC | NIQFNWATRVNICVGIARGLTYL |
| | | ATACCTCCATGAAGTTGTAAATCCCCACATTGTTCACCGGGATATCAAAGCAAGCAATATACTTCTAG | HEVVNPHIVHRDIKASNILLDKD |
| | | ATAAAGATCTTACGCCGAAAATATCTGATTTCGGTTTAGCAAAGCTTTTGCCTCCAGATGCATCACAT | LTPKISDFGLAKLLPPDASHVST |
| | | GTTAGCACAAGGGTTGCTGGAACATTAGGTTACTTGGCTCCAGAGTATGCCATTCGAGGACAAGTGAC | RVAGTLGYLAPEYAIRGQVTRKS |
| | | ACGGAAGTCAGATGTTTATAGTTTTGGTGTTCTGTTACTAGAAATAGTTAGTGGGAGATCCAACACCA | DVYSFGVLLLEIVSGRSNTNTRL |
| | | ATACAAGATTACCCTATGAAGATCAGATACTTCTTGAAAGGACATGGGTGCACTATGAAGAGGGTGAT | PYEDQILLERTWVHYEEGDLEKI |
| | | TTGGAGAAAATCATAGACGCTTCTTTGGGTGATGACTTGGATGTTGCACAAGCCTGTATGTTCCTGAA | IDASLGDDLDVAQACMFLKIGLL |
| | | AATTGGGCTTTTGTGTACACAAGATGTCACGAAACATCGACCCACAATGTCGATGGTTGTCCGCATGC | CTQDVTKHRPTMSMVVRMLTGEM |
| | | TAACAGGTGAAATGGATGTTGAGCTGGCGAAGATCAGCAAGCCCGCAATAATCAGTGACTTTATGGAT | DVELAKISKPAIISDFMDLKVRS |
| | | CTTAAGGTCAGGAGCATGAGGAAAGAAGTGGACATAGTTTCATCCTCAACATCCACATTGCTATCTTC | MRKEVDIVSSSTSTLLSSIMAHS |
| | | CATCATGGCACACTCTTCTCCTTTGTTGTCGCAAGAGACAACAGAAGCCTCCATGACATTCACTGCGA | SPLLSQETTEASMTFTAISDRE* |
| | | TATCAGATCGTGAGTGA SEQ ID NO: 81 | SEQ ID NO: 82 |
| 42 | Xa21_Os02g0181300 | ATGGATCCGTGGATTAGCAGCCAGCCTTCGCTGAGCCTGGACCTCCGCGTCGGGCTGCCGGCGACGGC | MDPWISSQPSLSLDLRVGLPATA |
| | | GGCCGTCGCCATGGTTAAGCCCAAGGTGCTCGTCGAGGAGGACTTCTTTCACCAGCAGCCTCTCAAGA | AVAMVKPKVLVEEDFFHQQPLKK |
| | | AAGACCCAGAGGTTGCGGCGCTGGAGGCGGAGCTGAAGCGGATGGGCGCGGAGAACCGGCAGCTGAGC | DPEVAALEAELKRMGAENRQLSE |
| | | GAGATGCTGGCGGCGGTGGCGGCCAAGTACGAGGCGCTGCAGAGCCAGTTCAGCGACATGGTCACCGC | MLAAVAAKYEALQSQFSDMVTAS |
| | | CAGCGCCAACAACGGCGGCGGCGGCGGCGGCAACAACCCGTCGTCCACCTCCGAGGGCGGCTCCGTCT | ANNGGGGGGNNPSSTSEGGSVSP |
| | | CGCCGTCGAGGAAGCGCAAGAGCGAGAGCCTCGACGACTCTCCCCCGCCGCCGCCGCCGCCGCACCCA | SRKRKSESLDDSPPPPPPPHPHA |
| | | CACGCGGCGCCGCACCACATGCACGTCATGCCCGGCGCCGCCGCCGCCGGCTACGCCGACCAGACCGA | APHHMHVMPGAAAAGYADQTECT |
| | | GTGCACCTCCGGCGAGCCCTGCAAGCGCATCCGCGAGGAGTGCAAGCCCAAGATCTCCAAGCTCTACG | SGEPCKRIREECKPKISKLYVHA |
| | | TCCACGCCGACCCATCCGACCTCAGCCTGGTGGTGAAAGATGGGTACCAATGGAGGAAGTATGGTCAG | DPSDLSLVVKDGYQWRKYGQKVT |
| | | AAGGTCACCAAGGACAACCCCTGCCCAAGAGCCTACTTCAGATGCTCATTTGCTCCGGCCTGCCCTGT | KDNPCPRAYFRCSFAPACPVKKK |
| | | CAAGAAGAAGGTTCAGAGAAGCGCGGAGGACAACACGATCCTCGTGGCGACGTACGAGGGGGAGCACA | VQRSAEDNTILVATYEGEHNHGQ |
| | | ACCACGGCCAGCCGCCGCCGCCGCTGCAGTCGGCGGCGCAGAACAGCGACGGCTCCGGCAAGAGCGCC | PPPPLQSAAQNSDGSGKSAGKPP |
| | | GGGAAGCCACCCCATGCGCCGGCGGCGGCGCCGGCGCCGGCGCCGGTGGTGCCGCACCGTCAGCACGA | HAPAAAPAPAPVVPHRQHEPAVV |
| | | ACCGGCCGTCGTCAACGGCGAGCAGCAGGCCGCGGCGGCGTCGGAGATGATCAGGCGGAACCTGGCCG | NGEQQAAAASEMIRRNLAEQMAM |
| | | AGCAGATGGCGATGACGCTGACGCGTGACCCAAGCTTCAAGGCGGCGCTCGTCACCGCCCTCTCCGGC | TLTRDPSFKAALVTALSGRILEL |
| | | CGCATCCTCGAGCTCTCGCCGACCAAGGATTGA | SPTKD* |
| | | SEQ ID NO: 83 | SEQ ID NO: 84 |
| 43 | Xa21_OsJ_05634 | ATGGCCTGGGAGGGGGTTGTCGGCGCGGTTGATGCAGCTAGAATGTCGAAGCAACAAGTAGTGGAAGC | MAWEGVVGAVDAARMSKQQVVEA |
| | | AGAGCACCTGATGAAAGCTCTTCTGGAGCAGAAGGATGGTTTAGCACGCAGAATATTTTCGAAAGCTG | EHLMKALLEQKDGLARRIFSKAG |
| | | GAATAGATAACACATCTGTCCTGCAAGCTACTGATGAGTTCATCTCTAGACAGCCAAAGGTTGTTGGC | IDNTSVLQATDEFISRQPKVVGD |
| | | GATACAAGTGGACCGATCATAGGGTCAAGCTTTGTATCAATTTTGGATAATGCAAGGAAGCATAAGAA | TSGPIIGSSFVSILDNARKHKKE |
| | | AGAATATGCTGATGAGTTTGTATCTGTTGAGCACATATTGCGAGCATTCACAGAAGATAAGAGGTTTG | YADEFVSVEHILRAFTEDKRFGQ |
| | | GACAACAGCTGTTCAGAGATCTCAAAATTGGGGAGAATGAGTTGAAGGAAGCTATCTCCGCTGTACGT | QLFRDLKIGENELKEAISAVRGS |
| | | GGCAGTCAGCGGGTCACAGATCAGATTGTTTTTCTACAGAGGTATCTCTTTGTCTATCTCTCTATTCA | QRVTDQIVFLQRYLFVYLSIHNL |
| | | CAACTTGAGAGGAGACTCTGCCGATGGCAATGATCCGGAGGGGAAGTACCAGGCTCTAGAGAAGTATG | RGDSADGNDPEGKYQALEKYGID |
| | | GTATTGACATGACAGAATTAGCTCGACGTGGAAAACTGGATCCTGTTATTGGCCGTGATGATGAAGTG | MTELARRGKLDPVIGRDDEVRRC |
| | | CGACGATGCATTCAGATCCTTTGCAGGAGAACTAAGAATAATCCTGTAATTATTGGTGAGCCAGGAGT | IQILCRRTKNNPVIIGEPGVGKT |
| | | TGGCAAAACTGCTATTGCTGAAGGATTGGCTCAACGTATTGTGCGGGGAGATGTTCCTGAACCTTTAC | AIAEGLAQRIVRGDVPEPLQNRK |
| | | AGAATAGAAAGCTTATTTCATTGGACATGGGAGCATTACTTGCTGGGGCTAAATTCCAAGGTCAATTT | LISLDMGALLAGAKFQGQFEERL |
| | | GAAGAGAGACTTAAGGCTGTTCTGAAGGAGGTCACTGCCTCGAATGGGCAAATCATCTTGTTCATTGA | KAVLKEVTASNGQIILFIDEIHT |
| | | TGAGATCCACACTATTGTTGGTGCAGGAGCAGCAGGTGGGGCAATGGATGCTGGTAATTTGCTGAAGC | IVGAGAAGGAMDAGNLLKPMLGR |
| | | CAATGTTAGGCCGGGGTGAACTCCGTTGCATTGGAGCAACAACATTAGATGAGTACAGGAAGTACATT | GELRCIGATTLDEYRKYIEKDAA |
| | | GAGAAGGATGCTGCTCTGGAGCGTAGGTTCCAGCAGGTTTATTGTGGTGAGCCAGCAGTTGAGGATAC | LERRFQQVYCGEPAVEDTISILR |
| | | AATCTCTATATTGCGTGGTCTAAGAGAGCGGTACGAACTGCATCATGGTGTCAAAATATCAGATGGCG | GLRERYELHHGVKISDGALVSAA |
| | | CTCTTGTTTCTGCAGCTGTTTTATCAGATCGCTACATCACTGGACGTTTTTTGCCTGATAAAGCTATT | VLSDRYITGRFLPDKAIDLVDEA |
| | | GACCTAGTTGATGAAGCAGCTGCTAAGCTGAAAATGGAGATAACATCGAAACCTATTGAGTTGGATGA | AAKLKMEITSKPIELDERLSKLE |
| | | GCGACTGAGCAAGCTTGAAGCTGATCTAGAATCTCTCAAGCAGAAGCAGAAAAACCTTTCAGAACATT | ADLESLKQKQKNLSEHWEYEKSL |
| | | GGGAATATGAGAAGTCCTTGATGACCCGCATAAGATCGATCAAGGAAGAGACTGACAGAGTTAACCTG | MTRIRSIKEETDRVNLEIEAAER |
| | | GAGATTGAAGCTGCTGAGAGAGAATATGATTTGAACCGTGCAGCTGAGCTCAAGTACGGAACACTTCT | EYDLNRAAELKYGTLLSLQKQLE |
| | | GTCGCTACAAAAACAGCTTGAAGAGGCTGAGAACAAACTCATGGAATTCCAGCAGTCAGGCAAGTCAA | EAENKLMEFQQSGKSMLREEVTD |
| | | TGCTTCGAGAAGAGGTGACTGATGTTGACATTGCTGAGATTGTTAGCAAGTGGACTGGTATTCCAGTG | VDIAEIVSKWTGIPVSNLQQSEK |
| | | TCAAACCTTCAACAATCTGAAAAGGAAAAGCTACTGCTATTGGAAGATGTACTCCACAAGAGGGTTAT | EKLLLLEDVLHKRVIGQDIAVKS |
| | | TGGCCAAGATATTGCAGTGAAGTCAGTGGCTAATGCAATCCGTCGTTCTAGGGCAGGCCTATCAGACC | VANAIRRSRAGLSDPNRPIASLM |
| | | CTAACCGTCCCATAGCAAGCTTAATGTTTATGGGTCCAACAGGTGTAGGAAAGACCGAACTGGGGAAA | FMGPTGVGKTELGKTLAEFLFNT |
| | | ACACTAGCTGAATTCCTTTTCAACACAGAGAATGCTCTAATACGGATTGATATGAGCGAGTACATGGA | ENALIRIDMSEYMEKHAVSRLVG |
| | | AAAGCATGCAGTCTCCCGTTTGGTTGGAGCCCCTCCTGGATATATTGGTTATGGGGAAGGGGGCCAAC | APPGYIGYGEGGQLTEAVRRRPY |
| | | TGACAGAAGCTGTTCGTCGTAGGCCTTATTCTGTGGTTCTTTTCGATGAAATTGAGAAAGCGCACCAG | SVVLFDEIEKAHQDVFNILLQLL |
| | | GATGTGTTCAACATCTTATTGCAACTTTTGGATGACGGAAGAATAACCGACTCTCAGGGCAGAACAGT | DDGRITDSQGRTVSFTNCVIIMT |
| | | CAGCTTTACTAACTGTGTTATCATCATGACTTCAAACATTGGGTCGTCATTGATTCTTGACACGCTTA | SNIGSSLILDTLRNTSDSKEAVY |
| | | GAAACACATCAGACAGCAAGGAGGCAGTTTATGAAATAATGAAGAAGCAAGTGATTGACATGGCTCGG | EIMKKQVIDMARQSFRPEFLNRI |
| | | CAGTCATTCCGGCCAGAGTTTTTGAATAGGATAGATGAGTACATTGTATTCCAGCCGCTGGACACCAC | DEYIVFQPLDTTEINRIVEIQLN |
| | | AGAGATAAATCGCATCGTGGAGATCCAGCTGAACCGAGTCAAGAACCGGCTGAGGCAACAGAAGATTC | RVKNRLRQQKIHLQYTPEAVEHL |
| | | ATCTGCAGTACACACCAGAAGCTGTGGAGCATCTTGGCTCTCTCGGCTTTGACCCTAACTACGGCGCT | GSLGFDPNYGARPVKRVIQQMVE |
| | | AGGCCTGTGAAGAGGGTGATCCAGCAGATGGTGGAGAACGAGATCGCCCTCAGTGTCCTGAAGGGCGA | NEIALSVLKGDFKEDDTILVDVS |
| | | CTTCAAAGAAGACGACACCATCCTCGTCGACGTGAGTTCCGTGGCCATCGCGAAAGGGCTTGCTCCAC | SVAIAKGLAPQKKLVLQRLENAN |
| | | AGAAGAAGCTTGTCCTCCAGAGGTTAGAGAATGCAAACTTAGAGTTAGTCGCCAATGACTAG | LELVAND* |
| | | SEQ ID NO: 85 | SEQ ID NO: 86 |
| 44 | Xa21_Os02g0267000 | ATGTCCTCCATCCACTTCTTCCCGGCCACCTCCCAGGCCGCCGCCCCGTCGCGCCGCCCCCTCACCAA | MSSIHFFPATSQAAAPSRRPLTK |
| | | ATCCTCCCCAAAACCCCCCGCCGCCGCCACCGCCGCGTTCCACCGCGGCGGAGGCAACGAGCCGGTGC | SSPKPPAAATAAFHRGGGNEPVL |
| | | TCTTCCCGCGCCTCTTCCTCAGGAGGCGTGCCCGGCCCGCCGCGGCGCCGGCCGAGGCGCCGCCGGTG | FPRLFLRRRARPAAAPAEAPPVG |
| | | GGCCCCGACGGCAGCAACGGCGGCGGGGGAGGAGGAGAGGCGGATGGTGGGGGCGGAGGGGACGACGA | PDGSNGGGGGGEADGGGGGDDEE |
| | | GGAGGATGAGGGGACGAGGAAGGGGCTGCTGCCGGAGTGGCTCAGCGTCACGACGGACGACGCCAAGA | DEGTRKGLLPEWLSVTTDDAKTV |
| | | CGGTGCTGGCCGCCATCGCCATCTCGCTCGCCTTCCGCTCCTTCGTCGCCGAGCCGCGCTTCATCCCC | LAAIAISLAFRSFVAEPRFIPSL |
| | | TCGCTCTCCATGTTCCCCACCTTCGACGTCGGCGACCGCATTGTCGCCGAGAAGGTTACATACTACTT | SMFPTFDVGDRIVAEKVTYYFRK |
| | | TAGAAAGCCATGTATAAATGACATAGTAATCTTCAAAAGTCCACCAGTGCTGCAGGAGGTCGGTTATA | PCINDIVIFKSPPVLQEVGYTDN |
| | | CTGACAATGATGTTTTCATCAAACGAATTGTTGCCAGGGAGGGAGATGTTGTTGAGGTTCATAAAGGA | DVFIKRIVAREGDVVEVHKGKLV |
| | | AAACTAGTTGTCAATGGTGAAGTTAGAAATGAAGAGTTCATTCTTGAGCCACCTTCTTATGACATGAA | VNGEVRNEEFILEPPSYDMNPVQ |
| | | CCCAGTGCAAGTACCTGAGAACTCAGTTTTTGTAATGGGAGATAACCGAAACAACAGTTACGATTCAC | VPENSVFVMGDNRNNSYDSHVWG |
| | | ATGTGTGGGGCCCTCTTCCTTCTAAGAACATATTGGGGAGATCCATTTTCCGGTATTGGCCTCCTGGC | PLPSKNILGRSIFRYWPPGRIGS |
| | | CGAATAGGGAGCACTACTACTGATTGCCTCATCCCTGAGACAAATCCAAGCTCGCTTATTGATGTCAA | TTTDCLIPETNPSSLIDVKLAK* |
| | | ACTGGCAAAGTAG SEQ ID NO: 87 | SEQ ID NO: 88 |
| 45 | Xa21_Os02g0327000 | ATGTTGGGGCATCTGGTTGGGCTGGTGAAGGTGCGGGTGGTGAGGGGCGTCAACCTCGCCGTCCGCGA | MLGHLVGLVKVRVVRGVNLAVRD |
| | | CCTCCGCTCCAGCGACCCCTACGTCATCGTCCGCATGGGCAAGCAGAAGTTGAAGACACGAGTCATAA | LRSSDPYVIVRMGKQKLKTRVIK |
| | | AAAAGACTACCAATCCGGAGTGGAACGATGAACTCACCCTCTCGATTGAAGATCCAGCAGTTCCTGTT | KTTNPEWNDELTLSIEDPAVPVR |
| | | AGATTGGAAGTGTATGACAAAGACACATTCATCGATGATGCAATGGGCAATGCGGAGCTGGACATTCG | LEVYDKDTFIDDAMGNAELDIRP |
| | | CCCATTGGTGGAGGTTGTCAAGATGAAGATTGAGGGTGTTGCTGACAACACGGTTGTGAAGAAAGTGG | LVEVVKMKIEGVADNTVVKKVVP |
| | | TACCCAATAGACAGAACTGCCTAGCTGAAGAGAGCACGATATATATCTCTGAGGGGAAGGTGAAGCAA | NRQNCLAEESTIYISEGKVKQDV |
| | | GACGTGGTTCTCAGACTAAGGGATGTGGAATGCGGGGAAATTGAGCTCCAGCTTCAGTGGGTCGACAT | VLRLRDVECGEIELQLQWVDIPG |
| | | CCCAGGTTCTAAGGGTGTATGA | SKGV* |
| | | SEQ ID NO: 89 | SEQ ID NO: 90 |
| 46 | Xa21_Os02g0661100 | ATGGATTTGAGCAATAGCTCACCTGTCATCACCGATCCGGTGGCGATCAGCCAGCCGTTGTTGGGCGG | MDLSNSSPVITDPVAISQPLLGG |
| | | CCTGCCTTCAAATCTGATGCAGTTTTCAGTCATGCCCGGTGGCTACTCCAGCTCTGGCATGAACGTTG | LPSNLMQFSVMPGGYSSSGMNVG |
| | | GTGTCAGTAGGCTCAAAATCGAGGAAGTCCTTGTCAATGGACTGCTTGATGCCATGAAATCCTCGTCA | VSRLKIEEVLVNGLLDAMKSSSP |
| | | CCTCGCAGGAGGCTGAATGTAGCATTTGGCGAGGACAATTCATCTGAAGAAGAAGACCCTGCTTACAG | RRRLNVAFGEDNSSEEEDPAYSA |
| | | CGCTTGGATGGCAAAATGTCCTTCTGCTTTGGCTTCCTTCAAGCAAATTGTAGCCAGTGCACAAGGGA | WMAKCPSALASFKQIVASAQGKK |
| | | AGAAGATTGCTGTGTTCCTAGACTACGACGGCACGCTGTCGCCTATTGTGGATGATCCTGACAAAGCA | IAVFLDYDGTLSPIVDDPDKAVM |
| | | GTGATGTCTCCCGTGATGAGAGCTGCTGTGAGAAATGTTGCGAAGTACTTCCCCACTGCAATTGTCAG | SPVMRAAVRNVAKYFPTAIVSGR |
| | | CGGAAGGTCCCGCAATAAGGTGTTTGAATTTGTAAAACTGAAGGAGCTTTATTATGCTGGAAGCCATG | SRNKVFEFVKLKELYYAGSHGMD |
| | | GTATGGACATAATGGCATCTTCAGCAAATCATGAGCATAGTGCTGAAAAGAGCAAACAGGCCAATCTC | IMASSANHEHSAEKSKQANLFQP |
| | | TTCCAACCTGCACGCGAGTTTCTGCCAATGATCGATGAGGTTACCAAGTCCCTCTTGCAAGTTGTCAG | AREFLPMIDEVTKSLLQVVSGIE |
| | | TGGAATTGAAGGTGCAACTGTTGAGAACAACAAATTCTGCGTTTCTGTACATTATCGCAACGTTGCAG | GATVENNKFCVSVHYRNVAEKRW |
| | | AGAAGCGTTGGAAACTGGTCGCACGGCTCGTAAACGAAGTGCTGGAGGCTTTTCCTCGTCTCAAAGTA | KLVARLVNEVLEAFPRLKVTNGR |
| | | ACCAATGGACGAATGGTTTTAGAGGTTCGTCCGGTGATCGACTGGGACAAGGGAAAGGCTGTGGAGTT | MVLEVRPVIDWDKGKAVEFLLQS |
| | | TCTGCTCCAGTCACTCGGGCTAGATGACTCTGAAAATGTGATTCCCATCTACATTGGAGACGACAGAA | LGLDDSENVIPIYIGDDRTDEDA |
| | | CTGACGAAGACGCTTTCAAGGTACTTCGACAGCGAAACTGCGGCTATGGAATACTAGTTTCACAGGTT | FKVLRQRNCGYGILVSQVPKETE |
| | | CCCAAGGAAACTGAAGCCTTCTACTCGCTAAGAGACCCATCTGAAGTGATGGAGTTCCTCAATTTCTT | AFYSLRDPSEVMEFLNFLVRWKK |
| | | GGTGAGATGGAAGAAGCACTCAGTGTGA | HSV* |
| | | SEQ ID NO: 91 | SEQ ID NO: 92 |
| 47 | Xa21_Os02g0664000 | ATGCCGTCTCGCACCGCCCCGTTCATCTCTCGCCTCCGGCTCCTCCGAACCGCCGCCGCCCTCTCCCC | MPSRTAPFISRLRLLRTAAALSP |
| | | GTCCTCGTCCTCCGCGCCGCCGCGGCACATCCCCCGCATCCTCCGCGCTAGCCCTGTCGGTGCGCCTC | SSSSAPPRHIPRILRASPVGAPH |
| | | ACCCGCCGCGCGTCTCCGCAGCCGTCTCGCCGCTGGTCCGTCCGGTCGCCGCCGGGTTCGCGCTGTTC | PPRVSAAVSPLVRPVAAGFALFS |
| | | AGCATGGCCACCGCCGCCTCCTCCGCCGCATCCGTCCACGACTTCACCGTCAAGGATGCAAGTGGCAA | MATAASSAASVHDFTVKDASGKD |
| | | AGATGTGGACCTGAGCACCTACAAGGGGAAAGTCCTCCTTATCGTCAATGTTGCATCTCAGTGTGGCT | VDLSTYKGKVLLIVNVASQCGLT |
| | | TGACCAATTCCAACTACACGGAGCTGAGCCAGTTGTATGAGAAGTACAAGGACCAGGGTTTTGAGATC | NSNYTELSQLYEKYKDQGFEILA |
| | | CTTGCTTTCCCATGCAACCAGTTTGGTGGACAGGAACCTGGCACTAATGAGGAGATTGTCCAATTTGC | FPCNQFGGQEPGTNEEIVQFACT |
| | | TTGCACCCGCTTCAAGGCCGAGTATCCAATTTTTGATAAGGTTGATGTTAATGGTGACAACACTGCAC | RFKAEYPIFDKVDVNGDNTAPIY |
| | | CCATCTACAAGTTTCTGAAGTCCAGCAAAGGTGGTCTTTTCGGTGACAATATCAAATGGAACTTCTCC | KFLKSSKGGLFGDNIKWNFSKFL |
| | | AAGTTCTTGGTCGACAAAGAGGGGCGTGTTGTGGAGCGCTATGCGCCAACTACTTCTCCACTCAGCAT | VDKEGRVVERYAPTTSPLSMEKD |
| | | GGAGAAGGACATCAAGAAGCTGCTTGGAAGTTCTTAA | IKKLLGSS* |
| | | SEQ ID NO: 93 | SEQ ID NO: 94 |
| 48 | Xa21_Os02g0671300 | ATGCGTTTGCACGCCGGCGCAGTGCAACTCCAGTATTTCATGCCCCAGCAGGGAGCCGCCGCCGCCGC | MRLHAGAVQLQYFMPQQGAAAAA |
| | | CGCCGCCGACCACCACCAGGCCGACTCGGCTACCGCGTGCAGCGCCTCCACTTCGCCGGCCGCCGCCG | AADHHQADSATACSASTSPAAAA |
| | | CCGCCACCATGTGGGAGTATCATCACCAGCTGTCGACGCACGCGGCCTTGCAGCCCTCGTCCTCTTTT | ATMWEYHHQLSTHAALQPSSSFP |
| | | CCCTACAGTTACTGGAGCCCCTACTCCGGGTCGACGGCGCTCGCCGGCTCGGCCTTCGCTGCCGACTC | YSYWSPYSGSTALAGSAFAADSS |
| | | CTCCTCGTCGTCGACTGACGTCATGCGTCTCCCGGCCGCCGGCGAGCACGCCCACGGACACGGCTGGA | SSSTDVMRLPAAGEHAHGHGWSH |
| | | GCCACGGCGAACTAAGCAACAGCACCACCGGCGGCTGCTACAGGGAGAACTTCCTCGATCTGCTCGCG | GELSNSTTGGCYRENFLDLLASK |
| | | TCGAAGAACGTGACGCCGGAAATGTTCGAGGAAGTCCCCGCCGCCGAGCACTACAACGTCGCGCCGGC | NVTPEMFEEVPAAEHYNVAPAGT |
| | | GGGGACGACCCTGACGACGGCACGGTCGTCGTTCGATCACGCTCGCTCCGACGTCTCGCCGATCAAGT | TLTTARSSFDHARSDVSPIKYEI |
| | | ACGAGATCGCCGGCTCGCCGTTGTACTTGGGTGGCACTAACACCGTCCTCCAGGTCCAGGACATGACC | AGSPLYLGGTNTVLQVQDMTMMS |
| | | ATGATGAGTAGCACGCCAGCATGCTACGGCAAACACCACCACCACCATCATCAGCTGACGAAGGAAGG | STPACYGKHHHHHHQLTKEGSCS |
| | | CAGCTGCAGCCATCAGCAGCAGGAGCAGCACGAGCTTGCGATTAGCCCCATGGCCTCATTTCTGCAGC | HQQQEQHELAISPMASFLQQISS |
| | | AAATAAGCTCCGGCAGTGCTAGTGTTGGCGTGCACAACAGTAGCCTTGATTATTCAGGCTTAGGAGAT | GSASVGVHNSSLDYSGLGDQPDK |
| | | CAGCCTGACAAGATTTGCTGCCAGGACGGCCGGGAAATGGAGGCTAGTCCTTTTAGCATGAGGAGCTT | ICCQDGREMEASPFSMRSLPDLG |
| | | GCCGGATCTTGGGTCATTCGCCGGCTACACGCCGGCCATCGAGTCGACGTCGGTGCAACCGTACATGA | SFAGYTPAIESTSVQPYMRCANS |
| | | GATGCGCCAATTCGTCTGACAGCAATCGACAAGAACAGGAGACTGTGCCGGCAAGGAGTAGTAGCAGC | SDSNRQEQETVPARSSSSGSGAA |
| | | GGCAGTGGAGCGGCGGCGACTGATCGTAAGAAGAGGAAGTTAGAGGAAAGGCAGGAAAGCACTGTCAA | ATDRKKRKLEERQESTVKKSKQE |
| | | GAAGTCCAAGCAAGAGGCTTCCAAAGCATCACCTCCTAAGCAACCAGTGCCTAAAGTAAAGATAGGGG | ASKASPPKQPVPKVKIGEKITAL |
| | | AGAAGATCACAGCACTTCAGCAAATCGTTTCGCCGTTTGGGAAGACGGATACAGCATCGGTACTGTTT | QQIVSPFGKTDTASVLFETIKYI |
| | | GAAACGATCAAGTATATCAAGTTTCTGCACGAACAAGTACAGTTGCTAAGTGAGCCGTACACGAACTC | KFLHEQVQLLSEPYTNSSRSNKQ |
| | | CAGCAGGAGCAACAAGCAGGGTAACAGCGTTCCATGGGGAGATCAAGCAGAAGCGAGCAAGGGAGAGA | GNSVPWGDQAEASKGETTEHDLR |
| | | CGACGGAGCATGACCTGAGGAACAGGGGGCTTTGCTTGGTGCCTGTCTCGTGGACGCCCGAAGTGTAC | NRGLCLVPVSWTPEVYRDGNAMD |
| | | CGGGACGGCAACGCGATGGACTATTGGACGCCGGCGTACCGGGGCTGCCTGTACCGGTGA | YWTPAYRGCLYR* |
| | | SEQ ID NO: 95 | SEQ ID NO: 96 |
| 49 | Xa21_Os02g0677300 | ATGGACGTTTCTGCTGCGCTCAGCAGCGACTACTCGTCGGGGACGCCGTCGCCGGTGGCGGCCGACGC | MDVSAALSSDYSSGTPSPVAADA |
| | | CGACGACGGCTCCTCCGCCTACATGACGGTGTCGTCGGCGCCGCCCAAGCGGCGGGCGGGGCGGACCA | DDGSSAYMTVSSAPPKRRAGRTK |
| | | AGTTCAAGGAGACGCGGCACCCCGTGTTCAAGGGCGTGCGCCGGAGGAACCCCGGGAGGTGGGTGTGC | FKETRHPVFKGVRRRNPGRWVCE |
| | | GAGGTGCGCGAGCCGCACGGCAAGCAGCGGATATGGCTCGGGACGTTCGAGACGGCCGAGATGGCGGC | VREPHGKQRIWLGTFETAEMAAR |
| | | GCGCGCGCACGACGTCGCCGCGCTCGCGCTCCGCGGCCGCGCCGCCTGCCTCAACTTCGCCGACTCGC | AHDVAALALRGRAACLNFADSPR |
| | | CGAGGCGCCTCCGCGTCCCGCCCATTGGCGCAAGCCACGACGACATACGGAGGGCGGCGGCTGAGGCG | RLRVPPIGASHDDIRRAAAEAAE |
| | | GCCGAGGCATTCCGGCCGCCACCAGATGAGAGCAATGCGGCCACCGAGGTGGCAGCCGCCGCATCGGG | AFRPPPDESNAATEVAAAASGAT |
| | | CGCCACCAATTGGAATGCCGAACAGTTCGCCTCCCACCCGTACTACGAGGTCATGGACGATGGGCTGG | NWNAEQFASHPYYEVMDDGLDLG |
| | | ACTTGGGGATGCAGGGCTACCTCGACATGGCGCAAGGGATGCTCATTGACCCGCCTCCAATGGCCGGT | MQGYLDMAQGMLIDPPPMAGDPA |
| | | GATCCTGCCGTAGGTGGCGGGGAAGACGACAACGATGGCGAGGTCCAGCTATGGAGCTACTGA | VGGGEDDNDGEVQLWSY* |
| | | SEQ ID NO: 97 | SEQ ID NO: 98 |
| 50 | Xa21_Os02g0681200 | ATGGGCTTCCCGGTGGGCTACTCGGAGCTGCTGCTCCCGAGGCTGCTGCTCCAGGTGCTCCTCCTCCT | MGFPVGYSELLLPRLLLQVLLLL |
| | | CGGCCACCTCCATCGCTTCCTCCTCTGGGCCTTCCACGCAGTCGGCCTCGGCGACCTCATCGACAACC | GHLHRFLLWAFHAVGLGDLIDNP |
| | | CGCCGGGGCTGGCGGCCACGGAGCAGGACCTGATGCTGCAGGGGCGCGGCGGCGGGATGGCGGAGGGT | PGLAATEQDLMLQGRGGGMAEGW |
| | | TGGGCGTCGTCGTCGTCGTCGGCGCTCCAGCACCGCCGGCCCGAGTTCAGGGCGATTCCGCCGATGGC | ASSSSSALQHRRPEFRAIPPMAI |
| | | CATCGAGGAGGCCCTCCCCGTGGTGAGGTTCGACGAGCTGGTGGCGTCCGCCCCCGCCGCGGTGTGCG | EEALPVVRFDELVASAPAAVCGG |
| | | GTGGCGGCGACTGCGCGGTCTGCCTCAGCGGCATCTGCGGCCGCGACGAGGTGCGGCGGCTGTCCAAC | GDCAVCLSGICGRDEVRRLSNCR |
| | | TGCCGCCACGTCTTCCACCGCGGCTGCCTCGACCGCTGGATGGCCCACGAGCAGCGCACCTGCCCGCT | HVFHRGCLDRWMAHEQRTCPLCR |
| | | CTGCCGCGCGCCGCTCATCCCCGACGAGCTCTTGCCCGCCGCCTCCGCCCTGCCTGACCCCTCCGACT | APLIPDELLPAASALPDPSDYDL |
| | | ACGACCTCTCGTACTACCCATCCCCGCTGCCACTGGCGCCCACCCCGACGCTGCTGCGACCACACGAG | SYYPSPLPLAPTPTLLRPHELLL |
| | | CTGCTGCTCAACGGCTTGGGCGGTTTCCAGTGA | NGLGGFQ* |
| | | SEQ ID NO: 99 | SEQ ID NO: 100 |
| 51 | Xa21_Os02g0682300 | ATGGGGTTCCCTCTGGTGTGCTACTGCATGGCGATCCCCAAGCCGCTCATCGCCCTGGCCAAGCTCCT | MGFPLVCYCMAIPKPLIALAKLL |
| | | CGCCGCCATCAGGGAGGCCCTCCAGCTGATGCTCTTCGTCGTCGGGATCTGCCACCACCCGGAGCGAT | AAIREALQLMLFVVGICHHPERS |
| | | CGGGCCGCCCGGCTGCCGTCGACGCCCCGCTGCCCGACGAGGTGAAGGACCGCCTCCCGCCCCTCGAG | GRPAAVDAPLPDEVKDRLPPLEF |
| | | TTCGCCCAGCTGCTCGCGGCCTCGGAGCACGGCTGTCACGGCTGCGACGACGACGAGGCGGTGGCGGG | AQLLAASEHGCHGCDDDEAVAGC |
| | | GTGCATCGTGTGCCTGGAGAGGCTGGAGGCGGATGACGTGGTGCGGCGGCTGGGCAACTGCGCGCACG | IVCLERLEADDVVRRLGNCAHAF |
| | | CGTTCCACCGCGGCTGCATCGACCGGTGGATCGACCTCGGCCGGTTGACGTGCCCGCTGTGCCGCTCC | HRGCIDRWIDLGRLTCPLCRSNL |
| | | AACCTGCTGCCGCGCGCGCGCCCCGCCGACGGCCCGCGCGGGCGGCTGGGCCGCCTCGCCACCCGCCT | LPRARPADGPRGRLGRLATRLTG |
| | | CACGGGCGTCGTCTGGTGA | VVW* |
| | | SEQ ID NO: 101 | SEQ ID NO: 102 |
| 52 | Xa21_Os02g0703600 | ATGCCCCTGCGTGATGGTGTCCAGCCCGGAGGCGGCGCGGTTCGTGCTGGTGACGCAGGCGCACCTCT | MPLRDGVQPGGGAVRAGDAGAPL |
| | | TCAAGCCCACCTTCCCGGCGAGCAAGGAGCGGATGCTGGGTCCCCAGGCCATCTTCTTCCAGCAGGGC | QAHLPGEQGADAGSPGHLLPAGR |
| | | GACTACCACGCCCACCTCCGCCGCATCGTCTCCCACGCCTTCTCCCCCGAGTCCATCCGCGCCTCCGT | LPRPPPPHRLPRLLPRVHPRLRP |
| | | CCCCGCCATCGAGGCCATCGCGCTCCGCTCCCTCCACTCCTGGGACGGCCAGTTCGTCAACACCTTCC | RHRGHRAPLPPLLGRPVRQHLPR |
| | | AAGAGATGAAGACTTACGCGCTGAATGTGGCATTGCTGTCCATCTTCGGGGAGGAGGAGATGCGCTAC | DEDLRAECGIAVHLRGGGDALHR |
| | | ATCGAGGAGCTGAAGCAGTGCTACCTGACGCTGGAGAAGGGGTACAACTCGATGCCGGTGAACCTGCC | GAEAVLPDAGEGVQLDAGEPAGH |
| | | GGGCACCCTGTTCCACAAGGCCATGAAGGCCCGGAAGCGGCTGGGCGCCATTGTGGCCCACATCATCT | PVPQGHEGPEAAGRHCGPHHLGQ |
| | | CGGCCAGGCGCGAGCGGCAACGCGGGAGCGACCTGCTGGGGTCGTTCGTGGACGGCCGCGAGGCCCTC | ARAATRERPAGVVRGRPRGPHRR |
| | | ACCGACGCCCAGATCGCCGACAACGTCATCGGCGTCATCTTCGCCGCCCGCGACACCACCGCCAGCGT | PDRRQRHRRHLRRPRHHRQRPHL |
| | | CCTCACCTGGATGGTCAAGTTCCTCGGCGACCACCACGCCGTCCTCAAGGCCGTCACCGAAGAGCAGC | DGQVPRRPPRRPQGRHRRAAADC |
| | | TGCAGATTGCCAAGGAGAAAGAGGCGTCGGGCGAGCCGCTGTCATGGGCGGACACGCGGCGGATGAAG | QGERGVGRAAVMGGHAADEDDEP |
| | | ATGACGAGCCGGGTCATCCAGGAGACGATGAGGGTGGCGTCCATCCTCTCCTTCACCTTCAGGGAGGC | GHPGDDEGGVHPLLHLQGGRGGR |
| | | CGTGGAGGACGTGGAATACCAAGGGTACCTGATCCCCAAGGGCTGGAAAGTGCTACCTCTGTTCCGCA | GIPRVPDPQGLESATSVPQHPPQ |
| | | ACATCCACCACAACCCCGCCCACTTCCCCTGCCCGGAAAAGTTCGACCCGTCCCGGTTCGAGGTCAGC | PRPLPLPGKVRPVPVRGQHHEPT |
| | | ATCACGAACCCACTCTTCCGTGTTTTTGTTCCCTTCCCTCCCTTTCCTTTCCCGGTGCGGCCGACTTC | LPCFCSLPSLSFPGAADFLAKSV |
| | | CTAGCCAAGTCGGTCCGCGGTTTGCGAGCAGTGGCCGGTATGGGTTTTCCTGACCCCGCGTCCGTCCC | RGLRAVAGMGFPDPASVPSLLQV |
| | | GTCTCTGTTGCAGGTGGCGCCCAAGCCCAACACGTTCATGCCGTTCGGGAACGGGACCCACTCGTGCC | APKPNTFMPFGNGTHSCPGNELA |
| | | CGGGCAACGAGCTCGCCAAGCTGGAGATGCTCGTGCTCTTCCACCACCTCGCCACCAAGTACAGGTGG | KLEMLVLFHHLATKYRWSTSKSE |
| | | TCCACGTCCAAGTCCGAGAGCGGCGTCCAGTTCGGCCCCTTCGCGCTGCCGCTCAACGGCCTCCCCAT | SGVQFGPFALPLNGLPMSFTRKN |
| | | GAGCTTCACCCGCAAGAACACCGAGCAGGAGTGA | TEQE* |
| | | SEQ ID NO: 103 | SEQ ID NO: 104 |
| 53 | Xa21_Os02g0704300 | ATGATTAGGGAAGTACTCTCTGAGGCACAGGTTGTGCTTTCTACTAACACAGGGGCAGCCGATCCACT | MIREVLSEAQVVLSTNTGAADPL |
| | | TATCAGGAGAACATGTTGTTTTGACTTGGTAATCATAGATGAAGCAGGGCAAGCAATAGAACCCTCCT | IRRTCCFDLVIIDEAGQAIEPSC |
| | | GCTGGATTCCCATATTGCAGGGAAAGCGTTGCATTCTCGCTGGTGATCATTGCCAGCTTGCACCAGTA | WIPILQGKRCILAGDHCQLAPVI |
| | | ATTTTGTCAAGAAAGGCCTTAGATGGAGGGCTTGGTAAATCTTTGTTGGAAAGAGCTTCGTCATTGCA | LSRKALDGGLGKSLLERASSLHN |
| | | CAATGGATTACTGACAACAAGGTTGACAGTGCAGCACAGAATGAATGACTCAATAGCTAGTTGGGCAT | GLLTTRLTVQHRMNDSIASWASK |
| | | CTAAAGAGATGTATCATGGACTGCTCAAATCTTCTCATTCTGTTGCTTCACAAACTCTTGCTGACTCT | EMYHGLLKSSHSVASQTLADSPV |
| | | CCAGTTGTGAAGGCTACCTGGATAACACGGTGCCCACTGCTTTTACTTGATACTCGAATGCCATATGG | VKATWITRCPLLLLDTRMPYGAL |
| | | GGCCCTGGACACTGGTTGTGGGGAACAAATAGACCTTTCTGGTACAGGCTCATTTTATAATGATGGAG | DTGCGEQIDLSGTGSFYNDGEAD |
| | | AGGCAGATATTGTGACACAACATGTTCTCAACCTTGTACATTGTGGAGTATCTCCAACAGCGATAGCC | IVTQHVLNLVHCGVSPTAIAVQS |
| | | GTTCAATCACCCTACATTGCTCAAGTGCAGTTGTTAAGAGAGAGGCTAGAAGAATATCCAGGGCTTTC | PYIAQVQLLRERLEEYPGLSGVE |
| | | TGGGGTTGAGGTTTCAACCATAGATAGCTTTCAAGGGAGGGAAGCTGATGCAGTTGTCATATCGATGG | VSTIDSFQGREADAVVISMVRSN |
| | | TTCGATCAAACCCACTGGGAGCGGTAGGTTTCCTTGGTGACAGCAGACGCATGAATGTGGCTATTACA | PLGAVGFLGDSRRMNVAITRACR |
| | | AGGGCATGCCGGCATGTCACCGTGGTATGTGACACCTCTACTATCTGTCACAGCACCTTCCTGGCAAG | HVTVVCDTSTICHSTFLARLLRH |
| | | GCTCCTTCGTCATATCAGGCGATATGGGCAAGTGAAACATGTAGTGCCTAGTTCTTTGGATGGGGTGT | IRRYGQVKHVVPSSLDGVSGLGF |
| | | CTGGCCTTGGCTTCAGTCAACCCACTCTGCCGTCCATCAGTTAG | SQPTLPSIS* |
| | | SEQ ID NO: 105 | SEQ ID NO: 106 |
| 54 | Xa21_Os02g0730800 | ATGGACGCCGCCCGCGCCTCCCTCCTCTTGGCCGGCGGCCTCGCCGTATCCACCTCCACCTCCGCCGT | MDAARASLLLAGGLAVSTSTSAV |
| | | CGCCACTGCCGCCCAAACCGTCTCGATTCCTCACCTATCGCCGCACACCCGCCAACGGCGACAGCGCC | ATAAQTVSIPHLSPHTRQRRQRR |
| | | GCTTCCTCCGCCTGGCCTCCGCTGCGGCGTCGTCTCCCCCGCCGCTCCCCGCTGCTTCCGCGCAGCCG | FLRLASAAASSPPPLPAASAQPH |
| | | CATTGCTCTAGGTGGGTCGTGGTGATGGAGAGACCCCCCGCGCCAGCGGGAGGCGGCGAGGTGTCCCG | CSRWVVVMERPPAPAGGGEVSRA |
| | | TGCGGAGGCCGTCGACCACTACGTCGCCACACTGGCCAGGGTATTGGGGAGCCAGGAGGAAGCACAGA | EAVDHYVATLARVLGSQEEAQMR |
| | | TGCGCATATACGATGCTTCGTGGGATGGGAGCTACGAGTTCAGCTGCGAGATCGACGATGAAGCGTCA | IYDASWDGSYEFSCEIDDEASRD |
| | | AGGGACCTTGCGAAAATGCCCGGGGTATTAGCTGTTAAGCCAGATATGGATAAGGTTGATATGTCAGA | LAKMPGVLAVKPDMDKVDMSEKD |
| | | GAAGGATAGTCATGGCTCAGGCCTCTCAGCAGCTAACCTAGGAAATTTCAGTGATGCTGTTTCCAATC | SHGSGLSAANLGNFSDAVSNHPS |
| | | ACCCTTCTTCTAGTGGGGAAAATGAGTTTTGGCTTGTGCGAATGGAGAAGCCTGGGGTTGAAGTTGTG | SSGENEFWLVRMEKPGVEVVTKA |
| | | ACAAAGGCACAAATGGTGGATCATTATACCCAAATCCTCATGAAAGTGTTGGGGAATGAAAAGGATGC | QMVDHYTQILMKVLGNEKDAQVS |
| | | ACAAGTCAGCATATATCACATTTCATGGGAGAGGGATTATGGCTTCTGCTGTCACATTGACGAAGAAT | IYHISWERDYGFCCHIDEECAKE |
| | | GTGCAAAGGAGCTAGCTGATGTTTCTGGAGTGTTATCTGTTCAACCGGACCCAAATTTTGGATCCGAC | LADVSGVLSVQPDPNFGSDNKNY |
| | | AACAAAAATTACAAAGGTGATGACAGCTTCAAATCGTCGGAAGCTACTCAGGCTAAAGTCAAAACTAA | KGDDSFKSSEATQAKVKTKRLFV |
| | | AAGGCTCTTTGTTACAGGTCTTTCATTTTATACATCTGAGAAAACTTTGCGGGCTGCCTTTGAGCCTT | TGLSFYTSEKTLRAAFEPFGELV |
| | | TTGGTGAACTAGTTGAAGTGAAGATAATAATGGACAAGATATCAAAAAGATCAAAAGGGTATGCTTTC | EVKIIMDKISKRSKGYAFIEYTT |
| | | ATAGAATATACCACAGAAGAAGCTGGAGGTGCTGCTCTGAAAGCAATGAACGGACAGATAATAAATGG | EEAGGAALKAMNGQIINGWMIVV |
| | | CTGGATGATAGTTGTCGATGTTGCTAAACATAGATCGAGGAACCGTCAACCCCCGTAG | DVAKHRSRNRQPP* |
| | | SEQ ID NO: 107 | SEQ ID NO: 108 |
| 55 | Xa21_Os02g0759400 | ATGCCAAGTTGCCGAGCTAGGATGCACGCGCACCAGCACGCGCTGCTCGCGGCGCTCCTCGCGTGCGC | MPSCRARMHAHQHALLAALLACA |
| | | GCTCGCGGCGTCGTCGTCGACGGCCGGAGCGCAGCCGGCGGGGCAGCAGGGGTATGTCTACGGCGACG | LAASSSTAGAQPAGQQGYVYGDV |
| | | TGTCCGGGCAGCAGGTGCACGTCAGCACGACGATGATCGTGCTGCTGGCCGCCGTCGTCGGGGTTTTC | SGQQVHVSTTMIVLLAAVVGVFL |
| | | CTGTTCATCGCGATCTCCACCATCTACCTCCGCCACTGCACCGGTTACGACCCCGCGACGGAGGGCGG | FIAISTIYLRHCTGYDPATEGGV |
| | | CGTCGGCGGGCGCCGCTCCATGATCCTACCTGCCAACAGCTTCGTCTCGAGGCGGCAGCGGCGGCCGC | GGRRSMILPANSFVSRRQRRPRG |
| | | GCGGGCTCGACTCGTCGGTGGTCCGGATGTTCCCCACCATGAAGTACGCCGAGGCGAAGGCGCTGCGG | LDSSVVRMFPTMKYAEAKALRVG |
| | | GTGGGGAAGGTCGCCGGCGCGGCGCTGGAGTGCGCGGTGTGCCTCAGTGAGTTCGAGGACGACGAGAT | KVAGAALECAVCLSEFEDDEMLR |
| | | GCTCAGGTTCTTGCCCAAGTGCAGCCACGCCTTCCACCCGGACTGCATCGGCCAGTGGCTCGCCAGCC | FLPKCSHAFHPDCIGQWLASHVT |
| | | ACGTGACATGCCCCGTCTGCCGCCGGAATCTTGACCCTAACAAGGACACCACCGAGGAGGTGATCATC | CPVCRRNLDPNKDTTEEVIIPAA |
| | | CCGGCCGCCGCCGCCGCCGCCGATCATCCGAACAGCACCTCCAGTGAAATAGTCGTGATACGGCAAGA | AAAADHPNSTSSEIVVIRQEDGA |
| | | AGACGGGGCGCACCCAGCAGCCGTGGTGATCGACGTGGTCACCGAGGAGGACGACGAGGAGCGGAGGA | HPAAVVIDVVTEEDDEERRKEEL |
| | | AGGAGGAGCTAGAGCTTCAGGCGATAGGCACCCAGCTCCGCGCGATGCGGTCACGGCCGGGGCTGCGG | ELQAIGTQLRAMRSRPGLRPKTS |
| | | CCAAAGACGTCGGCGGCGAAGCTTCCCCGGTCGCACTCCACTGGCCACTCCCTCGCCGTCCGGCTCGA | AAKLPRSHSTGHSLAVRLDGDLE |
| | | CGGCGACCTGGAGCGGTACACGCTGCGGCTGCCGGAGCACGTACACAGGGAGATGGTCTCGGCGGGGG | RYTLRLPEHVHREMVSAGEQSVR |
| | | AGCAGAGCGTGCGCCGCGGGAGGAGACTCGGGGAAGGGGTCGGCATGGGCGCTCGGTGCAGCCCCCGG | RGRRLGEGVGMGARCSPRFSRSG |
| | | TTTTCGCGGTCGGGCAGATGGTCATCTTTTCTCTCAAATTCTTTGTCAGGAAAGCTGTCTTTCCTCTC | RWSSFLSNSLSGKLSFLSPSSRR |
| | | CCCGTCGTCGAGGAGAACGCCGGATAGCACCCAGGTGGAAGTGTCATCATCCTCGTCGTCGTCGGCGA | TPDSTQVEVSSSSSSSATKVRGK |
| | | CGAAGGTGAGAGGGAAGCGCGTGGCCGCCGTCGATGTTGCCGACGACGGTTCTGCTCATGGGACTGCA | RVAAVDVADDGSAHGTAQYPGCT |
| | | CAGTACCCTGGTTGCACTGTGGCCAGCTCGGCGGCGGCGGCGGCTGTGGACGTGGAGAAGGCAGCTAC | VASSAAAAAVDVEKAATRQVRT* |
| | | CAGGCAAGTTCGAACATAG SEQ ID NO: 109 | SEQ ID NO: 110 |
| 56 | Xa21_OsJ_08589 | ATGAGGAGGATGTTGCGTTGCTTCTTCGGCGGCGGCGGCGGTGGCGACGAGACGGGAGGTGACGAGGA | MRRMLRCFFGGGGGGDETGGDEE |
| | | GAAGAAGGCGTCGGCGGCGGCGGTGGTGAAGAACAAGAAGGCGGTGAGGCGGATGCGGAGCGCGACGG | KKASAAAVVKNKKAVRRMRSATG |
| | | GGCGGCTCCGGTCGCTGTCGCTGGAGGACCTGTCGCGGACGCTGGCGCAGTCCGGGCTGCAGGCCTTC | RLRSLSLEDLSRTLAQSGLQAFT |
| | | ACGCTGGCGGAGCTCAAGGCCGCCACGCGCAGCTTCTCCGGCAGCAACTTCATCGGCGAGGGCGGATT | LAELKAATRSFSGSNFIGEGGFG |
| | | CGGCCCCGTCTACAAGGGCTTCATCGACGCCAAGCTCCGCCCCGGCATCCTCCAGCCCCAGCACGTCG | PVYKGFIDAKLRPGILQPQHVAV |
| | | CCGTCAAGTACCTCGACGGCGAAGGCGACCAGGGCCACCGCGAATGGCTGGCGGAGGTGGTGTACCTG | KYLDGEGDQGHREWLAEVVYLGM |
| | | GGGATGTTGAGCCATCCGCATCTGGTGAAGCTGATCGGCTACTGCTGCCAAGACGACCACCGGATGCT | LSHPHLVKLIGYCCQDDHRMLVY |
| | | CGTCTACGAGTACATGGCCAGGGGCAGCCTCGAGCACCACCTCTTCAAGAATTTGCTGTCGAGCTTGC | EYMARGSLEHHLFKNLLSSLPWA |
| | | CATGGGCGACGCGGCTGAAGATCGCGGTGGGCGCGGCCAAGGGGCTCGCGTTCCTGCACGACGCCGAC | TRLKIAVGAAKGLAFLHDADTPV |
| | | ACTCCGGTGATCTACCGAGACTTCAAGGCATCCAACATCCTGCTCGACTCGGATTACACGGCGAAGCT | IYRDFKASNILLDSDYTAKLSDF |
| | | CTCGGACTTCGGGCTGGCCAAGGAGGGCCCGCAGGGGGACGCGACCCACGTGACGACGCGCGTGATGG | GLAKEGPQGDATHVTTRVMGTHG |
| | | GGACCCACGGGTACGCGGCGCCGGAGTACATCCTGACGGGGCACCTGACGGCGAAGAGCGACGTGTAC | YAAPEYILTGHLTAKSDVYSFGV |
| | | AGCTTCGGGGTGGTGCTGCTGGAGCTGCTGACGGGGCGGCGGAGCGTGGACAAGCGGCGGCGCGGGCG | VLLELLTGRRSVDKRRRGREQNL |
| | | GGAGCAGAACCTCGTGGACTGGGCGCGGCCGTACCTCCGGCGGCCGGAGCGGCTGCACCGGGTGATGG | VDWARPYLRRPERLHRVMDPSLE |
| | | ACCCCAGCCTGGAGGGCGGCTACTCCGACAGGGCGGCGGCCAAGGCCGCCATGGTGGCGTACCACTGC | GGYSDRAAAKAAMVAYHCLHSVP |
| | | CTCCACAGCGTGCCCAAGTCCCGCCCCCACATGCGCGACGTCGTCGCCGCGCTCGAGCCGCTGCTGCA | KSRPHMRDVVAALEPLLQTSCGD |
| | | GACGTCGTGCGGCGACGTGCTCGCCGGGCCCTTCGTCTACACCGTCCCGTCCGCGCCGGCGCCCGCCG | VLAGPFVYTVPSAPAPAAVVVAK |
| | | CTGTTGTTGTCGCCAAGGACGACGAGAAGAAGGCGGCCGCGGCCGCGGGTGAGGACGGCGAGGAGGAG | DDEKKAAAAAGEDGEEEVAAVAK |
| | | GTGGCGGCGGTGGCCAAGGCGAAGAGGAGGTACGTGGCGTCGGCGGTGCACGCGGAGGGCGCGATGCG | AKRRYVASAVHAEGAMRKGEHRY |
| | | GAAGGGGGAGCACCGGTACGCGAGCTCCGTGTCCGGCTCGCCGAGGCAGAGCAGGGACAGGGGCGCGT | ASSVSGSPRQSRDRGA* |
| | | AG SEQ ID NO: 111 | SEQ ID NO: 112 |
| 57 | Xa21_Os02g0787800 | ATGGAGCGAGCGGAGGAGACGCCGAGGGCGCCGTCGACGGCGCCGTCGGCGAGGGTGTCGATCTGGGA | MERAEETPRAPSTAPSARVSIWE |
| | | GTCGGTGCGCGCGTGCGGGGTGTGGGGGAAGGAGGTGGACAAGGCCGAGCTGCGGAGGCAGGTCGTCA | SVRACGVWGKEVDKAELRRQVVM |
| | | TGCCGCTGTACGCGCGGCGTGCGGTCGCGGCCGCCGTGAAGGCCAAGGACGAGGCCGTCGGCGTGGCG | PLYARRAVAAAVKAKDEAVGVAA |
| | | GCGGCGGCGGAGAGGGGCGAGGAGGTGGAGGGGGAGGTGGAGGTGGAGGCGGCCGTGACGCCGGTGGT | AAERGEEVEGEVEVEAAVTPVVV |
| | | GGTGTTCGTCAACTCGCGGAGCGGTGGTAGGCACGGGCCCGAGCTCAAGGTGCGGCTCCACGAGCTCA | FVNSRSGGRHGPELKVRLHELIS |
| | | TCAGCGAGGAGCAGGTCTTTGATCTCTCTGTCGTGAAGCCTTCTGATTTCATTAACTACGGGTTGGGT | EEQVFDLSVVKPSDFINYGLGCL |
| | | TGTTTGGAGAAATTGGCCGAACAAGGTGACAATTGTGCGGAGACCATTCGAAAAAAATTGAGGATTAT | EKLAEQGDNCAETIRKKLRIMVA |
| | | GGTTGCTGGTGGTGATGGCACAGTTGGTTGGGTACTTGGCTGTCTCACAGATCTTTATAGGTTGAAAA | GGDGTVGWVLGCLTDLYRLKREP |
| | | GAGAACCAGTTCCACCCACAGGTATCATCCCACTTGGTACAGGAAATGATCTTGCCAGATCATTTGGA | VPPTGIIPLGTGNDLARSFGWGG |
| | | TGGGGTGGCTCTTTTCCCTTCGGTTGGCGTTCAGCTGTGAAGCGTTATCTCAGCAAGGCAGCTACAGC | SFPFGWRSAVKRYLSKAATAPTC |
| | | TCCCACATGCCGTCTTGATAGTTGGCAAGCTGTAGTTATGATGCCAGATGGAGAGATAAAAGAGTTGC | RLDSWQAVVMMPDGEIKELPYAL |
| | | CATATGCACTTAAGAAAACGGAACCTGCAGACTGCTTGGAACTTTGCCAGGAGAATGGCACTGAGTTA | KKTEPADCLELCQENGTELPEKA |
| | | CCTGAGAAAGCTTCTTGCTATAAAGGAGTATTCTACAACTACCTTAGCATTGGGATGGATGCCCAAGT | SCYKGVFYNYLSIGMDAQVAYGF |
| | | TGCTTATGGCTTCCACCATCTTCGTGATGAAAAGCCATATCTTGCTCAAGGGCCAGTTGCAAATAAGT | HHLRDEKPYLAQGPVANKLIYAG |
| | | TGATATATGCTGGTTATAGCTGCACACAGGGGTGGTTCTGCACACCTTGTACAGCAAGCCCTCAGTTA | YSCTQGWFCTPCTASPQLRGLKN |
| | | AGGGGGCTTAAGAACATTTTGCGACTTTACATTAAAAAAGTCAATTGTTCTGAATGGGAGCAAGTTAC | ILRLYIKKVNCSEWEQVTMPSSV |
| | | AATGCCTTCAAGTGTTAGATCCCTGGTGGTCTTAAATTTATACAACTACGGCAGTGGAAGGCATCCAT | RSLVVLNLYNYGSGRHPWGDLKP |
| | | GGGGTGATCTTAAACCTGATTACCTTGAAAAGAAAGGTTTTGTTGAAGCTCATTCCGATGATGGATTG | DYLEKKGFVEAHSDDGLLEIFGL |
| | | CTTGAAATATTCGGTCTGAAAGAAGGTTGGCATGCTTCATTCGTTATGGCTGAGCTAATTAAAGCAAA | KEGWHASFVMAELIKAKHIAQAA |
| | | GCACATTGCTCAGGCTGCAGCCATCAAGTTTGAAATGAGGGGTGGCCAATGGAATCGTGCGTATGTTC | AIKFEMRGGQWNRAYVQMDGEPW |
| | | AGATGGATGGAGAGCCATGGAAACAACCACTCCTACAGGAGCAGTCAACCATTATCGAGATTAACAAA | KQPLLQEQSTIIEINKVPYPSLM |
| | | GTTCCTTACCCTTCTCTGATGATAAATGGGGAGCAATGA | INGEQ* |
| | | SEQ ID NO: 113 | SEQ ID NO: 114 |
| 58 | Xa21_OsJ_09250 | ATGGCGGAGGCGGCGGGGGTGGGGGCGGGGGCCGGGCCGGCGAGGAGGACGACGAGGGTGGGGCGCTA | MAEAAGVGAGAGPARRTTRVGRY |
| | (former:Os03g0126800) | CGAGCTCGGCAAGACGATCGGGGAGGGGAGCTTCGCCAAGGTCAAGGTCGCCCGCGACACGCGCACCG | ELGKTIGEGSFAKVKVARDTRTG |
| | | GCGACACCCTCGCCATCAAGGTGCTCGACCGCAACCATGTGCTTCGCCACAAGATGGTCGAGCAGATC | DTLAIKVLDRNHVLRHKMVEQIK |
| | | AAACGGGAGATTTCCACAATGAAGCTAATAAAACATCCAAATGTGGTCCAGTTGCATGAGGTCATGGC | REISTMKLIKHPNVVQLHEVMAS |
| | | TAGCAAATCAAAGATATACATGGTTCTTGAATATGTTGATGGAGGCGAGCTTTTTGATAAGATTGTCA | KSKIYMVLEYVDGGELFDKIVNS |
| | | ATTCTGGGAGGCTAGGAGAAGATGAAGCAAGAAGATACTTCCATCAACTTATAAATGCAGTAGATTAT | GRLGEDEARRYFHQLINAVDYCH |
| | | TGCCACAGCAGAGGAGTGTACCATAGAGATCTGAAGCCAGAGAATCTGCTTCTTGATTCACATGGAGC | SRGVYHRDLKPENLLLDSHGALK |
| | | TCTTAAAGTTTCAGATTTCGGCCTTAGTGCGTTTGCTCCACAAACAAAAGAGGATGGACTTCTGCATA | VSDFGLSAFAPQTKEDGLLHTAC |
| | | CTGCTTGCGGAACACCAAATTATGTTGCACCTGAGGTGCTTGCTGATAAAGGTTATGATGGTATGGCT | GTPNYVAPEVLADKGYDGMAADV |
| | | GCTGATGTATGGTCCTGCGGCATAATCTTATTTGTCCTCATGGCTGGATATTTACCTTTCGATGACCC | WSCGIILFVLMAGYLPFDDPNLM |
| | | CAACCTAATGACCCTTTACAAACTGATCTGCAAGGCAAAAGTTTCTTGTCCACACTGGTTTTCTTCAG | TLYKLICKAKVSCPHWFSSGAKK |
| | | GTGCGAAGAAATTCATTAAGCGCATACTTGATCCTAATCCCTGCACAAGAATAACAATTGCACAAATT | FIKRILDPNPCTRITIAQILEDD |
| | | TTGGAAGATGATTGGTTCAAAAAGGATTACAAACCACCACTTTTTGAGCAAGGTGAAGATGTGAGCCT | WFKKDYKPPLFEQGEDVSLDDVD |
| | | TGATGATGTTGATGCTGCATTCGATTGTTCAGAGGAGAATCTCGTGGCAGAGAAGAGAGAAAAGCCTG | AAFDCSEENLVAEKREKPESMNA |
| | | AATCCATGAATGCATTTGCTCTAATTTCAAGGTCACAGGGATTCAACCTTGGAAATTTATTTGAGAAG | FALISRSQGFNLGNLFEKEMMGM |
| | | GAAATGATGGGAATGGTGAAGCGGGAAACTTCCTTCACATCTCAATGTACACCACAGGAGATCATGTC | VKRETSFTSQCTPQEIMSKIEEA |
| | | TAAAATAGAGGAAGCTTGTGGACCTCTTGGTTTCAATGTGCGGAAACAAAATTATAAGATGAAGTTAA | CGPLGFNVRKQNYKMKLKGDKTG |
| | | AAGGTGATAAGACTGGAAGAAAAGGCCATTTATCTGTAGCAACGGAGGTTTTCGAGGTTGCTCCATCA | RKGHLSVATEVFEVAPSLHMVEL |
| | | CTCCACATGGTTGAGCTTCGTAAAACTGGAGGGGACACGCTGGAGTTTCACAATTTCTACAACAATTT | RKTGGDTLEFHNFYNNFSSELKD |
| | | CTCGTCAGAGTTAAAAGACATAGTGTGGAAATCTGAATCTGACGCAAAAGCAGCAAAGAAGAGGTGA | IVWKSESDAKAAKKR* |
| | | SEQ ID NO: 115 | SEQ ID NO: 116 |
| 59 | Xa21_Os03g0180900 | ATGGCCGGTAGTAGTAGCGAGCAGCAGCTGGTCGCCAACGCGGCGGCTACGACGGTGGCCGGGAACGG | MAGSSSEQQLVANAAATTVAGNG |
| | | GAGCAGGTTCGCGGTGACGTGTGGCTTGCTCAGGCAGTACATGAAGGAGCACAGTGGAAGCAACGGCG | SRFAVTCGLLRQYMKEHSGSNGG |
| | | GCGGTGGCTTCTTGCCGGCGGTGACGGCCATGAGCCTCATGACCGGCGGCGCTGATGCGGAGGAGGCG | GGFLPAVTAMSLMTGGADAEEAA |
| | | GCGCCGGAGGTGAGGAAGACCATGGAGCTCTTCCCTCAGCAGGCTGGGACGCTGAAAGACTCGCAAGA | PEVRKTMELFPQQAGTLKDSQER |
| | | AAGGAAGGAGATCACTGAGAAGGCGGCGCAGCTGACCATCTTCTACGGCGGGAGCGTGGTGGTGTTCG | KEITEKAAQLTIFYGGSVVVFDD |
| | | ACGACTTCCCCGCCGAGATGGCCGGCGAGCTGATGAAGCTCGCCGGCTCCCGCGACAGCACGGCGGCG | FPAEMAGELMKLAGSRDSTAAAA |
| | | GCGGCCGTCTCCGACGCCGGCGCCGCAGCGGGGCAGCCTTGCCTACCAGACATGCCCATCGCGAGGAA | VSDAGAAAGQPCLPDMPIARKVS |
| | | GGTGTCGCTGCAGAGGTTCCTGGAGAAGAGGAAGAACAGGATCGTCGCGGCGGAGCCACTGCCGGAGT | LQRFLEKRKNRIVAAEPLPESEK |
| | | CGGAGAAGAAGGAGGCGGAGCCGAGCAAGCGTGCCAAGAAAGACGACGGCGGCGCGTCGTGGCTCCAG | KEAEPSKRAKKDDGGASWLQVNP |
| | | GTGAACCCCACTCTCAGCCTGTGA | TLSL* |
| | | SEQ ID NO: 117 | SEQ ID NO: 118 |
| 60 | Xa21_OsJ_09990 | ATGGAGCTAGGCGTGCCACTGCCACGACGGCCCGTGCCCGGTAGCTACGGCGTGCCGTTCGTCTCGGC | MELGVPLPRRPVPGSYGVPFVSA |
| | (former:Os03g0225900) | GGTGCGCGACCGCCTCGACTTCTACTACTTGCAGGGGCAGGACAAGTACTTCGAGTCGCGCGCCGAGA | VRDRLDFYYLQGQDKYFESRAER |
| | | GGTACGGCTCCACCGTCGTCCGCATCAACGTCCCGCCTGGCCCCTTCATGGCGCGCGACCCCCGCGTG | YGSTVVRINVPPGPFMARDPRVV |
| | | GTGGCGCTCCTCGACGCCAAGAGCTTCCCCGTCCTCTTCGACGTCGCCAAGGTCGAGAAGCGGGACGT | ALLDAKSFPVLFDVAKVEKRDVF |
| | | GTTCACCGGCACGTTCATGCCGTCCACCTCCCTCACAGGCGGCTACCGCGTCTGCGCCTACCTCGACC | TGTFMPSTSLTGGYRVCAYLDPS |
| | | CGTCCGAGCCCAACCACGCCAAGATCAAGCAGCTGCTCCTCTCCCTCCTGGTCTCTCGCAAGGACGCC | EPNHAKIKQLLLSLLVSRKDAFV |
| | | TTCGTCCCGGTCTTCCGCTCCAACTTCGGCGCGCTCCTCGACACCGTCGAGTTGCAGCTCGCCAGCGG | PVFRSNFGALLDTVELQLASGGG |
| | | CGGCGGCAAGTCGGACTTCACCGCCCTCAACGATGCCACCTCCTTCGAGTTCATCGGCGAGGCGTACT | KSDFTALNDATSFEFIGEAYFGV |
| | | TCGGCGTGCGTCCCTCCGCGTCGAGCTCCCTCGGCACCGGCGGGCCGACCAAGGCCGCCCTGTGGCTC | RPSASSSLGTGGPTKAALWLLWQ |
| | | CTATGGCAGCTCGCCCCGCTCACCACGCTCGGCCTGCCCATGATCATCGAGGATCCGCTCCTCCACAC | LAPLTTLGLPMIIEDPLLHTLPL |
| | | GCTGCCGCTGCCACCCTTCCTCATCAGCTCCGACTACAAGGCGCTGTACGCGTACTTCGCCGCCGCGG | PPFLISSDYKALYAYFAAAASQA |
| | | CGTCGCAGGCGCTCGACGCCGCCGAGGGCCTTGGCCTGTCGCGGGAGGAGGCCTGCCACAACCTGCTG | LDAAEGLGLSREEACHNLLFATA |
| | | TTCGCGACGGCCGGCGAGAAGCTCCACGAGAGGCTCGCCGCGGAGATACGAAGCGCGGTGGCCGACGC | GEKLHERLAAEIRSAVADAGGNV |
| | | CGGCGGCAACGTGACGCTGGCCGCTCTGGAGAAGATGGAGCTGACCAGGTCGGTGGTGTGGGAGGCGC | TLAALEKMELTRSVVWEALRLDP |
| | | TGCGGCTGGACCCGCCGGTCAGGTTCCAGTACGGGCGCGCCAAGGCCGACCTGGAGATCGAGAGCCAC | PVRFQYGRAKADLEIESHDASFA |
| | | GACGCGTCGTTCGCGATCAAGAAGGGGGAGATGCTGTTCGGCTACCAGCCGTGCGCCACCAGGGACCC | IKKGEMLFGYQPCATRDPRVFGA |
| | | GCGGGTGTTCGGCGCCACGGCGAGGGAGTTCGTTGGCGACCGGTTCGTCGGCGAGGAGGGGAGGAAGC | TAREFVGDRFVGEEGRKLLQYVY |
| | | TGCTGCAGTACGTGTACTGGTCGAATGGGCGAGAGACGGAGAACCCTAGCGTTGACAACAAGCAGTGC | WSNGRETENPSVDNKQCPGKNLV |
| | | CCCGGCAAGAATCTTGTGGTGCTCGTCGGTAGGCTGCTGCTCGTCGAGCTCTTCCTCCGGTACGACAC | VLVGRLLLVELFLRYDTFTAEAG |
| | | CTTCACCGCCGAGGCCGGCAAAAAGGTGGTCATCACCGGGGTCACCAAAGCTTCAACCTCCGTCGTCA | KKVVITGVTKASTSVVNRTA* |
| | | ATCGTACTGCTTAA SEQ ID NO: 119 | SEQ ID NO: 120 |
| 61* | Xa21_Os03g0241400 | ATGTCGAGGTGCTTCCCCTACCCGCCGCCGGGGTACGTGCGAAACCCAGTGGTGGCCGTGGCCGCGGC | MSRCFPYPPPGYVRNPVVAVAAA |
| | | CGAAGCGCAGGCGACCACTAAGCTCCAGAAAGAAAGGGAAAAGGCCGAAAAGAAGAAAGAGAAAAAGA | EAQATTKLQKEREKAEKKKEKKS |
| | | GTGACAGGAAAGCTCTTCCACATGGTGAGATATCCAAACATTCAAAGCGAACCCACAAGAAGAGAAAA | DRKALPHGEISKHSKRTHKKRKH |
| | | CATGAAGACATCAATAATGCTGATCAGAAGTCCCGGAAGGTTTCCTCCATGGAACCTGGTGAGCAATT | EDINNADQKSRKVSSMEPGEQLE |
| | | GGAGAAGAGTGGACTCTCAGAAGAGCATGGAGCTCCTTGCTTTACTCAGACAGTGCATGGCTCTCCAG | KSGLSEEHGAPCFTQTVHGSPES |
| | | AGAGTTCACAGGACAGCAGCAAGAGAAGAAAGGTTGTGGTACCCAGTCCTAGCCAAGCTAAGAATGGT | SQDSSKRRKVVVPSPSQAKNGNI |
| | | AACATCCTTCGAATAAAGATAAGAAGAGATCAAGATTCTTCAGCTTCCATTTCTGAGAAATCTAATGT | LRIKIRRDQDSSASISEKSNVVQ |
| | | TGTACAAACACCAGTTCATCAAATGGGTTCAGTTTCATCTCTGCCCAGTAAGAAAAACTCAATGCAAC | TPVHQMGSVSSLPSKKNSMQPHN |
| | | CACACAACACTGAAATGATGGTGAGGACAGCATCAACCCAGCAGCAAAGCATCAAAGGTGATTTTCAA | TEMMVRTASTQQQSIKGDFQAVP |
| | | GCAGTACCGAAACAAGGTATGCCAACCCCAGCAAAAGTCATGCCAAGAGTCGATGTTCCTCCTCCATC | KQGMPTPAKVMPRVDVPPPSMRA |
| | | TATGAGGGCATCAAAGGAAAGGGTTGGCCTTCGTCCTGCAGAGATGTTGGCCAATGTTGGTCCTTCAC | SKERVGLRPAEMLANVGPSPSKA |
| | | CCTCCAAGGCAAAACAAATTGTCCATCCCGCACCTGCTAAGGTTACACAAAGGGTTGATCCTCCACCT | KQIVHPAPAKVTQRVDPPPAKAS |
| | | GCTAAGGCATCTCAGAGAATTGATCCTCAGTTGCCATCCAAGGTTCATATAGATGCTACTCGATCTTT | QRIDPQLPSKVHIDATRSFTKVS |
| | | TACGAAGGTCTCCCAGACAGAGATCAAGCCGGAAGTACAGCCCCCAATTCCGAAGGTGCCTGTGGCTA | QTEIKPEVQPPIPKVPVAMPTIN |
| | | TGCCTACCATCAATCGTCAGCAGATTGACACCTTGCAGCCCAAAGAAGAGCCTTGCTCCTCTGGCAGG | RQQIDTLQPKEEPCSSGRNAEAA |
| | | AATGCTGAAGCTGCTTCAGTATCAGTAGAGAAGCAGTCCAAGTCAGATCGCAAAAAGAGTCGCAAAGC | SVSVEKQSKSDRKKSRKAEKKEK |
| | | TGAGAAGAAAGAGAAGAAGTTCAAAGATTTATTTGTTACCTGGGATCCTCCGTCTATGGAAATGGATG | KFKDLFVTWDPPSMEMDDMDLGN |
| | | ATATGGATCTTGGCAACCAGGATTGGCTGCTTGGTAGTACGAGGAAACCTGATGCTGGCATTGGCAAC | QDWLLGSTRKPDAGIGNCREIVD |
| | | TGCAGAGAAATTGTTGATCCACTTACTTCTCAATCAGCAGAGCAGTTCTCATTGCAGCCCAGGGCGAT | PLTSQSAEQFSLQPRAIHLPDLH |
| | | TCATTTACCAGACCTTCATGTCTATCAGTTGCCATATGTGGTTCCATTCTAG | VYQLPYVVPF* |
| | | SEQ ID NO: 121 | SEQ ID NO: 122 |
| 62 | Xa21_Os03g0265600 | ATGTCGTACTCCAGAGGATCAAGATATGAGTCCCACTCATATTCTACATCGCCCAGACGGAGCAGCCG | MSYSRGSRYESHSYSTSPRRSSR |
| | | TTCAAGGTATAGGAGTCGTTCTAGGAGTGTGGACTCAAGTGATGTTGAAAACCCTGGGAACAACCTGT | SRYRSRSRSVDSSDVENPGNNLY |
| | | ATGTTACTGGCTTATCTGCTCGTGTGACGGATCGAGATCTGGAGAAACACTTTTCTGCTGAGGGAGAG | VTGLSARVTDRDLEKHFSAEGEV |
| | | GTTATTGATGCTAGCATAGTACTTGATCCATGGACAAGGGAATCCCGGGGATTTGGATTTGTTACCAT | IDASIVLDPWTRESRGFGFVTMA |
| | | GGCTACTGTTAAGGAGGCTGATCTTTGCATCAAATATCTAGACCGCTCAGTGTTGGAAGGTCGTGTGA | TVKEADLCIKYLDRSVLEGRVIT |
| | | TAACTGTAGAGAAGCTTTTGTTGACAGCAGCAGGGTAG | VEKLLLTAAG* |
| | | SEQ ID NO: 123 | SEQ ID NO: 124 |
| 63 | Xa21_OsJ_10337 | ATGGCCGGCAAGGGAGACGGGCCGGCGATCGGCATCGACCTCGGGACGACCTACTCGTGCGTCGGCGT | MAGKGDGPAIGIDLGTTYSCVGV |
| | (Os03g0277300) | GTGGCAGCACGACCGCGTCGAGATCATCGCCAACGACCAGGGGAACCGCACCACGCCGTCGTATGTCG | WQHDRVEIIANDQGNRTTPSYVA |
| | | CGTTCACGGACTCGGAGAGGCTCATCGGCGACGCCGCCAAGAACCAGGTGGCCATGAACCCCATCAAC | FTDSERLIGDAAKNQVAMNPINT |
| | | ACCGTCTTCGATGCGAAGCGTCTCATCGGCCGGCGTTTCTCTGACACGTCGGTGCAGAGCGACGCGAA | VFDAKRLIGRRFSDTSVQSDAKL |
| | | GCTGTGGCCGTTCAAGGTGCTCCCCGGCCCCGGCGACAAGCCGATGATCGGCGTGCAGTACAAGGGCG | WPFKVLPGPGDKPMIGVQYKGEE |
| | | AGGAGAAGCAGTTCTCCGCCGAGGAGATCTCGTCCATGGTGCTCAACAAGATGAAGGAGACGGCCGAG | KQFSAEEISSMVLNKMKETAEAY |
| | | GCCTACCTCGGCAGCACCGTCAAGAACGCCGTCGTCACCGTCCCCGCCTACTTCAACGACTCCCAGCG | LGSTVKNAVVTVPAYFNDSQRQA |
| | | CCAGGCCACCAAGGACGCCGGCGTCATCTCCGGCCTCAACGTCATGCGCATCATCAACGAGCCCACCG | TKDAGVISGLNVMRIINEPTAAA |
| | | CCGCCGCCATCGCCTACGGCCTCGACAAGAAGGCCGCCAGCACCGGCGAGAAGAACGTGCTCATCTTC | IAYGLDKKAASTGEKNVLIFDLG |
| | | GACCTCGGCGGCGGCACCTTCGACGTCTCCATCCTCACCATCGAGGAGGGCATCTTCGAGGTCAAGGC | GGTFDVSILTIEEGIFEVKATAG |
| | | CACCGCCGGCGACACCCACCTGGGAGGCGAGGACTTCGACAACCGGATGGTGAACCACTTCGTGCAGG | DTHLGGEDFDNRMVNHFVQEFKR |
| | | AGTTCAAGAGGAAGCACAAGAAGGACATCACCGGCAACCCGAGGGCGCTCCGGCGGCTGAGGACGGCG | KHKKDITGNPRALRRLRTACERA |
| | | TGCGAGAGGGCGAAGAGGACGCTGTCCTCCACCGCACAGACCACCATCGAGATCGACTCGCTCTACGA | KRTLSSTAQTTIEIDSLYEGIDF |
| | | GGGCATCGACTTCTACGCCACCATCACCCGCGCCCGGTTCGAGGAGCTCAACATGGACCTCTTCCGCC | YATITRARFEELNMDLFRRCMEP |
| | | GGTGCATGGAGCCCGTGGAGAAGTGCCTCCGCGACGCCAAGATGGACAAGGCCCAGATCCACGACGTC | VEKCLRDAKMDKAQIHDVVLVGG |
| | | GTCCTCGTCGGAGGCTCCACCCGTATCCCCAAGGTGCAGCAGCTCCTGCAGGACTTCTTCAACGGGAA | STRIPKVQQLLQDFFNGKELCKS |
| | | GGAGCTCTGCAAGAGCATCAACCCCGACGAGGCCGTGGCGTACGGCGCCGCCGTCCAGGCCGCCATCC | INPDEAVAYGAAVQAAILSGEGN |
| | | TCAGCGGCGAGGGCAACCAGAGGGTGCAGGACCTGCTCCTCCTCGACGTCACGCCGCTCTCGCTCGGG | QRVQDLLLLDVTPLSLGLETAGG |
| | | TTGGAGACGGCCGGAGGCGTGATGACCGTGCTGATCCCGAGGAACACCACCATCCCCACCAAGAAGGA | VMTVLIPRNTTIPTKKEQVFSTY |
| | | GCAGGTCTTCTCCACCTACTCCGACAATCAGCCCGGCGTGCTCATCCAGGTGTACGAGGGCGAGAGGA | SDNQPGVLIQVYEGERTRTKDNN |
| | | CGAGGACCAAGGACAACAACCTGCTCGGCAAGTTCGAGCTCACCGGCATCCCTCCGGCGCCGAGGGGC | LLGKFELTGIPPAPRGVPQINVT |
| | | GTGCCCCAGATCAACGTGACGTTCGACATCGACGCGAACGGCATCCTGAACGTGTCGGCGGAGGACAA | FDIDANGILNVSAEDKTTGKKNK |
| | | GACGACGGGGAAGAAGAACAAGATCACCATCACCAACGACAAGGGGCGGCTGAGCAAGGAGGAGATCG | ITITNDKGRLSKEEIERMVQEAE |
| | | AGCGGATGGTGCAGGAGGCCGAGAAGTACAAGGCCGAGGACGAGCAGGTGCGGCACAAGGTGGAGGCC | KYKAEDEQVRHKVEARNALENYA |
| | | CGCAACGCGCTGGAGAACTACGCGTACAACATGCGCAACACGGTGCGCGACGACAAGATCGCCTCCAA | YNMRNTVRDDKIASKLPADDKKK |
| | | GCTCCCCGCCGACGACAAGAAGAAGATCGAGGACGCCATCGAGGATGCCATCAAGTGGCTCGACGGCA | IEDAIEDAIKWLDGNQLAEADEF |
| | | ACCAGCTCGCCGAGGCCGACGAGTTCGAGGACAAGATGAAGGAGCTCGAGAGCCTCTGCAACCCGATC | EDKMKELESLCNPIISKMYQGGA |
| | | ATCTCAAAGATGTACCAGGGCGGCGCCGGTGGTCCGGCGGGCATGGACGAAGACGCCCCCAACGGCGG | GGPAGMDEDAPNGGAGTGGGSGA |
| | | CGCCGGCACCGGCGGTGGCAGCGGCGCTGGCCCCAAGATCGAGGAAGTGGACTAA | GPKIEEVD* |
| | | SEQ ID NO: 125 | SEQ ID NO: 126 |
| 64 | Xa21_OsJ_10412 | ATGGACGGGGCGCCGGTGGCGGAGTTCAGGCCGACGATGACGCACGGCGGCCGGTACCTGCTCTACGA | MDGAPVAEFRPTMTHGGRYLLYD |
| | (Os03g0285800) | CATCTTCGGGAACAAGTTCGAGGTGACGAACAAGTACCAGCCGCCCATCATGCCCATCGGCCGCGGCG | IFGNKFEVTNKYQPPIMPIGRGA |
| | | CCTACGGGATCGTCTGCTCGGTGATGAACTTTGAGACGAGGGAGATGGTGGCGATAAAGAAGATCGCC | YGIVCSVMNFETREMVAIKKIAN |
| | | AACGCGTTCAACAACGACATGGACGCCAAGCGGACGCTCCGGGAGATCAAGCTCCTCAGGCACCTCGA | AFNNDMDAKRTLREIKLLRHLDH |
| | | CCACGAGAACATCATAGGCATCAGGGACGTGATCCCGCCGCCGATCCCTCAGGCGTTCAACGACGTCT | ENIIGIRDVIPPPIPQAFNDVYI |
| | | ACATCGCCACGGAGCTCATGGACACCGACCTCCATCACATCATCCGCTCCAACCAAGAACTGTCAGAA | ATELMDTDLHHIIRSNQELSEEH |
| | | GAGCACTGCCAGTATTTCCTGTATCAGATCCTGCGGGGGCTCAAGTACATCCACTCGGCGAACGTGAT | CQYFLYQILRGLKYIHSANVIHR |
| | | CCACCGCGACCTGAAGCCGAGCAACCTGCTGCTGAACGCCAACTGCGACCTCAAGATCTGCGACTTCG | DLKPSNLLLNANCDLKICDFGLA |
| | | GGCTGGCGCGGCCGTCGTCGGAGAGCGACATGATGACGGAGTACGTGGTCACCCGGTGGTACCGCGCG | RPSSESDMMTEYVVTRWYRAPEL |
| | | CCGGAGCTGCTGCTCAACTCCACCGACTACTCCGCCGCCATCGACGTCTGGTCCGTCGGCTGCATCTT | LLNSTDYSAAIDVWSVGCIFMEL |
| | | CATGGAGCTCATCAACCGCCAGCCGCTCTTCCCCGGCCGGGACCACATGCACCAGATGCGCCTCATCA | INRQPLFPGRDHMHQMRLITEVI |
| | | CCGAGGTGATCGGGACGCCGACGGACGACGAGCTGGGGTTCATACGGAACGAGGACGCGAGGAAGTAC | GTPTDDELGFIRNEDARKYMRHL |
| | | ATGAGGCACCTGCCGCAGTACCCGCGCCGGACGTTCGCGAGCATGTTCCCGCGGGTGCAGCCCGCCGC | PQYPRRTFASMFPRVQPAALDLI |
| | | GCTCGACCTCATCGAGAGGATGCTCACCTTCAACCCGCTGCAGAGGATCACAGTTGAGGAGGCGCTCG | ERMLTFNPLQRITVEEALDHPYL |
| | | ATCATCCTTACCTAGAGAGATTGCACGACATCGCCGATGAGCCCATCTGCCTGGAGCCCTTCTCCTTC | ERLHDIADEPICLEPFSFDFEQK |
| | | GACTTCGAGCAGAAGGCTCTAAACGAGGACCAGATGAAGCAGCTGATCTTCAACGAAGCGATCGAGAT | ALNEDQMKQLIFNEAIEMNPNFR |
| | | GAACCCAAACTTCCGGTACTAG | Y* |
| | | SEQ ID NO: 127 | SEQ ID NO: 128 |
| 65 | Xa21_OsJ_10445 | ATGGCACGGCTCGTGCTCTCCGAGTGCTGCGGCCTCACGCCGCTCCGCCTGCGCGGGCGCGGCGCCAT | MARLVLSECCGLTPLRLRGRGAI |
| | (Os03g0290300) | TGCGCTGCCGGCGCCTCCCAGCCTCACGGCGGGGCCGCGCCGCCCCGTGCCCGCCGCCGCCGCGGGGG | ALPAPPSLTAGPRRPVPAAAAGG |
| | | GAGCCATCCACCGCGAGTGGGCGCTCCGCGTCTCCGCCCCGACCCGCCTCACCTCCGTCGTCGAGGAG | AIHREWALRVSAPTRLTSVVEED |
| | | GATAATAGAGGGGAGGAGGTGGTGGAGGAGGAGGCCAGGGGTTCGTTGGCGGCCGCCGCCGTAGGAGG | NRGEEVVEEEARGSLAAAAVGGD |
| | | GGACGGAGACGGGTTCGACCCCGGGGCGCCGCCGCCGTTCGGGCTCGCGGAGATCCGCGCGGCCATCC | GDGFDPGAPPPFGLAEIRAAIPK |
| | | CCAAGCACTGCTGGGTGAAGGACCCCTGGAGGTCCATGAGCTACGTGCTGCGCGACGTCGTCGTCGTG | HCWVKDPWRSMSYVLRDVVVVLG |
| | | CTCGGCCTCGCCGCCGCCGCTGCGCGCGTCGACAGCTGGCTCGTCTGGCCGCTCTACTGGGCCGCGCA | LAAAAARVDSWLVWPLYWAAQGT |
| | | GGGCACCATGTTCTGGGCGCTCTTCGTGCTGGGTCACGACTGTGGCCATGGGAGCTTCTCAAGCAACG | MFWALFVLGHDCGHGSFSSNAKL |
| | | CCAAGTTGAACAGCGTGGTTGGACACATACTCCACTCCTCCATCCTTGTCCCATACCACGGCTGGAGG | NSVVGHILHSSILVPYHGWRISH |
| | | ATTAGCCACAGGACGCACCACCAGAACCACGGTCATGTCGAGAAAGACGAGTCCTGGCAACCGCTGTC | RTHHQNHGHVEKDESWQPLSERL |
| | | TGAGAGGCTGTACAATAGCCTGGACTATATGACGAAGAAATTGAGGTTCACCATGCCGTTTCCCATGC | YNSLDYMTKKLRFTMPFPMLAFP |
| | | TGGCATTCCCATTATACTTGTTTGCAAGGAGTCCAGGGAAAAAAGGTTCACACTTCAACCCAAGCAGT | LYLFARSPGKKGSHFNPSSDLFQ |
| | | GATCTGTTCCAACCAAATGAGAAGAAGGATGTGATAACATCAACTGCATCCTGGCTGGCAATGGTTGG | PNEKKDVITSTASWLAMVGILAG |
| | | GATTCTTGCTGGTCTGACCTTTGTGATGGGCCCCCTCAAAATGTTAAAGCTCTATGCTGTTCCATACG | LTFVMGPLKMLKLYAVPYVIFVM |
| | | TGATATTTGTTATGTGGCTTGATTTTGTCACATACTTGCACCATCATGGCCATGAGGACAAGCTTCCT | WLDFVTYLHHHGHEDKLPWYRGK |
| | | TGGTACCGTGGAAAGGAATGGAGCTATCTGCGCGGAGGATTGACAACACTTGATCGGGACTACGGGTG | EWSYLRGGLTTLDRDYGWINNVH |
| | | GATCAACAACGTACACCATGACATTGGAACCCATGTCATTCATCATCTTTTCCCACAAATCCCACATT | HDIGTHVIHHLFPQIPHYHLVEA |
| | | ACCATCTTGTCGAGGCGACTGAGGCCGCAAAACCAGTGCTGGGAAAATATTACAAAGAGCCAGAGAAA | TEAAKPVLGKYYKEPEKSAPLPF |
| | | TCAGCTCCCCTCCCATTCCACTTACTTGGAGTGCTAGCCAAGAGCTTAAACAGCGACCACTATGTTAG | HLLGVLAKSLNSDHYVSDTGDVV |
| | | TGACACCGGAGACGTAGTGTACTACCAAACTGACTTGAAAACTAGTTCTTCTGCACAAAGCTCGGATT | YYQTDLKTSSSAQSSD* |
| | | GA SEQ ID NO: 129 | SEQ ID NO: 130 |
| 66* | Xa21_OsI_11115 | ATGGCGCACGCGTCTCCCTCTCCCATGGACTGCCACCACCACCGCCGCCAGCTGCTGCTGCGCTCCCC | MAHASPSPMDCHHHRRQLLLRSP |
| | | GTCGCGGAGGTCCTCGCCGCCGGGGGCCCCGGCCCACGCCGACGAGCGCCTCCACCACCACCGCTTCC | SRRSSPPGAPAHADERLHHHRFL |
| | | TCCGCCCCGGCGCGCTCGCGCGGCTGCGCGACTCCAAGGTCATCGCCCGCTCGCTCCGGTCCGCCGCC | RPGALARLRDSKVIARSLRSAAA |
| | | GCCGCCGCGGCGGTGGCCGCGGTGGAATCGGATCCGAGGAACGAGGTGGAGGAGGACGGCGAGGAGTT | AAAVAAVESDPRNEVEEDGEEFA |
| | | CGCGGTGTACTGCCGGAGGGGAAAGGGGAGGAGAAGGGTGGAGATGGAGGACCGGCATGTGGCCAAGG | VYCRRGKGRRRVEMEDRHVAKVA |
| | | TCGCCCTCGGCGGAGACCCCAAAGTGGCCTTTTTTGGTGTATTCGATGGCCACGGCGGGAAAAGCGCG | LGGDPKVAFFGVFDGHGGKSAAE |
| | | GCGGAATTCGTCGCCGAGAACATGCCCAAGTTCATGGCCGAGGAGATGTGCAAGGTGGACGGCGGCGA | FVAENMPKFMAEEMCKVDGGDSG |
| | | CAGTGGCGAGACTGAGCAGGCAGTGAAGCGGTGCTACCTGAAGACGGACGAGGAATTCCTCAAGAGGG | ETEQAVKRCYLKTDEEFLKREES |
| | | AGGAGAGCGGGGGCGCCTGCTGCGTCACCGCTTTGCTGCAGAAAGACGGCCTCGTTGTGTCTAATGCC | GGACCVTALLQKDGLVVSNAGDC |
| | | GGAGATTGCCGTGCCGTTCTCAGCCGCGCCGGGAAGGCAGAGGCGCTCACCTCTGATCACCGGGCCTC | RAVLSRAGKAEALTSDHRASRED |
| | | GCGCGAGGATGAGAAGGAAAGAATTGAGAATCTGGGTGGATTCGTGGTGAATTACCGAGGAACATGGC | EKERIENLGGFVVNYRGTWRVQG |
| | | GAGTCCAGGGCTCCTTGGCAGTGTCAAGAGGCATCGGTGATGCACACCTCAAGCAGTGGGTGGTGTCT | SLAVSRGIGDAHLKQWVVSDPDT |
| | | GATCCAGACACCACAACTCTGGGTGTTGATTCGCAGTGCGAATTCTTGATTCTTGCCTCTGATGGTTT | TTLGVDSQCEFLILASDGLWDKV |
| | | GTGGGACAAGGTGGAGAACCAGGAGGCAGTTGACATTGCCAGGCCGCTTTGCATCAGCAACGACAAAG | ENQEAVDIARPLCISNDKASRMT |
| | | CTTCTCGCATGACTGCTTGCAGGAGGCTCGTCGAAACTGCGGTTACTAGGGGCTCGACTGACGATATC | ACRRLVETAVTRGSTDDISIVII |
| | | AGCATTGTGATCATACAGCTGCAGCAATTCTCAAGATAA | QLQQFSR* |
| | | SEQ ID NO: 131 | SEQ ID NO: 132 |
| 67 | Xa21_OsI_11192 | ATGGCGGGGTGTACTGTTGTTGTATTTGGGGTTCTGGTGCTGGTCGCGGGGATGGCGGCGGCGCAGGG | MAGCTVVVFGVLVLVAGMAAAQG |
| | (Os03g0301200) | GCAGGTGCCGTCGCCGACCCCCGCGGCGGCGGCGGCGGCACCGGCGCCCGCGCCGGACTGCAACGGCA | QVPSPTPAAAAAAPAPAPDCNGI |
| | | TCCTGCTCACCTACAACTTCGAGGGCCGCGCCAAGATCCGGCCCTACGTGGCGGACAGGAACAAGCAG | LLTYNFEGRAKIRPYVADRNKQP |
| | | CCCTACTCGTTCAGGGCCAACGCCACCGTGCTCAACTCCGGGACGCGCCCGCTCAAGTCGTGGGCGAT | YSFRANATVLNSGTRPLKSWAML |
| | | GCTCGTGACGTTCGGGTACAGGGAGATCCTCGTCGGCGTCGACGGCGCCGTGCTCACCGGCGGCGGCG | VTFGYREILVGVDGAVLTGGGEM |
| | | AGATGCCGTACAACACCACCCAGGACGCCGGCAACGCGACGTCGTTCTCCGGCTACCCGCAGACCGAC | PYNTTQDAGNATSFSGYPQTDLL |
| | | CTCCTCACCCCGATCGCCACCGCCGGGGACATCTCGCAGATCCAGGCTTCGGTCGGCATCGTCGGCAC | TPIATAGDISQIQASVGIVGTLF |
| | | GCTCTTCGCCGGGCCAGGGCCGTTCGTGCCGCTCCCCACGGCGCTGTCGCTCGACGACCCGGCGTACA | AGPGPFVPLPTALSLDDPAYRCP |
| | | GGTGCCCCCAGGCGACCAATGTTTCCTCCGGGGTGCTCTCCACCTGCTGCGTCCTCACCCCGGAGGCG | QATNVSSGVLSTCCVLTPEAEAN |
| | | GAGGCCAACGCCACCGTCATCGACGCCAACGCCACCGACCCGACCAAGAACTTCCTCCCCCGCGGCAC | ATVIDANATDPTKNFLPRGTGDL |
| | | CGGCGACCTCGTCATCACCTACGACGTGCTCCAGGCCTACCCCTCGAGCTACCTCGCGCTCGTCACGC | VITYDVLQAYPSSYLALVTLDNN |
| | | TCGACAACAACGCCAAGCTCGGCCGCCTCGACAACTGGCGGCTTTCCTGGGAGTGGCGGCGCGGCGAG | AKLGRLDNWRLSWEWRRGEFIYS |
| | | TTCATCTACTCGATGAAGGGTGCGTACCCATCGGAGAAGGACACGACCGGCTGCATCTACGGGGCGGC | MKGAYPSEKDTTGCIYGAAGQYY |
| | | AGGGCAGTACTATCAGAGCCTTGATTTCTCCCAGGTGCTCAACTGCGACAAGAAGCCGGTGATCCTTG | QSLDFSQVLNCDKKPVILDLPLS |
| | | ACCTGCCTCTCTCCAGGTACAATGACACCCAGATTGGGAAGATTGACCATTGCTGCAGGAATGGCACA | RYNDTQIGKIDHCCRNGTILPKS |
| | | ATCTTGCCCAAGTCCATGGATGAGACGCAGTCGAAATCAGCCTTCCAAATGCAGGTGTTCAAGATGCC | MDETQSKSAFQMQVFKMPPDLNR |
| | | ACCGGACCTGAACCGGACAAAGCTTTTTCCACCGGCCAATTTCAAGATAGCCGGTGCATCATCACTGA | TKLFPPANFKIAGASSLNPDYTC |
| | | ACCCAGACTACACCTGTGGCCAGCCGGTGCCCGTCAGTCCCACAGAGTTCCCTGATCCAAGCGGGCTT | GQPVPVSPTEFPDPSGLDSTTLA |
| | | GATTCAACGACTCTGGCGATCGCGACATGGCAGGTGGTTTGCAACATTACCACGAGTAAGGGGGCGAA | IATWQVVCNITTSKGAKPKCCVT |
| | | GCCGAAGTGCTGTGTGACCTTCTCGGCATACTACAATGATTCAGTGATCCCCTGCAACACCTGCGCCT | FSAYYNDSVIPCNTCACGCPANQ |
| | | GTGGGTGTCCGGCGAACCAGCGGGGTCCTACTTGCAGCACGACTGCTCAATCCATGCTCCTGCCACCC | RGPTCSTTAQSMLLPPEALLVPF |
| | | GAGGCGCTGCTTGTGCCGTTCAACAATCGCACACAGAAGGCATTGGCATGGGCCGAGTTGAAGCACTA | NNRTQKALAWAELKHYNVPKPMP |
| | | CAATGTGCCTAAGCCAATGCCTTGTGGCGATTACTGTGGTGTGAGCATCAACTGGCACATATCAACAG | CGDYCGVSINWHISTDYNKGWSA |
| | | ACTACAACAAGGGATGGAGCGCTCGGATGACATTGTTCAATTGGGACAATGTCGACTTGGCGAACTGG | RMTLFNWDNVDLANWFAAIVMDK |
| | | TTTGCTGCTATTGTCATGGACAAGGCTTATGATGGTTTTGAGAAGGCGTACTCTTTCAATAGCACATC | AYDGFEKAYSFNSTSVGKNTIFM |
| | | AGTTGGCAAGAACACCATCTTCATGCAAGGTCTTGAGGGGCTCAATTACCTGGTGAAGCAGACCAACA | QGLEGLNYLVKQTNMSGSDYLVP |
| | | TGAGTGGATCAGACTACCTTGTTCCTGGGAAGCAGCAGTCAGTGCTCTCATTCACCAAGAAGCTGACT | GKQQSVLSFTKKLTPSINVVAGD |
| | | CCTAGCATCAATGTTGTTGCTGGAGATGGTTTCCCTTCAAAGGTCTTCTTCAACGGCGACGAGTGCGC | GFPSKVFFNGDECAMPQRIPMSN |
| | | GATGCCTCAGAGGATTCCAATGAGCAATAGTGGATTCAGGACCCATCTCAGCAGTGTCCTCTCACTGG | SGFRTHLSSVLSLVLVLAASAFV |
| | | TTTTGGTCCTGGCTGCTTCGGCCTTCGTATTGCTTCAGCAATAG | LLQQ* |
| | | SEQ ID NO: 133 | SEQ ID NO: 134 |
| 68 | Xa21_Os03g0302200 | ATGGGGAAGGGAGGGGAAGGGGCGGTTCCCGTGGGGGAGAGCGGCGGGCGGCGGCGGAGGAGGCTGGG | MGKGGEGAVPVGESGGRRRRRLG |
| | | CGAGGACGGAGGCGACGACGACGACGAGGAGTATGTGGTGGAGGAGGATGAGGAGGAAGAGTGCGACG | EDGGDDDDEEYVVEEDEEEECDE |
| | | AGGATCTGTCTGCCTCGAGCGCCGGCGAGGGAGGAGGAGAGGGCACAGACGAGGAATACGAAGAGGGT | DLSASSAGEGGGEGTDEEYEEGD |
| | | GATGAGGACGAAGAGGAGGATGAGACCCCGCGGCCGAGGCAGCCTGTCAAGAGCCGCGAGAATGGGCA | EDEEEDETPRPRQPVKSRENGQK |
| | | GAAGGGGAAGGCAGACCCACCCGTGGCGCGATCTCGACGACGTAAGTACGAGGATGACGATGACTACT | GKADPPVARSRRRKYEDDDDYSE |
| | | CGGAAGAAGAAGACGATGGGGTCGACGAGTACGACGAGGATCTTGAGGAGGAGGAAGAGGAGGACGAT | EEDDGVDEYDEDLEEEEEEDDEA |
| | | GAGGCGCCACGATCCAAGCGCGTGAAGAAACGTGGTGGCCGCAACGTGGAGGGGAAGCTTTCTCCGGA | PRSKRVKKRGGRNVEGKLSPERS |
| | | GCGATCAAATCGCCGGAGGTACGAGGAGGACATGGACTTTGACCCTGACATGGATGAGGAGGAAGAGG | NRRRYEEDMDFDPDMDEEEEEDV |
| | | AGGATGTTGATTTCGATCCTGAAGTGGAGGATGAGGAGGAAGAAGATTTTGAGGATGAGGAAGAGGAT | DFDPEVEDEEEEDFEDEEEDELE |
| | | GAATTGGAAGCGACTAAGGTGAGGGTTAAGAATATGGGGCGGCAAAAGTCTGCTTTGAATCAGCGACG | ATKVRVKNMGRQKSALNQRRGKK |
| | | AGGGAAGAAGAAGAGCAGCTCTAAGGTGGCCAGCCGGAAGGTGGGTTCAGTGAAGGCAAGGAAGGCTG | KSSSKVASRKVGSVKARKAASAR |
| | | CTTCTGCAAGACGGAGGCAGAAAAAGCGTTCAATGCTTGATCGTTATGAGGATGACGACTTCATTGTG | RRQKKRSMLDRYEDDDFIVEDEV |
| | | GAGGACGAGGTTACGGCTGATTGGCAACCAAGGAAAAAGGCTAGAATTAGGAAGCAGATGGAGGTTGA | TADWQPRKKARIRKQMEVDPPTP |
| | | TCCTCCGACACCAGTTTTTGAGGCTGAGATATGGCCTACTATTGATTCAGACACGACAGACTTCGAAT | VFEAEIWPTIDSDTTDFEFVTSD |
| | | TTGTAACATCCGATGAGGAAGCAGCCATTGCTGAGCCTACTAGGGTTATTAAGAAGGGGAGGAAGAAA | EEAAIAEPTRVIKKGRKKRVFVS |
| | | AGGGTATTTGTGTCAGATTCATCCTCAGATTCTGAATTCGTTGTATCAGATAAGGAATTGGGGAATTT | DSSSDSEFVVSDKELGNLKESEP |
| | | GAAGGAGTCTGAGCCTCCAGAGTCTCTGAAAGTGCTGCCTTCATCACCCAGGAAGATTTCTGTTACAG | PESLKVLPSSPRKISVTGNGEHK |
| | | GTAATGGGGAGCACAAGGGGAAGGAGAAAAAGGAACCACAGGAGGCTGGAAGGGCAACGTGCGGGATT | GKEKKEPQEAGRATCGICLSEEQ |
| | | TGCCTTTCTGAAGAACAGAGAGTGACTGTGCAGGGTGTGCTGGATTGCTGTTCACACTATTTTTGCTT | RVTVQGVLDCCSHYFCFACIMQW |
| | | TGCATGCATCATGCAATGGTCTAAGGTTGAGTCAAGGTGTCCATTGTGTAAACGGCGGTTCACAACAA | SKVESRCPLCKRRFTTITKSSKE |
| | | TCACAAAGTCGTCAAAGGAGGATACTGGTTTGGAACTGACAAATTCTGTAATTAGGGTTGAAGAACGT | DTGLELTNSVIRVEERDQVYQPT |
| | | GATCAGGTTTATCAGCCTACGGAAGAGGAAATAAGACGTTGGCTAGATCCATACGAGAATGTTGTGTG | EEEIRRWLDPYENVVCIECNQGS |
| | | CATAGAGTGCAATCAAGGTAGTGATGACAGTCTCATGTTACTATGCGATATTTGTGACTCCTCAGCAC | DDSLMLLCDICDSSAHTYCVGLG |
| | | ATACTTACTGTGTTGGTTTGGGAAGAGAAGTACCTGAAGGAAATTGGTACTGTGGAGGTTGTAGACTC | REVPEGNWYCGGCRLDGEAHSYH |
| | | GATGGCGAAGCACATTCATACCATAATCATGTGAATGGCAATTCTGGGATGTTTGGTGCAATTTCACC | NHVNGNSGMFGAISPIGTFERQG |
| | | AATTGGCACTTTTGAAAGGCAGGGGATTGATCTAAATGTATCTCCAAGAGAGATTCCTAGGGGAAATC | IDLNVSPREIPRGNHSVESQAST |
| | | ATTCTGTTGAGTCTCAAGCTTCTACTGCAGGAGCATCAACTCCATCTGGAAGGCAGACAAACGCAACG | AGASTPSGRQTNATNFRRRQMHD |
| | | AATTTTAGAAGACGTCAAATGCATGACTGGATACGCAGTTTGCTTTCTAGACCAAGGACAACACTTGG | WIRSLLSRPRTTLGPVMHHNGVH |
| | | GCCAGTTATGCATCATAATGGTGTGCATCAGAGTGGTTTTGTACCAAGCACTGAACCAGACCACATGA | QSGFVPSTEPDHMNFCAPLESDT |
| | | ACTTTTGCGCACCATTAGAATCTGACACTTTACACAACACTGGGTCTGTACCACGAAGTGAACCAAGC | LHNTGSVPRSEPSQNFHVMSEAN |
| | | CAGAACTTCCATGTTATGTCAGAAGCCAACACTTCAGAGACTTCCTTTGGAAGGCATGCAGCTCTTTC | TSETSFGRHAALSERRQIYERFF |
| | | TGAAAGACGTCAAATTTATGAACGTTTTTTCATGTTGCTATCTAGACCAAGCCCGACTATTAGACCGG | MLLSRPSPTIRPDLCHNASEHGS |
| | | ACTTGTGCCATAATGCCTCAGAGCATGGTAGTTCTATACCAAGAGTTGAACCAAACCACATGAACTTT | SIPRVEPNHMNFHAPPVANSPQT |
| | | CATGCTCCGCCAGTAGCCAACAGTCCACAAACTCTGCTTGATGGCATTCCAAACCGCAGCAATGGTTT | LLDGIPNRSNGFSFTQAHSNFVD |
| | | TTCTTTTACTCAGGCCCACAGTAATTTTGTAGATGGAAACAACTTCCAAGGAACTGAAGGTGTCTAG | GNNFQGTEGV* |
| | | SEQ ID NO: 135 | SEQ ID NO: 136 |
| 69 | Xa21_Os03g0306700 | ATGTCAGTTGCGCCGGCGACGGAAGGGTCCGCACGCCGCTCGGCGGCGGCGGCGGCGGAGGAGGAGGA | MSVAPATEGSARRSAAAAAEEEE |
| | | GGAGCGGCAGCGCAACCGGAAGAGGTCGAACCGGCTGTCGGCGCAGAGGTCCCGCATGAAGAAGCAGC | ERQRNRKRSNRLSAQRSRMKKQQ |
| | | AGTACGTGGACGGGCTGGCCGTGGAGGCGGAGCAGCTCCGGCGGGAGAACGACGCCATGCGCGCCGCG | YVDGLAVEAEQLRRENDAMRAAA |
| | | GCCGGCGCCGTGCTGCAGCGGTGCCGCCTGGTCGAGCAGGAGAACCGGGTGCTCGCCGCGCACGCGCG | GAVLQRCRLVEQENRVLAAHARE |
| | | CGAGCTGTGCAGCGCGCTCCAGCTCCGCGCCTCGCAGCTCCCCCTGCTCGGGGAGGTGGCAGGCGTGC | LCSALQLRASQLPLLGEVAGVPL |
| | | CCCTCGACGTGCCCGACGTCGCCGACCACCTCGTGCAGCTCTACGGCGGCGGCCTGGGGATGACGCCG | DVPDVADHLVQLYGGGLGMTPLS |
| | | CTCTCGCCACCGCCGCTGCTGCCGCCGCTGCCGCCGCAGATCGAGATGCTGTTCTTCCAGCCAGACTC | PPPLLPPLPPQIEMLFFQPDSVM |
| | | GGTCATGGATCCGGTCAGTATGCTGCAGGGCTACGAAAACATATGA | DPVSMLQGYENI* |
| | | SEQ ID NO: 137 | SEQ ID NO: 138 |
| 70 | Xa21_Os03g0310800 | ATGTGTCCCGGAGGCAGATACGCGGGCCTCGACGTCCCCGCCTGCGCGGCGGCGGGAGACCTGAGGCC | MCPGGRYAGLDVPACAAAGDLRP |
| | | GGCGTTCGACGTGCTGGACGCGGACCGCGACGGCCGCATCAGCCGGGAGGACCTCAAGTCGTTCTACG | AFDVLDADRDGRISREDLKSFYA |
| | | CCAGCGGCGCGGCCACCAGCGAGCGGTTCGACGACGAGGACATCGCCGCCATGATCGCCGCAGCCGAC | SGAATSERFDDEDIAAMIAAADA |
| | | GCGGACAACGACGGGTTCGTGCAGTACGACGAGTTCGAGCGCCTGCTGGGGCGCGCCGCCGCGGGGGC | DNDGFVQYDEFERLLGRAAAGAG |
| | | GGGGTGCCGGCCCGCGATGGAGGACGTGCTCCGGGTGATGGACCGCGACGGGGACGGCAAGGTGGGGT | CRPAMEDVLRVMDRDGDGKVGFD |
| | | TCGACGACCTCAAGGCGTACCTCGGCTGGGCCGGGATGCCGGCGGCCGACGAGGAGATCCGCGCCATG | DLKAYLGWAGMPAADEEIRAMIR |
| | | ATACGCGTGGCCGGCGGCGGCGACGGCGATGGCTGCGTGGGGCTCGAGGAGCTCGCCATCGTCCTCGG | VAGGGDGDGCVGLEELAIVLGCS |
| | | CTGCTCGCCCACAAATTGGACCGCATCCTCTCAAACTCTGCATTGA | PTNWTASSQTLH* |
| | | SEQ ID NO: 139 | SEQ ID NO: 140 |
| 71 | Xa21_OsJ_10653 | ATGGCGGCGCAGGGGATCGCGGAGGCGGTGCAGGCCTACACGGGCCTCTCCCCGGCGGCGGCCGTCAC | MAAQGIAEAVQAYTGLSPAAAVT |
| | (Os03g0321000) | CATCCTGGCCCTCATGCTCGCCACCTACCTCCTCGTGTCCTCCCTCTTCGTGGGCGAGATCACGCTCG | ILALMLATYLLVSSLFVGEITLE |
| | | AGCAGCTCGCGGCCTACGACGGCAAGGACCCCGCGAAGCCGATCCTCATCGCCATCCGCGGCCACGTC | QLAAYDGKDPAKPILIAIRGHVY |
| | | TACGACGTCACGCGCGGGAGGCTCTTTTATGGTCCTCAAGGACCATATTCCTTGTTTGCTGGGAGGGA | DVTRGRLFYGPQGPYSLFAGRDA |
| | | TGCAACTCGGGCCCTTGCATTGATGTCGTTTGACCCTATTGACCTCACTGGAGATTTGGATGGCCTTG | TRALALMSFDPIDLTGDLDGLGP |
| | | GTCCAGACGAGCTAGAGGTCTTGCAAGATTGGGAAGATAAGTTTAAAGAGCGGTATCCTACGGTGGGC | DELEVLQDWEDKFKERYPTVGHL |
| | | CACCTTGCTTCAGAAAATGCAGCGGATGGTAACCACGGTGGCGCAGCTTGA | ASENAADGNHGGAA* |
| | | SEQ ID NO: 141 | SEQ ID NO: 142 |
| 72 | Xa21_OsI_11859 | ATGGCGGAGACGCGCAAGTTCTTGATGCCCAACCAGCCCCCAGATATTTCGCAGATGGTGCTGGAGGC | MAETRKFLMPNQPPDISQMVLEA |
| | | TAGGAAGCGATGGTTGCGACCCACTGAAATATGCGAAATACTTTCAAACTACAGGAGTTTCTCTCTCT | RKRWLRPTEICEILSNYRSFSLS |
| | | CGCCTGAGCCACCAAACAGGCCCGGAAGTGGCTCTCTTTTTCTATTTGACCGCAAAACATTAAGATAC | PEPPNRPGSGSLFLFDRKTLRYF |
| | | TTCAGAAAGGATGGGCATAACTGGAGGAAGAAAAAGGATGGGAAGACCGTTAAAGAAGCTCACGAGAA | RKDGHNWRKKKDGKTVKEAHEKL |
| | | ACTAAAAGCTGGCAGCATTGATGTTCTACACTGCTACTATGCTCATGGGGAGGAAAATGAGAACTTCC | KAGSIDVLHCYYAHGEENENFQR |
| | | AAAGAAGGACCTATTGGCTGTTGGAAGAGGATTTCACGCACATTGTTCTCGTACACTATCTTGAAGTC | RTYWLLEEDFTHIVLVHYLEVKG |
| | | AAGGGTGTAAAGCAAAGTTTTAGTCGTGCTAAAGAAGAAATTATGCAGTTATCTGGTGCTGATAGTCC | VKQSFSRAKEEIMQLSGADSPSC |
| | | ATCGTGTTCAAATTCGATTACTAGTCAGAACCAGATGACCCCACAAATAATGGATGCAGCTGAAAGCC | SNSITSQNQMTPQIMDAAESPIS |
| | | CAATTAGTGGGCAGATTTCAGAGTATGAAGGTGCTGAACCAGCGAAATTCGGTGCAGCAGATAATTGC | GQISEYEGAEPAKFGAADNCRAS |
| | | CGAGCAAGCTCCAGATACAACCCTCTCATTGAGATGCAGCAGCCTCTGGATGGAATCGTGATGGATAG | SRYNPLIEMQQPLDGIVMDNILY |
| | | CATTTTATACCCTTCTGCTTCAGCCATCTGTAACCAAGTTTCAGGTTATCATGGTGAATTGCCACCAG | PSSSAICNQVSGYHGELPPGTSN |
| | | GAACATCTAATCTAAATGGTCATACGTTTACTCACAGTGACATAGCTAGAATGTTTGATGATTCCAGC | LNGHTFSHSDIARMFDDSSSGLR |
| | | TCTGGGCTAAGAGACATTTCCAGAACCCTGTTTGATTCAATGCCTTACGATGAGCATTTATCAGGATA | DISRTLFDSMPYDEHFSGYANGF |
| | | TGCAAATGGATTCATGGAGCCAACTCTTCATTCTTCATTTTCCATGATAGAGGCTAACAATCTGGAGG | MEPTLHSSFSMIEANNLEDSSLL |
| | | ATAGCTCTCTTCTGGAGACATTTACATCTGAAGCTCTTTACACTAACAACCTCAGCCAGAAGGAAGCT | ETYTSEALYTNNLSQKEADALSF |
| | | GATGCACTAAGTTTTGCTGGCATATCATCGCCAGAGGTGAATGGTAATAAATATACTGAAGGAAGCAC | AGISSPEVNGNKYTEGSTKHPLL |
| | | CAAGCATCCACTCCTGAAGCAATTGTCATTAGATCTATTTAAGATTGAGTCTTCTGGTTTAAAGAAAC | KQLSLDLFKIESSGLKKHDSFSR |
| | | ATGACAGCTTTTCAAGGTGGATGAGTAAAGAACTTGGAGAGGTGGTTGATTTGGGTATCAAATCAAGT | WMSKELGEVVDLGIKSSSDALWS |
| | | TCTGATGCTCTCTGGAGTAGCATTGAGATTGTGAATGCTGCTGATGGTCCTAGTACACCAATTAATGA | SIEIVNAADGPSAPTNEQLDAYA |
| | | ACAGTTAGATGCTTATGCAGTGAGCCCTTCACTTGCGCAAGACCAACTATTCAGCATTCTTGACATTT | VSPSLAQDQLFSILDISPSCSYI |
| | | CACCAAGCTGCTCATATATTGGTTTGAAGACAAAGGTTCTGGTAACTGGTACATTCCTGGCAAGTAAA | GLKTKVLVTGTFLASKENVENCK |
| | | GAGAATGTGGAAAATTGCAACTGGTCATGCATGTTTGGGGATGTTGAAGTACCAGCTGAGGTTTTAGC | WSCMFGDVEVPAEVLADGSLRCY |
| | | TGATGGTTCCCTACGCTGCTATGCACCAGAACATCAATCTGGAAGAGTTCCCTTTTATGTGACATGCT | APEHQSGRVPFYVTCSNRIACSE |
| | | CAAACAGGATCGCTTGTAGTGAAGTGCGAGAATTCGAGTACCGGGATTCTGATGCTCAGTACATGGAG | VREFEYRDSDAQYMETSHSQANG |
| | | ACATCACATTCTCAAGCCAATGGTATAAATGAAATGCATCTACAAATTCGTCTTGAGAGGCTACTCTC | INEMHLQIRLEKLLTLGPDDNQL |
| | | TTTGGGGCCAGATGACAATCAACTGCTTGTTTGTGGAAATGAAAAACTTGAGTTAATCAATGCTATAA | LVCGNEKLELINAINSLMLDEKW |
| | | ACTCCCTGATGCTCGATGAGAAATGGTCAGATCAGGGATCGCCATCCGGCTCTAAAGATGTTGTCACC | SDQGSPSGSKDVVTPRNQSLKKL |
| | | CCAAGGAACCAAAGTTTAAAGAAATTGATGAAAGAAAAGCTTCATTGTTGGCTTATCTATAAAATAAA | MKEKLHCWLIYKIYDCEKGPNIL |
| | | TGATTGTGAGAAGGGCCCAAATATACTGGGAAAGGAAGGACAGGGTATAATTCATTTGGTGGCTGCAC | GKEGQGIIHLAAALGFDWAIRPI |
| | | TGGGCTTCGATTGGGCTATAAGGCCAATACTAGTTGCTGGTGTCAATGTGAACTTTCGGGATGCTCAT | LVAGVNVNFRDAHGWTALHWAAS |
| | | GGATGGACTGCTCTTCATTGGGCTGCGTCTTGTGGAAGAGAGCGAACAGTGGGCGTCCTTATAGCAAA | CGRERTVGVLIANGAAAGALTDP |
| | | TGGAGCTGCAGCAGGTGCATTGACAGATCCTACTTCTGAATTTCCTTCTGGAAGAACTCCAGCCGATT | TSEFPSGRTPADLASTNGHKGIA |
| | | TAGCCTCTACAAATGGACACAAGGGAATTGCTGGTTTTCTCGCGGAATCTGCTTTGACAAGCCATCTA | GFLAESALTSHLSALTLKESKDS |
| | | TCAGCACTATCTTTAAAGGACTCAAAAGATAGCAATGTGGAAGAAGCATGTGGATTAACAATTCCTGA | NADEACRLTIPEDLPEMNYGQLA |
| | | AGATCTCCCTGAAATGTATTCTGGTCAGCTTGCTGTACAGGATTCTCATGCTGAATCATTGAAGGATT | VQDSHAESLKDSLSAVRKSAQAA |
| | | CATTAAGTGCTGTTCGTAAATCCGCTCAAGCTGCAGCTAGGATATTCCAAGCCTTCAGGGTGGAATCA | ARIFQAFRVESFHRKKVVEYGDD |
| | | TTCCACAGAAAGAAGGTCGTGGAATATGGAGATGATGATTGTGGATTATCTGATGAACACACATTTTC | DCGLSDEHTFSLISLQKVKQGQH |
| | | ACTTATTTCTCTTCAAAAGGTCAAACAAGGGCAACATGATACACGTTTGCATTCTGCTGCTGTACGTA | DTRLHSAAVRIQNKFRGWKGRKE |
| | | TCCAAAATAAGTTCCGAGGATGGAAGGGAAGAAAGGAGTTTATGATCATTCGCCAGAGAATTGTGAAG | FMIIRQRIVKLQAHVRGHQVRKN |
| | | TTACAGGCTCATGTGCGAGGACATCAAGTGAGGAAAAACTATAAAAAAGTAGTCTGGTCAGTAGGTAT | YKKVVWSVGIVEKVILRWRRKGR |
| | | TGTCGAGAAAGTTATATTGAGATGGAGGAGAAAGGGACGTGGTTTGCGTGGCTTTCGACCTGAGAAAC | GLRGFRPEKQLEGQTQIQPAKTE |
| | | AATTAGAAGGCCAAACACAAATCCAACCTGCAAAAACAGAGGATGAATACGATTACTTGCAGGATGGC | DEYDYLQDGRRQAEGRLQRALDR |
| | | AGAAGACAGGCTGAAGGCAGACTACAGAGGGCACTGGACCGGGTGCGCTCTATGACACAATACCCAGA | VRSMTQYPEAREQYRRLTTCVAE |
| | | AGCAAGAGAACAGTACCGTAGGCTTACAACTTGTGTTGCTGAAATGCAACAGTCCAGGATGATGCAGG | MQQSRMMQDEMLSEAAGADGSDF |
| | | ACGAGATGTTAAGTGAGGCAGCTGGTGCTGACGGAAGTGATTTCATGAATGGACTGGAGGACTTGATA | MNGLEDLICRDDPQMTAIW* |
| | | TGCAGAGACGACCCCCAAATGTCTGCCATTTGGTGA SEQ ID NO: 143 | SEQ ID NO: 144 |
| 73 | Xa21_Os03g0645900 | ATGGCGACGATCACGACGCCGGGATATGCTCACATACAGCGGCAGCACGGCAGGTGCTCGACGACGGC | MATITTPGYAHIQRQHGRCSTTA |
| | | GGGAAGGCGTGGGGCGTCCAATTCGGTGAGATTCTCCGCGCGCGCGGTTAGCTCCGTGCCGCACGCGG | GRRGASNSVRFSARAVSSVPHAA |
| | | CGGCGGCGTCGTCATCGGCGCCGGCGTTCCTGCCGGTGCCGTTCGTGCCTGGGGCCGACGCGCCGTCG | AASSSAPAFLPVPFVPGADAPSP |
| | | CCGTCGGGGAAGAGTGCCATTGGCGTCCCGAAGGCGCCGAGGAAGGGGGAGGAGGGGAAGAGGCTCAA | SGKSAIGVPKAPRKGEEGKRLNF |
| | | CTTCTTCCAGCGCGCCGCGGCGATGGCGCTCGATGCGTTCGAGGAAGGGTTTGTGGCGAATGTCCTCG | FQRAAAMALDAFEEGFVANVLER |
| | | AGCGCCCGCACGGGCTGCCGAGCACGGCCGACCCCGCGGTGCAGATCGCCGGCAACTTCGCGCCGGTC | PHGLPSTADPAVQIAGNFAPVGE |
| | | GGTGAGACGCCGCCCGCGCGCGCGCTGCCGGTGTCGGGGCGCATCCCGCCCTTCATCAACGGCGTCTA | TPPARALPVSGRIPPFINGVYAR |
| | | CGCGCGCAACGGCGCCAACCCGCACTTCGACCCCGTCGCCGGGCACCACCTGTTCGACGGCGATGGCA | NGANPHFDPVAGHHLFDGDGMVH |
| | | TGGTGCACGCCATCAGGATACGCAACGGCGCCGCCGAGTCGTACGCGTGCCGGTTCACGGAGACCGCG | AIRIRNGAAESYACRFTETARLR |
| | | CGGCTGCGGCAGGAGCGCGCGATGGGGCGGCCCATGTTCCCCAAGGCCATTGGGGAGCTCCATGGCCA | QERAMGRPMFPKAIGELHGHSGI |
| | | CTCCGGCATCGCGCGCCTTGCTCTGTTCTACGCGCGCGCCGCGTGCGGCCTCCTCGACCCGTCGCATG | ARLALFYARAACGLLDPSHGTGV |
| | | GCACCGGCGTCGCCAACGCCGGCCTCGTCTACTTCAACGGCAGGCTCCTCGCCATGTCGGAGGACGAC | ANAGLVYFNGRLLAMSEDDLPYQ |
| | | CTCCCCTACCAGGTGCGCGTCACCGCCGACGGCGACCTCGAGACCGTCGGCCGCTACGACTTCGACGG | VRVTADGDLETVGRYDFDGQLGC |
| | | GCAGCTCGGCTGCGCCATGATCGCGCACCCCAAGCTCGACCCGGCCACCGGCGAGCTCCACGCGCTCA | AMIAHPKLDPATGELHALSYDVI |
| | | GCTACGACGTGATCAAGAAGCCGTACCTCAAGTACTTCTACTTCGCGCCCGACGGCACCAAGTCGGCC | KKPYLKYFYFAPDGTKSADVEIP |
| | | GACGTCGAGATCCCGCTCGACCAGCCCACCATGATCCACGACTTCGCCATCACCGAGAACTACGTGGT | LDQPTMIHDFAITENYVVVPDHQ |
| | | GGTGCCCGACCACCAGGTGGTGTTCAAGCTCCAGGAGATGCTCCGCGGCGGCTCGCCCGTGGTGCTCG | VVFKLQEMLRGGSPVVLDKEKTS |
| | | ACAAGGAGAAGACGTCGCGGTTCGGGGTGCTCCCCAAGCACGCCGCGGACGCGTCGGAGATGGTGTGG | RFGVLPKHAADASEMVWVDVPDC |
| | | GTGGACGTCCCGGACTGCTTCTGCTTCCACCTCTGGAACGCGTGGGAGGAGGCGGACACCGACGAGGT | FCFHLWNAWEEADTDEVVVIGSC |
| | | GGTGGTGATCGGCTCGTGCATGACCCCCGCCGACTCCATCTTCAACGAGTCCGACGACCGCCTCGAGA | MTPADSIFNESDDRLESVLTEIR |
| | | GCGTCCTCACCGAGATCCGCCTCAACACCCGCACCGGCGAGTCCACGCGGCGCGCCATCCTGCCGCCG | LNTRTGESTRRAILPPSRQVNLE |
| | | TCGAGGCAGGTGAACCTCGAGGTGGGCATGGTGAACCGCAACCTCCTCGGCCGCAAGACGCGGTACGC | VGMVNRNLLGRKTRYAYLAVAEP |
| | | CTACCTCGCCGTGGCCGAGCCGTGGCCCAAGGTGTCGGGCTTCGCCAAGGTGGACCTCGCCACGGGTG | WPKVSGFAKVDLATGELTKFEYG |
| | | AGCTCACCAAGTTCGAGTACGGCGAGGGCCGGTTCGGCGGCGAGCCCTGCTTCGTCCCCATGGACGCC | EGRFGGEPCFVPMDAAAATPRGE |
| | | GCCGCCGCCACGCCTCGCGGCGAGGACGACGGCTACATCCTGTCCTTCGTCCACGACGAGCGCGCCGG | DDGYILSFVHDERAGTSELLVVN |
| | | GACCTCCGAGCTCCTCGTCGTCAATGCCGCCGACATGCGCCTTGAGGCCACCGTGCAGCTGCCGTCCC | AADMRLEATVQLPSRVPYGFHGT |
| | | GCGTGCCGTACGGCTTCCACGGCACGTTCATCACCGGCGACGAGCTCACCACCCAGGCCTGA | FITGDELTTQA* |
| | | SEQ ID NO: 145 | SEQ ID NO: 146 |
| 74 | Xa21_Os03g49430 | ATGGACGTCCTGAAGCGCGAGCTGCAGCGGAAGCGCCAGCTGCTCGACGCCGACTTCGGCGGCCGCAA | MDVLKRELQRKRQLLDADFGGRK |
| | (Os03g0701100) | GCTCCTCCGCCGCGCCGAGATCGAGCGCCGCGAGATGGAGCGGATCCGGGAGGCCGAGCGCCGGCTCC | LLRRAEIERREMERIREAERRLL |
| | | TCCTCCAGAAGCAGCTCCGGTCCTCCTCCTCCCACCACCGCGCCGCGTCGCGCTCCACGACCACGCCG | LQKQLRSSSSHHRAASRSTTTPG |
| | | GGGGCTCCCGACGGCGACGAGTCGTCGCGGGCCGAGCACCGGGGCGACGGGGAGGAGAAGGAGGACGC | APDGDESSRAEHRGDGEEKEDAP |
| | | GCCGCTCCTCCCCAGGGAGGAGGTCATCCGGCGCCTGCGCGTGCTGCGCCAGCCGGCGACGCTCTTCG | LLPREEVIRRLRVLRQPATLFGE |
| | | GCGAGGACGACGCCGCGCGCCTCCGCCGCCTCCACGCCGTGCTCGAGGACCCCGCGGCGCTCGCCGAC | DDAARLRRLHAVLEDPAALADVD |
| | | GTCGACGCGGCGGAGATCGGGGAGGGCCAGACCAACGACTTCCTCCGCGACATCCAGGCGCTGCGGGC | AAEIGEGQTNDFLRDIQALRAKT |
| | | GAAGACCGTCGCGGCGTCGGTGGCAGCGAAGCCCAAGGCTGGCGCTGCAACTGCAGAGAGCGGGGGCG | VAASVAAKPKAGAATAESGGEAR |
| | | AGGCGAGGGAGGAGGTACCGTTCGAGGAGCTCTCCGACGAGGACAAGATCATGGCCTTCTTCAAGAGG | EEVPFEELSDEDKIMAFFKRLMS |
| | | CTGATGAGCGAGTGGAGCCAGGAGATGGACGAGATGCCCGAGGCGGAGAGGCGGACGGCGAAAGGGAA | EWSQEMDEMPEAERRTAKGKAAV |
| | | GGCTGCGGTGGCCACCTGCAAGCAGTGCGCGCGCTATCTCGAGCCGCTGTTCAAGCTGTGCAAGAAGA | ATCKQCARYLEPLFKLCKKKVCD |
| | | AGGTATGTGACAGTATTAATTCACGCACACACTTAAGAATCTGA | SINSRTHLRI* |
| | | SEQ ID NO: 147 | SEQ ID NO: 148 |
| 75 | Xa21_Os03g0703400 | ATGTTCTCGTGGAGCCGTAAGCAGTCGTCCTCCTCCGGCCGCCGCGGCGCGGACGCGTCGTCGATGGA | MFSWSRKQSSSSGRRGADASSMD |
| | | CTCCAGCAGCCGCGGCGGCGGAGGGGAAGGGAGCGGGAGCGGGAGCCGCGGGCGTAGCTCGCGGCTGG | SSSRGGGGEGSGSGSRGRSSRLE |
| | | AGCGCCGCAACGCGGTGAAGCACATCGACTACGAGGCGGCGGGGGCGGGGGCGGGGGCGTCGTCCGTG | RRNAVKHIDYEAAGAGAGASSVP |
| | | CCGGCGTGCGCGTCGTGGTCGTCGTCCATGTCGGCGGAGCGGTCGCTGGGGCTCCGGCCGTCGCGCTC | ACASWSSSMSAERSLGLRPSRSL |
| | | GCTCGACCTGGCCGTCGGGGGCGGCGGCACGGACATCCGCATCAGCGGCAGCGTGGAGGGGGAGGTCG | DLAVGGGGTDIRISGSVEGEVDE |
| | | ACGAGCTGTGCCGCAGCCTGGGTTTATCGGGGCCGGAGGAGTTCGCGATCCCCGTCGCCGCATGGGAA | LCRSLGLSGPEEFAIPVAAWEAR |
| | | GCCCGGAAAGAGCGTTCTAACTCCGACCTACTACCGCGCTCCCGCTTCGTCTCCTCGCCACCCGTCGA | KERSNSDLLPRSRFVSSPPVDDP |
| | | CGACCCCTCTCCCATGGCCCGCACCATTTCCGCCCCCGAGGTCATCCAGTGCGACCTTCCTCCCTCAT | SPMARTISAPEVIQCDLPPSFPA |
| | | TCCCTGCTTCAATTCCTGAGGAGTCCCTCAATTCTTCCTCCAATTCCACCGCAACTGATTCGGCGGAG | SIPEESLNSSSNSTATDSAEEPT |
| | | GAGCCTACCGCTGCGGCGCTCGGTCAGGAATCCCCCAAGGCTGCCCCTGCCGTTGCTGCTGTGGCACC | AAALGQESPKAAPAVAAVAPLAG |
| | | CCTCGCTGGATTGCCCCTGCTATCGCCAAAGAGAGGAGGCGGCGAAGTCGGTATCCGAGGAGCCCGAC | LPLLSPKRGGGEVGIRGARPPVL |
| | | CTCCTGTGCTCTCCCCGCCTCAGCCGCTCATGGCACTCGCACCGCCGCCAATGAGGCGTTCAATTGTG | SPPQPLMALAPPPMRRSIVAKDM |
| | | GCTAAAGACATGAGCGGGGTGTCGGCTTGGGATATTGTGAACTCGTTTGCTCCCAGTGAAGAGAAAAG | SGVSAWDIVNSFAPSEEKSEVRT |
| | | TGAAGTGAGAACAGATGATGAACGCGTGGACGCGTCTCACATGTCGGACACCGAGGAGGAGGAGGAGT | DDERVDASHMSDTEEEEEFADEG |
| | | TTGCTGACGAGGGAGTGGCAGGGGTGGACGGGGAGCTGAAAGAGTTAAGGATAGGAGAGACCTTTGAA | VAGVDGELKELRIGETFEGFTGT |
| | | GGGTTCACTGGGACATCATCATTGTCAACAACAAATGATGATGATGCATCCAGCACAACTACGGAGGC | SSLSTTNDDDASSTTTEAMFIIS |
| | | TATGTTCATCATCTCACCAAATGGAAAGTTCAAGAGGAAGATTAAGTCGTGGATGCGTGGTGCTCTTC | PNGKFKRKIKSWMRGALLGSGSF |
| | | TGGGGAGCGGCTCATTTGGGATGGTGTATGAGGGCATCAGTGATGAAGGAGCGTTTTTTGCTGTGAAG | GMVYEGISDEGAFFAVKEVSLLD |
| | | GAAGTATCTTTGCTTGACCAAGGAAGCAACGCACAACAATCTATTCTTGCACTTGAACAGGAAATTGC | QGSNAQQSILALEQEIALLSQFE |
| | | ACTCCTAAGTCAGTTTGAACATGAAAATATAGTCCAGTATTATGGAACTGACAAGGAAGAATCAAAAC | HENIVQYYGTDKEESKLYIFIEL |
| | | TGTACATTTTTATTGAGCTTGTTACACAAGGTTCTCTATCGTCCCTCTATCAAAAGTATAAACTACGA | VTQGSLSSLYQKYKLRDSQVAAY |
| | | GACTCACAAGTGGCAGCATACACAAGGCAGATTCTCAACGGATTGGTTTATCTCCATGAGCGAAATGT | TRQILNGLVYLHERNVVHRDIKC |
| | | GGTTCACAGAGACATTAAATGCGCAAATATACTGGTTCATGCAAATGGATCAGTAAAATTGGCAGACT | ANILVHANGSVKLADFGLAKEMS |
| | | TTGGGTTGGCGAAGGAGATGTCAAAAATTAACATGCTGAGATCATGCAAAGGAAGTGTTTATTGGATG | KINMLRSCKGSVYWMAPEVVNPK |
| | | GCACCTGAGGTTGTTAATCCTAAAAAAACATATGGGCCCCAGGCTGATATTTGGAGTCTTGGCTGCAC | KTYGPQADIWSLGCTVLEMLTRN |
| | | TGTATTGGAAATGCTTACTCGTAACATACCATATCCTAATGTGGAGTGGACAAATGCTTTTTTTATGA | IPYPNVEWTNAFFMIGKGERPQI |
| | | TTGGAAAAGGAGAACGACCTCAAATTCCCAGCTATTTGTCAAAAGATGCTCAAGATTTCATTAGCCAG | PSYLSKDAQDFISQCVQVDPEQR |
| | | TGTGTACAAGTTGATCCAGAGCAACGGCCTTCTGCATCACAACTTATGTCACACCCATTTGTTAATAG | PSASQLMSHPFVNRPLRASFESA |
| | | ACCGCTTCGAGCTTCTTTTGAATCTGCATCCCCTCCTGCCATCAGCTCGTATTGA | SPPAISSY* |
| | | SEQ ID NO: 149 | SEQ ID NO: 150 |
| 76 | Xa21_OsJ_12397 | ATGACCAAGCCAGAAGCCCTGCAACTTCACCATCTGCCCGATGATGTCCTGCACCGCATACTGTCTCG | MTKPEALQLHHLPDDVLHRILSR |
| | (Os03g0722800) | ATTAACATTCAGAGAGTCCTCACGGATGGGCCTTCTTTCTCGCAAGTGGCAGAAACTGTGGAGAAGCT | LTFRESSRMGLLSRKWQKLWRSC |
| | | GCTGCCCGAAATTGATCTTCACCAGGGCCACCATGTTCAAGCCTGGGAATAAAACCATAAGACGTACA | CPKLIFTRATMFKPGNKTIRRTR |
| | | AGGACCAACTTTGCCAGGAGAGTCAACAGTCTTTTGCGGCAGCTATGTGCACCTCCTACCCTGAATAA | TNFARRVNSLLRQLCAPPTLNKF |
| | | GTTTGTTGTTAAGTTTGGGCTGCGCAGGAAACATACCTGCCATGTCAACAGATGGGTTGGCTTCTGCT | VVKFGLRRKHTCHVNRWVGFCSK |
| | | CCAAGTCGAGAGCAAGGCACATCACTTTTGATTTCACTCCTGGAGTGAAGGGTATTTTCAGGGGCTTA | SRARHITFDFTPGVKGIFRGLAD |
| | | GCTGATGAAAAGTACATCTTCCACCTGCATGTTTTCAGTGTCCCTGACCGCTCCCCTGCTCATATCAA | EKYIFHLHVFSVPDRSPAHIKSL |
| | | ATCCCTTCATCTGAGCTACGTCTGGTTGAACACAGCAACCACTGGTTTCACTGGCTTTGCAAACCTCA | HLSYVWLNTATTGFTGFANLKKL |
| | | AGAAGCTCACCCTGCACAAAGTTTCCTTTTTAAATGACTTCCAACATCTGATGCTGTCGGAGTGCACT | TLHKVSFLNDFQHLMLSECTALE |
| | | GCTCTTGAGTGGCTGAGCATCAGCTGCTCTTCCTTCACTGAGCTAACCTTGTGCAAGCCACTGCGCCG | WLSISCSSFTELTLCKPLRRLRY |
| | | TCTGCGTTATCTTTCTTTGCACTACTGCTACATGGAAAAGGTTGAGCTGGAAGCTCCAAACCTCACAT | LSLHYCYMEKVELEAPNLTSVDL |
| | | CGGTCGATTTGACCAATCGGCCAATTCCGTTGGCACTAAGTGAATCTCTGAAGGTGATGGAGGCAAAC | TNRPIPLALSESLKVMEANIKLL |
| | | ATTAAACTGCTACATAAGAGCGTGCTTTATGGTGATAATCTGGACTACATCTGCACCGAGCTTCCTGC | HKSVLYGDNLDYICTELPAALPH |
| | | CGCTCTTCCGCATGTGCAGAAACTCTCCATTACCTCGACACTCTGCATCTACGATGAGTTGCAAAGTT | VQKLSITSTLCIYDELQSFAKTS |
| | | TTGCCAAAACCTCAGTGAGATTCATCAATTTGAGGCATCTGAGCTTGTATTTGCCTCTTTATGGGGAC | VRFINLRHLSLYLPLYGDGRSVG |
| | | GGCAGGTCAGTTGGTGGGATTCTTCGTTTGGCCTACCTCTTGGAGCTCGCTCCAGTTCTAGAGGAACT | GILRLAYLLELAPVLEELELHFR |
| | | GGAACTGCACTTTCGCTTCTCTGATTTCGTCATCAGACAGGCGATCAGAGTGGATATGCTACCGTATC | FSDFVIRQAIRVDMLPYRHDKLK |
| | | GGCATGACAAGCTCAAGAGGGTGGTCATGTCTGGAGCCTGTCATTGGCAGGGGCTGATCGAGCTAGCA | RVVMSGACHWQGLIELAHHIRRC |
| | | CACCACATTCGTCGTTGTGCTAGTAGACTTGACTGTATGATCATGGATCCGATGGTTAGAATTAAGGG | ASRLDCMIMDPMVRIKGLPTVDW |
| | | TCTCCCGACGGTTGATTGGTTGGAGGAAAGAGGCCGCAGGATTGCCAAAGAGCTTCTTAAGAGGCAGG | LEERGRRIAKELLKRQEFQGVLT |
| | | AGTTTCAGGGTGTCCTCACTGTTTTGTGA SEQ ID NO: 151 | VL* SEQ ID NO: 152 |
| 77 | Xa21_OsJ_12449 | ATGGCTGCTCACCGCGTGGGGCGATGCATGCTCCTGCAACCACCGCGCCTCGCCACCTGCGTCCTCTT | MAAHRVGRCMLLQPPRLATCVLF |
| | (Os03g0730400) | CCTCCTGCTACCGCTGCTGCTGCTGCCGTGCTCGGCGTCGTCGTCGGTGATCACCCACCTCCCGGGCT | LLLPLLLLPCSASSSVITHLPGY |
| | | ACCACGGCCGCTTGCCGTTCCACCTTGAGACCGGGTACGTTGGTGTGGATGAGGAGACGGGCACTGAG | HGRLPFHLETGYVGVDEETGTEL |
| | | CTCTTCTACTACTTCGTTGAATCGGAGAGGAGCCCAAGTACAGACCCTGTGATCCTCTGGTTGACAGG | FYYFVESERSPSTDPVILWLTGG |
| | | CGGGCCTGGCTGCTCCGGATTCAGCGGTGTCGTCTTTGAAGTAGGACCTATGAAATACGTGTTGGAGC | PGCSGFSGVVFEVGPMKYVLEPY |
| | | CTTATAATGGTAGTTTGCCACAATTGGTTTATAATCGGTATTCATGGACACAGATGGCAAGCATCCTC | NGSLPQLVYNRYSWTQMASILFL |
| | | TTCTTGGACACTCCTGTTGGTTCTGGTTTCTCATACGCTCATGATCCTAAAGGTTATAATGTCGGGGA | DTPVGSGFSYAHDPKGYNVGDIS |
| | | CATATCATCGTCTTTGCAAGTCGTCACATTTCTGAAGAAGTGGTTCAATGATCACCCACGCTATCTCT | SSLQVVTFLKKWFNDHPRYLSNH |
| | | CAAATCATTTCTATGTTGGAGGAAGCTCATACGCTGGAAAGGTGATTCCAATTATCATGAAGTACATT | FYVGGSSYAGKVIPIIMKYISEV |
| | | TCAGAAGTTACAGGGGTAGTTGGCAACCCCATTAGAGGCTCGAAGATTGATGATAATTTCAAAATTCC | TGVVGNPIRGSKIDDNFKIPYSH |
| | | ATATTCTCATGGTGTTGGGATCATATCTGATCAATTATACGAGGCTGCAGTAGCAAACTGCAATGGAG | GVGIISDQLYEAAVANCNGDYVT |
| | | ATTATGTGACCACAACAAATGAATTATGTGCTAAGGCCCTAAATGCTATTGATAATCTCATGTCTGAA | TTNELCAKALNAIDNLMSEVDDG |
| | | GTTGATGATGGAAATATCTTGGATGATAAATGTGTTCGTGCTACACCAAAACCCATAAATGATGTTTC | NILDDKCVRATPKPINDVSRSRS |
| | | AAGAAGTAGGAGTCTGCTAGAAGATTACATTCGGCAAAGTGAGCCGACTGTTCGACCTACTATCAATT | LLEDYIRQSEPTVRPTINCFSYR |
| | | GTTTTTCATACCGTTACTATCTATCATTCTTGTGGATGAACAACAATATGACTAGAGAAGCTCTTAAG | YYLSFLWMNNNMTREALKIKKGT |
| | | ATCAAGAAGGGAACAGTCGGCGAATGGATAAGATGCAAGACAGGGCTACCCTATGTTAAAGATGTCGC | VGEWIRCKTGLPYVKDVASSIKY |
| | | AAGCAGCATCAAGTATCATTTTGACCTCACCACAGGAGGTTACCGGGCACTTGTGTTCAGTATTCCAT | HFDLTTGGYRALVFSIPCLKNYY |
| | | GTCTTAAAAATTATTATGACATTTATGAATGCAGTGGTGACCATGATCTCACGTTGCCATTTCTGAGC | DIYECSGDHDLTLPFLSTQAWIR |
| | | ACCCAGGCATGGATAAGATCCTTGAACTTCTCCATAGTTGATGAGTGGAGAGCATGGCATGTGGATGG | SLNFSIVDEWRAWHVDGQAAGFT |
| | | CCAGGCTGCAGGATTTACAATTTTGTACGCCAACAACTTGACCTTTGCCACAGTAAAGTGTCCAGCCC | ILYANNLTFATVKCPALHNSHTG |
| | | TTCACAATTCACATACTGGCTTTCTGAAGAATCCTAAACAAGGTTTATGA | FLKNPKQGL* |
| | | SEQ ID NO: 153 | SEQ ID NO: 154 |
| 78 | Xa21_Os03g0741100 | ATGCAAATGGAGTCGTACTACGGCGCGTTCCACGCCGACGAGGCCGCGTTCTTCTTCCCCCACCACGT | MQMESYYGAFHADEAAFFFPHHV |
| | | GCCGGCGTCGCCGGAGCTGCCGTTCGGCCTCATTGCGTCGCCGGAGCCGGAGCCGGAGCCGGCGGCGG | PASPELPFGLIASPEPEPEPAAA |
| | | CGGAGGAGGCGAGGCAGAGCGCGTTCCGGGAGTACGGCGGCGCGGTGCACGCCGGTGCGCCGGCGGCG | EEARQSAFREYGGAVHAGAPAAA |
| | | GCGGGGGCTGTCACCACCGGTGGGACGAACATCCACCGGAGGGTGATGGACGTGCTGGGGAGGATGGG | GAVTTGGTNIHRRVMDVLGRMGG |
| | | CGGCGGCGGCGGCGGCGGCGGAGGGGAGAAGGGGGAGGGGGAGGAGATGGAGGAGGAGGAGGAGGTGC | GGGGGGGEKGEGEEMEEEEEVPQ |
| | | CGCAGCGGCGGCGGCGCGGGCAGGGCGCCGACGTGGAGAGCAGCCGCGGTTTCCGCCACATGATGCGC | RRRRGQGADVESSRGFRHMMRER |
| | | GAGCGCCAGCGCCGCGAGAAGCTCAGCCAGAGCTACGCCGACCTCTACGCCATGGTCTCCTCTCGCTC | QRREKLSQSYADLYAMVSSRSKG |
| | | CAAGGGGGACAAGAACTCGATCGTGCAGTCGGCGGCGATCTACATCCACGAGCTGAAGGTCGCGAGGG | DKNSIVQSAAIYIHELKVARDQL |
| | | ACCAGCTGCAGAGGAGGAACGATGAGCTCAAGGCCCAGATCATGGGCCACGACGAGCAGCAGCCGTGC | QRRNDELKAQIMGHDEQQPCVTV |
| | | GTCACGGTGCAGTTCGAGGTCGACGAGCCGTCGTCGTCGATCGACTCCATGATCGCCGCGCTCCGGCG | QFEVDEPSSSIDSMIAALRRLKG |
| | | GCTCAAGGGCATGAGCGTCAAGGCGCGCGGGATCCGGTCGAGCATGTCCGGGAACAGGCTGTGGACGG | MSVKARGIRSSMSGNRLWTEMNV |
| | | AGATGAACGTCGAGACCACGATTGCGGCTTGTGAAGTGGAAAAGGCAGTTGAAGAGGCTCTCAAGGAA | ETTIAACEVEKAVEEALKEVERN |
| | | GTAGAGAGGAACCAGCCTGACAGCGATGCCCCATTTCCTGGAAGCAAAGGCTGGACACAGACATCTCA | QPDSDAPFPGSKGWTQTSHVQNV |
| | | TGTGCAAAATGTGTTTTGA SEQ ID NO: 155 | F* SEQ ID NO: 156 |
| 79 | Xa21_Os03g0743500 | ATGGAAGGGCTGACGAGCGAGCAGATGGTGGCGTTCCAGGAGGCGTTCTTGCTCTTCGACAAGAACGG | MEGLTSEQMVAFQEAFLLFDKNG |
| | | CGATGGATGCATCACCTTGGAAGAGCTGGCTGCGGTGACTCGTTCTCTTGGCCTTGAGCCGACCGATC | DGCITLEELAAVTRSLGLEPTDQ |
| | | AGGAGCTCAACGACATGATGAGGGAAGTTGATACGGATGGCAATGGGATCATAGATTTTCAGGAGTTC | ELNDMMREVDTDGNGIIDFQEFL |
| | | CTGAGTCTCATTGCCAGGAAGATGAAGGATGGGGATGGAGACGAAGAGCTGAAAGAAGCTTTCGAGGT | SLIARKMKDGDGDEELKEAFEVL |
| | | CCTAGACAAGGATCAGAATGGTTTTATCTCCCCAACTGAGCTGAGGACAGTGATGACCAATCTTGGGG | DKDQNGFISPTELRTVMTNLGEK |
| | | AGAAGATGACAGACGAGGAGGTTGAGCAGATGATAAGGGAGGCGGATACCGATGGCGACGGGCAGGTG | MTDEEVEQMIREADTDGDGQVNY |
| | | AACTACGATGAATTTGTGATAATGATGAAAAACGCTGAACGGAAGATATCTGGGTGA | DEFVIMMKNAERKISG* |
| | | SEQ ID NO: 157 | SEQ ID NO: 158 |
| 80 | Xa21_Os03g0748900 | ATGGAGGGCGAGAGGAGGGCGGTGAGGACGGTGCGGGTGCGGAACATCTCAGATCTGGCAGGGGAGCG | MEGERRAVRTVRVRNISDLAGER |
| | | GGAGGTGCGGGAGTTCTTCTCCTTCTCCGGCGAAATCGAGCACGTCGACATCAGATGCGATGGGGTGG | EVREFFSFSGEIEHVDIRCDGVA |
| | | CGACGGGGAGGACGGCCTACGTCACGTTCAAGGATCCCAAGGCCCTAGAGATCGCGCTGCTGCTGTCG | TGRTAYVTFKDPKALEIALLLSG |
| | | GGAGCAACAATTGTGGATCGGGTTGTCAACATAACCCCTGCTGAAGACTACATCTACATCCCTGTTTC | ATIVDRVVNITPAEDYIYIPVSD |
| | | TGACCAGCAACTTGTGGTCAGTGAGGTGGTCGGTGAGGTGACTAGTACGGCTCCTAATGCAGATTTGG | QQLVVSEVVGEVTSTAPNADLDQ |
| | | ACCAACCTACCGAGGCCAATGCCAGCCCCACTACTGGCCGTGTTTATGTCAGCAAGGCTCATGATGTC | PTEANASPTTGRVYVSKAHDVMT |
| | | ATGACAACTGTGATTGCAAGGGGTTCAGCAATGCGACAAGATGCTGTGAACAAAGCTAAAGCATTCGA | TVIARGSAMRQDAVNKAKAFDEK |
| | | TGAGAAACATCAATTGAGGGCAAATGCATCGGCCAAGATCAATTCCTTTGATAAGCGTGTTGGAATTT | HQLRANASAKINSFDKRVGISEK |
| | | CAGAGAAGATAAACAGCGGGATAACAGTTGTAAATGAGAAAGTGAAATCTGTAGACCAAAGGCTACAT | INSGITVVNEKVKSVDQRLHVSD |
| | | GTTTCTGACAAAACAATGGCTGCTTTGCTGGCTGCAGAGCGCAAGCTAAATGACACTGGCTCTGCTGT | KTMAALLAAERKLNDTGSAVKTN |
| | | GAAAACCAACAGGTATGTGTCTGCTGGAACAAGTTGGCTAAATGGTGCCTTCAGCAAGGTTGCTAAGG | RYVSAGTSWLNGAFSKVAKAGHV |
| | | CTGGTCATGTTGCTGGCTCAAGAACAAGAGAGAAGTTCCAAATAGCTGTGTCCAATCTAACAGCGAAA | AGSRTREKFQIAVSNLTAKGPAV |
| | | GGTCCTGCTGTTGTTGCTTGA SEQ ID NO: 159 | VA* SEQ ID NO: 160 |
| 81 | Xa21_SORBIDRAFT_01g006820 | ATGGCGCCGTGGAGCGGCTTATGGGGCGGGAAGTTGGCCGCCGCCGGCGGCGGCGGCGGCGACCACTA | MAPWSGLWGGKLAAAGGGGGDHY |
| | (Os03g0770800) | CCGGGGCACGCCGGTGGTGGTCAAGATGGAGAACCCCAACTGGTCCATCTCCGAGATCTCCTCGCCGG | RGTPVVVKMENPNWSISEISSPD |
| | | ATGACGACGACGACGAGGAGTTCTTGGTCGGCGGCCGCCGCAAGGGGGGGCGCGGCAAGAACGCCAAG | DDDDEEFLVGGRRKGGRGKNAKQ |
| | | CAGATCACCTGGGTGCTGCTGCTCAAGGCGCACCGCGCCGCCGGGTGCCTCGCGTCGCTGGCCTCCGC | ITWVLLLKAHRAAGCLASLASAA |
| | | GGCCGTCGCGCTCGGCGCCGCCGCCAGGCGGAGGGTCGCCGCCGGGAGGACGGACGCCGACGCCTCCG | VALGAAARRRVAAGRTDADASAA |
| | | CCGCCGCCGCCGGCGAGAGCCCCGTGCTCCGCTCCCGCTTCTACGCGTTCATCAGGGCATTCGTCGTC | AAGESPVLRSRFYAFIRAFVVLS |
| | | CTCTCGGTGCTGCTCTTCATTGTCGAGCTCGGCGCCTACATCAATGGCTGGGACGACCTCGCCGCCTC | VLLFIVELGAYINGWDDLAASAL |
| | | CGCGCTCGCCCTCCCCGTCATCGGCGTCGAGTCGCTCTACGCGTCGTGGCTGCGCTTCCGCGCCACCT | ALPVIGVESLYASWLRFRATYVA |
| | | ATGTCGCCCCATTCATCCAGTTCCTGACCGACGCATGCGTCGTGCTCTTCCTCATCCAGAGCGCCGAC | PFIQFLTDACVVLFLIQSADRLI |
| | | CGCCTCATCCAGTGCCTCGGCTGCTTCTATATCCATCTCAAGCGCATCAAGCCTAACCCCAAGTCACC | QCLGCFYIHLKRIKPNPKSPALP |
| | | GGCATTGCCCGACGCCGAAGACCCCGACGCCGCGTACTACCCCATGGTACTCGTGCAGATACCAATGT | DAEDPDAAYYPMVLVQIPMCNEK |
| | | GCAACGAGAAGGAGGTTTATCAGCAATCAATTGCGGCCGTGTGCAATCTTGATTGGCCGAGGTCCAAC | EVYQQSIAAVCNLDWPRSNFLVQ |
| | | TTCTTGGTTCAGGTATTGGATGATTCGGACGATCCGACGACGCAGACGCTGATCAGAGAAGAGGTGCT | VLDDSDDPTTQTLIREEVLKWQQ |
| | | CAAGTGGCAGCAGAACGGCGCTCGGATTGTGTACCGGCATCGTGTCCTTAGGGATGGTTATAAGGCTG | NGARIVYRHRVLRDGYKAGNLKS |
| | | GAAATCTCAAGTCGGCGATGAGCTGCAGCTATGTCAAGGACTATGAGTTTGTTGCCATCTTTGATGCG | AMSCSYVKDYEFVAIFDADFQPN |
| | | GACTTCCAACCAAATCCGGACTTCTTGAAGCGAACCGTGCCACATTTCAAGGACAATGATGAATTGGG | PDFLKRTVPHFKDNDELGLVQAR |
| | | GCTAGTGCAAGCAAGATGGTCATTTGTGAATAAGGATGAGAACCTATTGACTCGTCTGCAAAACATTA | WSFVNKDENLLTRLQNINLCFHF |
| | | ATCTTTGCTTCCACTTTGAGGTAGAACAGCAAGTGAATGGGATATTCCTAAACTTCTTCGGGTTCAAT | EVEQQVNGIFLNFFGFNGTAGVW |
| | | GGCACTGCTGGTGTGTGGAGAATCAAGGCATTGGATGACTCGGGAGGGTGGATGGAGCGTACCACAGT | RIKALDDSGGWMERTTVEDMDIA |
| | | GGAGGACATGGACATTGCAGTCCGGGCACACCTCAGGGGCTGGAAATTTATCTTCCTAAATGATGTCG | VRAHLRGWKFIFLNDVECELPES |
| | | AGTGTGAATTGCCAGAGTCATACGAAGCTTATCGGAAGCAGCAGCATCGGTGGCATTCTGGTCCAATG | YEAYRKQQHRWHSGPMQLFRLCL |
| | | CAGCTATTCAGACTCTGCTTACCAGACATTATCAAATGCAAGATTGCATTTTGGAAGAAAGCCAACTT | PDIIKCKIAFWKKANLIFLFFLL |
| | | GATCTTCCTTTTCTTCCTTCTCCGGAAGCTCATACTACCATTCTACTCTTTCACGCTCTTCTGCATCA | RKLILPFYSFTLFCIILPMTMFV |
| | | TCCTCCCAATGACGATGTTTGTGCCTGAAGCCGAGCTCCCTGACTGGGTCGTATGCTACATTCCTGCC | PEAELPDWVVCYIPALMSLLNIL |
| | | CTGATGTCCTTGCTCAACATCCTGCCTTCTCCGAAGTCTTTCCCTTTCATCATCCCATACCTACTTTT | PSPKSFPFIIPYLLFENTMSVTK |
| | | CGAGAACACCATGTCCGTGACAAAATTCAATGCCATGATCTCTGGTCTGTTCCAGCTTGGAAGTGCAT | FNAMISGLFQLGSAYEWVVTKKS |
| | | ATGAGTGGGTGGTGACCAAGAAGTCAGGCCGATCATCGGAGGGCGACCTCATTTCTTTGGCACCCAAA | GRSSEGDLISLAPKELKHQKTES |
| | | GAGTTGAAACACCAAAAAACTGAATCGGCGCCTAACCTCGACACTATTGCAAAGGAGCAGTCCGCTCC | APNLDTIAKEQSAPRKDVKKKHN |
| | | TAGAAAGGACGTAAAGAAGAAGCACAACCGGATATACAAGAAAGAGCTTGCACTCTCATTGCTCCTCC | RIYKKELALSLLLLTAAARSLLS |
| | | TAACCGCTGCGGCTCGCAGTTTGCTCTCGAAACAGGGTATACATTTCTACTTCCTCCTGTTCCAGGGC | KQGIHFYFLLFQGISFLLVGLDL |
| | | ATATCTTTCTTGTTAGTAGGCCTTGATCTCATAGGCGAGCAGATTGAGTAA | IGEQIE* |
| | | SEQ ID NO: 161 | SEQ ID NO: 162 |
| 82 | Xa21_Os03g0788500 | ATGGGGAACACATGCGTCGGCCCCAGCATCAGCAAGAACGGCTTCTTCCAGTCCGTCTCCACGGTGCT | MGNTCVGPSISKNGFFQSVSTVL |
| | | CTGGAAGGCGCGCCAGGACGGCGACGACGTGCTGCCCGGCGCCAATGGCGGCCCTGACGGCGGCGGCC | WKARQDGDDVLPGANGGPDGGGQ |
| | | AGGGCCGGTTGCCGGCGCCGCCGCCGCCGACTTCCGACGCGCCGCTCGCCGTCCAAAACAAGCCCCCC | GRLPAPPPPTSDAPLAVQNKPPE |
| | | GAGCACGTCAAGATCGTCTCGACCACGGACACCGCCTCCGCCGAGCAGGACGCCTCCAAGTCGAGCGC | HVKIVSTTDTASAEQDASKSSAG |
| | | CGGCTCGGACTCCGGCGAGGCCGCCAGGCCGCGGCCGAGGGTGCCGCCGGTGAAGCGCGTCTCCAGCG | SDSGEAARPRPRVPPVKRVSSAG |
| | | CCGGGCTGCTCGTCGGCTCGGTGCTGAAGCGCAAGACGGAGAGTCTCAAGGACAAGTACAGCCTGGGG | LLVGSVLKRKTESLKDKYSLGRK |
| | | CGGAAGCTCGGGCAGGGGCAGTTCGGGACGACGTACCTGTGCGTGGAGCGCGCCACCGGCAAGGAGTT | LGQGQFGTTYLCVERATGKEFAC |
| | | CGCCTGCAAGTCCATCCTGAAGCGGAAGCTGGTCACCGACGACGACGTCGAGGACGTCCGCCGCGAGA | KSILKRKLVTDDDVEDVRREIQI |
| | | TCCAGATAATGTACCACCTCGCCGGCCACCCCAACGTGATCTCCATCAGGGGCGCCTACGAGGACGCC | MYHLAGHPNVISIRGAYEDAVAV |
| | | GTCGCCGTGCACCTCGTCATGGAGCTCTGCGCCGGCGGCGAGCTGTTCGACCGGATCGTCCAGAAGGG | HLVMELCAGGELFDRIVQKGHYT |
| | | GCACTACACCGAGCGGAAGGCGGCGGAGCTCGCCAGGGTCATCGTCGGCGTCGTCGAGGTGTGCCACT | ERKAAELARVIVGVVEVCHSMGV |
| | | CCATGGGCGTGATGCACCGCGATCTCAAGCCCGAGAACTTCCTCTTCGCCGACCAGACGGAGGAGGCC | MHRDLKPENFLFADQTEEAALKT |
| | | GCTCTCAAGACCATTGACTTTGGTCTCTCCATTTTCTTTCGCCCAGGTCAAGTATTCACTGATGTTGT | IDFGLSIFFRPGQVFTDVVGSPY |
| | | TGGTAGCCCTTACTATGTCGCGCCAGAAGTTCTGAAAAAGAAATATGGTCAAGAGGCAGATGTCTGGA | YVAPEVLKKKYGQEADVWSAGVI |
| | | GCGCTGGTGTGATAATTTACATCTTGTTGTGTGGTGTGCCGCCATTTTGGGCAGAGAACGAGCAGGGT | IYILLCGVPPFWAENEQGIFEEV |
| | | ATATTTGAAGAGGTTTTGCATGGTAGACTTGACTTTCAGTCAGAACCATGGCCTAGCATCTCTGAAGG | LHGRLDFQSEPWPSISEGAKDLV |
| | | TGCCAAAGATCTCGTAAGGAGAATGCTTGTTAGGGACCCGAAGAAGAGATTGACAGCTCATGAAGTTT | RRMLVRDPKKRLTAHEVLRHPWV |
| | | TAAGGCATCCATGGGTCCAGGTTGGTGGTTTGGCTCCTGACAAGCCCCTGGATTCTGCTGTTCTATCT | QVGGLAPDKPLDSAVLSRMKQFS |
| | | CGTATGAAGCAATTCTCAGCTATGAATAAGCTGAAAAAGATGGCTCTTAGGGTCATTGCAGAGAACCT | AMNKLKKMALRVIAENLSEDEIA |
| | | ATCTGAAGACGAAATCGCCGGCCTGAAAGAAATGTTCAAGATGATCGACACAGACAACAGTGGACAGA | GLKEMFKMIDTDNSGQITFEELK |
| | | TCACCTTCGAAGAACTCAAGGTTGGGCTGAAAAAAGTTGGTGCTAACCTCCAAGAATCCGAGATCTAC | VGLKKVGANLQESEIYVLMQAAD |
| | | GTACTCATGCAAGCCGCGGATGTAGATAACAGCGGCACGATAGATTACGGGGAGTTCATCGCTGCAAC | VDNSGTIDYGEFIAATLHMNKIE |
| | | TCTGCATATGAACAAGATCGAGCGGGAGGATCACCTGTTCGCGGCGTTCCAATACTTCGACAAGGACG | REDHLFAAFQYFDKDGSGYITAD |
| | | GCAGCGGCTACATCACGGCCGATGAGCTGCAGCTCGCCTGCGAGGAGTTCGGCTTGGGAGACGTTCAG | ELQLACEEFGLGDVQLEEMIREV |
| | | CTCGAGGAGATGATCAGAGAAGTCGACGAAGACAACGATGGTCGCATAGATTACAACGAGTTCGTGGC | DEDNDGRIDYNEFVAMMQKPTMG |
| | | GATGATGCAGAAGCCGACGATGGGGCTCCCGGCGAAGAAGTCCGGCGGGCTGCAGAACAGTTTCAGCA | LPAKKSGGLQNSFSIGFREALRM |
| | | TCGGCTTCAGGGAGGCCTTGAGGATGTCTTGA SEQ ID NO: 163 | S* SEQ ID NO: 164 |
| 83 | Xa21_Os04g0301500 | ATGGACGCCGAGATGGCCATGGGCGAGAGCTTCGCCTACTACTGGGAGACGCAGCGCTACCTCGAGTC | MDAEMAMGESFAYYWETQRYLES |
| | | CGAGGAGCTCGACAGCATGTACCTGCCGACGCAGGATGACTCCAACTACGAGTCGAGCTCGCCGGACG | EELDSMYLPTQDDSNYESSSPDG |
| | | GGTCGCACTCGTCGTCGGCGACCGCGGCGGCCGTCGGCGGGGACGCCGCCGCCGCGGTGGCTGGTGGT | SHSSSATAAAVGGDAAAAVAGGG |
| | | GGCGGCGGGATGATGATGATGATGGGCGGCGGCAGCAGCGGTGGTGGCGACGACGCCGGTGGCGCGAA | GGMMMMMGGGSSGGGDDAGGANK |
| | | CAAGAACATCCTCATGGAGCGCGACCGCCGCCGCAAGCTCAACGAGAAGCTCTACGCGCTCCGCAGCG | NILMERDRRRKLNEKLYALRSVV |
| | | TCGTGCCCAACATCACCAAGATGGACAAGGCGTCGATCATCAAGGACGCGATCGAGTACATCCAGCGC | PNITKMDKASIIKDAIEYIQRLQ |
| | | CTGCAGGCGGAGGAGCAGCAGATGCTCCGGGAGGTCGCCGCGCTCGAGTCCGCCGCCGCGGCGAGCGC | AEEQQMLREVAALESAAAASAAP |
| | | CGCCCCGGCAGCGGCGAACCCGTTCGCCGGCCTCGGCGCCGACGAGGAACACGAGTACGGCCACCACC | AAANPFAGLGADEEHEYGHHHPS |
| | | ACCCCTCGTCGTCGTCGGAGCGGACGAAGAAGGTGAAGCGGGCGCTGTCCGTGTCGTCCATCAGCGAC | SSSERTKKVKRALSVSSISDALL |
| | | GCCCTGCTCGCCGCGGCGGCGCCGCCGCCGCCGGTGGAGATCCAGGAGCTGCGCGTGTCGGAGGTGGG | AAAAPPPPVEIQELRVSEVGDRV |
| | | CGACAGGGTGCTGGTGGTGAGCGTGACGTGCAGCAAGCGCCGCGACGCCATGGCCCGGGTGTGCCGCG | LVVSVTCSKRRDAMARVCRALEE |
| | | CCCTCGAGGAGCTCCGCCTCCGCGTCATCACCGCCAACATCACCTCCGTCGCCGGCTGCCTCATGCAC | LRLRVITANITSVAGCLMHTLFV |
| | | ACACTCTTCGTCGAGGTAGATCACATGGACAGTGTCCAAATGAAGCAGATGGTGGAGGCTGCTCTCTC | EVDHMDSVQMKQMVEAALSQLVA |
| | | CCAGCTTGTGGCGACCGGCAGTCCGTTGAGCTCCATGAGCTATTAG | TGSPLSSMSY* |
| | | SEQ ID NO: 165 | SEQ ID NO: 166 |
| 84 | Xa21_Os04g0398800 | ATGGCGGAGGCGGGGCCTAGCTGGCCGGCCGCCGCCGCCGGCGACTTCCCGGAGCTCCTCGCCTTCTG | MAEAGPSWPAAAAGDFPELLAFC |
| | | CGCCCGCGCGGAGGCCCTCATCGCCGAGCTCCTCCTCCTCTCCGACCGCGCCCCGCCGCAGTTCGCCG | ARAEALIAELLLLSDRAPPQFAD |
| | | ACCGCCGCTTCCACCCCGTCCTCTTCGACTTCCGATACTTCGACTCCCCGGGCGAATTCGAGGCGAGG | RRFHPVLFDFRYFDSPGEFEARI |
| | | ATCGAGGGCAACATCGAGCTGGAGGCCCTGGAAGACGAGCTCCGCGATTCCTGCGGCTCCTACATGCG | EGNIELEALEDELRDSCGSYMRR |
| | | GAGGTTCTTCGCCTTGCTGGATGCTGCCGTCGCTTACCACGCCGAGCTGTGCAGCTACCTCAACGACC | FFALLDAAVAYHAELCSYLNDLQ |
| | | TGCAGGAAGGGCTGTATGTGCATTGCACATTGGATGGTGTACTGGAGAGCAACTGGGCTTGCCAATTG | EGLYVHCTLDGVLESNWACQLLT |
| | | CTTACAGAGTCGATGACTTTGTTTGGGTGTATGGTTCTGCTAATGGAGCACAGGATTAGCGGATTGCT | ESMTLFGCMVLLMEHRISGLLRE |
| | | GCGCGAGAGACTTCTGGTGGCGTATATGCGGCACGAGCGTTGCTTCAGCTTCCCTAATGTGGAGCGCA | RLLVAYMRHERCFSFPNVERICE |
| | | TATGCGAGCTCTGTCGTCAGCATGCGACTGCACCTCCTTCGCCTGGTGCCTCCGGTTCATCGTTGCAT | LCRQHATAPPSPGASGSSLHSAE |
| | | AGTGCGGAAATCATCTCTGTACAGAAGCCTGAGGACCTGCTGAGGAGGTTTCAGTTTCCCGAGCTCGT | IISVQKPEDLLRRFQFPELVVDA |
| | | TGTCGATGCTGTCATAACTTGCTTGAGAAATGGTGATGTTTACAACAATGTCCGGTTCTACCCTGATC | VITCLRNGDVYNNVRFYPDPQHR |
| | | CGCAGCACCGGACAACCGCTCTCTCATTGCAAGGTGGACAAATGTATGTGTTGTTATTTTATTCTCGT | TTALSLQGGQMYVLLFYSRDLLH |
| | | GACCTTCTACACAACGGTTTGGCTATGCGGGAGATTATTGATCGCTTCTTTAAGGACAACTGGGTGGT | NGLAMREIIDRFFKDNWVVPIFL |
| | | TCCCATCTTCTTGCATTTCTCGGTAGATTTACTTGTCTCATGGGATGCATTCAAAGAAGCGAAATCAT | HFSVDLLVSWDAFKEAKSSLVSC |
| | | CATTAGTTTCTTGCCTTTCTCCCACCTTCATACGTGATCGCAGTCTATACCACTATACAAAGGTATCT | LSPTFIRDRSLYHYTKVSSLLAD |
| | | TCTCTTCTAGCTGACCTGGATAGTCATATGCATGCGGTAAACAAGGAATATGTTCTGGATAATTCTCT | LDSHMHAVNKEYVLDNSLNLLSI |
| | | GAATCTTCTTTCTATCATCAGAGAGTGCAATTGCACACTGCGTTGGCTTTTGCTTCACCGCATGACAA | IRECNCTLRWLLLHRMTNDKKAR |
| | | ATGATAAGAAGGCAAGGGACCTAGTGATCTGTTTAGGGTCATCACAACATGCTGACGAAGGGAAGCTG | DLVICLGSSQHADEGKLLQLLMK |
| | | TTACAGCTCCTAATGAAGACTGCTAAACTTGAGTTTGAGGTGAAGGAACTGCATGCTGAACTATTGAA | TAKLEFEVKELHAELLKTRKSMW |
| | | AACCAGAAAAAGTATGTGGTATGAGAAGAGGCATGATGCTTTGGAATGTATGAAGGACCTGTCTCAGA | YEKRHDALECMKDLSQNYLGIWA |
| | | ACTATCTTGGGATTTGGGCTGCTTCATGCAAATTAAAAAACAAGAGCATCAAAGACTGGTTGGAGCAC | ASCKLKNKSIKDWLEHLSSEVSS |
| | | CTGTCATCAGAGGTTAGCTCCCTGGATTATGCCACAATTGGAAACTCCGGCAGGATAATTCATCGTGT | LDYATIGNSGRIIHRVLSTLKDI |
| | | GCTATCTACGCTAAAAGATATTGAGCTACTTCACCAAATCAAAGAGAATATACAGATAAAGCATGGCT | ELLHQIKENIQIKHGFSKIQKNL |
| | | TCTCCAAAATACAGAAGAATCTTCATGATATGATAAAAGTTCTAAACCTAAACCAAGAGTCCATAAGT | HDMIKVLNLNQESISVFSVITDG |
| | | GTTTTCTCAGTGATTACTGATGGTAAGTATGCATGGGGTTATCTTACTTATTTTGAGGAACTTCTTAA | KYAWGYLTYFEELLKKKISQDPS |
| | | GAAGAAGATCAGTCAAGATCCATCAGAGTCTTTATTTTTGCATACGATGTTTCTTAAATTTCAATCAT | ESLFLHTMFLKFQSWLDAPLQRI |
| | | GGTTGGATGCACCGCTGCAGAGGATCAAGCAGTACGAAAGTCCAGATCTTCAATATGTCTCAACCTAC | KQYESPDLQYVSTYYVSKYAAKI |
| | | TATGTTTCAAAGTATGCAGCTAAAATATTTGCAGTTCTAGATATTATCCCGGCTATATTGCTTAAAAT | FAVLDIIPAILLKISIAVDYINA |
| | | TTCTATTGCTGTGGATTACATCAATGCTGAACAGTCCACTCATCTAATAAATCGCATCAACAAGGAAA | EQSTHLINRINKETLEDLMQLDQ |
| | | CATTGGAAGATCTCATGCAGTTGGATCAACAACTATGTCAAGCACAGCAAGCTTCCAAACTATGCATT | QLCQAQQASKLCIVSEGLLNMSK |
| | | GTTTCAGAGGGCCTTTTGAACATGTCAAAGAACTTTGATGGCCTTATAGATCTGAATTTAGGTGGATG | NFDGLIDLNLGGWLKQMIKKELV |
| | | GCTTAAGCAAATGATAAAAAAGGAGTTAGTAAGCCAACTACAAGGCAAGCTGAAAGCCTTATCATTGC | SQLQGKLKALSLLIYGDIEGNLM |
| | | TCATTTATGGTGATATAGAAGGCAATCTCATGTCATTGTCAAATTATATGCTTTCACAGATGCAGAGA | SLSNYMLSQMQRMEFLQHILHIE |
| | | ATGGAATTCCTACAGCACATACTGCACATTGAGGGGTGTTCAATCTGGGAAGAAACATTGACTGCTGT | GCSIWEETLTAVLEECAKREVLE |
| | | GCTAGAAGAATGTGCTAAAAGGGAGGTCTTAGAGTTTATGGGATGCATGCAACCATCCACTAATATGG | FMGCMQPSTNMVKPSNHMSNPGT |
| | | TGAAACCATCGAATCATATGTCCAATCCGGGCACTTTTTTTGGTAATATTTTAGATTAA | FFGNILD* |
| | | SEQ ID NO: 167 | SEQ ID NO: 168 |
| 85 | Xa21_Os04g0459600 | ATGGCGGCGCGGCGGAGGCACGCGGCTGTGGTGGTTGCAGGGGTGGCGGCTGCGGGGGAGTGGGTGGT | MAARRRHAAVVVAGVAAAGEWVV |
| | | GGTGACCGCCTGGGCAGGGAACCGGCTTGGGCGTTGTCGCCGGCGGAGGTGGAGGTGGAGGAGGTGGG | VTAWAGNRLGRCRRRRWRWRRWG |
| | | GACGAACCAGGAGCTGCTGCGGCGGACGGGGCTCCCGCCACGGGACCGCGCCATCGTCGTCAACCTGG | RTRSCCGGRGSRHGTAPSSSTWT |
| | | ACCGTCGCGCCGCTTGTCCGGGAGCTCCGCTCCCGCCTCTCCGCCTCTCCAACCCCCGCACAGTCGCC | VAPLVRELRSRLSASPTPAQSPS |
| | | GTCGCCGCCGCAGAAGAATATAAAGGATGCTGTCACCCAACGGTGCTGGAATGCTGAGGACTCAGGAC | PPQKNIKDAVTQRCWNAEDSGLS |
| | | TGAGTGAGGTGTTCGTTGACCCGGCTCGCGACAAGAGCCTCATCTTCGACCTGCTCGACCTCAAGGGC | EVFVDPARDKSLIFDLLDLKGEV |
| | | GAGGTGGAGGACGGCAACAGTGCACTTTGGTTCCTGCGCGACATCGCCAATGAGCAGGATGCGGGGGA | EDGNSALWFLRDIANEQDAGDNL |
| | | CAACTTGGTGGTTGAGCATTCTGGGATGATTGAGCTAAGTGGTCTGCGATTTGGAGATGCTCCTGCAG | VVEHSGMIELSGLRFGDAPAVAG |
| | | TGGCTGGAACTGCGGTTGGTCAGCTGGCTATCTCAAAAGGAAGGCAAGGCAGAGAAGCACAGAACATT | TAVGQLAISKGRQGREAQNIVRL |
| | | GTTCGACTTTACTTGGCCAATATACGCCTCAAGAATGCAGCTACTGATGTAGTTATTACTGCATATGA | YLANIRLKNAATDVVITAYEPLL |
| | | GCCACTGTTGATAAACCCCTTGAGTGAAAGCGCCAGTGCAGTTGCAGCCGGTCCAGCAGTACCAGCAG | INPLSESASAVAAGPAVPAEQAG |
| | | AACAAGCAGGATGCTTAGCAATGTCTGAGATCTTCAAGCTCGCCGTGATGAACTTCAATGTCCATGAC | CLAMSEIFKLAVMNFNVHDWNLF |
| | | TGGAATCTTTTCAATGGCAGCAGTTGA SEQ ID NO: 169 | NGSS* SEQ ID NO: 170 |
| 86 | Xa21_Os04g0464966 | ATGCGACGCGAGCGCGACGCGACGCAAATCCCGGAGAACCCTATGGAGATGGAGGGGATCCCCCAGAC | MRRERDATQIPENPMEMEGIPQT |
| | | CGCCGCGGCGGCGGCGGCGGAGGCATCTGAGCCACCGCGCAAGCGCGCCCGCGTCGACGACGACGGCG | AAAAAAEASEPPRKRARVDDDGG |
| | | GCGGCGGCGCGGGGGAGGAGGAGGAGGACCGGCTGAGCGAACTGCCGGACTGCCTCCTGGAGGACATC | GGAGEEEEDRLSELPDCLLEDIL |
| | | CTGGCGCATCTCGGGTCCCGCCAGGCCGTGCAGACGAGCGTGCTGTCGCGGCGGTGGCGGAACCTCTG | AHLGSRQAVQTSVLSRRWRNLWR |
| | | GCGCGGCGTGCGGGTCGTGGACATCGACGTGGGGTCGTTCCGCCTCCCGGGCGCCGACGGCGACCCGC | GVRVVDIDVGSFRLPGADGDPPR |
| | | CGCGGTTCCGCCTGGATCGCATCGAGGACTTCGCGGACGGCGTCCTGTCGCCGTCGCTCCACCCGGGC | FRLDRIEDFADGVLSPSLHPGAA |
| | | GCCGCGCGGGAGCTGGACGCGCTCCGGATGCGCCTGGACGAGGACGCGGTGACCACCAACTTCCAGAG | RELDALRMRLDEDAVTTNFQRWI |
| | | GTGGATCCGCCGCGCCCTCTGGCGCCGCCCCGCCACCGTCGACCTCTACTACCTCCCCCGCCGCTCCT | RRALWRRPATVDLYYLPRRSFSW |
| | | TCAGCTGGCCGCCCGCCGTGCCCCTGACCCCGGTCGCCGCCGTCAGCCGCCTCAAGACCCTGCGCATC | PPAVPLTPVAAVSRLKTLRIFGL |
| | | TTCGGGCTCCGCCCGACGGTCGTCTTCGGCGCCGACGAGTTCCCGGCCCTCGAGGACCTGCACATCGA | RPTVVFGADEFPALEDLHIERCS |
| | | GAGGTGCTCGTACGCGCACGGCACCATCGCCTCGCCCACTCTCAAGCGGCTCGCCCTCGTCTCCCCAA | YAHGTIASPTLKRLALVSPINGC |
| | | TCAACGGCTGCTTCGTCCGCGAGCAGCGCCTCACGGCTCCAGGCCTCACCTCGCTGCGCCTCGTCCTC | FVREQRLTAPGLTSLRLVLPYSR |
| | | CCGTACAGCCGGGAGGAAGGGGTTAGGGTGATCACCGACGCGCCGCTCACATCGCTCGTCGACGCGTC | EEGVRVITDAPLTSLVDASITIV |
| | | GATCACCATAGTCGACACCGATCCTGGGGATCCGCGGAACCGACGGGTGAACCAATTCAAGGTTGACT | DTDPGDPRNRRVNQFKVDFLVAI |
| | | TCTTGGTTGCCATCAGCAACCTGCTTGGCCGCCTCACCAGCGTCAGAAATCTCGACCTGACTGGGCTC | SNLLGRLTSVRNLDLTGLNATAL |
| | | AATGCAACGGCACTGCTGGATAATAAATCCCAGGAATTCCCGATGTTCCCCTACTTGACAACATTGCT | LDNKSQEFPMFPYLTTLLLNECD |
| | | CCTCAACGAGTGTGACATTGGATACAAGTACCACGTGTTGAGGAGCATCCTTCAGAACGCCCCAAATC | IGYKYHVLRSILQNAPNLEQLRL |
| | | TGGAGCAGCTTAGACTACACAATTGCAAGTTTGTAGGTAAATCGAGGAGGAAGGCTGGGCAAACCCAG | HNCKFVGKSRRKAGQTQSKEKTS |
| | | TCGAAGGAGAAAACTTCAAAATGCTCTAGCTCAACATTGTCTTCCGCGTGCAGCAGTTTGAAGTCAGT | KCSSSTLSSACSSLKSVEIKHPH |
| | | GGAAATCAAGCACCCGCACGGTGAACCTTCTCATGATCTTTTACATGAATTTCTGAAGGAAATACCAC | GEPSHDLLHEFLKEIPHNQWRKR |
| | | ACAATCAATGGAGGAAGAGATCGATTGATGAGGAGACAATATCAATCGAGCTCAATAGAAAGTAG | SIDEETISIELNRK* |
| | | SEQ ID NO: 171 | SEQ ID NO: 172 |
| 87 | Xa21_Os04g0491100 | ATGCTTGTCCATAGAAGCATTGAGAAAGATGTATTTGAGCCACTTGTGATGCGTATTTCTGAAATTGC | MLVHRSIEKDVFEPLVMRISEIA |
| | | GCAACACTATTTTGGGACAGTGGGCTCATCTGAAACTGGTGAGAAGGGTCCTACAGTGCCTGAATTTC | QHYFGTVGSSETGEKGPTVPEFP |
| | | CAGAGAAGATCAAATGGGAAGTGCAACATCTCGAAGAATTGTTTGAGGGCATTAAAGAAGACAAGGAG | EKIKWEVQHLEELFEGIKEDKEE |
| | | GAGGTCTATGAGGGTTTTAAGTCTGTTAGCCTTGCAATCTCCGAGTGGCAGAGAAGATTGGCTATAGC | VYEGFKSVSLAISEWQRRLAIAY |
| | | ATACCAAAATGCAGCTAGAGATCCTCAACCTTTTGAGGGAATGAAATGGGCAATGGAGTATCATGAGA | QNAARDPQPFEGMKWAMEYHEMW |
| | | TGTGGGTTGAAGACAATAATATAATAGGAGCTGGTGATGAGATATTGGATTTTGATGAACATGAGCTT | VEDNNIIGAGDEILDFDEHELFE |
| | | TTTGAATCACTTAGATATGTCAAGACTGCACAAGGTGGTTCAGAAGCTCATCTCCTGGAAAGTATCAA | SLRYVKTAQGGSEAHLLESIKSG |
| | | GAGTGGGATGCAGTGCATAAAGAATGTGTTAGCCACCATTCGATCAAGAAAGGAAGCAGACAACAGGA | MQCIKNVLATIRSRKEADNRSWC |
| | | GCTGGTGCATTGTGGAACAGGTCTTCTCCCCGCTACTGAAACTTTTGAAAACTATCAATCATCTAGTA | IVEQVFSPLLKLLKTINHLVSEA |
| | | TCAGAAGCGGCTGCCCGCAACAACAAGTCTGAGAACTATAAGATCCTTGTCAAGATCGATGCTGAAGT | AARNNKSENYKILVKIDAEVNCL |
| | | CAACTGCTTACAGGATGCACTAGATTTGATTGATAGAAATAAGAATGAGGTTTATGAGAACTTCAGAT | QDALDLIDRNKNEVYENFRLIED |
| | | TAATAGAAGATCTCATACTACCATTATTAACATGCCTTAAGGCCACATACAATGATCAATCAGAATCT | LILPLLTCLKATYNDQSESLSFL |
| | | TTGAGCTTCCTAGATGCAGTCAAACACGGAGTGAATTACTTGGAAGGTGTACTTGACAAGATTGAGCA | DAVKHGVNYLEGVLDKIEQKQRD |
| | | GAAGCAGCGGGATGGAAATGATAATTTCCACATTGTCAAAGCAGCCTTCTCACCATTGTTGACATGCA | GNDNFHIVKAAFSPLLTCMYTFR |
| | | TGTACACCTTCAGGCGCATCTCGTTGGAAACCTTAGCCCATGAAGATAAATCAGATGCTTTTATTCTG | RISLETLAHEDKSDAFILLDRIR |
| | | TTGGACAGGATCAGAGATGATTTGTCACAGCTCAAAGATGTACTTCAGATGGTTCAAGAGAAAGAGAA | DDLSQLKDVLQMVQEKENGIYSN |
| | | CGGAATATACAGTAACTTTGATGCAATTGAAGAACATATTGATGAGATTTATGACGGACATATGAATG | FDAIEEHIDEIYDGHMNVEGSLK |
| | | TTGAAGGTTCCTTAAAACTTAACCAGATGGGTGGTCTGCGAGACAAGCTCCAGTTGATTCATGAAGAG | LNQMGGLRDKLQLIHEEITNIRG |
| | | ATAACAAATATACGGGGAAAGGTGGATGACTCTTTCAAGGTCCAGGAGGTCTCATGCCATGTCATGCG | KVDDSFKVQEVSCHVMRMAAAHE |
| | | CATGGCTGCAGCACATGAAGCTTCATCTTCTCATCAGTTGTCAGCATCAAATACCTTCTGCATTACCA | ASSSHQLSASNTFCITMESAQMW |
| | | TGGAAAGTGCGCAGATGTGGCAGCTCAAAGTTATAATTGATGAACTAGAGACAAGATTGAGACACTGC | QLKVIIDELETRLRHCLLCLAVF |
| | | CTTCTATGCCTTGCTGTGTTCCCAGTGGATGCCATCATCAAGAAGAGACTGTTAATTCACTGGTGGAT | PVDAIIKKRLLIHWWIGEGFVTS |
| | | TGGAGAAGGTTTTGTAACTTCGGTTTCTGAAGGCAAGAGCTTCTTTAATAAATTGCTGCTGTCCAATG | VSEGKSFFNKLLLSNGFITPVKK |
| | | GGTTCATTACGCCAGTAAAGAAATACCACTGTGACAAAGTCCACTCGTGCAAGGTGCAACCTTGGATC | YHCDKVHSCKVQPWIRGLLIEAA |
| | | CGTGGACTACTGATTGAAGCTGCAAAGAGCAAAGCATTTGTTGAGCTCAGTTCAGATGGCAGCCCCAG | KSKAFVELSSDGSPRNDFTRTRR |
| | | GAATGACTTCACCAGGACCCGCCGTGCATGCTTGCATGCTGGAAAGATCCTTACAAACTTCCATCCTG | ACLHAGKILTNFHPDVLTIYNIK |
| | | ATGTTTTGACGATTTACAATATCAAGCAACAGTATGTAGAGTTAAACAAGACATGGTTTTCTGAAAAG | QQYVELNKTWFSEKNRLTTLQLG |
| | | AACCGTTTGACCACTTTGCAGTTAGGACAATGGCAGGATGCAAGTTATGACCCTCGGGCACATCACGT | QWQDASYDPRAHHVEINNAKFLK |
| | | TGAGATTAACAATGCCAAGTTCCTAAAGCAGGTCAAATCCTGCAAGCAGTTGAAGTACCTGAGTTTGA | QVKSCKQLKYLSLRGISRIEALP |
| | | GAGGCATATCAAGAATTGAGGCCCTTCCGAACTCAATTGGAAAGCTCTCAAGACTTGTCATTCTTGAT | NSIGKLSRLVILDLKACHNLEDL |
| | | CTCAAAGCGTGCCATAATCTAGAGGATCTTCCTAAAGAAATAGTAAAACTAGTGAAGCTGGAGTACTT | PKEIVKLVKLEYLDVSDCYLLSG |
| | | GGACGTTTCTGATTGTTACCTGCTCTCAGGTATGCCTAAAGGACTAGGCAAGCTTTTTCAACTTGAAG | MPKGLGKLFQLEVLKGFVLSNAK |
| | | TTCTGAAGGGATTTGTTCTATCAAACGCCAAGAGCAAAGATCCATGCCATCTCAATGAACTTGTCATG | SKDPCHLNELVMLKKLRKLSIRI |
| | | CTAAAAAAGTTACGAAAGCTGAGCATTAGAATTGGTTATAGCATAGATAGCGGTCAGTTTGCGAATTT | GYSIDSGQFANFGELCALRSLTL |
| | | TGGTGAGCTCTGTGCTCTCCGGTCGCTTACACTTATTTGGGGGGCACACCCAATATCTACACACGGGA | IWGAHPISTHGSSPSHAAPHAMP |
| | | GTTCTCCAAGTCATGCTGCTCCTCATGCAATGCCTTGTGTTCTCCCCTTAGGTCTTGAGAAGCTGGAG | CVLPLGLEKLELRCFPLVELPHW |
| | | CTCCGTTGTTTTCCCCTTGTAGAATTGCCTCACTGGGTTAGTCCTGAAAAGCTGAGAAAATTGAAGAA | VSPEKLRKLKKLYISGGNISDLG |
| | | GCTCTACATCAGCGGAGGTAACATCAGTGACCTGGGTGATTTGAAGTCCTGGGAGGTGACTGTACTTC | DLKSWEVTVLRLRFLKHMNYSWT |
| | | GGCTAAGGTTCCTCAAACATATGAACTATTCATGGACTGCCCTGCATGATTCATTCAGGAAACTCGAT | ALHDSFRKLDVLEAHECENLQPW |
| | | GTTTTGGAGGCACATGAATGTGAAAATCTACAGCCCTGGCCTTCCTGCGGAAAAGGGTTATGGCGGAA | PSCGKGLWRKEPNGTIAPVLT* |
| | | AGAACCGAATGGAACGATTGCACCTGTGTTGACCTGA SEQ ID NO: 173 | SEQ ID NO: 174 |
| 88 | Xa21_Os04g0509600 | ATGGCGACGTGCGCGGCGGACCTGGCGCCGCTGCTGGGGCCGGTGGCGGCGAACGCGACGGACTACCT | MATCAADLAPLLGPVAANATDYL |
| | | GTGCAACCGGTTCGCCGACACGACGTCGGCGGTGGACGCGACGTACCTGCTCTTCTCGGCGTACCTCG | CNRFADTTSAVDATYLLFSAYLV |
| | | TGTTCGCCATGCAGCTCGGGTTCGCGATGCTCTGCGCCGGGTCGGTGCGGGCCAAGAACACGATGAAC | FAMQLGFAMLCAGSVRAKNTMNI |
| | | ATCATGCTCACCAACGTGCTCGACGCCGCGGCCGGGGCGCTCTTCTACTACCTCTTCGGCTTCGCCTT | MLTNVLDAAAGALFYYLFGFAFA |
| | | CGCCTTCGGCACGCCGTCCAACGGCTTCATCGGGAAGCAGTTCTTCGGCCTCAAGCACATGCCGCAGA | FGTPSNGFIGKQFFGLKHMPQTG |
| | | CCGGGTTCGACTACGACTTCTTCCTCTTCCAGTGGGCCTTCGCCATCGCCGCCGCCGGGATCACGTCG | FDYDFFLFQWAFAIAAAGITSGS |
| | | GGCTCCATCGCCGAGAGGACGCAGTTCGTCGCCTACCTCATCTACTCCGCCTTCCTCACCGGGTTCGT | IAERTQFVAYLIYSAFLTGFVYP |
| | | CTACCCGGTGGTGTCCCACTGGATCTGGTCCGCCGATGGGTGGGCCTCTGCCTCCCGCACGTCCGGAC | VVSHWIWSADGWASASRTSGPLL |
| | | CTCTGCTGTTCGGCTCCGGTGTCATCGACTTCGCCGGCTCCGGCGTCGTCCACATGGTCGGCGGTGTC | FGSGVIDFAGSGVVHMVGGVAGL |
| | | GCCGGGCTCTGGGGCGCTCTCATCGAGGGACCCCGCATCGGGAGGTTCGACCACGCCGGCCGATCGGT | WGALIEGPRIGRFDHAGRSVALK |
| | | GGCGCTCAAGGGCCACAGCGCGTCGCTCGTCGTGCTCGGCACCTTCCTGCTGTGGTTCGGCTGGTACG | GHSASLVVLGTFLLWFGWYGFNP |
| | | GATTCAACCCCGGGTCGTTCACCACCATCCTCAAGACGTACGGCCCCGCCGGCGGCATCAACGGGCAG | GSFTTILKTYGPAGGINGQWSGV |
| | | TGGTCCGGCGTTGGCCGCACCGCCGTGACGACGACCCTGGCCGGCAGCGTGGCGGCGCTCACCACGCT | GRTAVTTTLAGSVAALTTLFGKR |
| | | GTTCGGGAAGCGGCTCCAGACGGGGCACTGGAACGTGGTCGACGTCTGCAACGGCCTCCTCGGCGGGT | LQTGHWNVVDVCNGLLGGFAAIT |
| | | TCGCCGCCATCACCGCCGGGTGCAGCGTCGTCGACCCGTGGGCCGCGATCATCTGCGGGTTCGTCTCG | AGCSVVDPWAAIICGFVSAWVLI |
| | | GCGTGGGTGCTCATCGGCCTCAACGCGCTCGCCGCGCGCCTCAAGTTCGACGACCCGCTCGAGGCCGC | GLNALAARLKFDDPLEAAQLHGG |
| | | CCAGCTCCACGGCGGGTGCGGCGCGTGGGGGATCCTCTTCACCGCGCTCTTCGCGAGGCAGAAGTACG | CGAWGILFTALFARQKYVEEIYG |
| | | TCGAGGAGATCTACGGCGCCGGCCGGCCGTACGGCCTGTTCATGGGCGGCGGCGGCAAGCTGCTCGCC | AGRPYGLFMGGGGKLLAAHVIQI |
| | | GCTCACGTCATCCAGATCCTGGTCATCTTCGGGTGGGTCAGCTGCACCATGGGGCCTCTCTTCTACGG | LVIFGWVSCTMGPLFYGLKKLGL |
| | | GCTCAAGAAGCTCGGCCTGCTCCGCATCTCCGCCGAGGACGAGACGTCCGGGATGGACCTGACACGGC | LRISAEDETSGMDLTRHGGFAYV |
| | | ACGGCGGGTTCGCGTACGTCTACCACGACGAGGACGAGCACGACAAGTCTGGGGTTGGTGGGTTCATG | YHDEDEHDKSGVGGFMLRSAQTR |
| | | CTCCGGTCCGCGCAGACCCGCGTCGAGCCGGCGGCGGCGGCTGCCTCCAACAGCAACAACCAAGTGTA | VEPAAAAASNSNNQV* |
| | | A SEQ ID NO: 175 | SEQ ID NO: 176 |
| 89 | Xa21_OsI_16631 | ATGGCCGACGCCATCTCATGCCTGCAGCCGTTGTGCGAGTGCTTGGATGGCACCGGCCTGTTCGATGC | MADAISCLQPLCECLDGTGLFDA |
| | | TGCAGCTAGGGATGTCGCCTCTTTCCTTCGGCTCAAATCGAATTGGGGTGATCTTGACAAGGCCAGGG | AARDVASFLRLKSNWGDLDKARE |
| | | AGAGCCTGGGGGCTGTTGAGAGGATGGTTCGAGGGCGGGTAACTGCGGAGTTAAACAAGCTGAACGTC | SLGAVERMVRGRVTAELNKLNVC |
| | | TGTGATCCTCAGGTGGAGCTGTGGCTGAGGCGTGTCGATGAGCTGAAGCTGGGTGCCATTGATGAGGA | DPQVELWLRRVDELKLGAIDEDY |
| | | CTATAGCAGTTTGATGAATTACTCTAGCATTTGCCAGTGCACCAGGCATGCTGCTCGGCGTTCATGGA | SSLMNYSSICQCTRHAARRSWIG |
| | | TTGGCAAGCGTATCGTTGAGGCGCTGGATGAGGTGAATAAACTGATCGAGGAAGGGAGGCAGTTTAAG | KRIVEALDEVNKLIEEGRQFKKF |
| | | AAGTTTGGATTCAAGCCCTCACCGGAGATTGTCGAGCGCTTGCCTCAAACCAAGACATTTGGATTGGA | GFKPSPEIVERLPQTKTFGLETM |
| | | GACCATGCTGGTTCAGCTGCATGATCTGCTTGAGAAGGCTGACTCAAACATAATTGGTATTTGGGGTC | LVQLHDLLEKADSNIIGIWGQGG |
| | | AAGGGGGTATTGGTAAAACGACACTACTACATGCCTTCAACAATGATCTTGAGAAGAAGGTCCATAAC | IGKTTLLHAFNNDLEKKVHNYQV |
| | | TACCAGGTTGTTATATTTATTGAAGTATCCAATTCAGAGACACTTGACACATTGGAGATGCAGAAGAC | VIFIEVSNSETLDTLEMQKTISE |
| | | CATCTCCGAACGTCTTAATTTACCATGGAATGAAGCAGAGACGACTGTGAAACGGGCCAGATTCCTTG | RLNLPWNEAETTVKRARFLVKAL |
| | | TTAAGGCCTTGTCCAGGAAAAGATTTGTACTGCTTCTTGATGATGTAAGGAAGAAATTCCGACTAGAG | SRKRFVLLLDDVRKKFRLEDVGI |
| | | GATGTTGGTATCCCAACTCCAGACACCAACAGCCAAAGCAAGCTGATCCTAACTTCACGTTTCCAAGA | PTPDTNSQSKLILTSRFQELSTE |
| | | ACTAAGCACCGAGGCTTGTGCAGCAGTTGAATCACCTAGTCCCAGCAATGTTGTTAGGGATCATGCCA | ACAAVESPSPSNVVRDHAIAIAQ |
| | | TAGCAATAGCTCAAAGTTGTGGAGGTCTACCACTTGCGCTCAATGTCATTGGGACTGCTGTGGCAGGC | SCGGLPLALNVIGTAVAGYEEPR |
| | | TATGAAGAACCAAGAGATTGGAATTCAGCTGCTGATGCAATCAAAGAGAATATGAAGTTTGAAGGTGT | DWNSAADAIKENMKFEGVDEMFA |
| | | TGATGAAATGTTTGCTACGCTGAAATACAGCTTCGACAGGCTCACACCTACTCAGCAACAGTGCTTTC | TLKYSFDRLTPTQQQCFLYCTLF |
| | | TGTATTGCACTCTTTTTCCAGAGTATGGATCAATTAGTAAAGAGCAACTTGTTGATTATTGGTTGGCT | PEYGSISKEQLVDYWLAEGLLLD |
| | | GAAGGTTTGCTACTTGATGATCGCGAGAAGGGTAATCAGATAATCCGAAGTCTTATTTCAGCATGCTT | DREKGNQIIRSLISACLLQTTSS |
| | | GTTGCAGACCACTAGTTCAATGTCATCAAAGGTGAAAATGCACCACATAATCAGACATTTGGGGCTTT | MSSKVKMHHIIRHLGLWLVNRED |
| | | GGTTGGTTAACAGGGAAGATAGAAGTTTTGTTGTTAAAGCAGGGATGGCCTTAGATAATGCTCCACCA | RSFVVKAGMALDNAPPAIEWKEA |
| | | GCAATAGAATGGAAAGAAGCTACAAGGATCTCCATTATGTCTAATAACATCACAGAGCTTTCTTTCTC | TRISIMSNNITELSFSPKCENLT |
| | | ACCAAAATGCGAAAACCTCACCACACTGTTAATCCAGAATAACCCAAAGTTGAACAAGCTAGGCTGGG | TLLIQNNPKLNKLGWGFFKYMRS |
| | | GTTTTTTCAAATATATGCGATCCTTGAAAGTGCTAGATCTATCACACACTGCAATTACTTCCATTCCA | LKVLDLSHTAITSIPECDTLVAL |
| | | GAATGTGATACATTAGTTGCATTGCAGCATCTCGACCTGTCATACACACATATTATGAGATTACCTGA | QHLDLSYTHIMRLPERLWLLKEL |
| | | GCGCCTATGGTTATTGAAAGAGTTGAGGCATCTGGACCTCAGTGTGACTGTTGCACTTGAAGATACTT | RHLDLSVTVALEDTLNNCSKLHK |
| | | TGAACAACTGCTCAAAGTTACACAAGTTGAGAGTGCTGAATCTTTTCCGCAGCCATTACGGGATTCGT | LRVLNLFRSHYGIRDVDDLNLDS |
| | | GATGTTGATGACCTGAATCTGGATTCACTGAGGTATCTATTGTTTCTTGGTATCACTATTTATTCACA | LRYLLFLGITIYSQDVLKKLNET |
| | | AGACGTGCTTAAGAAATTGAACGAGACCCATCCTTTGGCAAAATCAACGCATCGCTTGAACCTGAAGT | HPLAKSTHRLNLKYCGDMQSIKI |
| | | ATTGTGGAGATATGCAATCAATCAAAATTTCTGACTTTAACCATATGAAGCACCTTGAGGAGATGCAT | SDFNHMKHLEEMHVESCYDLNTL |
| | | GTCGAATCATGCTATGACCTGAATACACTGGTTGCCGATACTGAGTTGACGACTTCTTGCCTACAAGC | VADTELTTSCLQALTLSVLPSLE |
| | | ACTGACCCTCTCCGTCCTTCCCTCATTAGAAAATGTCCTTGTTGCACCAATGCCCCATAATTTTCGGT | NVLVAPMPHNFRYVRKLSISQCP |
| | | ACGTTCGCAAATTGTCCATTTCACAATGCCCCAAGTTGTTGAACATCACATGGGTCCGAAGACTTGAA | KLLNITWVRRLELLERLVISNCD |
| | | CTGCTTGAGAGGCTTGTCATATCCAATTGTGATGAGATGCTGACTATTGTTGAAGAAGCTAACAGTAC | EMLTIVEEANSTEEQQYGAQTIK |
| | | TGAAGAGCAGCAATATGGAGCACAAACAATAAAAATGCAGGGTTATTATTCTGAAGAACAAGACGATC | MQGYYSEEQDDHAMAESSRNEWN |
| | | ATGCAATGGCTGAGAGTTCACGCAATGAGTGGAATGATGATTACCAAAGCGTCAATAGAGAATCAACC | DDYQSVNRESTNGATRQPDFPKL |
| | | AATGGCGCAACGAGGCAACCTGACTTCCCAAAGTTGAGATCAATTGTGCTGACTGATGTGAAGAAGCT | RSIVLTDVKKLRSICTPRVFPCL |
| | | GAGAAGCATTTGCACGCCAAGAGTTTTTCCCTGCCTTGAGACTCTTCGAGTGGAAGACTGCCCAAATT | ETLRVEDCPNLRRIPLCSTHNCG |
| | | TGAGACGCATCCCACTGTGCAGCACTCACAACTGTGGTAAACTGAAGCAAATTTGTGGTTCATCTGAT | KLKQICGSSDWWKKLQWEDKEAA |
| | | TGGTGGAAGAAACTGCAGTGGGAGGACAAGGAGGCGGCGGCACATATGGAGAGCAAGTACTTCATTCC | AHMESKYFIPI* |
| | | AATCTGA SEQ ID NO: 177 | SEQ ID NO: 178 |
| 90 | Xa21_Os04g0521700 | ATGGAGCTCGACCTCCCGCCGAAGCAACGAGGCCCGCCGCTCGCCGACCTCGCCGTTGCCATCCACCG | MELDLPPKQRGPPLADLAVAIHR |
| | | GGCCGCCGAGGCCACCGCCGCGCTCTCCGCACCCTCCTCCTCTTCCCAAGCCACCGCTGCTGTGGCCG | AAEATAALSAPSSSSQATAAVAA |
| | | CCCTCCGTGACGCCCATTCCGCTATAGGCGGCTTCCTCTCTCGCCTCGAGGCCTCCGTCCCTGCCCCT | LRDAHSAIGGFLSRLEASVPAPS |
| | | TCTTCGTCCAGCGACGATCAGCCGATGGCGGACGGCTGCGAGGAGGAGGGGGAGGGGGAGCAGCAGAT | SSSDDQPMADGCEEEGEGEQQMV |
| | | GGTAGAGGAGGTGGAGGAGGGTCTCCGGGAGTGCGCCCTGCAGGGTAGCAAGCGGCGGAAGCGGCCTG | EEVEEGLRECALQGSKRRKRPVP |
| | | TGCCGCCGTCGTGGCCCCTAGGGCGCCGCTCGAGTGGCGGTTGCGTGGTGCCGGAGGCCGCGCCGGCT | PSWPLGRRSSGGCVVPEAAPAPL |
| | | CCTCTGGACGTGGAGGGGCGGCGGCGCGCTGCCATGGATCTCTTGCTACAGTTCCATGGGTAG | DVEGRRRAAMDLLLQFHG* |
| | | SEQ ID NO: 179 | SEQ ID NO: 180 |
| 91 | Xa21_Os04g0556400 | ATGGAGCCGGTGGCCGTCGTGGCGGTGCCGTTCCCGGCGCAGGGCCACCTCAACCAGCTCCTGCACCT | MEPVAVVAVPFPAQGHLNQLLHL |
| | | GTCGCTGCAGCTCGCCTCGCGCGGCGGGGTGGACGTGCACTACGCGGCGCCCGCGGCGCACGTCCGCC | SLQLASRGGVDVHYAAPAAHVRQ |
| | | AGGCCCGCGCGCGGGTGCAGGGCTGGGACGACGCGGCGCTCCGCTCCGTCCGGTTCCACGACCTCGGC | ARARVQGWDDAALRSVRFHDLGI |
| | | ATCTCCACGTACGCCTCCCCGCCCCCCGACCCCGCCGCCGCCTCGCCCTTCCCCTCCCACCTCATGCC | STYASPPPDPAAASPFPSHLMPL |
| | | CCTCTGGGAGGCGTACACCGCCGGCGCGCCCGCCCCGCTCGCGGCTCTCCTCGGCAAGCTCTCCGCCT | WEAYTAGAPAPLAALLGKLSASY |
| | | CCTACCGCCGCGTCGTCGTCGTGTACGACCGCATCAACGACTTCGCTGCACAGGAGGCGGCGCGGCTG | RRVVVVYDRINDFAAQEAARLRN |
| | | CGCAACGGCGAGGCGTTCGTCATGTACTGCCTGGCCGTGTCGATGCTCGCTAGGAGGATTGCTCCCCA | GEAFVMYCLAVSMLARRIAPQEH |
| | | GGAGCACCAACGGATCCTGCGCGAGAACGGCCTCACCAACATCACCGTGGAGGACTGTGCAACGGAGG | QRILRENGLTNITVEDCATEEFV |
| | | AGTTCGTGGATTACATCAGACGGACGAGGGCGACCAAGGAGATGTCGCCTGCCAGAGGTATCCTGACC | DYIRRTRATKEMSPARGILTNTC |
| | | AACACGTGTCGCGCGCTCGAGGGCGAGTTCGTCGACGCCGTCGCGGGGAACCTGGCCGCCGACGGCAA | RALEGEFVDAVAGNLAADGKKVF |
| | | GAAAGTTTTCACCGTCGGACCGTTGAACCCACTGCTGCATGCACGCGAACGCGTGGAAGCAGGGCGAC | TVGPLNPLLHARERVEAGRPAAQ |
| | | CAGCGGCGCAGCGCCACGAGTGCCTGGACTGGCTCGACAAGCAGCCCCCGGCGTCAGTGCTGTACGTC | RHECLDWLDKQPPASVLYVSFGT |
| | | TCCTTCGGCACGACATCGTCGCTGCGCGCGGAGCAAATCGAAGAGCTCGCATCGGCGCTGCGCGGCAG | TSSLRAEQIEELASALRGSNQRF |
| | | CAATCAGCGGTTCATCTGGGTGCTGCGCGACGCCGACCGCGGCGACATATTCGCGGACTCCGGCGAGA | IWVLRDADRGDIFADSGEIISRH |
| | | TCATCAGCCGCCACGCGAAGCTGCTGCGCGAGTTCACGCAGCACACCGAAGGGAGTACGGGGCTGGTG | AKLLREFTQHTEGSTGLVITGWA |
| | | ATCACCGGGTGGGCGCCGCAGCTGGAGATCCTCGCGCACGGCGCCACGGCGGCGTTCATGAGCCACTG | PQLEILAHGATAAFMSHCGWNST |
| | | CGGCTGGAACTCCACCATGGAGAGCCTGAGCCACGGGAAGCCGATTCTTGCCTGGCCCATGCACTGCG | MESLSHGKPILAWPMHCDQPWDA |
| | | ACCAGCCATGGGACGCCGAGCTCCTCTGCAAGTACCTCAAGGCCGGCGTCCTCGTGCGGCCATGGGAG | ELLCKYLKAGVLVRPWEKHNEIT |
| | | AAGCACAACGAGATCACGCTGGCGAAGGACATCCAGGAAGCCATCGAGGAGGCCATGCTTTCTGACGG | LAKDIQEAIEEAMLSDGGVAMRQ |
| | | AGGAGTAGCCATGCGACAACGTGCACGGGAGCTCGGAGATGCCATCCGTTCCTCCGTGGCCGCCGCCG | RARELGDAIRSSVAAAGSSRKDL |
| | | GCTCGTCGCGCAAAGACCTGGATGACTTCGTTGCTTACATCACGAGGTAA | DDFVAYITR* |
| | | SEQ ID NO: 181 | SEQ ID NO: 182 |
| 92 | Xa21_Os04g0571600 | ATGACGACCTGCGCGGATGACCAGACGGGCTGCGCCTTCTTCGCCCCCTTGCTGTCCTCCAAGGGGGC | MTTCADDQTGCAFFAPLLSSKGA |
| | | GGAGGTCGTCATCTTGGTGGCCGGCGATGAGGCGGAGGAGCAGCAGCCGGCGCCGGTGCTGACGAGCA | EVVILVAGDEAEEQQPAPVLTSK |
| | | AGCCGCCGGGTCGGCTCGCCAAGGCGGTGAACGAAGCCTGGTCCGTTTCTTTGGGCGTCGCGTTCCCG | PPGRLAKAVNEAWSVSLGVAFPV |
| | | GTGACGCCGTCCATGTTCACCTGCTCGGCACGGGGCGAGGCGCGGTCCATTCTTGGCCTCGCATTCCC | TPSMFTCSARGEARSILGLAFPM |
| | | GATGATACTGACGGGTTTGCTGCTCTATCTCCGGTCGATGATCTCGATGCTATTCCTTGGTCACCTCG | ILTGLLLYLRSMISMLFLGHLGG |
| | | GCGGGCTGGCCCTCGCCGGCGGGTCCCTCGCCATCGGATTCGCCAATATTACCGGCTACTCTGTCCTC | LALAGGSLAIGFANITGYSVLSG |
| | | TCCGGCCTCGCCATGGGCATGGAGCCGATATGCGGTCAGGCCTTCGGCGCGGGCAATTACGCGCTCCT | LAMGMEPICGQAFGAGNYALLGV |
| | | GGGCGTCACCATGCAGCGGACGGTGCTCCTCCTCATCGCGGCCGCCATCCCCATAGGTGGCCTGTGGG | TMQRTVLLLIAAAIPIGGLWVQM |
| | | TGCAGATGCGGCCTCTCCTCCTCTTCTGTGGCCAGGACGCCGCCATCGCGGCGGTCGCCGAGACCTAC | RPLLLFCGQDAAIAAVAETYIFA |
| | | ATTTTCGCGTCTCTCCCTGACCTCGTTCTCCAGGCATTCCTCCACCCGGTGAGGATATACCTCAGGAC | SLPDLVLQAFLHPVRIYLRTQSI |
| | | GCAATCCATCAACCTGCCCCTCACCGTGTGCGCCGGCCTGGCCATTGCCATCCACCTCCCCATCAACT | NLPLTVCAGLAIAIHLPINYVLV |
| | | ACGTTCTCGTAGTCGTCCTCGGCCTCGGCGTAAAGGGGGTCGCATTGGCGTCCGTGCTGGCCAACTTG | VVLGLGVKGVALASVLANLNLVL |
| | | AACCTCGTGCTCTTCCTCCTCGCCTACATCTTCTTGAAAGGCGTCCACAAGCGCACCGGCGGTTTCCT | FLLAYIFLKGVHKRTGGFLLSAE |
| | | TCTGTCCGCCGAGAGCTTCCGTGGCTGGGGCGAGCTCATCAGCCTCGCGCTGCCGAGCTGCGTGAGCG | SFRGWGELISLALPSCVSVCLEW |
| | | TCTGCCTCGAGTGGTGGTGGTACGAGATCATGATCCTGCTGTGCGGCCTGCTGCTGAACCCGCAGGCG | WWYEIMILLCGLLLNPQATVASM |
| | | ACGGTGGCGTCCATGGGCATACTGATCCAGACCACGTCGCTCATATACATCTTCCCTTCGTCGCTCAG | GILIQTTSLIYIFPSSLSFGVST |
| | | CTTCGGCGTGTCCACCCGCGTGAGCAACGAGCTAGGCGCGGGGCAGCCCGAGGAGGCGAGCCGCGCAG | RVSNELGAGQPEEASRAATVGLV |
| | | CCACGGTGGGGCTCGTGCTCGGCTTCGGGTTCGGCGCCTTCGCCTCCGCGTTCGCGTTCCTCGTGCGC | LGFGFGAFASAFAFLVRNVWASM |
| | | AACGTGTGGGCGAGCATGTTCACGGCCGACCCGGCCATCGTCGCGCTCACCGCGTCCGTGCTGCCCAT | FTADPAIVALTASVLPILGLCEL |
| | | CCTGGGCTTGTGCGAGCTCGGCAACTGCCCGCAGACGACGGGGTGCGGGGTGCTGCGCGGCAGCGCGC | GNCPQTTGCGVLRGSARPKDAAS |
| | | GGCCCAAGGACGCCGCGAGCATCAACCTCCGCTCCTTCTACCTCGTCGGGACGCCCGTGGCGCTCGTC | INLRSFYLVGTPVALVMAFWFHL |
| | | ATGGCGTTCTGGTTCCACTTGGACTTCAGGGGCCTCTGGTTCGGCCTGCTCGCGGCGCAGGCTACCTG | DFRGLWFGLLAAQATCTVRMLLV |
| | | CACGGTGCGCATGCTGCTGGTCATCGGCCGGACGGACTGGGCGGCCGAGGCCAAGCGGTCCAAGCAGC | IGRTDWAAEAKRSKQLTGAGAAN |
| | | TCACCGGAGCGGGCGCCGCCAACATGGAGAGTGACGACAGAGTTGCCGCGGACGAGAAGTTGCGCTTA | MESDDRVAADEKLRLPVDTDVER |
| | | CCCGTCGACACGGACGTGGAGCGATCGAGCGATCACACTGATCGGTGTTGA | SSDHTDRC* |
| | | SEQ ID NO: 183 | SEQ ID NO: 184 |
| 93 | Xa21_Os04g0572400 | ATGGAGTGGGCGTACTACGGCAGTGGGTACTCCTCGTCGGGGTCGCCGTCGCCGGTGGGCGGCGACGG | MEWAYYGSGYSSSGSPSPVGGDG |
| | | GGACGAGGACTCGTACATGACGGTGTCGTCGGCGCCGCCCAAGCGGCGGGCGGGGAGGACCAAGTTCA | DEDSYMTVSSAPPKRRAGRTKFK |
| | | AGGAGACGAGGCACCCGGTGTACAAGGGCGTGCGCAGCAGGAACCCCGGGAGGTGGGTCTGCGAGGTG | ETRHPVYKGVRSRNPGRWVCEVR |
| | | CGCGAGCCGCACGGCAAGCAGAGGATCTGGCTCGGCACCTTCGAGACCGCCGAGATGGCGGCGCGCGC | EPHGKQRIWLGTFETAEMAARAH |
| | | GCACGACGTCGCGGCGATGGCGCTGCGCGGCCGCGCCGCCTGCCTCAACTTCGCCGACTCGCCGCGCA | DVAAMALRGRAACLNFADSPRRL |
| | | GGCTCCGCGTGCCGCCGCTGGGGGCCGGGCACGAGGAGATACGCCGCGCCGCGGTCGAGGCCGCCGAG | RVPPLGAGHEEIRRAAVEAAELF |
| | | CTGTTCCGCCCGGCGCCCGGGCAGCACAATGCGGCCGCCGAGGCGGCGGCCGCTGTCGCTGCGCAGGC | RPAPGQHNAAAEAAAAVAAQATA |
| | | AACCGCGGCGAGCGCGGAGCTCTTTGCCGATTTCCCTTGCTACCCGATGGACGGACTGGAATTCGAAA | ASAELFADFPCYPMDGLEFEMQG |
| | | TGCAGGGGTACCTCGACATGGCACAGGGAATGCTCATCGAGCCGCCGCCAGTGACAGGGCAATCGACG | YLDMAQGMLIEPPPVTGQSTWAE |
| | | TGGGCAGAGGAGGACTACGACTGCGAGGTCAATCTATGGAGCTACTGA | EDYDCEVNLWSY* |
| | | SEQ ID NO: 185 | SEQ ID NO: 186 |
| 94 | Xa21_Os04g0584600 | ATGGGCAACGCATGCGGCGGTTCCCTTAGATCCAAGTACCTGAGCTTCAAGCAGACCGCGTCGCAGCG | MGNACGGSLRSKYLSFKQTASQR |
| | | CCACGACACCGACGTCGACAACAACAACGCCGCCGCGGCCGACTCGCCGAAGAAGCCCTCCCGCCCTC | HDTDVDNNNAAAADSPKKPSRPP |
| | | CAGCAGCCGCCAAGACGGACCTCTACGCCATGGGCAGGAAGCTCGGGCAGGGGCAGTTCGGCACCACC | AAAKTDLYAMGRKLGQGQFGTTY |
| | | TACCTCTGCACCGAGCTGTCCACTGGGGTGGACTACGCCTGCAAGTCCATTTCCAAGCGCAAGCTCAT | LCTELSTGVDYACKSISKRKLIT |
| | | CACCAAGGAGGACATCGAAGACGTTCGCCGCGAGATCCAGATCATGCACCATCTCTCGGGCCACAAGA | KEDIEDVRREIQIMHHLSGHKNV |
| | | ACGTCGTCGCCATCAAGGGCGCCTACGAGGACCAGCTATACGTGCACATCGTCATGGAGCTCTGCGCC | VAIKGAYEDQLYVHIVMELCAGG |
| | | GGCGGCGAGCTCTTCGATCGCATCATACAGCGCGGCCACTACAGCGAGCGGAAGGCCGCCGAGCTCAC | ELFDRIIQRGHYSERKAAELTRI |
| | | GCGGATCATCGTTGGGGTCGTCGAGGCCTGCCACTCGCTCGGGGTCATGCACCGGGACCTCAAGCCAG | IVGVVEACHSLGVMHRDLKPENF |
| | | AAAACTTCCTGCTTGCCAACAAGGACGATGACCTCTCGCTCAAGGCCATAGATTTTGGTCTCTCAGTC | LLANKDDDLSLKAIDFGLSVFFK |
| | | TTCTTCAAGCCCGGTCAAACTTTTACCGATGTTGTCGGAAGCCCATATTACGTAGCTCCAGAAGTGTT | PGQTFTDVVGSPYYVAPEVLLKH |
| | | GTTGAAACACTATGGACCAGAAGCTGATGTATGGACTGCAGGTGTCATTCTCTACATTTTACTAAGTG | YGPEADVWTAGVILYILLSGVPP |
| | | GTGTACCTCCATTCTGGGCAGAGACACAACAAGGAATATTTGATGCTGTACTAAAAGGCTTCATTGAT | FWAETQQGIFDAVLKGFIDFDSD |
| | | TTTGATTCTGATCCTTGGCCTGTGATCTCTGATAGTGCAAAGGATCTTATAACAAAAATGCTCAATCC | PWPVISDSAKDLITKMLNPRPKE |
| | | TCGCCCAAAGGAACGCTTAACAGCACATGAAGTTCTATGCCATCCATGGATTCGTGATCACGGAGTTG | RLTAHEVLCHPWIRDHGVAPDRP |
| | | CTCCAGATCGGCCTCTTGATCCAGCTGTCCTATCTCGCATCAAGCAGTTCTCTGCAATGAATAAGTTG | LDPAVLSRIKQFSAMNKLKKMAL |
| | | AAGAAGATGGCTTTGCGGGTAATAGCTGAGAGTCTCTCAGAGGAGGAAATCGCTGGGTTGAAGGAAAT | RVIAESLSEEEIAGLKEMFQTMD |
| | | GTTTCAGACTATGGATGCTGATAACAGTGGTGCGATCACATATGATGAGCTTAAAGAAGGCTTGAGAA | ADNSGAITYDELKEGLRKYGSTL |
| | | AATATGGCTCAACACTTAAGGACACCGAGATTCGTGATCTTATGGATGCAGCAGATATAGACAACAGC | KDTEIRDLMDAADIDNSGTIDYI |
| | | GGAACAATTGATTATATCGAATTCATTGCTGCAACATTGCATCTCAATAAACTGGAGCGAGAGGAACA | EFIAATLHLNKLEREEHLVAAFS |
| | | TCTTGTGGCAGCTTTTTCATATTTTGACAAAGATGGAAGTGGTTACATCACAGTGGATGAACTGCAGC | YFDKDGSGYITVDELQQACKEHN |
| | | AAGCTTGCAAAGAGCATAACATGCCCGATGCTTTTCTTGATGATGTCATCAATGAAGCTGATCAGGAC | MPDAFLDDVINEADQDNDGRIDY |
| | | AATGATGGGCGCATTGACTATGGAGAATTTGTTGCCATGATGACCAAGGGCAATATGGGAGTCGGACG | GEFVAMMTKGNMGVGRRTMRNSL |
| | | AAGAACAATGAGAAACAGCTTGAATATCAGCATGAGGGACGCACCTGGTGCACTCTAAG | NISMRDAPGAL* |
| | | SEQ ID NO: 187 | SEQ ID NO: 188 |
| 95 | Xa21_Os04g0610400 | ATGGCTCCCAGGAACGCCGCCGAGGCCGTCGCCGTCGCCGTGGCGGAGGGCGGAGGGGCCGGCATGGA | MAPRNAAEAVAVAVAEGGGAGME |
| | | GCCCAGGTTCCGCGGCGTGAGGAAGCGCCCGTGGGGCAGGTACGCGGCGGAGATCCGCGACCCGGCGA | PRFRGVRKRPWGRYAAEIRDPAR |
| | | GGAAGGCGCGGGTGTGGCTCGGCACCTTCGACACCGCCGAGGCCGCGGCGCGCGCCTACGACAGCGCC | KARVWLGTFDTAEAAARAYDSAA |
| | | GCGCTCCACTTCCGCGGGCCCAAGGCCAAGACCAACTTCCCCGTCGCCTTCGCGCACGCCCACCACCA | LHFRGPKAKTNFPVAFAHAHHHA |
| | | CGCCCCGCCGCCGCCGCTGCCCAAGGCGGCGGCGCTGGCCGTCGTCAGCCCGACCAGCAGCACGGTCG | PPPPLPKAAALAVVSPTSSTVES |
| | | AGTCGTCCTCCCGGGACACGCCCGCCGCCGCGCCGGTGGTGGCCGCGGCCAAGGCCCAGGTGCCCGCC | SSRDTPAAAPVVAAAKAQVPAAP |
| | | GCGCCCTCGCTCGATCTGAGCCTCGGGATGTCGGCCATGGTGGCCGCACAGCCGTTCCTGTTCCTCGA | SLDLSLGMSAMVAAQPFLFLDPR |
| | | CCCAAGGGTCGCGGTGACCGTGGCCGTCGCGGCGCCGGTGCCTCGCCGGCCGGCCGTCGTCAGCGTCA | VAVTVAVAAPVPRRPAVVSVKKE |
| | | AGAAGGAGGTAGCTCGCCTGGACGAGCAGAGCGACACCGGCTCGTCGTCATCCGTGGTGGACGCCTCG | VARLDEQSDTGSSSSVVDASPAV |
| | | CCGGCCGTCGGCGTGGGGCTCGACCTGAACCTGCCGCCGCCGACCGAGGAGGCGTAG | GVGLDLNLPPPTEEA* |
| | | SEQ ID NO: 189 | SEQ ID NO: 190 |
| 96 | Xa21_Os04g0635100 | ATGGCGGGTGCAGCGAAGGCGTCGTGGATGGTGGCGATGAGCGTGGGCGCCGTGGAGGCGCTCAAGGA | MAGAAKASWMVAMSVGAVEALKD |
| | | CCAGGGCGGACTCTGCCGCTGGAACTACGCGCTGCGGTCCATCCACAAAGCAGCCAAGGCCAACGCCG | QGGLCRWNYALRSIHKAAKANAA |
| | | CCGGCGTCTCGCAGGGCAAGAAGCTGCCTGCTTCTGCGGCGGCAGTGGCGGAGAGGAGGAGAGCGGAG | GVSQGKKLPASAAAVAERRRAEK |
| | | AAAGCCGAGGAGGGGTTGAGGACAGTCATGTACCTCAGCTGCTGGGGTCCCAATTAG | AEEGLRTVMYLSCWGPN* |
| | | SEQ ID NO: 191 | SEQ ID NO: 192 |
| 97 | Xa21_Os04g0639200 | ATGGCGGCGGCGGCGGCGCGCGCGATCATATGCGAGATGGCACCGCAGAGGGGGGCGGCGGCGGCGGC | MAAAAARAIICEMAPQRGAAAAA |
| | | GCCGCCGGCGCAGCAGCAGGCGACGACGAGACGAGACGGTGGGAAGATCATGCTCCAGCCACGCCTCT | PPAQQQATTRRDGGKIMLQPRLC |
| | | GCACGCTCCGGTCGTACGGCGCCGGCAGCGGCGTGGTGGCGCGGAGGCGGGTGGTGGAGGAGGAGGAG | TLRSYGAGSGVVARRRVVEEEES |
| | | AGCGGCGGCGGCGGCGCGGGGTCCTCCCCGTTCTTCGCGTCGCTCGCCGACTACATCGAGAGCACCCG | GGGGAGSSPFFASLADYIESTRK |
| | | CAAGAGCCAGGACTTCGAGACCATCTCCGGCCGCCTCGCCATGGTGGCGTTCGCGACGGCGGTGGCGG | SQDFETISGRLAMVAFATAVAVE |
| | | TGGAGTTGACGACGGGGAACTCGCTGTTCAAGAAGCTGGACATGCAGGAGATCGAGGAGGCGGCCGGG | LTTGNSLFKKLDMQEIEEAAGVC |
| | | GTATGCCTCGCCGTGGTCGCCGGCGCCGCCGCGTTCGCGTGGGTGTCCAGCGCGCGCACCAGGATCGG | LAVVAGAAAFAWVSSARTRIGQM |
| | | CCAGATGTTCACGCTGGGTTGCAGCGCCTTCGTCGACTCGCTCATCGACAACATCGTCGAAGCTCTCT | FTLGCSAFVDSLIDNIVEALFSE |
| | | TCTCCGAGGGCGAGCTGCAGGACTGGTCCGACGACGTATGA | GELQDWSDDV* |
| | | SEQ ID NO: 193 | SEQ ID NO: 194 |
| 98 | Xa21_Os04g0685300 | ATGGGTTCGGCGAGCCGCGCGGTGTCGTGCCTGTGCTGCCCGTGCAAGTGCCTGGCGTGCGGCCTCTT | MGSASRAVSCLCCPCKCLACGLF |
| | | CAGCTGCCTCTGCAGCATCCTCATCTCCCTCCTCGTCACCCTCGGCGTCCTCGCCCTCATCTTCTACC | SCLCSILISLLVTLGVLALIFYL |
| | | TCATCTTCCGCCCCCACATGATCGCCGCCACCGTCGACTCCGCCGCCCTCACCCAGTTCACCCTCTCC | IFRPHMIAATVDSAALTQFTLST |
| | | ACCAACTCCGCCCTCGCCTACAGCCTCACCGTCGACCTCACCGTCCGCAACCCCAACAAGCGCGTCGG | NSALAYSLTVDLTVRNPNKRVGL |
| | | CCTCTACTACGACAACGTCGAGTCCCTCGCCCTCTTCGACGGCCAGCGCTTCGGCTACGTGCCCCTCG | YYDNVESLALFDGQRFGYVPLDS |
| | | ACTCCTTCTACCAGTCCACCGAGGCATCCACCAAGCTCTCCCCCGCCTTCAAGGGCCAGCAGCCGCTC | FYQSTEASTKLSPAFKGQQPLQG |
| | | CAAGGCGACATCACCGCCGCCAACTTCAGGACGCAGCAGGCCGCCGGCAAGTTCGACATCGAGGTCAA | DITAANFRTQQAAGKFDIEVKLN |
| | | GCTCAACGCCAAGCTCAGGGTCAAGGTCTGGGCCTTCAAGGTTCCCGGACCCAAGGCCAAGATCAGCT | AKLRVKVWAFKVPGPKAKISCPI |
| | | GCCCCATCACCGTCCCAGCGTCCGCCCCCAACGCCCCCGCATTCCAGCGCACCGACTGCAAAGTATGG | TVPASAPNAPAFQRTDCKVWF* |
| | | TTCTGA SEQ ID NO: 195 | SEQ ID NO: 196 |
| 99 | Xa21_OsJ_24689 | ATGGCTCCTCTCTTCTTCCTCCTCCTCTTCAGCCAAATCTTTCTCTGCACCGCGGTGGACACCATCAA | MAPLFFLLLFSQIFLCTAVDTIN |
| | (Os07g0553633) | CTCCACCGCGCCGCTGTCCGGGACGCAGAAGATCGTCTCCAAGGGCGGCAGGTTCGCCCTTGGATTCT | STAPLSGTQKIVSKGGRFALGFY |
| | | ACACCCCGCCACAAGGTAACAACACCGCCTCCGGCACCGGCAACTACTACATAGCTATATGGTACAAC | TPPQGNNTASGTGNYYIAIWYNN |
| | | AACATACCGTTGCAGACGACGGTGTGGACGGCCAACAGCGACGTGCCGGTGTCTGACCCGACCACCGC | IPLQTTVWTANSDVPVSDPTTAS |
| | | ATCCTTGTCCATCGGCAGCGATGGCAATCTTGTCCTCCTTGATCAATCCAAGAACAGGCAGCTGTGGT | LSIGSDGNLVLLDQSKNRQLWST |
| | | CGACCAACGTAAGTGTGGCCTCCAACTCCACCGTTGCCGTCATCCAGGACGGCGGTAGCCTCGACCTC | NVSVASNSTVAVIQDGGSLDLMD |
| | | ATGGATGCCACCAATTCATCCATAGTCTACTGGAGGAGCATAGATCATCCGACAAACACCTGGCTTCC | ATNSSIVYWRSIDHPTNTWLPGG |
| | | GGGCGGCAAGCTGGGGCTGAACAAGACCACTGGTGTGAGCCAGCGGCTTGTCCCATGGAGGAACAATG | KLGLNKTTGVSQRLVPWRNNANP |
| | | CAAACCCGTCCCCTGGGCTGTTCTCCCTTGAGCTGGATCCCAACGGCACAACGCAGTACTTCATCCAG | SPGLFSLELDPNGTTQYFIQWND |
| | | TGGAACGACTCCATCACTTACTGGACCAGCGGCCCTTGGAACGGTAATATCTTCAGTCTTGTGCCCGA | SITYWTSGPWNGNIFSLVPEMTA |
| | | GATGACGGCCGGTTACAACTACAACTTCCGGTTCATTAACAATGTCTCGGAGAGCTACTTCATATACT | GYNYNFRFINNVSESYFIYSMKD |
| | | CCATGAAGGATGACTCGATCATCTCGCGGTTCACCATTGATGTGAATGGGCAGATCAAGCAGTGGACA | DSIISRFTIDVNGQIKQWTWVPA |
| | | TGGGTACCAGCCTCGGAGAATTGGATCTTGTTCTGGTCCCAGCCACGGACGCAGTGTGAGGTGTACGG | SENWILFWSQPRTQCEVYGLCGA |
| | | GCTCTGCGGCGCATATGGAAGTTGCAACCTCAATGTCCTGCCGTTCTGCAACTGCATCAAGGGGTTCA | YGSCNLNVLPFCNCIKGFSQKFQ |
| | | GCCAGAAGTTTCAGAGTGATTGGGACCTCCAGGATTTTACTGGTGGGTGCAAGAGGAACGTACCGTTG | SDWDLQDFTGGCKRNVPLQCQTN |
| | | CAGTGCCAGACCAACTCAAGCTCTGCACAGACTCAGCCTGATAAGTTCTATTCCATGGTTAGTGTGAG | SSSAQTQPDKFYSMVSVRLPDNA |
| | | GCTACCTGATAATGCTCAGAGTGCAGTGGCCGCTAGTTCTCAGGCCTGTCAGGTTGCTTGCCTGAATA | QSAVAASSQACQVACLNNCSCNA |
| | | ATTGCTCATGTAATGCTTATACTTACAATAGTAGTGGCTGCTTTGTTTGGCACGGGGACTTGATTAAC | YTYNSSGCFVWHGDLINLQDQYN |
| | | CTCCAAGATCAATACAATGGGAATGGAGGAGGCACACTCTTCCTCAGGCTTGCAGCGTCTGAACTTCC | GNGGGTLFLRLAASELPDSKNSK |
| | | AGACTCTAAAAATAGCAAGAAAGTGATCATTGGTGCAGTTGTAGGTGGAGTTGCTGCAGCTTTGATAA | KVIIGAVVGGVAAALIILAIVLF |
| | | TCCTTGCAATTGTGTTGTTCATCGTATTCCAGAAATGCCGCCGAGACAGGACTCTTCGCATATCCAAG | IVFQKCRRDRTLRISKTTGGALI |
| | | ACTACTGGGGGTGCACTGATTGCCTTCCGGTACAGTGATCTGCAGCATGTCACCAGCAACTTCTCAGA | AFRYSDLQHVTSNFSEKLGGGAF |
| | | GAAGCTGGGTGGAGGTGCCTTTGGCACAGTATTTAAAGGGAAGCTCCCAGATTCAACAGCCATTGCAG | GTVFKGKLPDSTAIAVKRLDGLS |
| | | TGAAGAGGCTTGATGGACTGAGCCAGGGGGAGAAGCAATTCCGTGCTGAGGTAAGCACCATCGGGACC | QGEKQFRAEVSTIGTIQHVNLVR |
| | | ATCCAACATGTCAATCTGGTTCGCCTACTTGGTTTCTGCTCTGAAGGATCAAGAAGACTGCTTGTCTA | LLGFCSEGSRRLLVYEYMPKGSL |
| | | TGAGTACATGCCAAAGGGCTCCCTGGAATTGCAGCTGTTCCATGGTGAGACAACGGCACTGAACTGGG | ELQLFHGETTALNWAIRYQIALG |
| | | CCATAAGGTATCAAATTGCACTTGGAACAGCAAGGGGCCTAAATTACCTGCATGAAAAATGTAGGGAT | TARGLNYLHEKCRDCIIHCDVKP |
| | | TGCATAATACACTGTGATGTTAAGCCAGATAACATACTTTTGGATGAGTCATTTGTACCCAAAGTGTC | DNILLDESFVPKVSDFGLAKLLG |
| | | TGACTTTGGTCTAGCAAAGCTCTTAGGGCGGGATTTCAGCCGTGTTTTGACAACAATGAGAGGGACAA | RDFSRVLTTMRGTRGYLAPEWIS |
| | | GGGGTTACCTTGCGCCTGAGTGGATCAGTGGTGTGCCGATTACTCCTAAGGCAGATGTCTTCAGCTAT | GVPITPKADVFSYGMMLFELISG |
| | | GGCATGATGCTCTTCGAACTCATATCAGGCAGAAGGAATGCTGATCTAGGGGAGGAGGGAAAGTCCTC | RRNADLGEEGKSSFFPTLAVNKL |
| | | TTTCTTCCCAACTTTGGCTGTAAACAAACTACAGGAAGGTGATGTGCAGACCTTGTTAGACCCCAGGT | QEGDVQTLLDPRLNGDASADELT |
| | | TGAATGGAGATGCCAGCGCTGATGAGCTCACAAAAGCTTGCAAGGTTGCCTGCTGGTGTATCCAAGAT | KACKVACWCIQDDENARPTMGQV |
| | | GATGAAAATGCCAGACCTACAATGGGTCAGGTCGTCCAAATTTTAGAGGGATTCCTTGATGTCAACAT | VQILEGFLDVNMPPVPRSLKVLN |
| | | GCCTCCTGTTCCAAGGTCACTGAAGGTTCTTAATGAAAGCCCTGATGTCATAAATTTTTTCTCAGATG | ESPDVINFFSDVSSSQTSQMHNS |
| | | TTTCATCAAGTCAAACTTCGCAGATGCATAATAGCACAGCTTCTTCGCAGACAAAGAGTTCTACTTCA | TASSQTKSSTSGGSQFQSS* |
| | | GGTGGTTCACAATTTCAGAGCTCATAA SEQ ID NO: 197 | SEQ ID NO: 198 |
| 100 | Xa21_Os05g0322900 | ATGACGTCGTCGATGTCGCCGGCGCCGGCGCCGCCGTACGCGCAGGTGATGGAGGACATGGAGAAGGG | MTSSMSPAPAPPYAQVMEDMEKG |
| | | GAAGGAGCTGGCGGCGCAGCTGCAGGGGCTCCTCCGCGACTCGCCGGAGGCCGGCCGCATCGTCGACC | KELAAQLQGLLRDSPEAGRIVDQ |
| | | AGATCCTCCACACCTTCTCCCGGGCGATGCGGGCGCTCGACAAGGCGGCGGTCTCCGCCGCCGGAGGA | ILHTFSRAMRALDKAAVSAAGGE |
| | | GAAGGGTCGGAGGTGCAGAGCGAGGTCACCTGCGGGGGCGGGGCCAGCGCCGGCGGGAAGAGGAAAGC | GSEVQSEVTCGGGASAGGKRKAP |
| | | CCCCGCCGCCGACCGGAAGGCCAACTGCCGCAGGAGGACGCAGCAATCGTCCGGGAATACGGTGGTCG | AADRKANCRRRTQQSSGNTVVVK |
| | | TCAAGAACCTCGACGACGGCCAGGCATGGCGCAAGTACGGGCAGAAGGAGATCCAAAACTCCAAGCAC | NLDDGQAWRKYGQKEIQNSKHPK |
| | | CCAAAGGCCTACTTCCGGTGCACGCACAAGTACGACCAGCTGTGCACGGCGCAGCGGCAGGTGCAGCG | AYFRCTHKYDQLCTAQRQVQRCD |
| | | CTGCGACGACGACCCGGCGAGCTACAGGGTCACCTACATCGGCGAGCACACCTGCCGGGACCCGGCCA | DDPASYRVTYIGEHTCRDPATAP |
| | | CCGCCCCCATCATCGCGGCGCACGTCATCCACCAGGTCGCCGCCGGCGACGACGACGACGGCTGCGGC | IIAAHVIHQVAAGDDDDGCGGLH |
| | | GGCCTCCACGCGGGGTCCCGCCTCATCAGCTTCGTCGCCGCGCCGGCGGCGCCAGTAGACGCTGCCGC | AGSRLISFVAAPAAPVDAAAAPT |
| | | GGCGCCGACGACCAGCACGATCACCACGGTCACCGCGCCGGGCCCGCTGCAGCAGCCGCTCAAGGTGG | TSTITTVTAPGPLQQPLKVEGGV |
| | | AGGGCGGCGTCGGCTCGTCCGACCAGGAGGAGGTGCTGAGCAGCCTCACGCCCGGCAGCTCCGCGGTG | GSSDQEEVLSSLTPGSSAVRGGG |
| | | CGCGGCGGCGGCGGGGGCGCGGGTCCCTTCGGGCCGGACCAGGGCGATGTCACGTCCTCGCTGCACTG | GGAGPFGPDQGDVTSSLHCSYDA |
| | | CAGCTACGACGCCGACGCCTTCGCCGTCGCCGGCATGGAGTTCTTCAAGGACGACGAGGTTGTCTTCA | DAFAVAGMEFFKDDEVVFNLDDI |
| | | ATCTGGACGACATTATGGGTTTGAGCTTTTGA SEQ ID NO: 199 | MGLSF* SEQ ID NO: 200 |
| 101 | Xa21_OsI_19719 | ATGGGCGGGTGCTTGTCCGGCGACGTGCGCGGCGGCATGGAGGCCGTGGGCGGGAGCGGTGGCCGCGG | MGGCLSGDVRGGMEAVGGSGGRG |
| | (Os05g0373300) | GGCGGCGGGGACCGGAGGAGGAGGGGGAGGAGTGGCGGGGCAGGGCGGCGGGGCGAACGAGGCGGTGG | AAGTGGGGGGVAGQGGGANEAVD |
| | | ACCACTTCTTCAACGCCGCCGGCCTCCGCGGCCTCTACTCGCCTCTCGAGCTATCCTTTTCAGCATCA | HFFNAAGLRGLYSPLELSFSASK |
| | | AAGTTGAGGAATATGGATGCTTTTTCCAAGAGTGATCCAATGTTAGTGATCTACATAAGAAAGGACAC | LRNMDAFSKSDPMLVIYIRKDTR |
| | | AAGGCTAGAAGAGATTGGCCGTACTGAAGTGATACTGAACTCTCTGGAACCCTCGTGGATCACAAAGG | LEEIGRTEVILNSLEPSWITKAT |
| | | CTACAATAAGTTACCAATTTGAGATTATACAGCCGCTTGTATTCAAGATATATGACATTGACACAAGG | ISYQFEIIQPLVFKIYDIDTRYH |
| | | TACCATAACACGCCAGTCAAGACGCTGAACTTGGCTCAGCAAGATTTTCTTGGAGAAGCATGCTGCAA | NTPVKTLNLAQQDFLGEACCNLS |
| | | TTTGTCAGAGATTGTCACAAACAATCACAGCCTGACACTTAACCTCAGAGATAGTTGTGGACATGCCC | EIVTNNHSLTLNLRDSCGHALLG |
| | | TTCTAGGAACAGTGACAGTACATGCTGAAGAAAGTAATTCGTCAAGGATGGCAGTTGAGATGACACTC | TVTVHAEESNSSRMAVEMTLHAL |
| | | CATGCCCTAAACTTGGAAAACAAAGATGTATTTTCCAAAAGTGATAACCCATTGCTAGTTGAGTGTTT | NLENKDVFSKSDNPLLVECFDFD |
| | | TGATTTTGATTCAAGTGGTGACCACGAACTTATTGGGGCCTTCCAGACAACTATCACTCAACTTGAGA | SSGDHELIGAFQTTITQLENLYN |
| | | ATCTATATAATTCAAAGTCTGGAGCAAACTTTTTCAGCCATAAGGGACAGAAAAAGTTGAAGGGACAA | SKSGANFFSHKGQKKLKGQLFVD |
| | | TTGTTTGTGGATAAGTTCCAGGAAAAAGTTCAACATACTTTCTTGGACTACATTTCCAGTGGTTTTGA | KFQEKVQHTFLDYISSGFELNFM |
| | | GCTAAATTTTATGGTGGCTGTAGATTTCACGGCTTCAAATGGTGACCCCCGTTCACCTCAGTCTTTGC | VAVDFTASNGDPRSPQSLHYIDP |
| | | ACTACATTGACCCCTCTGGCAGACCGAACTCATACCAGCAGGCAATACTAGGGGTTGGCGAAGTTCTG | SGRPNSYQQAILGVGEVLQFYDN |
| | | CAGTTTTATGACAATGATAGGCGATTCCCGGCATGGGGTTTTGGAGCGAAGACACCACAGGGCTACAT | DRRFPAWGFGAKTPQGYISHCFN |
| | | ATCCCACTGTTTTAATCTGAATGCAACTACCAATGATTGCGAGGTCGTCGGAGTTGAAGGCATCATGT | LNATTNDCEVVGVEGIMSAYTST |
| | | CAGCATACACCTCTACCCTTTACAGTGTCACCCTTGCTGGGCCAACTCTGTTTGGGCCAATTATCAAC | LYSVTLAGPTLFGPIINKAAEIA |
| | | AAAGCTGCCGAGATTGCCAGCCACTCCCTTCAATACGGAAACAACAAATACTTTGTCCTACTCATCAT | SHSLQYGNNKYFVLLIITDGVLT |
| | | CACGGATGGAGTTCTCACTGACATTCAAGAAACAAAAGATTCTATCGTGAGGGCATCTGATCTGCCAT | DIQETKDSIVRASDLPLSILIVG |
| | | TGTCAATCCTCATCGTGGGCGTTGGAAATGCTGATTTCAAGCAAATGGAGATCCTGGATGGAGATAAC | VGNADFKQMEILDGDNGKRLESS |
| | | GGCAAGCGGCTGGAGAGCTCGACCGGCAGGATCGCGACGCGGGACATCGTCCAGTTCGTCCCCATGAG | TGRIATRDIVQFVPMRDVQGGQI |
| | | GGATGTCCAAGGCGGGCAGATCTCCGTCGTCCAGTCGCTGCTGGAGGAGCTCCCGGGCCAGTTCCTGG | SVVQSLLEELPGQFLAYMRSRDI |
| | | CCTACATGCGCAGCCGGGACATCAAGCCGCGAGCCCCGCTGCAGCACGACAACGCCTCTTCCGCTCCG | KPRAPLQHDNASSAPPLYPPTK* |
| | | CCCCTTTACCCTCCAACCAAGTGA SEQ ID NO: 201 | SEQ ID NO: 202 |
| 102 | Xa21_Os05g0453700 | ATGGCCGGTGGCGGCAGGGCCGATGACGAGCGAAGGATCGGGGTGGCGATGGACTACTCGGCGAGCAG | MAGGGRADDERRIGVAMDYSASS |
| | | CAAGAGGGCGCTGGACTGGGCCATCGCCAACCTGCTCCGCCGGGGCGACCACCTCGTCGTCCTCCACG | KRALDWAIANLLRRGDHLVVLHV |
| | | TCCTGCATCACGGCGGGGAGGAGGCCAAGCACGCCCTGTGGGGCAAGTCCGGATCCCCGCTGATCCCT | LHHGGEEAKHALWGKSGSPLIPL |
| | | CTCTCCGAGTTCCGGGATCCGACGGCGATGCAGCAGTACGGCGTGCACTGCGACGCCGAGGTGCTCGA | SEFRDPTAMQQYGVHCDAEVLDM |
| | | CATGCTCGACACGGCGGCGCGCCAGTTGGAGCTTACCGTAGTGGCGAAGCTGTACTGGGGCGACGCCA | LDTAARQLELTVVAKLYWGDARE |
| | | GGGAGAAGCTCTGCGACGCCGTCGAGGAGCAGAAGATCGACACGCTCGTCATGGGCAGCCGCGGCCTC | KLCDAVEEQKIDTLVMGSRGLGS |
| | | GGCTCGATCCAGAGGATTCTTCTGGGGAGTGTGACAAACTACGTGCTGTCAAACGCATCATGCCCTGT | IQRILLGSVTNYVLSNASCPVTV |
| | | AACAGTCGTCAAGGGGAAGTGA SEQ ID NO: 203 | VKGK* SEQ ID NO: 204 |
| 103 | Xa21_OsI_20201 | ATGGAGGAGGATGGGGGAGGCGCGGGGAGGAGGAAGGAGCGGGTGGTGCTGATGTGGGGGTACCTCCC | MEEDGGGAGRRKERVVLMWGYLP |
| | | CGGGGTGTCGCAGCAGCGGTCGCCGCTGCTGGGGCCGGTGCCCGTGAGGATGCCGGGCGCGGCCGCGG | GVSQQRSPLLGPVPVRMPGAAAA |
| | | CGGCCGGTGATGCGTGGAGGGATGTGGCCGGCGGCGGGTGCGGCTTCGCGATGGCCGTCTCCGAGCAA | AGDAWRDVAGGGCGFAMAVSEQG |
| | | GGAAAACTCTTCACTTGGGGCTCTACCGATGATATGGGACAAAGTTACGTGACTGCTGGGAAGCATGA | KLFTWGSTDDMGQSYVTAGKHEE |
| | | GGAAACTCCGGAGGCATTTCCTCTTCCTAGTGATATTGCAATTGTAAGGGCTGACGCTGGATGGGCAC | TPEAFPLPSDIAIVRADAGWAHC |
| | | ATTGTGTTGCAATAACAGGTAAAGGTGATGTCTACACATGGGGTTGGAAAGAATGCGTTCCAACTGGA | VAITGKGDVYTWGWKECVPTGRV |
| | | AGGGTCATTGCAGACCATTCTGCAACTGGGGCTATGGATTGTGATAAAAGGCAAAGTGGGGTTGCCAC | IADHSATGAMDCDKRQSGVATDQ |
| | | TGACCAAGGAAACTCGATTACAGTGAGCCCCAGGTCAAAGGTGTCTAGGACAAACACCAAGGCAGCAT | GNSITVSPRSKVSRTNTKAASGP |
| | | CTGGTCCTGCTGAAAGCCGAAGTTCTGAGGATAGCACAAAGCGACGGAGGTTATCTTCATCGAAACAC | AESRSSEDSTKRRRLSSSKHGAE |
| | | GGGGCTGAAAGTGAATCATCTAGTGATGAAAATCTTTCAGCACCACCTTGTGTTGTGACATTTAATAC | SESSSDENLSAPPCVVTFNTGVK |
| | | AGGAGTGAAGATAGCGGAAGTAGCTGCAGGTGGCCGTCATACATTAGTGCTGTCAGATTTTGGTCAGG | IAEVAAGGRHTLVLSDFGQVWGW |
| | | TCTGGGGTTGGGGCTATGGTGGGGAAGGACAACTAGGTTTAGGCTCTAGGATCCGGACAGTTTCCTCC | GYGGEGQLGLGSRIRTVSSPHPI |
| | | CCACATCCCATACCTTGTGTTCAATCGGCGTTCTACAGCAAGGACCGTACAGGAATTGTGATGGGTAG | PCVQSAFYSKDRTGIVMGSRTLE |
| | | CAGAACTTTGGAAGGAACAAATGATAGAGCAGCAACTTGCGTGAAAGCAATTGCCTGTGGAGGCCGTC | GTNDRAATCVKAIACGGRHSAVV |
| | | ATAGTGCTGTGGTGACAGATTCAGGAGCACTTCTTACATTTGGATGGGGATTATATGGACAGTGTGGA | TDSGALLTFGWGLYGQCGQGNTD |
| | | CAAGGGAACACGGATGATATTTTAAGCCCAACGTGTGTTTCGGCTATTATGGGAGTGAAGATACAACG | DILSPTCVSAIMGVKIQRVAAGL |
| | | TGTAGCAGCTGGGCTATGGCATACTGTATGTACATCTGCTGATGGTGATGTATATTCCTTTGGAGGTA | WHTVCTSADGDVYSFGGNQFGQL |
| | | ACCAGTTTGGGCAGCTGGGAACTGGATCAGATCAAGCTGAGATTGTTCCAAAGTTGGTAGAAGCCTCG | GTGSDQAEIVPKLVEASKLENKH |
| | | AAATTGGAAAATAAGCATGCAAGAGGTGTTTCGTGTGGAGCCCGACACAGTGCAGTCATAACAGATAA | ARGVSCGARHSAVITDKGDVFCW |
| | | AGGAGATGTCTTCTGCTGGGGTTGGAACAAGTACGGTCAGCTTGGTTTGGGCGACTCAACAGACAGAA | GWNKYGQLGLGDSTDRNVPCQVP |
| | | ACGTACCATGCCAAGTGCCCGTGGAAGCGTACAACCCGCTAAACGTTTCATGCGGTTGGTGGCACACG | VEAYNPLNVSCGWWHTLVLAEYP |
| | | TTGGTTCTCGCGGAATATCCTACCTGA SEQ ID NO: 205 | T* SEQ ID NO: 206 |
| 104 | Xa21_OsJ_19053 | ATGGTGGGGAGCTACGCGGCGGGAGGGGGGCGGGGCGCGGCGGTGGCGGCGGTGGCGGAGGGGAAGCT | MVGSYAAGGGRGAAVAAVAEGKL |
| | (Os05g0495700) | GGACGAGCTTCGGCGGAGGATGGGGAAGGCGGACGGCGACCTGCTGAGGATCGTCGGGGTGGGGGGCG | DELRRRMGKADGDLLRIVGVGGG |
| | | GGGCGTGGGGGAGCGCCTTCTGCGCGCTGCTGCAGGACGCGTACGGGCGGCACCGCGACAAGGCGCAG | AWGSAFCALLQDAYGRHRDKAQV |
| | | GTGCGGGTGTGGCGGAGGCCCGGGCGCGCCGTCGACCGCGCCACCGCCGAGCACCTCTTCGAGGTCAT | RVWRRPGRAVDRATAEHLFEVIN |
| | | CAACTCCCGGGAGGACGTCCTCCGCAGGCTCATCCGCCGCTGCGCCTACCTCAAGTACGTCGAGGCCC | SREDVLRRLIRRCAYLKYVEARL |
| | | GCCTCGGCGACCGCACGCTCTACGCCGACGAGATCCTCCGCGACGGCTTCTGCCTCAACATGGTCGAC | GDRTLYADEILRDGFCLNMVDTP |
| | | ACGCCGCTCTGCCCGCTCAAGGTCGTCACCAACCTGCAGGAGGCGGTCTGGGACGCCGACATAGTCAT | LCPLKVVTNLQEAVWDADIVING |
| | | CAACGGCTTGCCCTCCACCGAGACCAGGGAGGTGTTCGGGGAGATCGGGAGGTATTGGAAGGAGAGGA | LPSTETREVFGEIGRYWKERIRP |
| | | TCAGACCCCCCGTGATCATCTCGCTGGCCAAGGGGATCGAGGCGTCGATCGACCCCGTGCCCCGGATC | PVIISLAKGIEASIDPVPRIITP |
| | | ATTACCCCGACACAGATGATCAGCAATGCAACTGGAGTTCCATTGGAGAACATTCTATATCTTGGAGG | TQMISNATGVPLENILYLGGPNI |
| | | TCCTAACATTGCATCTGAGATATATAACAAAGAATATGCAAATGCTCGCATATGTGGAGCTGACAAGT | ASEIYNKEYANARICGADKWRKP |
| | | GGAGAAAACCTCTAGCTAAATTTCTGAGGCAGCCCCATTTTATTGTGTGGGATAATAGTGATCTTATC | LAKFLRQPHFIVWDNSDLITHEV |
| | | ACTCATGAAGTAATGGGTGGCTTAAAGAACATATATGCCATTGGTGCTGGTATGGTGGCTGCACTAAC | MGGLKNIYAIGAGMVAALTNESA |
| | | CAATGAGAGTGCAACCAGCAAATCGGTTTACTTTTCACTTTGCACGTCTGAAATGATATACATCACCC | TSKSVYFSLCTSEMIYITHLLAE |
| | | ATCTCCTAGCCGAAGACCCTGAGAAACTTGCTGGACCTTTATTAGCCGATACCTATGTAACACTGCTG | DPEKLAGPLLADTYVTLLKGRNA |
| | | AAAGGTCGTAATGCATGGTATGGGCAGAAGTTAGCTAAGGGTGAGCTGACTCTTGAAATGGGGGACAG | WYGQKLAKGELTLEMGDSIKGKG |
| | | TATCAAGGGCAAAGGGACTATCCAGGGTGTCTCTGCAGTCCATGCATTTTATGAGCTCTTGAGCCAGA | TIQGVSAVHAFYELLSQSSLSVT |
| | | GTAGCTTAAGCGTGACACATCCAGAAGTGAAGAAGCTTGTTGCTCCTGTCGAGCTGTGCCCAATACTC | HPEVKKLVAPVELCPILKTLYKI |
| | | AAAACACTTTATAAAATCCTAATCAAGAGGGAGCTTGCCACTGACTCCATTCTCCAGGCAATACGAGA | LIKRELATDSILQAIRDESMYDP |
| | | TGAATCAATGTATGATCCACGGGAGAGGATTGAGATGTCTCAGCGCCAGTGTCTTTACCGACCATCTC | RERIEMSQRQCLYRPSLLGLPKV |
| | | TTCTTGGCCTACCTAAAGTAGATATTACCCAGGCTTAG SEQ ID NO: 207 | DITQA* SEQ ID NO: 208 |
| 105 | Xa21_Os05g0530400 | ATGGAGAGTTCCAACCTGGGCGGCGGCGGCGGCGGAGGCGGCGGCGGTGGGCCGCCGCCGTTCTTGAT | MESSNLGGGGGGGGGGGPPPFLI |
| | | CAAGACGTACGAGATGGTGGAGGACGCGGCGACCAACCACGTCGTGTCGTGGGGCCCCGGCGGCGCCA | KTYEMVEDAATNHVVSWGPGGAS |
| | | GCTTCGTCGTGTGGAACCCGCTCGACTTCTCCCGTGACCTGCTGCCCAAGTACTTCAAGCACAACAAC | FVVWNPLDFSRDLLPKYFKHNNF |
| | | TTCTCCAGCTTCATCAGGCAGCTCAACACCTACGGTTTTCGAAAAATCGATCCTGAGAGATGGGAGTT | SSFIRQLNTYGFRKIDPERWEFA |
| | | CGCAAACGAGGATTTCATAAGAGGGCACACGCACCTTCTGAAGAACATCCATCGACGCAAGCCCGTGC | NEDFIRGHTHLLKNIHRRKPVHS |
| | | ACAGCCACTCCCTCCAGAACCAGATAAACGGACCACTCGCCGAATCGGAGAGGCGCGAGCTCGAAGAA | HSLQNQINGPLAESERRELEEEI |
| | | GAGATCAACCGGCTCAAGTACGAGAAGAGCATCCTCGTCGCGGATCTCCAGAGGCAGAACCAGCAGCA | NRLKYEKSILVADLQRQNQQQYV |
| | | GTACGTGATCAACTGGCAGATGCAGGCGATGGAAGGCAGGCTGGTGGCGATGGAGCAACGGCAGAAGA | INWQMQAMEGRLVAMEQRQKNIV |
| | | ACATCGTGGCCTCCCTGTGCGAGATTCTGCAGAGACGCGGCGGCGCCGTGTCGAGCTCGCTGCTGGAG | ASLCEILQRRGGAVSSSLLESDH |
| | | TCCGACCATTTCAGCAAGAAGAGGAGGGTCCCGAAGATGGATCTCTTCGTCGACGATTGCGCGGCGGG | FSKKRRVPKMDLFVDDCAAGEEQ |
| | | CGAAGAACAGAAGGTGTTCCAGTTCCAGGGAATTGGGACGGATGCACCGGCCATGCCTCCCGTGCTTC | KVFQFQGIGTDAPAMPPVLPVTN |
| | | CTGTGACCAATGGTGAGGCTTTTGACAGGGTTGAGCTGAGCTTGGTCTCCCTGGAGAAACTCTTCCAG | GEAFDRVELSLVSLEKLFQRAND |
| | | AGAGCAAATGATGCTTGCACAGCTGCTGAAGAAATGTACTCCCATGGTCATGGTGGTACTGAACCCAG | ACTAAEEMYSHGHGGTEPSTAIC |
| | | CACTGCTATATGTCCTGAAGAGATGAACACTGCTCCAATGGAGACAGGCATCGATCTTCAGTTACCAG | PEEMNTAPMETGIDLQLPASLHP |
| | | CTAGCCTCCATCCCAGATCACCCAGCACAGGGAATGCCCATCTCCATTTATCCACTGAACTCACAGAG | RSPSTGNAHLHLSTELTESPGFV |
| | | TCTCCAGGTTTTGTGCAGAGTCCAGAGCTGCCAATGGCAGAGATTCGTGAAGATATCCATGTGACAAG | QSPELPMAEIREDIHVTRYPTQA |
| | | ATACCCAACACAAGCTGATGTAAATTCTGAGATTGCCTCCTCCACTGATACTTCACAAGACGGCACGT | DVNSEIASSTDTSQDGTSETEAS |
| | | CAGAAACTGAAGCTTCGCATGGACCGACCAACGATGTGTTTTGGGAGCGGTTCCTCACAGAGACTCCA | HGPTNDVFWERFLTETPRSCLDE |
| | | CGGTCATGTTTGGATGAGTCAGAAAGACAAGAGAGTCTCAAGGACGATGTAAAAGCAGAATTAGGCTG | SERQESLKDDVKAELGCNGFHHR |
| | | CAATGGCTTCCATCACCGGGAGAAGGTTGATCAGATCACCGAGCAAATGGGGCACCTTGCTTCAGCCG | EKVDQITEQMGHLASAEQTLHT* |
| | | AGCAGACTCTGCATACCTGA SEQ ID NO: 209 | SEQ ID NO: 210 |
| 106 | Xa21_Os05g0542500 | ATGGCTTCCCACCAGGACCAGGCTAGCTACCGCGCCGGCGAGACCAAGGCCCACACCGAGGAGAAGGC | MASHQDQASYRAGETKAHTEEKA |
| | | GGGGCAGGTGATGGGGGCGAGCAAGGAGAAGGCGAGCGAGGCGAAGGACAGGGCGTCGGAGGCGGCGG | GQVMGASKEKASEAKDRASEAAG |
| | | GGCACGCCGCCGGCAAGGGGCAGGACACCAAGGAGGCGACGAAGGAGAGGGCGTCGGAGACGGCGCAG | HAAGKGQDTKEATKERASETAQA |
| | | GCGGCGAAGGACAAGACCTCCGGCACGGCGCAGGCGGCGAGGGAGAGGGCGTCGGAGACGGCGCAGGC | AKDKTSGTAQAARERASETAQAA |
| | | GGCGAAGGACAAGACCTCCGGCACGGCGCAGGCGGCGAGGGACAAGGCCGCCGAGAGCAAGGAACAGA | KDKTSGTAQAARDKAAESKEQTG |
| | | CCGGCGGCTTCCTCGGCGAGAAGACAGAGCAGGCCAAGCAGAAGGCCGCAGAGACCGCCGGCGCAGCC | GFLGEKTEQAKQKAAETAGAAKQ |
| | | AAGCAGAAGACCGCCGAGACGGCGCAGTACACCAAGGACTCCGCCGTCGCCGGCAAGGACAAGACCGG | KTAETAQYTKDSAVAGKDKTGSV |
| | | CAGCGTCCTCCAACAGGCGAGTGAGCAGGTGAAGAGCACGGTGGTCGGCGCCAAGGACGCGGTGATGA | LQQASEQVKSTVVGAKDAVMSTL |
| | | GCACGCTGGGGATGACCGGAGACAAGGCCGGCACCGACGACGGCGCCAACAAAGACACCTCCGCCGCC | GMTGDKAGTDDGANKDTSAATET |
| | | ACGGAGACGACGGCGAGGGATCACTAG SEQ ID NO: 211 | TARDH* SEQ ID NO: 212 |
| 107 | Xa21_OsI_16826 | ATGGATCGGGGTGACCACCACCACCTCCAGCAGCAGCACCAGTTCTTGATGCCACCACCTGCGCCGGT | MDRGDHHHLQQQHQFLMPPPAPV |
| | (Os04g0541700) | GGTGCCGCCGCAGCTGTGCATGCCGGCGATGATGGCGGACGAGCAGTACATGGACCTGGGCGGTGGCG | VPPQLCMPAMMADEQYMDLGGGG |
| | | GGGCGGCGGCGGGGAGGGGCGGCGGCGGGGAGAGGAAGCGGCGGTTCACGGAGGTGCAGATACGGTCG | AAAGRGGGGERKRRFTEVQIRSL |
| | | CTGGAGTCCATGTTCCACGCGCACCACGCCAAGCTGGAGCCCCGCGAGAAGGCGGAGCTGGCGCGCGA | ESMFHAHHAKLEPREKAELAREL |
| | | GCTGGGCCTGCAGCCGCGGCAGGTGGCCATCTGGTTCCAGAACAAGCGCGCGCGGTGGCGCTCCAAGC | GLQPRQVAIWFQNKRARWRSKQL |
| | | AGCTCGAGCACGACTACGCCGCACTCCGCTCCAAGTACGACGCCCTCCACTCCCGCGTCGAGTCCCTC | EHDYAALRSKYDALHSRVESLKQ |
| | | AAGCAAGAGAAGCTCGCTCTCACCGTCCAGTTGCACGAGCTAAGAGTGAGGCTGAGGGAACGGGAGGA | EKLALTVQLHELRVRLREREERS |
| | | GAGGAGCGGCAACGGTGGTGCCGCCACGACGGCAGCAAGCAGCAGCAGCTGCAACGGATCAGGCAGCG | GNGGAATTAASSSSCNGSGSEEV |
| | | AGGAGGTCGACGACGACAAGAGGAACGTCGCCGCGGGGTGCCTGGACCTCGAGCCGCCGGAGAGCTGC | DDDKRNVAAGCLDLEPPESCVLG |
| | | GTGCTCGGCGGCGCGACGTGCGCCACGCCGGCGGACGTGTCGGTGGAGTCGGATCAGTGCGACGACCA | GATCATPADVSVESDQCDDQLDY |
| | | GCTTGACTACGAGGAGGGCTTGTTCCCGGAGTCGTTCTGCGCCACGCCGGAGCTGTGGGAGCCATGGC | EEGLFPESFCATPELWEPWPLVE |
| | | CGCTAGTCGAGTGGAATGCGGTGGCTTGA SEQ ID NO: 213 | WNAVA* SEQ ID NO: 214 |
| 108 | Xa21_Os05g0495300upstream | ATGGCTGTTTGGTTGGTTTTCACTTTCCAGCAGTTGTGCACTCAAAAGGATTTGTCAGTTCCAGATCT | MAVWLVFTFQQLCTQKDLSVPDL |
| | | TGCTGCCGGTTACACTGTTGTTTCCGGAGCCAGGCCTAAGTGTTGGCAGCGGCAGTGCAGAGTAAGAC | AAGYTVVSGARPKCWQRQCRVRQ |
| | | AGAATTCAGATGCAGTGTTGGCGCGGCTAGCAGGCAACGGTGATCCTGTGCGGCAAGGGACGAGCAAA | NSDAVLARLAGNGDPVRQGTSKV |
| | | GTTTGTGGGCAGCAGGATGAAACAATAGTGGCCTTGTGCACTGATAGTATTTTTGAAGGGACTGCACT | CGQQDETIVALCTDSIFEGTALI |
| | | GATATGA SEQ ID NO: 215 | * SEQ ID NO: 216 |
| 109 | Xa21_OsJ_19472 | ATGGCGGAGCCGGCAAGATGGGCTCTCTTGCTCCTCCTTGGCGTCGTACTCCTGGCGCCCGTTACGCT | MAEPARWALLLLLGVVLLAPVTL |
| | (Os05g0552600) | GGCGGTGCGTGTTGAAGATGGCCCGAAGCCACCGACTCATCCGAATAATGGTGATAATGGGAAGCACG | AVRVEDGPKPPTHPNNGDNGKHE |
| | | AGAACCGCGTCACCGCCCCCACCCGTATACTCGCCGCCGCCACCGCCGCGTTCATCTCCTCCACCACC | NRVTAPTRILAAATAAFISSTTG |
| | | GGTTCCCACGTCGCCGCCGCCATCACCGCTGCCATCGCCTCCTCCACCTGTGCCCTCGTCACCGCCAC | SHVAAAITAAIASSTCALVTATT |
| | | CACCAGTGTCATCACCTCCACCACCGGTGCCCTCTTCGCCACCGCCGCCAGTGGTGCCATCGCCTCCT | SVITSTTGALFATAASGAIASST |
| | | CCACCGGTGCTCTCCTCGCCGCCACCACCAGTGGTGGCATCCCCTCCACCACCGGTAGTACCATCACC | GALLAATTSGGIPSTTGSTITSS |
| | | TCCTCCACCAGGAGCCAGCGACGTGGTCTACTGCACGAACAAGACGAGGTACCCCACCTGCACCTCGC | TRSQRRGLLHEQDEVPHLHLAGV |
| | | CGGCGTACTGCCCCAGCAGGTGCCCCAAATCCTGCCACATGGACTGCGCCACCTGCAAGACCACTGCA | LPQQVPQILPHGLRHLQDHCNLP |
| | | ACCTGCCGGGCGCGGTGTGCCAGGACCCGCGGTTCATCGGCGGCGACGGCAACACGTTCTACTTCCAC | GAVCQDPRFIGGDGNTFYFHGRR |
| | | GGCCGCCGTGACCGCGACTTCTGCCTGCTCTCCGACGCAAACCTGCACATCAACGGCCACTTCATCGG | DRDFCLLSDANLHINGHFIGNHV |
| | | CAACCACGTCCCGGGCCTCAAGAGGGACCCGACCTGGGTGCAGGCGATCGCCGTGCAGTTCTCCGGCG | PGLKRDPTWVQAIAVQFSGGHRL |
| | | GCCACCGCCTCTACGTCGGCGCGCGCAGGACGGCCGTGTGGGACGACGACTCCGACCGCCTCGCCGTC | YVGARRTAVWDDDSDRLAVVFDG |
| | | GTGTTCGACGGCGAGACCGTGCAGCTGCAGCGCGTCGCGCACGCCAGGTGGGAGTCGGGTTCCGGCCT | ETVQLQRVAHARWESGSGLSVTR |
| | | GTCCGTGACGCGGACCAAGGCGGCCAACGGCGTCCTCGTGGAGCTCGACGGCGTGTTCAAGATCACCG | TKAANGVLVELDGVFKITANVVP |
| | | CGAACGTGGTGCCCATCACCAAGGAGGACTCGAGGATCCACAGGTACGGGGTCACCGACGACGACTGC | ITKEDSRIHRYGVTDDDCLAHLD |
| | | CTCGCCCACCTCGACCTGGCGTTCAAGTTCTACGCCCTCACCGACGACGTCCACGGCGTGCTGGGCCA | LAFKFYALTDDVHGVLGQTYRSS |
| | | GACGTACAGGAGCAGCTACGTGAACCGCCTCGACGTGTCGGCGAAGATGCCCGTCATGGGAGGCGAGA | YVNRLDVSAKMPVMGGEKQFTSS |
| | | AGCAGTTCACCTCGTCGGGTCTTTTCGCCGCTGACTGCGCCGTCGCGCGGTTCGGGCGCGCCGGCGAT | GLFAADCAVARFGRAGDAGAVAI |
| | | GCCGGCGCCGTTGCCATTGCCTCCGAGGAGCTGGTCGACGTCAAGTGCTCCACCGGCCTCGACGGCGT | ASEELVDVKCSTGLDGVGVVCKK |
| | | CGGGGTGGTGTGCAAGAAGTAG SEQ ID NO: 217 | * SEQ ID NO: 218 |
| 110 | Xa21_Os06g0127100 | ATGGAGTACGACGAGCAGGAGGAGTACGCGACGGTGACGTCGGCGCCGCCGAAGCGGCCGGCGGGGAG | MEYDEQEEYATVTSAPPKRPAGR |
| | | GACCAAGTTCAGGGAGACGAGGCACCCGGTGTACCGCGGCGTGCGGCGGCGGGGGCCCGCGGGGCGGT | TKFRETRHPVYRGVRRRGPAGRW |
| | | GGGTGTGCGAGGTCAGGGAGCCCAACAAGAAGTCCCGCATCTGGCTCGGCACCTTCGCCACCGCCGAG | VCEVREPNKKSRIWLGTFATAEA |
| | | GCCGCCGCGCGCGCCCACGACGTCGCCGCGCTCGCCCTCCGCGGCCGCGGCGCGTGCCTCAACTTCGC | AARAHDVAALALRGRGACLNFAD |
| | | CGACTCCGCCCGCCTCCTCCGCGTCGACCCGGCCACCCTCGCCACCCCCGACGACATCCGCCGCGCCG | SARLLRVDPATLATPDDIRRAAI |
| | | CCATCGAGCTCGCCGAGTCATGCCCGCACGACGCCGCCGCCACCGCCGCCGCTGCCTCCAGCTCTGCC | ELAESCPHDAAATAAAASSSAAA |
| | | GCCGCCGTCGAGGCCGCCGCCGCCGCCGCGCCCGCCATGATGATGCAGTACCAGGACGACATGGCGGC | VEAAAAAAPAMMMQYQDDMAATP |
| | | GACGCCGTCCAGCTACGACTACGCGTACTACGGCAACATGGACTTCGACCAGCCGTCCTACTACTACG | SSYDYAYYGNMDFDQPSYYYDGM |
| | | ACGGGATGGGCGGCACCGCCGGCGGCGGCGGCGAGTACCAGAGCTGGCAGATGGACGGCGACGACGAT | GGTAGGGGEYQSWQMDGDDDGSA |
| | | GGCAGCGCCGGTGGCTACGGCGGCGGCGACGTCACACTCTGGAGCTACTGA | GGYGGGDVTLWSY* |
| | | SEQ ID NO: 219 | SEQ ID NO: 220 |
| 111 | Xa21_Os06g0281800 | ATGAAACTAAGCAACATGACAGCCCTTTTGTCACTTCTGATCTCCATCCTTCTGGTTGCGATAGAGGC | MKLSNMTALLSLLISILLVAIEA |
| | | CAGAGGTTTGGTGGGAGTGGAACGGGTGGAGCTGCCAAATCCTGCATCTATGGTAACAGCATACTGGA | RGLVGVERVELPNPASMVTAYWK |
| | | AGGAGATGCTTCCCCACTCTCCAATGCCGACTGCTATACTCGAGCTTTTGAACCCTCCTACTGATGTT | EMLPHSPMPTAILELLNPPTDVN |
| | | AATCAAGGTGTTTATGGCAATGGCTATGCTCAAGTTTATGGCAACGGATATGATGAAGGCTACCTTAA | QGVYGNGYAQVYGNGYDEGYLNG |
| | | CGGATATAGCCATTCCTATGGTAATGGCTATTCCAATGGCTATTTTCACAAGGCAAATCTCCACTTCT | YSHSYGNGYSNGYFHKANLHFLE |
| | | TAGAAGATGCCTTAAAACCGGGGTCCATAATCACTCCTTACATCACGGGGATAGCTACACGTGCTCCA | DALKPGSIITPYITGIATRAPFL |
| | | TTTCTTCGCCGGGACATTGCGGATTCAATCTCGATGAGCACAAAGAACTTTGCCGACATCCTAAAAAT | RRDIADSISMSTKNFADILKMFS |
| | | GTTTTCGCCTATATCCCTCGTCATGGCCGACGGTATACAGTCGGCATTAGACACATGTGAGCACCACC | PISLVMADGIQSALDTCEHHHPI |
| | | ACCCTATCGAGGGCGAGGAACGGGCGTGTGCCACATCTATCGAGTCGGTGGTTGAGTTTGTTATGTCT | EGEERACATSIESVVEFVMSVLG |
| | | GTGCTCGGGACACATGACCTCCGAGCCTTCTCCCCCGATGTCCCAACAGATGGCGTCATGTCAGGAAA | THDLRAFSPDVPTDGVMSGNKYK |
| | | CAAGTACAAGGTGGCAGCGGTAAGGACAGTTGCGGGCTCAAAGGGGGACACTGTGACGTGCCACACCA | VAAVRTVAGSKGDTVTCHTMRFP |
| | | TGAGGTTCCCGTTCGCTGTGTTCTACTGCCACGCGATCAACCCTACCCGGGTGTACGCGGTGGTGCTG | FAVFYCHAINPTRVYAVVLESEE |
| | | GAGAGCGAGGAGGATGGCAGCGGCACACCGGAGAAGATGGAGGCGCTCGCCGTGTGCCACCTGGACAC | DGSGTPEKMEALAVCHLDTSRFD |
| | | GTCACGTTTCGACCCCAAGACTCCATTGTTCGTCGAGCACAATCTCAGGCCGGGAGATGCGTCGGTGT | PKTPLFVEHNLRPGDASVCHFVS |
| | | GCCACTTCGTTAGCAGGGACAGCGTGATCTGGGCACCTGTAGCTGCTGTGATAACACATGGAGATGAA | RDSVIWAPVAAVITHGDEQVSIA |
| | | CAAGTCTCAATTGCCGAGTAG SEQ ID NO: 221 | E* SEQ ID NO: 222 |
| 112 | Xa21_Os06g0313440 | ATGAAGACTCTGATACAGGAGGCAGTCACAGGCTTATGCACAAGCTCATGTCCTCACCCAAAGAACAT | MKTLIQEAVTGLCTSSCPHPKNM |
| | | GGTGATCGCGGACTTGGGCTGCTCCTCCGGACCAAACGCGCTCACGCTCGTCTCTGCCGCCGTCGACG | VIADLGCSSGPNALTLVSAAVDA |
| | | CCATCCACCGCTACTGCGCGCAGCACGAGCAACTGCCGCCGGAGATGTGCGTGCTCCTGAACGATCTG | IHRYCAQHEQLPPEMCVLLNDLP |
| | | CCTGGCAACGATTTCAACACCGTGGCGAAGAGCTTGGACACACTGAAGCACAGTGGTGGTGATGCTCT | GNDFNTVAKSLDTLKHSGGDALA |
| | | CGCTCAACCTGCTGTTGTGATCACCGGTATGGTACCTGGGTCGTTCTACGAGAGGCTATTCGCTCGTG | QPAVVITGMVPGSFYERLFARGS |
| | | GCTCCTTGCATTTGGTTTGCTCGGCTAACAGCTTGCATTGGTTATCCGAGGCACCTGAAGATCTCAAG | LHLVCSANSLHWLSEAPEDLKKS |
| | | AAGAGTAGAATCCCGATGCATGATAGTGATGAACAGTTGAGATCATCAAGGCATCAAATTGTCGCGGA | RIPMHDSDEQLRSSRHQIVADSY |
| | | TTCATATGCTCGGCAGTTTAGGAAGGATTTCATGCGATTCTTGAGCTTGAGGGCACAGGAGATTGTCC | ARQFRKDFMRFLSLRAQEIVPGG |
| | | CTGGAGGCCGAATGGTTGTCTCACTGTTGGTCAAGCGCTCGGACAAACCTGACACTGAGTTAATTCAA | RMVVSLLVKRSDKPDTELIQPWT |
| | | CCATGGACGCCGGCGGTGACGGCTTTAAGTGACATGGCATTAAGGGGTGTCATCAGCAAAGAAAAGCT | PAVTALSDMALRGVISKEKLDSF |
| | | TGACTCCTTCTACATTCCCTTGTGCTGCCCTATGGACAGTGAAGTTAACAAGATCATAGAAGAGGAGG | YIPLCCPMDSEVNKIIEEEGSFE |
| | | GTTCATTTGAGATCAACAAGATGGTGATGCACGATCCCTATGGCGGCACGGGCAGAGCCTTGCTCGAT | INKMVMHDPYGGTGRALLDLKMV |
| | | CTGAAGATGGTAGCCTTGCGCGTAAGAGCTGTATTTGAGCCAATAATAGTGCAGCATTTTGCAGCGTC | ALRVRAVFEPIIVQHFAASNEIM |
| | | AAATGAAATCATGGACGACTTCGTCAGAGCAGTGGAGCGGCACTTGATCAGTTCTGGGGCTCTGGAAG | DDFVRAVERHLISSGALEARLSG |
| | | CTAGGCTGTCCGGCCAACACTCGTTTGCCTTCTTGTGTTTGTCTCTCACTAGAGCGATGTGA | QHSFAFLCLSLTRAM* |
| | | SEQ ID NO: 223 | SEQ ID NO: 224 |
| 113 | Xa21_OsI_22710 | ATGGCTTCTAGGTCTCTTGTGTTGATGTTTGTGTTGATGGTTTCTCTTGTCATCGTCTCTCAGGGTGT | MASRSLVLMFVLMVSLVIVSQGV |
| | (Os06g0313500) | CCTTGCTGCAAGGGAGCTTGCAGAGAAAACCAAAGTGGATGTAGATGGAATGAATGTCGACCTTCCTA | LAARELAEKTKVDVDGMNVDLPK |
| | | AAGGCCGCAAAGGTGCAGTTGCAGTCTCAGGGTACGGTGGTGGTTGGTCAGGGTCATACGGTGGCAGA | GRKGAVAVSGYGGGWSGSYGGRH |
| | | CACTATCCATGA SEQ ID NO: 225 | YP* SEQ ID NO: 226 |
| 114 | Xa21_Os06g0318600 | ATGCCGACGGCTGCGTCGGAGCGCGACGGCGGCGGCCGTGCCGCGCTGGGGAAGGCGGCGGCGGGGCT | MPTAASERDGGGRAALGKAAAGL |
| | | CGCCGCGGCGGCCGTGGTGTCGCTGACCGGGCTCGCCGCGGAGCCCCTCTCGCCGCCGCCGCCGGCGC | AAAAVVSLTGLAAEPLSPPPPAR |
| | | GGGCCGAGTCGCTCACCGTCGCGTTCCCTGTCTCCAAGGCCCGTGAGGTGAACCGGGTGCAGAGGACG | AESLTVAFPVSKAREVNRVQRTL |
| | | CTGGTGGAGGCGTGGGGGCTGATCCGTGAGACCTTCGTGGATCCCACCTTCAATCACCAAGATTGGGA | VEAWGLIRETFVDPTFNHQDWDM |
| | | CATGAGACTGCAGCAGACCATGGTGGAGATGTTCCCTCTGAAATCGGAGGATGCCGCCTATGGTAAGA | RLQQTMVEMFPLKSEDAAYGKIS |
| | | TCAGTGGGATGCTGTCCACACTCGGCGATCCGTTCACCAAGATCATCAGCCCCAAGGAATATCAGAGT | GMLSTLGDPFTKIISPKEYQSFR |
| | | TTCAGGATTGGAAGCGATGGGAGTGTCCAAGGGGTTGGGGTGTTCATAAACAAGGAGCCTAGCTCTGG | IGSDGSVQGVGVFINKEPSSGRL |
| | | ACGCTTGCTTGTTATGGACTGCATTGAAGGTGGCCCAGCAGATCGAGCAGGCCTGCATGGAGGCGATG | LVMDCIEGGPADRAGLHGGDELV |
| | | AACTAGTTGAGATCGATGGGAAGAGTGTTTCTGGGTTGGATGGAGAAGCTGCAGCTCAGAGACTTAGA | EIDGKSVSGLDGEAAAQRLRGRV |
| | | GGTCGTGTTGGAACAACTGTGAAAGTGAAAGTGTTGGATGGCACTGAAAACGAAAGGAATGGTAGGAT | GTTVKVKVLDGTENERNGRIRQK |
| | | AAGACAAAAGGAGGTCCAGCTATCTCGTGAGGTTATCAATCTTTCACCTCTATCAACAGCAATTATCT | EVQLSREVINLSPLSTAIISHRS |
| | | CTCATAGGTCGGACGATGGCCGTGAGTGCAAAACTGGGTATGTTAGGCTAGCTGCATTCTCTCAGACT | DDGRECKTGYVRLAAFSQTAAAE |
| | | GCTGCAGCTGAAATGGAAAGTGCCATTAAGAAGATGGAGGATGAGGGTGTCCAGTCATATATTTTAGA | MESAIKKMEDEGVQSYILDLRNN |
| | | TCTGCGAAATAATCCTGGTGGTCTAGTAAAAGCAGGTCTTGACGTGGCTCAAATGTGGTTGGATGGAA | PGGLVKAGLDVAQMWLDGNETLV |
| | | ATGAAACTCTTGTCAACACTGTTGACCGTGAGGGAAATGTCCTACCAATAAATATGGCCCGGGGCCAT | NTVDREGNVLPINMARGHSLTHD |
| | | TCTTTAACACATGATCCCCTTGTCGTACTTGTCAATGAAGGAAGTGCAAGTGCAAGTGAAATCTTGGC | PLVVLVNEGSASASEILAGALHD |
| | | AGGAGCATTACATGACAATGGTCGTGCTATTTTGGTCGGCCACAGAACTTTTGGAAAAGGAAAAATAC | NGRAILVGHRTFGKGKIQSVTEL |
| | | AGAGTGTGACTGAATTGGATGATGGATCTGCTCTGTTTATCACGGTCGCAAAGTATCTCTCTCCAGCA | DDGSALFITVAKYLSPALHEIDQ |
| | | CTGCATGAAATCGACCAAGTAGGCATCCAACCTGACATACAATGCACCCCTGAAATGTTATCTTTGCC | VGIQPDIQCTPEMLSLPRAPSLK |
| | | AAGAGCACCTTCGCTAAAAGAGGACGACAAAGCCACAAATCTGGAGATGGATTCATGTATCATGGTGG | EDDKATNLEMDSCIMVAEQALEI |
| | | CAGAGCAAGCATTGGAAATTGAGAAATCAAAGGGATCTGCTTCGTAG | EKSKGSAS* |
| | | SEQ ID NO: 227 | SEQ ID NO: 228 |
| 115 | Xa21_OsI_23134 | ATGGCGTCCAAGCGGATCCTCAAGGAGCTCAAGGACCTGCAGAAGGATCCCCCAACCTCCTGCAGCGC | MASKRILKELKDLQKDPPTSCSA |
| | (Os06g0506600) | TGGCCCTGTGGGAGAAGATATGTTCCACTGGCAAGCTACGATTATGGGTCCAGCAGATAGTCCATATG | GPVGEDMFHWQATIMGPADSPYA |
| | | CAGGTGGCGTCTTCTTGGTTTCTATTCACTTCCCTCCAGACTATCCGTTCAAACCGCCCAAGGTTGCA | GGVFLVSIHFPPDYPFKPPKVAF |
| | | TTTAAGACAAAGGTTTTCCACCCAAACATCAACAGTAACGGAAGCATCTGCCTTGATATCCTGAAGGA | KTKVFHPNINSNGSICLDILKEQ |
| | | ACAGTGGAGCCCTGCATTGACAGTGTCAAAGGTACTCCTCTCAATTTGTTCTCTACTGACGGACCCAA | WSPALTVSKVLLSICSLLTDPNP |
| | | ACCCAGACGACCCGCTAGTTCCGGAGATTGCGCACATGTACAAGACTGATCGGGCCAAATATGAATCA | DDPLVPEIAHMYKTDRAKYESTA |
| | | ACTGCGAGGGGCTGGACCCAGAAATATGCAATGGGCTGA | RGWTQKYAMG* |
| | | SEQ ID NO: 229 | SEQ ID NO: 230 |
| 116 | Xa21_OsJ_22168 | ATGGATTCGTGGATTGAGCAGACTTCCCTGAGCTTGGACCTTAACGTCGGCTTGCCGTCGACGGCGAG | MDSWIEQTSLSLDLNVGLPSTAR |
| | (Os06g0649000) | GAGATCATCGGCTCCGGCGGCGCCGATTAAGGTTCTCGTGGAGGAGAACTTCTTGTCCGTCAAGAAGG | RSSAPAAPIKVLVEENFLSVKKD |
| | | ATCACGAGGTTGAGGCGCTGGAGGCGGAGCTCCGGCGAGCGAGCGAGGAGAACAAGAAGCTGACCGAG | HEVEALEAELRRASEENKKLTEM |
| | | ATGCTTCGGGCGGTGGTGGCCAAGTACACCGAGCTGCAGGGACAGGTCAACGACATGATGTCGGCGGC | LRAVVAKYTELQGQVNDMMSAAT |
| | | GACGGCGGCGGCGAACGCCGGGAACCACCACTCGTCCACGTCGGAGGGCGGCTCGATCTCGCCGTCGA | AAANAGNHHSSTSEGGSISPSRK |
| | | GGAAGCGGATCCGTAGCGTCGACAGCCTCGATGACGCCGCCCCCCACCACCACCCCCACCGCAAGCCG | RIRSVDSLDDAAPHHHPHRKPSP |
| | | TCCCCGCCGTTCGTCGCCGCCGCCGCCGCCGCAGCCTACACCTCCCCCGACCAGATGGAGTGCACGTC | PFVAAAAAAAYTSPDQMECTSAS |
| | | GGCGTCCGCCGCCGCCGCCAAGCGCGTCGTCCGCGAGGACTGCAAGCCCAAGGTGTCCAAGCGCTTCG | AAAAKRVVREDCKPKVSKRFVHA |
| | | TCCACGCCGACCCCTCCGACCTCAGCCTCGTGGTGAAGGATGGGTATCAATGGCGGAAGTACGGGCAG | DPSDLSLVVKDGYQWRKYGQKVT |
| | | AAGGTGACGAAGGACAACCCGTGCCCGCGGGCCTACTTCAGGTGCTCGTTCGCGCCGGCGTGCCCGGT | KDNPCPRAYFRCSFAPACPVKKK |
| | | GAAGAAGAAGGTGCAGCGCAGCGCCGACGACAACACCGTCCTCGTCGCCACGTACGAGGGCGAGCACA | VQRSADDNTVLVATYEGEHNHAQ |
| | | ACCACGCCCAGCCGCCGCACCACGACGCCGCCGGCAGCAAGACCGCCGCCGCCGCCAAGCACTCACAG | PPHHDAAGSKTAAAAKHSQHQPP |
| | | CACCAGCCGCCACCGAGCGCCGCCGCCGCCGTCGTCCGGCAGCAGCAAGAGCAGGCGGCGGCGGCCGG | PSAAAAVVRQQQEQAAAAGPSTE |
| | | GCCGTCGACGGAGGTGGCGGCGAGGAAGAACCTGGCCGAGCAGATGGCGGCGACGCTGACGAGGGACC | VAARKNLAEQMAATLTRDPGFKA |
| | | CCGGGTTCAAGGCGGCGCTCGTCACGGCGCTCTCCGGCCGGATCCTCGAGCTCTCGCCGACCAAGAAC | ALVTALSGRILELSPTKN* |
| | | TAA SEQ ID NO: 231 | SEQ ID NO: 232 |
| 117 | Xa21_Os07g0114300 | ATGGGCGGCGGCAATGGGCAGAAGTCGAAGATGGCGAGGGAGAGGAACATGGAGAAGAACAAGGGCGC | MGGGNGQKSKMARERNMEKNKGA |
| | | CAAGGGGAGCCAGCTTGAGGCCAACAAGAAGGCCATGAACATCCAGTGCAAGATATGCATGCAGACTT | KGSQLEANKKAMNIQCKICMQTF |
| | | TCATCTGCACCACCTCTGAGACCAAGTGCAAAGAGCATGCTGAGGCGAAGCATCCGAAGAGCGACCTC | ICTTSETKCKEHAEAKHPKSDLT |
| | | ACCGCGTGCTTCCCTCACCTCAAGAAATAA | ACFPHLKK* |
| | | SEQ ID NO: 233 | SEQ ID NO: 234 |
| 118 | Xa21_OsJ_22964 | ATGTCCTTTCTGCCACTCATCTTCTTCCTTGGCCTTAGCCTAGCATCCATCACCAACGGTGACGATAA | MSFLPLIFFLGLSLASITNGDDN |
| | (Os07g0129900) | CCACCAGTTCGTCTACAATGGCTTCACTAGTGCTAACAACCTCTCCCTTGACGGCGTGGCCATGGTGA | HQFVYNGFTSANNLSLDGVAMVT |
| | | CTCCTGATGGCCTCGTCGAGCTCACCAACGATGGAATCCGGATGAAAGGCCATGCTTTCTACCCGTCT | PDGLVELTNDGIRMKGHAFYPSP |
| | | CCTCTGCACTTCCGCCAGTCTCCTAATGGCATGGTGCGATCTTTCTCTGTCTTCTTCGTGTTCGGCAT | LHFRQSPNGMVRSFSVFFVFGIV |
| | | CGTCCCTACCTTCTCTGACCTGAACAGTGGCCATGGCATCGCCTTCGTCATTGCACCAAGCAAGAATT | PTFSDLNSGHGIAFVIAPSKNFS |
| | | TCTCAGATGCTATCGCGGCACAATACTTTGGGCTCTTCAATAGTGAGACCGATGGCAATGATAGAGGC | DAIAAQYFGLFNSETDGNDRGHI |
| | | CACATCTTTGCAATTGAGCTTGACACCGTTAAGAACACAGAGTTTGGGGACATGAATGATAATCATGT | FAIELDTVKNTEFGDMNDNHVGI |
| | | TGGGATCGATATCAATAATCTCACCTCTTTGCGATCCTACCCCGCTGGATACTATGAAGAGAGTGGAA | DINNLTSLRSYPAGYYEESGRFK |
| | | GGTTCAAAAACCTGACCCTTGCATCCATGGAGGCTATACAAGTGTGGGTTGATTATGACAGAGAAGCT | NLTLASMEAIQVWVDYDREATRI |
| | | ACTCGGATCAACGTGACCATGGCTCCTCTTGCCATGGCCAAACCTGTGAGGCCATTGTTGTCTGCTAC | NVTMAPLAMAKPVRPLLSATYNL |
| | | CTACAACCTCTCAGGATTGCTCATGGAGCGTTCATACATCGGCTTCTCTTCCTCGACAGGCGCAACTA | SGLLMERSYIGFSSSTGATSARH |
| | | GTGCACGACACTACCTTCTTGGCTGGAGCTTCAGTATGAATGGAGGAACTGCGCCGGCCATCGACATT | YLLGWSFSMNGGTAPAIDIAKLP |
| | | GCCAAGCTGCCAAAGCTACCTCGTGTTGGTCCTAAGTCTGATCCATCCAATCTGTTGCAAATCATATT | KLPRVGPKSDPSNLLQIILPVAT |
| | | GCCGGTGGCAACCGCAGCATTCCTCGTGGCTGCGGGTGCTACTGTTTTTCTCCTTGTGCGAAGGCGTA | AAFLVAAGATVFLLVRRRMRYTE |
| | | TGAGGTACACTGAGTTGCGAGAAGATTGGGAGATCGATTTTGGGCCACACCGGTTCGCATATAAGGAT | LREDWEIDFGPHRFAYKDLFHAT |
| | | TTGTTCCATGCCACGGAAGGGTTCCAGAACAAGAATTTGCTAGGCACAGGAGGTGCTGGGAGGGTATA | EGFQNKNLLGTGGAGRVYKGMLL |
| | | CAAGGGGATGCTTCTAGGGTCCAAACAAGAGATCGCAGTGAAAAAGATACCACAAAATTCGAAGGAAA | GSKQEIAVKKIPQNSKESMKQFV |
| | | GCATGAAACAGTTTGTAGCGGAGATTATTAGCATAGGCCGCCTTGACCACCGCAATCTTGTTCACCTC | AEIISIGRLDHRNLVHLLGYSRR |
| | | CTCGGTTATTCCCGACGTAAAGGTGAATTGATTTTAGTGTACGAGTATATGTCAAATGGAAGCTTGGA | KGELILVYEYMSNGSLEKYLYGQ |
| | | AAAGTACTTGTATGGTCAAGATGGTAGGTGCACTTTGGATTGGGGTCAACGGTTTCAAATCATCAAAG | DGRCTLDWGQRFQIIKGIASGLL |
| | | GGATCGCGTCTGGGTTGCTTTACCTCCATGAAGAATGGGAGAAAGTAGTGATTCATCGAGATGTGAAG | YLHEEWEKVVIHRDVKPSNILLD |
| | | CCAAGTAACATACTCCTCGACAACAAGATGAATGCAAAGATAGGTGACTTTGGCCTCTCAAGGTTGCA | NKMNAKIGDFGLSRLHDHGANPQ |
| | | CGATCATGGAGCCAATCCACAAACTACACACGTTGTTGGCACCATAGGGTACTTAGCTCCAGAAATAG | TTHVVGTIGYLAPEIALTGKVTP |
| | | CATTGACCGGCAAGGTGACTCCACTCGCCGACGTATTTTCCTTCGGCATATTAGCCCTAGAGATTACA | LADVFSFGILALEITCGQKPMKQ |
| | | TGTGGGCAGAAACCTATGAAGCAAAATGCACAAGGCATCCAACAAACATTGGTTGGTTGGGTGCTAGA | NAQGIQQTLVGWVLECWKKGSVV |
| | | ATGTTGGAAAAAAGGATCAGTTGTTGATGCAGTTGATGCCAACCTCCAAGCTGACTATGATATTGCTG | DAVDANLQADYDIAEASLVLKLG |
| | | AAGCAAGTTTAGTGCTAAAATTAGGATTGCTATGCTCACATCCATCTGAACACTCAAGACCGAATATG | LLCSHPSEHSRPNMRQVTQYLNG |
| | | AGACAAGTGACGCAGTATCTCAATGGTGACATGCCACTACCAGAAACTATTTCAATTCAAACCCAGGA | DMPLPETISIQTQDLACFT* |
| | | TTTGGCTTGTTTCACTTGA SEQ ID NO: 235 | SEQ ID NO: 236 |
| 119 | Xa21_OsJ_23939 | ATGGCCTACGACCAGGCCGCGCTCTCGGCGCGGGGCGCGGCCGCGGCGCTCAACTTCCCCGTGGAACG | MAYDQAALSARGAAAALNFPVER |
| | (Os07g0410300) | CGTGCGCGAGTCGCTCCGCGCGCTCGCGCTCGGCGCGGCCGGCGGCTCGCCCGTGCTGGCGCTGAAGG | VRESLRALALGAAGGSPVLALKG |
| | | GGCGGCACTCCAAGCGAAAGCGGCGGAAGAGATCAGAGCTGGCCGGTGCCACGGCGACGGCGAACAAG | RHSKRKRRKRSELAGATATANKT |
| | | ACGACGACGACGACGACGTCCGCCACGACCGGCGGGGAGCACAAGCAGATCGTCCTCGAGCTGGAGGA | TTTTTSATTGGEHKQIVLELEDL |
| | | CCTCGGCGCCGACTACCTAGAGGAGCTCCTCTGGATGTCCGAGCAAACTAGCCATTGA | GADYLEELLWMSEQTSH* |
| | | SEQ ID NO: 237 | SEQ ID NO: 238 |
| 120 | Xa21_Os07g0529800 | ATGTCTGATCTCGACATTCAGATCCCAACTGCCTTCGATCCCTTTGCTGAGGCCAATGCTGGAGACTC | MSDLDIQIPTAFDPFAEANAGDS |
| | | TGGTGCGGCTGCAGGATCAAAGGACTACGTTCATGTACGCATCCAGCAGCGTAATGGCCGTAAGAGCC | GAAAGSKDYVHVRIQQRNGRKSL |
| | | TGACCACTGTCCAGGGATTGAAGAAGGAATTCAGCTACAACAAGATCCTCAAAGATCTCAAGAAAGAG | TTVQGLKKEFSYNKILKDLKKEF |
| | | TTCTGCTGCAATGGTACTGTTGTCCAGGACCCAGAGCTTGGCCAGGTCATTCAACTTCAGGGTGATCA | CCNGTVVQDPELGQVIQLQGDQR |
| | | GAGGAAGAACGTATCAAATTTTCTTGTTCAGGCCGGCATTGTGAAGAAGGAACACATCAAGATTCATG | KNVSNFLVQAGIVKKEHIKIHGF |
| | | GTTTCTGA SEQ ID NO: 239 | * SEQ ID NO: 240 |
| 121 | Xa21_Os07g0545800 | ATGGACTTGCACCAGTTATTAAAGTACAGATTGACTGGCGCTAACGTTGTGTACGAAATTCCTACAGA | MDLHQLLKYRLTGANVVYEIPTE |
| | | GAACAACTTGCAAAACTCTCCCTGGCAAGCTAACCCACTGAAGTACGAGTTCAGCGATTCCCCATACA | NNLQNSPWQANPLKYEFSDSPYT |
| | | CCCCTCTCTCTTCCCAATTTGAGTGTGACAATTTGTCTGCTCTTACCAACACTCCAGATAACCAGAGC | PLSSQFECDNLSALTNTPDNQSS |
| | | TCTACAGAAACCATTTCAGCCCAACCAATCTCCCCATTGGAAGCAGACAGCTCATATAGACAGGCGGG | TETISAQPISPLEADSSYRQAGI |
| | | TATTCTTCTCCAGGAGAACATTCAAGTGGGAGCTGATCCGTTGTATGCTACATCAAGACATAATATGC | LLQENIQVGADPLYATSRHNMQH |
| | | AACACGCCTTACGGGAGATTGAGACTGTTCTGATGGCACCTGATACAGATGATGCAACAACTAGCACC | ALREIETVLMAPDTDDATTSTKH |
| | | AAGCATGAGTTTGAGGAAATCAAGCCTGCTCAGCTTGTGAGGCAGCGGTCGAGAACATGGAGTCATGA | EFEEIKPAQLVRQRSRTWSHESR |
| | | ATCACGGCAGCCGTTACCTGGAGTTGGTCGGTCACAGTTTGCATCTGGTGGATACCCCACAGCAAGCT | QPLPGVGRSQFASGGYPTASYEF |
| | | ATGAATTTCGTCCAGAGAAACGGCAAAGGGAGTTAAGGGAAGACCCTCAGATAATTGTGAAGCAGCTA | RPEKRQRELREDPQIIVKQLLTR |
| | | TTAACCAGGTGTGCTGAGGCTCTGAGTGAGGACAGGACAGAGGAGTTCCATAAGCTTGTTCAGGAGGC | CAEALSEDRTEEFHKLVQEARGV |
| | | TCGTGGAGTGGTCTCAATCAACGGGGAACCAATCCAACGTCTAGGTGCTTACCTACTGGAGGGTTTGG | VSINGEPIQRLGAYLLEGLVARH |
| | | TTGCTAGACATGGAAACTCTGGCACAAACATCTACCGTGCTCTGAAGTGCCGTGAGCCAGAGAGCAAG | GNSGTNIYRALKCREPESKELLS |
| | | GAGCTCCTGTCCTACATGAGAATTCTATACAATATCTGCCCTTACTTCAAGTTTGGCTATATGGCAGC | YMRILYNICPYFKFGYMAANGAI |
| | | CAATGGGGCGATTGCAGAAGCATTGAGAACTGAGAACAATATCCACATAATTGATTTTCAGATTGCTC | AEALRTENNIHIIDFQIAQGTQW |
| | | AAGGGACTCAATGGATCACACTGATACAAGCATTAGCTGCAAGGCCTGGTGGTCCTCCTCGTGTGCGG | ITLIQALAARPGGPPRVRITGID |
| | | ATCACCGGGATAGATGACCCAGTGTCAGAGTATGCTCGTGGTGAAGGTCTTGACATTGTGGGGAAAAT | DPVSEYARGEGLDIVGKMLKSMS |
| | | GTTGAAAAGCATGTCTGAAGAATTCAAAATACCTCTGGAGTTTACGCCTCTGTCTGTCTATGCCACAC | EEFKIPLEFTPLSVYATQVTKEM |
| | | AAGTCACGAAAGAGATGCTTGAGATCAGGCCAGGTGAAGCACTGGCTGTAAACTTCACACTCCAGCTA | LEIRPGEALAVNFTLQLHHTPDE |
| | | CACCACACCCCGGACGAGAGCGTGGATGTCAACAACCCACGCGATGGTCTGCTCCGGATGGTGAAAGG | SVDVNNPRDGLLRMVKGLSPKVT |
| | | GCTGTCCCCGAAGGTGACTACTTTGGTAGAGCAGGAGTCACACACCAACACAACGCCCTTCTTGATGA | TLVEQESHTNTTPFLMRFGETME |
| | | GGTTTGGGGAGACCATGGAGTACTACTCCGCCATGTTCGAGTCGATCGACGCCAACCTGCCGCGGGAC | YYSAMFESIDANLPRDNKERISV |
| | | AACAAGGAGAGGATCAGCGTGGAGCAGCACTGCCTCGCCAAGGACATCGTCAACATCATCGCCTGCGA | EQHCLAKDIVNIIACEGKDRVER |
| | | GGGGAAGGACAGGGTGGAGAGGCATGAGCTGCTTGGCAAGTGGAAGTCGAGGCTGACCATGGCCGGCT | HELLGKWKSRLTMAGFRPYPLSS |
| | | TCAGGCCTTACCCGCTGAGCTCCTACGTCAACTCGGTGATAAGGAAGCTTCTCGCCTGCTACTCCGAT | YVNSVIRKLLACYSDKYTLDEKD |
| | | AAATACACACTGGATGAGAAGGACGGCGCGATGCTTCTCGGCTGGAGGAGCAGAAAACTGATATCTGC | GAMLLGWRSRKLISASAWH* |
| | | TTCTGCGTGGCACTGA SEQ ID NO: 241 | SEQ ID NO: 242 |
| 122 | Xa21_Os07g0581000 | ATGGAGGCGGAGAAGAAGCCGCCGGCGGTGTCCGACGTCGGGGCGTGGGCGATGAACGTCGTCAGCTC | MEAEKKPPAVSDVGAWAMNVVSS |
| | | CGTCGGCATCATCATGGCAAACAAGCAGCTCATGTCCTCCTCCGGCTACGCCTTCTCCTTCGCCACGA | VGIIMANKQLMSSSGYAFSFATT |
| | | CGCTGACCGGATTCCACTTCACCGTCACGGCGCTCGTCGGATGGATCTCCAACGCCACCGGCTACTCC | LTGFHFTVTALVGWISNATGYSV |
| | | GTATCCAAGCACGTCCCGCTCTGGGAGCTCGTCTGGTTCTCCCTCGTCGCCAACACGTCCATCACCGG | SKHVPLWELVWFSLVANTSITGM |
| | | GATGAACCTTAGTCTCATGCTCAACTCCGTCGGTTTCTATCAGATCTCGAAATTGAGCATGATCCCGG | NLSLMLNSVGFYQISKLSMIPVV |
| | | TGGTTTGCTTGATGGAATGGGTGCTCAACAGCAAGCACTACACCACCAAGGTTATATCGGCCGTGGTC | CLMEWVLNSKHYTTKVISAVVVV |
| | | GTCGTGGCGGCGGGTGTTGGGATTTGCACGGTCACGGATGTGGAGGTCAATGCCAAGGGATTCATCTG | AAGVGICTVTDVEVNAKGFICAC |
| | | CGCTTGCGTGGCTGTCTTCTGCACGTCGCTTCAACAGATTACAATTGGCTCCTTTCAGAAGAAGTACA | VAVFCTSLQQITIGSFQKKYNIG |
| | | ACATAGGGTCATTTGAACTTCTCAGCAAAACTGCACCAATACAGGCTGTGTCTCTTATTATACTTGGT | SFELLSKTAPIQAVSLIILGPFA |
| | | CCCTTTGCTGACTATTACCTCAATGGGCGATGGTTATTAAACTACAACTTCTCAACTGGGGCAACTTT | DYYLNGRWLLNYNFSTGATFFIL |
| | | CTTCATATTGCTTTCTTGCTCCTTGGCTGTCTTCTGCAACATGAGCCAGTACCTCTGCATCGGCCGGT | LSCSLAVFCNMSQYLCIGRFSAT |
| | | TCTCTGCAACTTCGTTCCAGGTCCTGGGCCACATGAAGACCGTGTGCGTGCTGATTCTTGGCTGGATC | SFQVLGHMKTVCVLILGWILFDS |
| | | CTGTTCGACTCGGCCCTCACCGTGAAGAACATCCTCGGGATGCTGCTCGCCGTGATGGGCATGGTGGT | ALTVKNILGMLLAVMGMVVYSWA |
| | | CTACAGCTGGGCCGTGGAGACCGAGAAGAAGGCCTCCGCCCCAATCCCCCGGAACAAGAGCGACATGC | VETEKKASAPIPRNKSDMLDDSE |
| | | TGGACGACTCCGAGGACGTTCCCCTCAAGGCCAGGGTGAGCGGCCTCCCCTCGTCCGACCTCGAGGAA | DVPLKARVSGLPSSDLEEGEMKS |
| | | GGCGAGATGAAGAGCTAG SEQ ID NO: 243 | * SEQ ID NO: 244 |
| 123 | Xa21_Os07g0583600 | ATGGCTGATACTCCAACTTCCCGAATGATTCATCCCTTCAACAATATACCGAGCCAAAACCTGAAGCA | MADTPTSRMIHPFNNIPSQNLKQ |
| | | GTTCCAGTATTCTGATAATCCTCAGCACCCTTGCCACCCTTATAGGGCACCTTCAGACACCCACGTTG | FQYSDNPQHPCHPYRAPSDTHVV |
| | | TGCCACATCATTATGGCCTAAAGTCACATTCACCGGATGCTGGTTATGAAAGCCAGGCTACTCCTAAC | PHHYGLKSHSPDAGYESQATPNK |
| | | AAGTACACATTGGACTCCTCTGAAGGTGCTGGTTGTATGAGACATGATTCTCCTTCCAGTCAAAGTTT | YTLDSSEGAGCMRHDSPSSQSFT |
| | | CACAACCAGAAGTGGTAGCCCTCTATCTCAGGAAGACAGCCACTCTGACTCGACTGATGGATCTCCTG | TRSGSPLSQEDSHSDSTDGSPVG |
| | | TAGGCGCCTCCTGTGTCACTGAGGATCCTAATGATCTGAAGCAAAAACTGAAGGATCTCGAGGCCGTT | ASCVTEDPNDLKQKLKDLEAVML |
| | | ATGCTTGGGCCAGACTCGGAGATAGTCAACAGCCTCGAGAATTCTGTGGCAAACCAACTTTCCTTGGA | GPDSEIVNSLENSVANQLSLEPE |
| | | GCCGGAGAAGTGGGTGCGCATGATGGGCATTCCCAGAGGCAACTTGAAAGAGCTACTGATTGCCTGTG | KWVRMMGIPRGNLKELLIACARA |
| | | CTAGAGCTGTGGAAGAGAAGAATAGCATTGCTATTGATATGATGATTCCAGAGCTGAGAAAAATGGTT | VEEKNSIAIDMMIPELRKMVSVS |
| | | TCAGTATCTGGTGAGCCACTTGAGAGGTTGGGAGCCTACATGGTAGAAGGGCTTGTTGCCAGGCTTGC | GEPLERLGAYMVEGLVARLASSG |
| | | CTCCTCTGGCAATTCAATCTACAAAGCTTTGAAATGCAAGGAACCAAAGAGCTCTGACCTTCTGTCTT | NSIYKALKCKEPKSSDLLSYMHF |
| | | ACATGCACTTCCTGTATGAGGCCTGCCCCTACTTCAAGTTCGGCTATATGTCGGCGAATGGTGCTATT | LYEACPYFKFGYMSANGAIAEAV |
| | | GCAGAGGCTGTCAAGGGAGAAGACAGGATTCATATCATCGACTTCCATATCTCTCAAGGGGCTCAGTG | KGEDRIHIIDFHISQGAQWISLL |
| | | GATCTCTCTCCTTCAGGCCCTTGCAGCCAGGCCTGGTGGACCACCAACTGTAAGGATCACTGGTATTG | QALAARPGGPPTVRITGIDDSVS |
| | | ATGACTCAGTGTCAGCTTACGCACGAGGCGGCGGGCTAGAGCTGGTCGGAAGGAGGCTGTCCCACATC | AYARGGGLELVGRRLSHIAGLCK |
| | | GCCGGCCTGTGCAAGGTGCCCTTTGAATTCCACCCGCTTGCTATCTCCGGCAGCAAGGTGGAGGCGGC | VPFEFHPLAISGSKVEAAHLGVI |
| | | GCATCTCGGAGTCATCCCCGGGGAAGCCCTCGCCGTGAACTTCACCCTGGAGCTGCACCACATTCCAG | PGEALAVNFTLELHHIPDESVST |
| | | ACGAATCGGTGAGCACGGCGAACCACCGTGATCGCCTCCTGAGGATGGTGAAGAGCCTGTCGCCGAAG | ANHRDRLLRMVKSLSPKVLTLVE |
| | | GTGCTCACCCTGGTGGAGATGGAGTCCAACACGAACACCGCGCCGTTCCCGCAGAGGTTCGCGGAGAC | MESNTNTAPFPQRFAETLDYYTA |
| | | GCTGGACTACTACACCGCCATCTTCGAGTCCATCGACCTGACGCTGCCGAGGGGCGACAGGGAGAGGA | IFESIDLTLPRGDRERINMEQHC |
| | | TCAACATGGAGCAGCACTGCCTCGCGAGGGAGATCGTCAACCTCATCGCGTGCGAGGGGGAGGAGAGG | LAREIVNLIACEGEERAERYEPF |
| | | GCGGAGCGGTACGAGCCCTTCGGCAAGTGGAAGGCGCGCCTCACCATGGCCGGGTTCAGGCCGTCGCC | GKWKARLTMAGFRPSPLSSLVNA |
| | | GCTCAGCTCGCTGGTGAACGCCACCATCAGGACGCTGCTGCAGAGCTACTCGGACAACTACAAGCTCG | TIRTLLQSYSDNYKLAERDGALY |
| | | CCGAGAGGGACGGGGCGCTCTACCTCGGGTGGAAGAGCAGGCCACTGGTGGTCTCGTCGGCGTGGCAC | LGWKSRPLVVSSAWH* |
| | | TAG SEQ ID NO: 245 | SEQ ID NO: 246 |
| 124 | Xa21_OsJ_25599 | ATGGGTCCCCCGGATGTTATCTCTGAAAATGATGAGTTTGATTTCTCTGATGTTTTTGGCACCACTCC | MGPPDVISENDEFDFSDVFGTTP |
| | (Os07g0680900) | AGTTCAGACGCCAACAGGCATTTCGGTAGCTGGTCCAGACAGTCCTGCACCTTTGGCTGAGTCCAATG | VQTPTGISVAGPDSPAPLAESNE |
| | | AGGGGGTTTACAATGATCCTATTGTCATCATCAAGCGATCTCATTCTCTTGTTGGTCCAACATCACTT | GVYNDPIVIIKRSHSLVGPTSLV |
| | | GTTAGCCAGTCCCTGCGACTTAGCAATCTCACTCTAAATAAAACAGAGGGGTCATCAGAACCTGCAGA | SQSLRLSNLTLNKTEGSSEPAEE |
| | | AGAAAAGGAAAGGAACCTAGGGCAGCTCAGTGATGAAGAGTTTGACAATGCCACAACAGAAAATGAAG | KERNLGQLSDEEFDNATTENEGI |
| | | GTATTGGGCTTGATGACTTTGAAATCTTGAAGCTTGTAGGCCAAGGGGCATTTGGGAAAGTCTTTCAG | GLDDFEILKLVGQGAFGKVFQVR |
| | | GTGAGAAAGAAAGGTACTTCAGAGATATACGCGATGAAAGTTATGAGGAAGGATAAGATCTTGGAAAA | KKGTSEIYAMKVMRKDKILEKNH |
| | | GAACCATGCGGAGTACATGAAAGCCGAAAGAGATATATTAACAAAAGTTGACCATCCTTTTGTAGTGC | AEYMKAERDILTKVDHPFVVQLR |
| | | AGCTCAGGTACTCCTTTCAGACAAAATATCGACTTTATCTTGTCCTGGACTTCATAAATGGGGGACAT | YSFQTKYRLYLVLDFINGGHLFF |
| | | CTCTTCTTCCAGCTCTACAAGCAAGGATTGTTCAGGGAGGAACTTGCACGGATCTATACTGCTGAAAT | QLYKQGLFREELARIYTAEIVSA |
| | | TGTATCTGCTGTAGCTCACCTTCACGACAACGGGATTATGCATAGAGATCTCAAGCCTGAAAATATCC | VAHLHDNGIMHRDLKPENILLDA |
| | | TTTTGGATGCTGATGGCCATGCCATGCTCACTGACTTTGGCCTTGCCAAAGAATTTAATGAGAATACA | DGHAMLTDFGLAKEFNENTRSNS |
| | | AGATCAAACTCGATGTGTGGAACGGTTGAGTATATGGCCCCAGAGATTATTCTTGGTCGGGGGCATGA | MCGTVEYMAPEIILGRGHDKAAD |
| | | TAAGGCAGCTGACTGGTGGAGTGTTGGAATCCTCCTCTTTGAAATGCTCACGGGAAAGCCTCCGTTTG | WWSVGILLFEMLTGKPPFVGNRD |
| | | TTGGGAACAGGGATAAAGTTCAGCAGAAGATAGTGAAAGAGAAGCTGAAGCTTCCATCGTTTTTGTCC | KVQQKIVKEKLKLPSFLSSEAHS |
| | | AGTGAAGCTCATTCCCTCTTGAAAGGACTGCTCCACAAAGAAGGTGGCAAGCGGCTAGGAAGCGGTCC | LLKGLLHKEGGKRLGSGPGGSDE |
| | | GGGCGGCAGCGATGAGATAAAGAGGCACAAATGGCTCAAACCGATCAACTGGAGGAAGCTGGAGGCCC | IKRHKWLKPINWRKLEARQIQPS |
| | | GGCAGATCCAGCCGAGCTTCCGACCCAACGTGAGCGGGCTCACCTGCATCGCCAACTTCGACGAGTGC | FRPNVSGLTCIANFDECWTSMPV |
| | | TGGACCAGCATGCCGGTGCTGGACTCGCCGGTGGCCACCCCCGTCGCCGGCGGCGCCGGCCACAGCAG | LDSPVATPVAGGAGHSSFAGFTY |
| | | CTTCGCCGGGTTCACCTACGTCAGGCCGGCTCCGTTCCTCCAGGATGTCAAGCCTCCCTCTTCCTCTA | VRPAPFLQDVKPPSSSRLKD* |
| | | GGCTGAAAGACTAG SEQ ID NO: 247 | SEQ ID NO: 248 |
| 125 | Xa21_Os08g0163400 | ATGACGGCGACGCCGGCGGTGATCGGGCTGAGCGCGGGGAACAGGCTGCTGAGCGCGTCGTTCGGGCC | MTATPAVIGLSAGNRLLSASFGP |
| | | GACCGAACTGATGCCGGACAAGGTCAGCCTCGGCGTCGGGGGAGGAGGAGGTGGTGGTGGAGGTGGGG | TELMPDKVSLGVGGGGGGGGGGD |
| | | ACGCGATGTCGTTCGCGCCGCCGGCGCCCGCGACGCCGAAGCTCACCGCGGTCGCCGCGCACAGGCTC | AMSFAPPAPATPKLTAVAAHRLK |
| | | AAGCTCTCCCCGCATGGCCGCGCGCAGGTCATGCGCGCGCTCAGGCACCACTCCTCCGCGGCGGCCGC | LSPHGRAQVMRALRHHSSAAAAL |
| | | GCTCGCGCCCCCGCCCCCGCCCCCCCCGACGCCGTCGCCCGCCTCTCGCGCCGCCCACGCCCATGACC | APPPPPPPTPSPASRAAHAHDLE |
| | | TCGAGTCCTCCCTCGAGGCCATCGTGCTGCTGCAGAGGTCCATGCTCGAGAAGCAGTGGGAGCTCCCC | SSLEAIVLLQRSMLEKQWELPFD |
| | | TTCGACGACGAAGACCACGTCTCCGACGACGACGACAACCACGCCATGGCCATCGGCTTAGCCGAGGA | DEDHVSDDDDNHAMAIGLAEDDS |
| | | TGACAGCAAGGACGCCAACAAGGCCACCGTCGTCGTGGCGCGGTCCAGTGTCTCGGCGAGGCAGAGGA | KDANKATVVVARSSVSARQRRMS |
| | | GGATGAGCGGGCGGCGGCGGGGGAGGACCAAGAACGGCGCGGCGCACTTCGCCGTCAGCCCCGAGCTG | GRRRGRTKNGAAHFAVSPELMQS |
| | | ATGCAGAGCAGGAACAGGATCTACCTCAGGGGCACCGTCAGCAAGGAGCTCCTCACGCACAAGCAGGT | RNRIYLRGTVSKELLTHKQVVHL |
| | | CGTCCACCTCTCCCACAAGATCAAGGATGGCATCTGGTTGCAGCAGCAAAGATCAAAGCTGAAAGAGA | SHKIKDGIWLQQQRSKLKEKLGN |
| | | AGCTGGGAAATGAGCCCTCATACAAACAGCTGGCGCATTCGCTGAAGATATCACCGCCCGAGCTACGA | EPSYKQLAHSLKISPPELRSRMR |
| | | TCAAGGATGCGCGAGTCGTTTCTCGCAAGGGAGATGCTAACAATGAGCAACCTCCGTTTGGTCATTTC | ESFLAREMLTMSNLRLVISIAQK |
| | | CATTGCCCAGAAGTATGATAACCTTGGTGTTGAGTTGGCTGACCTCATTCAGGGTGGTCTGATAGGGT | YDNLGVELADLIQGGLIGLLRGI |
| | | TACTCCGTGGGATCGAAAAATTTGATGCATCCAGGGGCTTTAGAATTTCCACGTATGTATACTGGTGG | EKFDASRGFRISTYVYWWIRQGV |
| | | ATACGCCAGGGTGTTTCAAGAGCATTAGCTGAGAACTCAAAAACATTCAGGCTTCCAACTTATCTGCA | SRALAENSKTFRLPTYLHERLIA |
| | | TGAGCGGCTGATTGCGATTCGTGGTGCAAAGTATGCGTTGGAAGATCAAGGAATTGCTCCAACAATAG | IRGAKYALEDQGIAPTIENIAGS |
| | | AAAACATAGCAGGGTCACTCAACATATCAGAAAAGAAAGTACTCAATGCTACAGAGGCTGTGAACAAG | LNISEKKVLNATEAVNKVLSLDQ |
| | | GTTCTTTCGTTGGATCAACAAGCATTTCCATCCTTAAACGGCTTACCTGGAGAGACACTGCACAGTTA | QAFPSLNGLPGETLHSYIEDQNV |
| | | CATAGAGGATCAAAATGTCGCAAATGATCCATGGCATGGATTTGAGGAGTGGTATCTCAAGGAAGAAG | ANDPWHGFEEWYLKEEVNKLLNS |
| | | TCAACAAGCTTTTAAACTCAACTCTCAACGAGCGTGAGAGGGACATTATCCGGTTATATCATGGCATA | TLNERERDIIRLYHGIGKQCHTW |
| | | GGAAAACAATGCCATACATGGGAGGACATTAGCAGACAATTCGGGTTGTCGAGGGAGAGGGTGAGGCA | EDISRQFGLSRERVRQVGLIAME |
| | | AGTAGGACTCATTGCAATGGAGAAGCTGAAGCATGCTGCGAGGAGGAAAAATTTGGAGGCATTGCTTG | KLKHAARRKNLEALLEDY* |
| | | AGGATTATTGA SEQ ID NO: 249 | SEQ ID NO: 250 |
| 126 | Xa21_OsJ_26407 | ATGAGCTACCAGACTCCTCCTCCTGCTGCCACAGGGTACCCCTACCCGGCGCAAGAAGCAGGGCAGCA | MSYQTPPPAATGYPYPAQEAGQQ |
| | (Os08g0205700) | GCAGCAGCAGGCGTACCCTGCCTACGTGGCGCCGCCGCCGGCCGGCTACCCGACCAAGGACGGCCCGC | QQQAYPAYVAPPPAGYPTKDGPQ |
| | | AGGAGCAGTACCCCGCCGGCGCCGGCGCCGGCGAGACCACCAGCCGCGGCCACCACCACCACCACCAC | EQYPAGAGAGETTSRGHHHHHHH |
| | | CACCACGGCGACGGATTCTGGAAAGGATGCTGCGCTGCACTCTGCTGCTGCTGCCTCCTCGACATGTG | HGDGFWKGCCAALCCCCLLDMCF |
| | | CTTCTGA SEQ ID NO: 251 | * SEQ ID NO: 252 |
| 127 | Xa21_OsI_29768 | ATGGCCTCGGAGACGGAGACGTTCGCCTTCCAGGCGGAGATCAACCAGCTGCTGTCGCTCATCATCAA | MASETETFAFQAEINQLLSLIIN |
| | (Os08g0500700) | CACGTTCTACTCCAACAAGGAGATCTTCCTCCGCGAGCTCATCTCCAACTCCTCCGATGCGTTGGACA | TFYSNKEIFLRELISNSSDALDK |
| | | AGATCAGGTTCGAGAGCTTGACGGACAAGAGCAAGCTGGATGCTCAGCCAGAGCTGTTCATCCATATT | IRFESLTDKSKLDAQPELFIHIV |
| | | GTTCCTGACAAGGCCTCCAACACACTCTCGATCATCGACAGTGGTATTGGTATGACCAAGTCAGATCT | PDKASNTLSIIDSGIGMTKSDLV |
| | | TGTGAACAACCTGGGTACCATTGCCAGGTCAGGGACCAAGGAGTTCATGGAGGCACTGGCTGCTGGTG | NNLGTIARSGTKEFMEALAAGAD |
| | | CTGATGTGTCCATGATTGGTCAGTTCGGTGTTGGATTCTACTCTGCCTACCTTGTTGCCGAGAGAGTG | VSMIGQFGVGFYSAYLVAERVVV |
| | | GTTGTGACCACCAAGCACAACGATGACGAGCAGTATGTGTGGGAGTCTCAGGCTGGTGGGTCCTTCAC | TTKHNDDEQYVWESQAGGSFTVT |
| | | TGTCACACGTGACACATCTGGGGAGCAGCTTGGGAGGGGTACTAAGATCACCCTCTACCTCAAGGACG | RDTSGEQLGRGTKITLYLKDDQL |
| | | ATCAGTTGGAATACCTTGAAGAGCGCCGCCTCAAGGACCTGATCAAGAAGCACTCTGAGTTTATCAGC | EYLEERRLKDLIKKHSEFISYPI |
| | | TACCCTATCTCTCTATGGACTGAGAAGACCACCGAGAAGGAAATTTCTGATGATGAAGATGAGGAAGA | SLWTEKTTEKEISDDEDEEEKKD |
| | | GAAGAAGGATGCTGAGGAGGGCAAGGTTGAGGATGTTGATGAAGAGAAGGAAGAAAAGGAGAAGAAAA | AEEGKVEDVDEEKEEKEKKKKKI |
| | | AGAAGAAGATCAAGGAGGTTTCTCATGAGTGGTCCCTGGTCAACAAACAGAAGCCTATCTGGATGAGG | KEVSHEWSLVNKQKPIWMRKPEE |
| | | AAGCCTGAGGAGATCACTAAGGAGGAGTATGCTGCTTTCTACAAGTCCCTGACAAACGACTGGGAGGA | ITKEEYAAFYKSLTNDWEEHLAV |
| | | GCATCTTGCTGTCAAGCACTTCTCCGTGGAGGGTCAGCTGGAGTTCAAAGCTGTTCTCTTTGTTCCCA | KHFSVEGQLEFKAVLFVPKRAPF |
| | | AGAGGGCACCATTCGACCTCTTTGATACGAGGAAGAAGCTCAACAACATCAAGCTCTATGTCCGCCGA | DLFDTRKKLNNIKLYVRRVFIMD |
| | | GTCTTCATCATGGACAACTGTGAGGAGCTGATCCCTGAGTGGCTGAGCTTTGTCAAGGGCATTGTCGA | NCEELIPEWLSFVKGIVDSEDLP |
| | | CTCCGAGGATCTTCCCCTCAACATCTCCCGTGAGATGCTGCAGCAGAACAAGATTCTCAAGGTCATCC | LNISREMLQQNKILKVIRKNLVK |
| | | GGAAGAACCTTGTCAAGAAGTGCGTTGAGCTCTTCTTCGAGATTGCTGAGAACAAGGAGGACTACAAC | KCVELFFEIAENKEDYNKFYEAF |
| | | AAGTTCTATGAGGCCTTCTCCAAGAACCTCAAGCTCGGTATCCACGAGGACTCCACCAACAGAAACAA | SKNLKLGIHEDSTNRNKIAELLR |
| | | GATTGCCGAGCTCCTGAGGTACCACTCCACCAAGAGCGGCGATGAGCTGACCAGCCTCAAGGATTACG | YHSTKSGDELTSLKDYVTRMKEG |
| | | TGACGAGGATGAAGGAAGGCCAGAACGACATATACTACATCACCGGCGAGAGCAAGAAGGCCGTGGAG | QNDIYYITGESKKAVENSPFLEK |
| | | AACTCCCCCTTCTTGGAGAAGCTGAAGAAGAAGGGTTACGAGGTCCTCTACATGGTTGATGCCATCGA | LKKKGYEVLYMVDAIDEYAVGQL |
| | | CGAGTACGCTGTTGGCCAGCTCAAGGAGTTTGAGGGCAAGAAGCTCGTCTCTGCCACCAAGGAGGGAC | KEFEGKKLVSATKEGLKLDESED |
| | | TCAAGCTTGATGAGAGCGAGGACGAGAAGAAGCGGAAGGAGGAGCTCAAGGAGAAGTTTGAGGGGCTC | EKKRKEELKEKFEGLCKVIKEVL |
| | | TGCAAGGTCATCAAGGAGGTGCTCGGAGACAAGGTCGAGAAGGTGGTGGTCTCGGACCGCGTGGTGGA | GDKVEKVVVSDRVVDSPCCLVTG |
| | | CTCCCCGTGCTGCCTCGTCACCGGCGAGTACGGCTGGACGGCCAACATGGAGAGGATCATGAAGGCCC | EYGWTANMERIMKAQALRDSSMA |
| | | AGGCGCTGAGGGACTCGAGCATGGCCGGGTACATGTCGAGCAAGAAGACGATGGAGATCAACCCGGAG | GYMSSKKTMEINPENAIMEELRK |
| | | AACGCCATCATGGAGGAGCTCCGCAAGCGCGCCGACGCCGACAAGAACGACAAGTCCGTCAAGGACCT | RADADKNDKSVKDLVLLLFETAL |
| | | CGTCTTGCTCCTCTTCGAGACGGCCCTCCTCACCTCCGGCTTCAGCCTCGACGACCCCAACACCTTCG | LTSGFSLDDPNTFGSRIHRMLKL |
| | | GCAGCAGGATCCACCGCATGCTCAAGCTTGGCCTGAGCATCGACGAGGATGAGACGGCGGAGGCCGAC | GLSIDEDETAEADTDMPPLEDDA |
| | | ACCGACATGCCGCCGCTCGAGGACGACGCCGGCGAGAGCAAGATGGAGGAGGTCGACTAA | GESKMEEVD* |
| | | SEQ ID NO: 253 | SEQ ID NO: 254 |
| 128 | Xa21_Os08g0520000 | ATGGGCATGGAGGAGGAGGGGGTGGCGCGCGGCGGCGGCGGCGGCAAGAAGCTCGCGCTCGCCAGCAT | MGMEEEGVARGGGGGKKLALASI |
| | | CGGCTTCGCGGACGTCCGTGTCGGCGCCGTGGCTGGCGGCGGCGGCGGGTACAAGGAAGACTTGGTAG | GFADVRVGAVAGGGGGYKEDLVV |
| | | TCGTGGGCCTGCCCAAGGACGACGACTTCGATGTCGCCAAGGTCGTCGGGGACGTGGCCGTCGGGCTG | VGLPKDDDFDVAKVVGDVAVGLP |
| | | CCGGACGTCGGCGGCGCCGTCAGGAATTTCCTGAGGAATAGAGAAGTTGGAGAATTCATGAGTGGGGC | DVGGAVRNFLRNREVGEFMSGAL |
| | | ATTAGCTGGTGCTATGACTAAAGCTGTTCTTGCTCCTCTAGAAACTATCAGGACAAGAATGGTTGTTG | AGAMTKAVLAPLETIRTRMVVGV |
| | | GGGTAGGATCAAAACACATTTTTGGTAGTTTTGTGGAGATCGTCGAACAAAATGGCTGGCAAGGACTC | GSKHIFGSFVEIVEQNGWQGLWA |
| | | TGGGCAGGTAATACAATAAATATGCTCCGGATTATTCCAACTCAAGCACTTGAGCTTGGCACATTTGA | GNTINMLRIIPTQALELGTFECV |
| | | GTGTGTCAAAAGGAGCATGACAGAAGCACAAGAGAAATGGAAGGAAGATGGGTGCCCAAAGATACAAA | KRSMTEAQEKWKEDGCPKIQIGN |
| | | TTGGCAATTTGAAAATTGAGCTGCCGCTTCACTTGCTCTCTCCAATTGCTGTTGGTGGTGCGGCTGCT | LKIELPLHLLSPIAVGGAAAGIV |
| | | GGAATAGTTAGCACCTTAGTGTGCCATCCTCTTGAGGTTCTTAAGGACCGCCTGACTATCAACCGTGA | STLVCHPLEVLKDRLTINREAYP |
| | | GGCTTATCCAAGTATCGGCCTTGCCTTCAACAAGATATACCAAACTGATGGTATTGGTGGTCTCTATG | SIGLAFNKIYQTDGIGGLYAGLC |
| | | CTGGCCTCTGCCCAACACTAGTTGGCATGCTTCCTTATAGCACCTGCTACTATTTCATGTATGAGACG | PTLVGMLPYSTCYYFMYETIKTS |
| | | ATCAAGACTTCTTATTGCCGTGCACATAAAAAGAAGTCCTTGACCCGTCCTGAGCTACTCATAATTGG | YCRAHKKKSLTRPELLIIGALSG |
| | | AGCTCTTTCAGGTCTAACTGCAAGTACAATAAGCTTCCCTCTGGAAGTGGCGAGGAAGCGGCTTATGG | LTASTISFPLEVARKRLMVGTLQ |
| | | TTGGAACCCTGCAAGGGAAATGCCCGCCCCACATGATCGCGGCCTTAGCTGAGGTGTTCCAAGAGGAG | GKCPPHMIAALAEVFQEEGIKGL |
| | | GGCATCAAGGGACTTTACCGCGGATGGGCCGCAAGCTCCCTGAAGGTGATGCCGACCTCCGGCATTAC | YRGWAASSLKVMPTSGITWMFYE |
| | | CTGGATGTTCTATGAGGCATGGAAGGACATTCTTTTGGCTCCAGAGCTGCACACCTAG | AWKDILLAPELHT* |
| | | SEQ ID NO: 255 | SEQ ID NO: 256 |
| 129 | Xa21_Os08g0526100 | ATGATGCCGCACTGCGGCGTGGTGGACGCGGCGGCGAAGGGGGTGAAGCTGGGGGGCGGTGGTGGCGG | MMPHCGVVDAAAKGVKLGGGGGG |
| | | GGCGCTGATGGTGCGCCGGGTGGCGAGCGGCAAGCTCCTGTCGGCGTCGTCGCACCTGCTGTTCCGCG | ALMVRRVASGKLLSASSHLLFRA |
| | | CCACCGTACTCGCCACGCTGGCGCTTGTGTTCCTCTTCACGTTCCACTACCCGTCGCTGCTGTCGCGC | TVLATLALVFLFTFHYPSLLSRS |
| | | TCCTTCACGCTCTCCTCCGGCGCCGGCGGCGGCGAGGGGGGAGCCGCGGCGCACGCCTCGCACCGGAG | FTLSSGAGGGEGGAAAHASHRSL |
| | | CTTGCTCATGTCGTCGTCGTCGGCGTCGGCGGCGTCGGTGTACGGGGGCGCGGCGTGGGAGAAGGAGG | LMSSSSASAASVYGGAAWEKEVR |
| | | TGAGGCGGAGCGCGAAGCCGAGGAAGGACGGGGGGATCGCCGTGCTGGTGACCGGCGCGGCGGGGTTC | RSAKPRKDGGIAVLVTGAAGFVG |
| | | GTCGGCACGCACTGCTCGCTGGCGCTCAGGGCGCGCGGCGACGGCGTGCTGGGGCTCGACAACTTCAA | THCSLALRARGDGVLGLDNFNAY |
| | | CGCCTACTACGACCCGGAGCTGAAGCGCGCGCGGCAGAGGCTCCTCGCCGGCCGCGGCGTGCTCGTGC | YDPELKRARQRLLAGRGVLVLDA |
| | | TCGACGCCGACATCAACGACGCGCTCCTGCTCGAGAAGCTGTTCGACCTGGTGCCGTTCACCCACGTG | DINDALLLEKLFDLVPFTHVLHL |
| | | CTGCACCTCGCCGCGCAGGCCGGCGTGCGCTACGCCATGGAGGCGCCGCAGACGTACGTGGCGTCCAA | AAQAGVRYAMEAPQTYVASNVAG |
| | | CGTCGCGGGGCTCGTGACCGTCCTCGAGGTCGCTGCCAAGCACGCCGACCCGCAGCCGGCGATCGTCT | LVTVLEVAAKHADPQPAIVWASS |
| | | GGGCGTCGTCGTCGTCGGTGTACGGGCTCAACACCGACGCGCCCTTCTCCGAGGAGCACCGCACCGAC | SSVYGLNTDAPFSEEHRTDRPAS |
| | | CGGCCGGCGTCGCTGTACGCGGCGACCAAGAAGGCCGGCGAGGCCATCGCCCACGCGTACAACCACAT | LYAATKKAGEAIAHAYNHIYGLS |
| | | CTACGGCCTCTCCATCACCGGCCTCCGCTTCTTCACCGTGTACGGTCCATGGGGCCGCCCCGACATGG | ITGLRFFTVYGPWGRPDMAYFSF |
| | | CCTACTTCTCCTTCGCCCGCAGCATCGTCGCCGGCGAGCCCATCACGCTGTTCCGCACCGCCGACGGC | ARSIVAGEPITLFRTADGADARR |
| | | GCCGACGCGCGCCGCGACTTCACCTACATCGACGACGTCGTCAAGGGCTGCCTCGGCGCGCTCGACAC | DFTYIDDVVKGCLGALDTAGEST |
| | | GGCCGGCGAGAGCACCGGCACCAAGTCCGGCAAGAAGCGCGGCCCGGCGCCGCTCCGCGTCTACAACC | GTKSGKKRGPAPLRVYNLGNTSP |
| | | TCGGCAACACCTCGCCGGTGCCGGTCACCCGCATGGTCGCCATCCTCGAGAAGCTCCTCGGCAGGAAG | VPVTRMVAILEKLLGRKANKRVV |
| | | GCGAACAAGCGCGTCGTCACCATGCCGAGCAATGGCGACGTGCCGTTCACCCACGCCAACGTCAGCCA | TMPSNGDVPFTHANVSHAARDFG |
| | | CGCCGCGCGCGACTTCGGCTACCGCCCGGCGACCTCCCTCGACGCCGGCCTCCGCCGCTTCGTCGACT | YRPATSLDAGLRRFVDWFVHYYK |
| | | GGTTCGTCCACTACTACAAGCTCGACACGGCCAAGATCGCCAAGGGCAAGAGGAAATCCATGGCCATG | LDTAKIAKGKRKSMAMSAAS* |
| | | TCCGCCGCGTCGTGA SEQ ID NO: 257 | SEQ ID NO: 258 |
| 130 | Xa21_Os09g0115500 | ATGGACGCCACGCTGCTCCGCTCCGCCGATGCCGCCGTCGCCGTCCGCTCCACCACCATTGCGCCTCC | MDATLLRSADAAVAVRSTTIAPP |
| | | GCCTCGCCCTCGCCTCCACCGCCCCCTCCCGTCCTCCTGCGCCTGCGGGCCCCTCCGCGCCTACGCCG | PRPRLHRPLPSSCACGPLRAYAA |
| | | CTCCCGCTGCCCCCGCGCCCGCCGCGCACAACAACGGAGTCTACACAGTTGGTGACTTCATGACCAAA | PAAPAPAAHNNGVYTVGDFMTKR |
| | | AGGCCTAATCTCCATGTCGTTACACCTGCAACCTCGGTTGATGAAGCTCTCGAGACGCTGGTGCAACA | PNLHVVTPATSVDEALETLVQHK |
| | | CAAGATCTCCGGTTTCCCTGTTGTTGATGACACCGGAAAGTTGGTGGGTGTTGTCTCAGATTATGATC | ISGFPVVDDTGKLVGVVSDYDLL |
| | | TGTTAGCACTTGACTCAATATCAGGAAGTGGACTGACTGGCACAAACACAAGCATGTTTCCTGAGGTG | ALDSISGSGLTGTNTSMFPEVDS |
| | | GATAGTACTTGGAAGACATTTCGCGAGATACAGAGACTCTTGAGCAAAACCAATGGCAAAGTCATCGC | TWKTFREIQRLLSKTNGKVIADV |
| | | TGATGTGATGACTTATTCGCCTCTTGCGGTTCGTGAAAGTACCAACCTTGATGCTGCTACAAGGTTGC | MTYSPLAVRESTNLDAATRLLLE |
| | | TACTTGAAACCAAATATCGCAGATTGCCTGTTGTTGATAGCACTGGTAAATTGGTTGGGATGATCACA | TKYRRLPVVDSTGKLVGMITRGT |
| | | AGGGGGACTGTTGTCAGAGCTGCCCTCAAAATAAAGAAGACAGCTGAAGAAACTGCTTAA | VVRAALKIKKTAEETA* |
| | | SEQ ID NO: 259 | SEQ ID NO: 260 |
| 131 | Xa21_Os09g0334500 | ATGGAGAGCATGGAGGGCAATGGAGGAGGGCGGCTGGTGGTGACCGAGCTGAGCCACATCAAGGAGCT | MESMEGNGGGRLVVTELSHIKEL |
| | | CGTGAGGCAGCTGGAGGGGCACCTGGGTGGCTCCGGCTCCCCCGACCTCTGCAAGCACCTGGCCTCGC | VRQLEGHLGGSGSPDLCKHLASQ |
| | | AGATCTTCTCCGTCACCGAGCGCTCCATCGGCATGATCAGGTCCGGCCACTTCGACGGCCACCGCAAG | IFSVTERSIGMIRSGHFDGHRKR |
| | | CGCTCCGCCGCCGCTGTCGCCGCCGGTGACCTCGACTCGGCGACCCCCAGCCCCCTCAGCGACGTCTC | SAAAVAAGDLDSATPSPLSDVSD |
| | | CGACTTGCCTTTCAAGGCCACCAAGAAGAGGAAGACGTCGACGGAGAAGAAGAGGCATCAGATTAGGG | LPFKATKKRKTSTEKKRHQIRVS |
| | | TGAGCTCGACGGGAGGGGTGGAGAACCCACCGGTGGATGATGGCCATAGCTGGAGAAAGTATGGGCAG | STGGVENPPVDDGHSWRKYGQKE |
| | | AAAGAGATTCTTGGAGCCAAGCACCCAAGGGGTTACTACCGTTGCACCCACCGCAACTCGCAGGGATG | ILGAKHPRGYYRCTHRNSQGCMA |
| | | CATGGCGACGAAGCAGGTGCAGCGCACCGACGAGGACCCGACGGTGTTCGACGTGATCTACCACGGCG | TKQVQRTDEDPTVFDVIYHGEHT |
| | | AGCACACGTGCGTCCACAAGGCGGTGGCCGCCGGCGCCGGCGCCGGCAAGCCGGAGACGGAGACGGAC | CVHKAVAAGAGAGKPETETDTNA |
| | | ACGAACGCCGCCGCCGAGAGCCGCCTCCATGACATGAGCTGCGGCCTGACGGTGAAGATCGAGGGGCT | AAESRLHDMSCGLTVKIEGLTAT |
| | | GACGGCGACGCCGCCGCAGGGCGGCGGCGGCTGGAACGCCATGCCGCCGTTCTGCCTCACGTCGCCGG | PPQGGGGWNAMPPFCLTSPVSGL |
| | | TGAGCGGCCTGGCGCCGCCGGATCAGCACAACCCGTTCTCGGCGCCGTCGACGCCGGAGAACCGCCTC | APPDQHNPFSAPSTPENRLAAAA |
| | | GCCGCCGCCGCGTCGTCGGCGGCCTCGCCGGCGACCTCCGACTCGATGGCCGCCGCGCCGTTCCACCA | SSAASPATSDSMAAAPFHQAAAG |
| | | GGCGGCGGCCGGCGGCGGCGACGCGGCGTGGCGGGACGCCGAGCTCCAGGAGGTGGTGTCCGCGCTCG | GGDAAWRDAELQEVVSALVAATT |
| | | TCGCCGCGACGACGACGACGACGGCTACCGCGCAGCCGGCGCCCGCCTCCGCCATGGTCGACGCCGAC | TTTATAQPAPASAMVDADLSALD |
| | | CTCTCCGCCCTCGACGCCTTCGAGTTCGACCCCGGATTCACCATTGATATCACCAGCTTCTTCGCATG | AFEFDPGFTIDITSFFA* |
| | | A SEQ ID NO: 261 | SEQ ID NO: 262 |
| 132 | Xa21_Os09g0378700 | ATGTCGTCGCCGCCGCCGCCGCCGAGCAAGCCGAAGCGCCGGCGGCTACTGTCTCTTCCGGCGGTCTA | MSSPPPPPSKPKRRRLLSLPAVY |
| | | CCCGTGCGAGGACATCACGCCGGCGCCGCTGCTCGCCTCGCTGCTCTCCCTCGCGGCGGATGTCGCCT | PCEDITPAPLLASLLSLAADVAC |
| | | GCCGTCGGGCGGCCGACGTCGACGCCTTCCCCGTTCTCCGCCGCGGCGTGCGCAAGGCCGTGCGGCTC | RRAADVDAFPVLRRGVRKAVRLA |
| | | GCCGGGATCCTGCTCGCGTTCCTCGAGGAGGTTCAGGATGCGGCAGCGGCGGGGAAGGCGACGGCGAT | GILLAFLEEVQDAAAAGKATAMP |
| | | GCCGTCGTCTGCGGTGCTTGGGCTGACGGAGCTGCACGTGGCGATGCAGAAGCTGCGGTTCCTGCTCG | SSAVLGLTELHVAMQKLRFLLAD |
| | | CGGACTGCGCGCGGCGGGGCGCGCGGCTGTGGGTGCTCGTGAACGCCGGGATGGTGGCGTCGGAGCTC | CARRGARLWVLVNAGMVASELRL |
| | | AGGTTGGTGCTCGGGTCGGTCGCGGCGGCCATGGACGCGCTTCCGAGGAGCGTCGCGGAGGCGTCGGT | VLGSVAAAMDALPRSVAEASVDA |
| | | GGATGCCGGGGAGCTCGCGCGGGTGGTATCGGAGCAGGCGTGGCGCGCGGTGGTGCGGCCCGACGGCG | GELARVVSEQAWRAVVRPDGADE |
| | | CAGACGAACGCGCGGCGCGGAGCGTGCGGTCGATCCTGGATCAGTTCAAGGACGGCGTCGCGCCGGAC | RAARSVRSILDQFKDGVAPDADD |
| | | GCCGACGACGTGAGGCGGGTGCTCCGCCGGGTCAGGGTCGGGAGCTGGTCGGATTGCTCCGAGGAGAT | VRRVLRRVRVGSWSDCSEEIAFL |
| | | CGCGTTCCTGGAGAGCGAGATTTGCGCGCGGCTGGACGCCGGCGACGAGAACAGCAATGACGTTATCG | ESEICARLDAGDENSNDVIVMNS |
| | | TCATGAACAGCTTAATGGCGTTCCTGGTGTACTGCCGCGTCGTGCTGTTCGACCACATTGACGCGAGC | LMAFLVYCRVVLFDHIDASKSQP |
| | | AAGTCGCAACCGGCGGCGGCGGCGGCGCCGGCGCCGGCGAGGTGCCCGGAATGGATCAGACCGGAAGC | AAAAAPAPARCPEWIRPEALQCP |
| | | GCTGCAGTGCCCGATCACTCTGGACCTCATGACCGATCCGGTGACCGTGTCCACCGGCCAGACATACG | ITLDLMTDPVTVSTGQTYDRASI |
| | | ACCGGGCGTCGATCACCCGGTGGATGAAGGCCGGTTGCCGCACGTGCCCGGTCACCGGCGAGAGGCTG | TRWMKAGCRTCPVTGERLSTADL |
| | | AGCACCGCCGACCTCGTCCCCAACACCGTGCTCCGCGGGATCATCGAGCGGATGCTTCTCATCAATGG | VPNTVLRGIIERMLLINGVTLPE |
| | | CGTCACCCTCCCGGAGCCGAGCGCCGCCGGCGGCGGTGGCCACCGCCACGGCGCCGTCGCGAACACGG | PSAAGGGGHRHGAVANTAVPFGL |
| | | CCGTGCCGTTCGGCCTGGCCGCCGCGGGGGCCGCGCGCCTCGCCGTCGCGCACATCGTGGCGCAGCTC | AAAGAARLAVAHIVAQLSRGSTE |
| | | TCGAGGGGGTCGACGGAGGAGCGGAGGAAGGCGACGTCGGAGGCACGGAAGCTGTCGAAGCACAGCGT | ERRKATSEARKLSKHSVFYRACL |
| | | GTTCTACCGGGCTTGCCTCGTCGACGCCAACGCCGTGCCGTGGCTGCTGTGCCTCCTCTCCTCCACCG | VDANAVPWLLCLLSSTDAAVQDN |
| | | ACGCCGCCGTGCAGGACAATGCCGTGGCGTCGCTGCTCAACCTCTCGAAGCACCCCGCCGGCAGGACG | AVASLLNLSKHPAGRTAIVEVGG |
| | | GCCATCGTGGAGGTCGGCGGCGTCGGCCTCGTCGTCGACGTGATCAACGTCGGCGCCAAGGCCGAGGC | VGLVVDVINVGAKAEAQHNAAAV |
| | | GCAGCACAACGCGGCGGCCGTCCTCTTCTACCTCTCGTCGAACAGCCCGGACTCCGCCGAGGAGATCG | LFYLSSNSPDSAEEIGRIPEAIP |
| | | GCCGCATCCCGGAGGCGATCCCGACGCTGGTCCAGCTCATCCGGGACGGCGCGTACCGCGGGCGGAAG | TLVQLIRDGAYRGRKNAMVSLYG |
| | | AACGCCATGGTCAGCCTGTACGGGCTGCTCCAGAGCGCCGCCAACCACGGCAGGGCCATCGCCGCGGG | LLQSAANHGRAIAAGAVSALAAL |
| | | CGCCGTGTCAGCGCTCGCCGCGCTGCTGCTCTCCGCCGACCGCGACGACCTCGCCGGGGACAGCGTCG | LLSADRDDLAGDSVALLARIAEQ |
| | | CGCTGCTGGCGAGGATCGCCGAGCAGCCGTCTGGCGCCGCGGCCGTCCTCTCGCAGCCGGGGCTCGTC | PSGAAAVLSQPGLVARLAEALAS |
| | | GCTCGCCTCGCCGAGGCGCTCGCCTCCGCCTCGGCGTCGTCGTCCCGGTCGGCGAGAGACCACAGCGT | ASASSSRSARDHSVSLLASLCRH |
| | | GTCGCTCCTCGCCTCGCTGTGCCGGCACGGCGGCGCCAAGGTGGTCGCCGTGCTGGGGCGGATGCCGG | GGAKVVAVLGRMPGLMASLYSLV |
| | | GGCTGATGGCGTCGCTCTACTCGCTCGTCGCCGACGGGGGCAGCCCGCAGACGAGCAAGAAGGCGCGG | ADGGSPQTSKKARALLNEIHRHY |
| | | GCGCTGCTCAACGAGATCCACCGGCATTACGAGGTGGCGCCGCCGCCGCCGGCGTCGTCGGCGTCGTC | EVAPPPPASSASSDAGGDRVVRV |
| | | GGACGCCGGCGGTGACCGTGTCGTTCGCGTGCTATAG SEQ ID NO: 263 | L* SEQ ID NO: 264 |
| 133 | Xa21_Os09g0491756 | ATGGCCGAGCACCTCGCTTCCATCTTTGGCACGGAGAAGGACCGCGTGAACTGCCCCTTCTACTTCAA | MAEHLASIFGTEKDRVNCPFYFK |
| | | GATCGGCGCGTGCCGCCATGGCGACCGATGCTCCCGCCTGCACAACAAGCCGTCGGTCTCGCCGACGC | IGACRHGDRCSRLHNKPSVSPTL |
| | | TTCTGCTCTCCAACATGTACCTGCGCCCGGACATGATCACCCCCGGAATCGACGCGCAGGGCAACCCC | LLSNMYLRPDMITPGIDAQGNPI |
| | | ATCGACCCCGAGAAGATCCAGGCCGACTTCGAGGATTTCTACGAGGACATCTTTGAGGAACTCAGCAA | DPEKIQADFEDFYEDIFEELSKY |
| | | GTACGGCGAGATCGAGAGCCTCCACGTGTGTGACAACTTCGCTGACCACATGATCGGCAATGTGTATG | GEIESLHVCDNFADHMIGNVYVQ |
| | | TTCAATTCAGGGAGGAGGATCAGGCCGCCAGAGCGCTGCAGGCGCTGACGGGGCGGTACTACTCCGGC | FREEDQAARALQALTGRYYSGRP |
| | | CGCCCTATCATTGTCGAGTTCTCGTCGGTGACCGATTTCCGGGAGGCCACCTGCCGGCAATATGAAGA | IIVEFSSVTDFREATCRQYEENS |
| | | GAACAGCTGCAACCGTGGTGGGTACTGCAACTTCATGCACGTCAAGGAGATCGGCAGAGACCTGAGGA | CNRGGYCNFMHVKEIGRDLRKRL |
| | | AGAGGTTGTTTGGGCACCTGCATAGGTCCCGGAGAAGCCATAGCCATGGCCGGAGCCGCAGCCCGAGC | FGHLHRSRRSHSHGRSRSPSPYH |
| | | CCGTACCATTACCGGAGGGACTATGATCGGAGGAGCTCATCGCGGTCCAGGGACCGTGATGATTACTA | YRRDYDRRSSSRSRDRDDYYRGG |
| | | CCGTGGAGGTAGCTGGAGGAGCAGCGAGAGGCACCGCAGCAGCTACGACAGCGATGGCAGCAGGCGGC | SWRSSERHRSSYDSDGSRRRHRS |
| | | GGCACAGGTCCAGGACTAGGAGTCCTGTCAGGGATGGTAGCGAGGAGAGGAGAGCTCGGATTGAGCAG | RTRSPVRDGSEERRARIEQWNRE |
| | | TGGAACCGTGAAAGGGAGGCTGCACATGTGTGA SEQ ID NO: 265 | REAAHV* SEQ ID NO: 266 |
| 134 | Xa21_Os09g0522000 | ATGGAGGCGGAGGAGGCGACGTACAGGACGGTGTGGTCGGAGCCGCCGAAGAGGCCGGCGGGGAGGAC | MEAEEATYRTVWSEPPKRPAGRT |
| | | CAAGTTCAGGGAGACGAGGCACCCGGTGTACCGCGGCGTGCGGCGGCGCGGGGGGCGGCCGGGCGCGG | KFRETRHPVYRGVRRRGGRPGAA |
| | | CGGGGCGGTGGGTGTGCGAGGTGCGGGTGCCCGGGGCGCGCGGCTCCAGGCTGTGGCTCGGCACGTTC | GRWVCEVRVPGARGSRLWLGTFA |
| | | GCCACCGCCGAGGCGGCGGCGCGCGCGCACGACGCCGCCGCGCTGGCGCTCCGCGGCAGGGCCGCCTG | TAEAAARAHDAAALALRGRAACL |
| | | CCTCAACTTCGCCGACTCCGCGTGGCGGATGCCGCCCGTCCCCGCGTCCGCCGCGCTCGCCGGCGCGA | NFADSAWRMPPVPASAALAGARG |
| | | GGGGGGTCAAGGCCGCCGTCGCCGAGGCCGTCGAGGCGTTCCAGCGCCAGTCGCAGCCCGTGTCCACG | VKAAVAEAVEAFQRQSQPVSTAP |
| | | GCGCCGTCGTCTCCGGCGGAGACCTTCGCCGACTACGGCGACGAAGAGGAAGACAACAAGGACGTGTT | SSPAETFADYGDEEEDNKDVLPA |
| | | GCCGGCGGTGGCGACGGTGCCCGGCGACGCGGGGGCGTTCGAGCTCGATGACGGGTTCAGGTTCGGCG | VATVPGDAGAFELDDGFRFGGMD |
| | | GGATGGACGCCGGGTCGTACTACGCGAACTTGGCGCAGGGGCTGCTCGTCGAGCCGCCGGGCGCCGGA | AGSYYANLAQGLLVEPPGAGAWW |
| | | GCGTGGTGGGAGGACGGCGAGCTCGCCGGCTCCGACATGCCGCTCTGGAGCTACTAA | EDGELAGSDMPLWSY* |
| | | SEQ ID NO: 267 | SEQ ID NO: 268 |
| 135 | Xa21_Os09g0526600 | ATGGCGGAGCAGGGGGCGGGAGAGGCGGATGCGGGCGGCGGTGGCGGGGGAGGGGAGCCGCCGCCGCC | MAEQGAGEADAGGGGGGGEPPPP |
| | | GGCGGTGATGACGGCGGCGGCGGAGGCGCTGGCGGGGCAGAGGTCGCTGCCGACGCCGTTCCTGACGA | AVMTAAAEALAGQRSLPTPFLTK |
| | | AGACGTACCAGCTGGTGGAGGACCCGGCGGTGGACGACGTGATCTCGTGGAACGAGGACGGGTCGACG | TYQLVEDPAVDDVISWNEDGSTF |
| | | TTCGTGGTGTGGCGGCCGGCGGAGTTCGCGCGGGACCTCCTCCCCAAGTACTTCAAGCACAACAACTT | VVWRPAEFARDLLPKYFKHNNFS |
| | | CTCCAGCTTCGTGCGCCAGCTCAACACCTATGGATTTAGGAAGATCGTGCCGGACCGGTGGGAGTTCG | SFVRQLNTYGFRKIVPDRWEFAN |
| | | CCAACGACTGCTTCCGGCGAGGGGAGAAGCGTTTGTTGTGTGATATACACCGCCGGAAGGTGGCGGCG | DCFRRGEKRLLCDIHRRKVAAAA |
| | | GCGGCGGCGGCGGCGCCGACGGCAGCTGCCGCGGTGGCTTCGGGAGCTGTGACGGTGGCCGCGGCGCC | AAAPTAAAAVASGAVTVAAAPIP |
| | | GATACCTATGGCGCTGCCGGTGACGCGGGCGGGCTCGCCGGCGCACTCCTCGGAGGAGCAGGTGCTGT | MALPVTRAGSPAHSSEEQVLSSN |
| | | CGTCAAACTCCGGGTCGGGGGAGGAGCACCGGCAAGCGTCCGGGTCCGGCTCTGCGCCCGGCGGCGGC | SGSGEEHRQASGSGSAPGGGGGG |
| | | GGCGGCGGCTCCGCGTCCGGCGGCGACATGGGCGAGGAGAACGAGCGGCTCCGGCGGGAGAACGCGCG | SASGGDMGEENERLRRENARLTR |
| | | GCTGACGCGGGAGCTCGGCCACATGAAGAAGCTCTGTAACAACATCCTCCTCCTCATGTCCAAGTACG | ELGHMKKLCNNILLLMSKYAATQ |
| | | CCGCCACCCAGCACGTGGAGGGCTCCGCGGGGATATCCTCCATAGCGAACTGCTCCGGCGAGTCGTCG | HVEGSAGISSIANCSGESSEAVP |
| | | GAGGCCGTTCCCCCGCCGCCGCCTCTCCCCCCGGCGATACTCGATCTAATGCCCTCCTGCCCCGCCCT | PPPPLPPAILDLMPSCPALATAA |
| | | GGCCACCGCCGCCGCCGCCGCGGGGCTCGCCATAGACGTCGAGCCCGACCCGAGCGCGCGCCTGTTCG | AAAGLAIDVEPDPSARLFGVSIG |
| | | GCGTCTCCATCGGCCTGAAGCGGACTCGAGGCGACGCCGCCGCCGACGACGAAGACGGCGGCGGCGAG | LKRTRGDAAADDEDGGGEDQAEH |
| | | GATCAGGCCGAGCACGGCGGCGCGGACGTGAAGCCGGAGGCGGCGGATCCGCATCCCGCCGGCGGCGG | GGADVKPEAADPHPAGGGGGSST |
| | | CGGCGGAAGCAGCACGGAGGCGTCGCCGGAGTCGCACCCGTGGCCCATATACCGGCCAACCCCCATGT | EASPESHPWPIYRPTPMYHAVRP |
| | | ACCACGCGGTCAGGCCCACTTGCAACGGGCCGGACAGGGCCGGGTCCGACCAAGACGGGTCCAGCTCC | TCNGPDRAGSDQDGSSSSQTMGP |
| | | AGCCAAACGATGGGGCCTGATGAATTCGACGGTCCGCGAAAGACGTTGGTGGTGCAACAGAGCAACTT | DEFDGPRKTLVVQQSNFMMHWGH |
| | | CATGATGCACTGGGGGCATTCGGAATGCGGTAGTGGCGCAAGGGGTTTTGGATGGTGA | SECGSGARGFGW* |
| | | SEQ ID NO: 269 | SEQ ID NO: 270 |
| 136 | Xa21_Os09g0565300 | ATGTTCTTTTCTACTTTCTTGCTGCTGTTCTGGACCATTTATTCTTGGACTGTACGCCTGAATAGAGA | MFFSTFLLLFWTIYSWTVRLNRD |
| | | TGAGATCGGCAACCTGAGGATCAACAACTCCTGGGATTCGCAGAGATATGATACCTTGGATCAGGTTA | EIGNLRINNSWDSQRYDTLDQVK |
| | | AGGAAGCTCTTGAAAAGGTCGGTCTCGAGTCGTCCAATATTATCATTGGAGTGGATTTCACCAAAAGC | EALEKVGLESSNIIIGVDFTKSN |
| | | AACGAGTGGACAGGGAAACATTGTTTCAACGGGAGAAGCCTGCATCACATTTCAGAGGACTCCCTCAA | EWTGKHCFNGRSLHHISEDSLNP |
| | | CCCGTACGAGCAAGCGATTAGTATCATCGGCAAGACCTTATCGACGTTCGACGAAGACAACAGGATCC | YEQAISIIGKTLSTFDEDNRIPC |
| | | CATGTTTTGGATTTGGGGACAGTAAGCAAAAGCTTCTCCGTCGTCTTCTAGTTCTTTCTCTCTGCATT | FGFGDSKQKLLRRLLVLSLCISL |
| | | TCTCTGTTGAGAAATGAAGTGAGATACTGA SEQ ID NO: 271 | LRNEVRY* SEQ ID NO: 272 |
| 137 | Xa21_Os10g0497700 | ATGGCGATTGGTGTTGGTGGCTGCTGCGCCGTGCTGCTCGCGGCGACGCTGCTCTTCTCCTCTCCGGT | MAIGVGGCCAVLLAATLLFSSPV |
| | | CACCTCATATGCTTATGATTCCCTGGATCCAAAAGGCAACATCACGATAAAATGGGATGTCATGCAAT | TSYAYDSLDPKGNITIKWDVMQW |
| | | GGACTCCTGATGGCTATGCTGCCGTTGTCACACTGTCCAACTACCAGCAATTCCGGCACATCCAGCCA | TPDGYAAVVTLSNYQQFRHIQPP |
| | | CCGGGGTGGCAGCTGGGGTGGACATGGCAGCAGAAGGAGGTGATCTGGTCCATGTACGGCGCGCTGGC | GWQLGWTWQQKEVIWSMYGALAT |
| | | CACCGAGCAGGGCGACTGCTCGATGTCCAAGGAGGGCAGCAATGTCCCCCACAGCTGCAAGAAGGATC | EQGDCSMSKEGSNVPHSCKKDPT |
| | | CCACCGTCGTCGACCTCCTCCCGGGCACCCCAATCGACCTGCAGATCGCCAACTGCTGCAAGGCTGGA | VVDLLPGTPIDLQIANCCKAGSL |
| | | TCACTGAGCGCATTCAGCCAGGACCCGGCGAATTCTGCCGCGGCGTTTCAGATCATTGTAGGTCATTC | SAFSQDPANSAAAFQIIVGHSGN |
| | | TGGGAACAGCAATGAGACGGTTAGGGTGCCGAAGAACTTCAGTCTTATGGCGCCTGGTCCGGGGTATA | SNETVRVPKNFSLMAPGPGYTCS |
| | | CCTGTAGCCGTGCTATGATTGTGAAGCCTAGTAGGTTTTTAAGTCAGGATGGGAGGAGGGCGACTCAA | RAMIVKPSRFLSQDGRRATQALM |
| | | GCTCTGATGACATGGAATGTGATCTGCACATACTCCCAATTTCTTGCACAAAAGGTTCCATCCTGCTG | TWNVICTYSQFLAQKVPSCCVSL |
| | | TGTTTCTCTCTCGTCGTTCGACAATGACAAAACTGTTGACTGCCCAACATGCTCTTGTGGCTGCCAAA | SSFDNDKTVDCPTCSCGCQNEKS |
| | | ATGAAAAATCTACTACAGGAAAATGTGTAAAGAAGAATGCACCTGATTTGCAATCCATCATTCACGGC | TTGKCVKKNAPDLQSIIHGPGRW |
| | | CCTGGCAGATGGACCTGGCAGCCTCTTCTCCAGTGCACTTCACACATGTGCCCAGTGAAAATTAACTG | TWQPLLQCTSHMCPVKINWHLML |
| | | GCACTTGATGCTCAAAGACAAGGAACACTACAGAGTGAAAATCACTGTCACTAACTTGAACTACCGCA | KDKEHYRVKITVTNLNYRMNFTE |
| | | TGAATTTCACCGAATGGAACCTGGTTGTTCAGAATCCCAACTTAGATAGTATCACCCAGATATCTGGC | WNLVVQNPNLDSITQISGFNYKP |
| | | TTCAATTACAAGCCTCTTATGCCATATGGAGGCGGCATAAATGATACTACGATGCTATGGGGAGTAAA | LMPYGGGINDTTMLWGVKSYNDL |
| | | GTCCTACAATGATTTGCTCATGCAAGCTGGTCCGCTTGGAAATGTGCAAGGAGAACTGATTGTGAGAA | LMQAGPLGNVQGELIVRKDSRAS |
| | | AGGATTCCAGGGCTTCTTCCACTACTAACAAGGGATGGGCCTTCCCTACCCGTGTGTACTTCAACGGT | STTNKGWAFPTRVYFNGDNCVMP |
| | | GATAACTGTGTGATGCCGCCTCCGGATGCATATCCGGTTAGTATTACGGCTTAG | PPDAYPVSITA* |
| | | SEQ ID NO: 273 | SEQ ID NO: 274 |
| 138 | Xa21_OsJ_34925 | ATGCTTGTCAGGTTAGGCGTCGTTGTTGTGGCCTCCGTCGCAGCTTTAACTCTTAAGAGAGCCAATAG | MLVRLGVVVVASVAALTLKRANS |
| | (Os12g0105375) | CGGCAGCAAAGATGGCCAAGCAAGGAAAAGAAAGGATAAAACTCGTTACTCTGAACATGGGGAGAAAG | GSKDGQARKRKDKTRYSEHGEKE |
| | | AAGAAGAGAAGGAAGAGGTTAAAACAATCAGCGGCATAATCAATTCAGCTCTTTCAGATGATGACGAC | EEKEEVKTISGIINSALSDDDDM |
| | | ATGCTCTCCGAGATCGAGAGCCTCCTATCAGGGGAGATCGACATCCCCCTACCAAGTGACAGGTTCGA | LSEIESLLSGEIDIPLPSDRFDV |
| | | CGTCAAAGAACGCTCTCGGTACAACAGTGTTAACTCCGAGCTGGAGAGGCTGCGTGGACTTGTGCGTG | KERSRYNSVNSELERLRGLVREL |
| | | AGCTGGAGGAGAGGGAGGTGAAGCTTGAGGGAGAGCTGCTGGAGTACTATGGCCTCAAGGAACAGGAG | EEREVKLEGELLEYYGLKEQETD |
| | | ACTGATGTTGTAGAGCTACACAGACAGCTCAAGATCAAGATGGTGGAGATCGACATGCTCAAAATGAC | VVELHRQLKIKMVEIDMLKMTIN |
| | | CATCAACTCGCTGCAAGAGGAGAGGAAGAAGCTGCAGGATGACGTCGCCCGTGGCACAGGAGCCAAGA | SLQEERKKLQDDVARGTGAKREL |
| | | GGGAGCTGGAGGCTGCGAGGAACAAGATCAAGGAGCTGCAGCGACAGATACAGATGGAGGCGAACCAG | EAARNKIKELQRQIQMEANQTKG |
| | | ACGAAAGGGCAGCTGATGCTTCTAAAGAACCAGGTGATCGCGCTGAAGTCCAAGGAGGAGGAGGCAGC | QLMLLKNQVIALKSKEEEAAVKD |
| | | CGTCAAGGACGCAGAGGTGCAGAGGAAGCTGAAGAAGCTCAAGGAGCTGGAAGTGGAGGTAGTTGAGC | AEVQRKLKKLKELEVEVVELRRK |
| | | TGAGGAGGAAGAACAAGGAGCTCTTGTATGAGAAAAGGGATCTCATCGTTAAGCTGGATGCAGCACAA | NKELLYEKRDLIVKLDAAQGKIT |
| | | GGAAAAATAACAGAGAGTGATGTAGTTTCCCATGCAAGGGAGGAGATCAACAAGCTGAGGCATGCAAA | ESDVVSHAREEINKLRHANEDLT |
| | | TGAAGACCTTACCAAGCAAGTAGAAGGCCTACAAATGAACAGATTCAGTGAAGTAGAAGAGTTGGTTT | KQVEGLQMNRFSEVEELVYLRWV |
| | | ACCTGCGTTGGGTTAATGCTTGTCTGAGATATGAGCTCCGCAACTACCAGGCACCGTCTGAGAAAATT | NACLRYELRNYQAPSEKISARDL |
| | | TCTGCTCGTGACCTTAACAAGACCCTGAGCCCAAAATCGCGTGAGAGGGCCAAACTTCTAATGCTGGA | NKTLSPKSRERAKLLMLEYAGSE |
| | | ATATGCAGGATCAGAACGAGGACAGGGCGACACTGACCTTGAAACTGCTTCTTCTGCACCTTCTTCAC | RGQGDTDLETASSAPSSPRSEDF |
| | | CCAGAAGTGAAGACTTCGACAATGTTTCAGTTGACAGTTCTTCTAGCAGATACAGCTTCTTTGGCAAA | DNVSVDSSSSRYSFFGKRPNLMQ |
| | | AGGCCCAATCTGATGCAAAAGCTCAAGAAGTGGGGAAGGGGCAAGGATGACGGAAGCAGCTTAGCTTC | KLKKWGRGKDDGSSLASPTQSFT |
| | | ACCGACACAGTCCTTCACTAGTGACTCCCCAAAGAGTACCAGCCAGAAACCAAAGGGGCCCCTAGAGG | SDSPKSTSQKPKGPLEALMLRNA |
| | | CTCTCATGCTCAGAAATGCAGGAGATGGTGTGGGCATTACAACCTTTGGAAAGAGGGAACAAGACCCC | GDGVGITTFGKREQDPSDIMDEA |
| | | AGTGATATCATGGATGAGGCAAATGTTGCATCTTCATTCCATTTGATGTCAAAGACTGTACAAGGTTT | NVASSFHLMSKTVQGFADDKYPA |
| | | TGCTGATGACAAGTACCCCGCTTACAAAGATAGGCATAAACTCGCCACAGAACGGGAGAAGGCAATAA | YKDRHKLATEREKAIKEKAEKAR |
| | | AAGAGAAGGCCGAGAAAGCTAGGGTACAAAGATATGGCGGTGTCAACAGTTCAGGTATTGTGCCATCT | VQRYGGVNSSGIVPSPRSALPPK |
| | | CCAAGATCTGCACTCCCTCCAAAACTTGCTCAAATAAAGGAGAAGGCTTCTACAGCTAATGCTGAATC | LAQIKEKASTANAESSDQPSDNQ |
| | | CAGTGACCAACCTAGTGATAACCAGAACAACCCTCTAGTTGTGACACAACTGAAACTTGCAAATATTG | NNPLVVTQLKLANIEKRAPRVPR |
| | | AGAAGAGAGCTCCAAGAGTTCCTAGGCCACCTCCTGCACCATCAGCCACTGCCACAGGAGCTGCCAAC | PPPAPSATATGAANTASALPPPP |
| | | ACTGCAAGTGCGTTACCTCCACCACCGCCACGCCCTCCAGGTGCACCTCCTCCACCACCGCCTCCTGG | PRPPGAPPPPPPPGKPGGPPPPP |
| | | CAAACCTGGTGGCCCGCCACCACCGCCACCACCCCCTGGTTCCCTACCTAGGAATCTTGCTGGTGGTG | PPPGSLPRNLAGGDKVHRAPEVV |
| | | ACAAGGTACACCGTGCTCCAGAGGTTGTAGAGTTTTATCAAAGTCTCATGAAGCGTGAAGCCAAGAAG | EFYQSLMKREAKKDTTSLGSTTS |
| | | GACACAACTTCTCTGGGATCAACAACATCAAGTGTCTCTGATGTGAGAAGCAACATGATTGGAGAGAT | SVSDVRSNMIGEIENRSTFLLAV |
| | | TGAGAATAGATCAACATTCCTCTTAGCTGTCAAAGCGGATGTGGAGACACAAGGAGACTTTGTTGAGT | KADVETQGDFVESLANEVRAASF |
| | | CTCTAGCAAATGAGGTCCGAGCAGCTAGTTTTGTAAATATCGACGACGTTGTTGCATTTGTAAATTGG | VNIDDVVAFVNWLDEELSFLVDE |
| | | CTTGATGAGGAACTATCCTTCTTGGTCGATGAGCGGGCAGTACTAAAGCACTTTGATTGGCCAGAGAG | RAVLKHFDWPESKTDALREAAFE |
| | | CAAAACTGATGCACTAAGAGAGGCAGCCTTTGAGTATCAAGACCTGCTAAAATTAGAACACAAGGTTT | YQDLLKLEHKVSSFTDDPKLACE |
| | | CATCGTTTACTGATGATCCAAAGCTAGCATGTGAAGAAGCTCTCAAGAAGATGTATTCCTTGCTTGAA | EALKKMYSLLEKVEQSVYALLRT |
| | | AAAGTGGAGCAGAGTGTTTATGCGCTACTTCGTACTAGAGACATGGCCATATCACGCTACAGGGAGTA | RDMAISRYREYGIPVDWLSDSGV |
| | | CGGAATACCGGTGGATTGGCTGTCTGATTCCGGGGTAGTTGGCAAGATCAAATTGGCATCTGTTCAGC | VGKIKLASVQLAKKYMKRVATEL |
| | | TGGCGAAGAAGTATATGAAGAGGGTTGCCACAGAGCTTGATGCCTTGCAAGGCACCGAGAAAGAGCCC | DALQGTEKEPNREFLLLQGVRFA |
| | | AACAGAGAGTTCTTGCTTCTTCAGGGTGTGAGATTTGCTTTCCGAGTTCATCAGTTTGCTGGAGGGTT | FRVHQFAGGFDEESMKAFEELRS |
| | | CGATGAGGAAAGCATGAAGGCATTTGAAGAGCTAAGAAGCAAGATGTCTACACAGACATCTGCTCCAC | KMSTQTSAPQISDV* |
| | | AAATTTCCGATGTGTAG SEQ ID NO: 275 | SEQ ID NO: 276 |
| 139 | Xa21_Os11g0213800 | ATGGCGGTGGCGGAAGTCGGGGGAATGCTTGCATCTGCCGTCCTCAAGGTGGCGACCCAGAAGCTGGG | MAVAEVGGMLASAVLKVATQKLG |
| | | CGCCACCATAGCGGGCCGGGTCATGCTCCAGTGGAACTTCGACAAGGATCTGGAGGGGATGAAGGCCA | ATIAGRVMLQWNFDKDLEGMKAT |
| | | CGCTGGAGTCTGTGGACGCGGTGCTCAGGGACGCCGAGAGGCGGTCGATCCGCGACGCGGCGGTGCGG | LESVDAVLRDAERRSIRDAAVRL |
| | | CTGTGGCTGAGGAGGCTCAAGGACGCCGCCTACGACATCTCCGACATGCTCGACGACCTGGAGGCCAT | WLRRLKDAAYDISDMLDDLEAIT |
| | | CACTTCCAAATCGGATGCGGGGAAGTTGGGATGTATGATGATCCCCAATCTGACTATTGCTCACAAGA | SKSDAGKLGCMMIPNLTIAHKIT |
| | | TTACCCTAGCTAATAAGATGAAGACGATAAGTGATGAACTGAAGGAGATCACTAATCAACATCTTAGC | LANKMKTISDELKEITNQHLSFR |
| | | TTCAGATTCACGGAAGATAGCAGCTATAAGGAGCACAGAGTTACTGATAAACGGGAAACATCGTCCAA | FTEDSSYKEHRVTDKRETSSKVE |
| | | GGTGGAAGAAGCACGAGTTGTTGGGAGGACTGCAGAGAAGCGGATAATTATATCTTCTCTGTCCAAGA | EARVVGRTAEKRIIISSLSKRMT |
| | | GAATGACTGAAGAGACCATAATCCTTCCTATATATGGCATTGGAGGCATAGGCAAGACAACCTTGGCG | EETIILPIYGIGGIGKTTLAKLV |
| | | AAACTGGTTTTCAACGCTTCCAAATTCAGAGACTACTCTAAGGTCTGGATATATGTGTCTCAGACATT | FNASKFRDYSKVWIYVSQTFDLN |
| | | TGACTTGAACAAAATTAGCAACTCTATAATTTCACAAGTATCGGAAAGTGAGAGCCAGTTGACTGAAA | KISNSIISQVSESESQLTEREMI |
| | | GGGAGATGATAAATAGGCGTCTTGATGAGCTACTTTCCGGTAAGAAGATTCTAATTGTTTTAGATGAC | NRRLDELLSGKKILIVLDDLWER |
| | | CTATGGGAGAGGGATCAATTTCAGTTAGATGATTTGAAGACTATGTTAAAGGTTGGCAGGGGCAGCAG | DQFQLDDLKTMLKVGRGSRVIAI |
| | | GGTGATTGCCATCGTAACCACCCGAGATAAGGACATTGCAGAGAAGATTTGCACCACTGAACCGTACA | VTTRDKDIAEKICTTEPYKLEPL |
| | | AACTAGAACCCCTGACAGATGACATGTGCTGGAAAATAATAAAAGAAAAGAGTGTCTTTGAAGCAAGA | TDDMCWKIIKEKSVFEARDDKEQ |
| | | GATGACAAAGAACAATTGGAGAATATAGGAAGGGAAATAGCATCTAAGTGTGGAGGTGTGGCTTTAGC | LENIGREIASKCGGVALAAKSLG |
| | | AGCCAAATCACTTGGATATACTCTGCAACCCATGAAGTTTGATGAATGGGTATCAGTGAGAAACAACG | YTLQPMKFDEWVSVRNNDIWKAS |
| | | ATATCTGGAAGGCATCTACTTTCGAATGTACATCTTTGCCATATCATAATGTGCTTGCATCCTTGCAA | TFECTSLPYHNVLASLQLSCSNM |
| | | TTGAGCTGCAGCAATATGCCTCCAAACCTGAGGTTATGCTTTGCATATTGTGCAATCTTTCCAAAAGG | PPNLRLCFAYCAIFPKGHIIVKD |
| | | TCACATCATAGTAAAAGACCAAATAATTCACCAATGGAATGCTCTTGGTTTCATAGAGCAATCTGACA | QIIHQWNALGFIEQSDIFSTRQL |
| | | TATTCTCTACTAGGCAGCTTGGTGAGGCATATGTTAGACAGCTTTTGGGGTTGTGCTTCCTTCAACAA | GEAYVRQLLGLCFLQQTKAPSVS |
| | | ACAAAGGCTCCTTCAGTGAGTTTCTACTTACCACATGCATCTATAGCTGAACAGCTTTATCATTTCTT | FYLPHASIAEQLYHFFISL* |
| | | TATTAGCCTATAG SEQ ID NO: 277 | SEQ ID NO: 278 |
| 140 | Xa21_OsI_00118 | ATGCACCCGAAGCGTCCGCTGCCGGCACAGGCGCCACCAGCTGGGCTCGTCCTGGTGCCCGCGCCCAA | MHPKRPLPAQAPPAGLVLVPAPK |
| | (Os11g0669100) | GCGCCCGCATGTGGATGCTGCTGCCGGCGGCGGCGTGGCGTCGCCGCGGGGGAAGCGGCAGCTTCGCT | RPHVDAAAGGGVASPRGKRQLRS |
| | | CCGGCATGCTCGTCCTCTTCTTCGTCGCGCAAGTCAAGGAGGAGATGAGATACAACCAGCGACTTCGT | GMLVLFFVAQVKEEMRYNQRLRR |
| | | CGAGTGATACGTGGAGAGAATGCTATTTCCCAGCAAAGGGCAATCCAAGCATTTGATTGTGTCTTTCA | VIRGENAISQQRAIQAFDCVFQK |
| | | GAAAGCATTTGACAATGCCTTCCAGAAGCATCTTGATCCGATTTACCGCTCCCTTCAATCCCTTAACA | AFDNAFQKHLDPIYRSLQSLNKR |
| | | AGCGCACTGATATTCTCAGCCATGAAGTGGAACAAATCAAACATTCAAATTCTAACCATCATGCTAAT | TDILSHEVEQIKHSNSNHHANQQ |
| | | CAGCAATACAGATCAAAGGTAAATCAAGAGTCTGCTGCCATCACTGAGGAAGTGAATCAAGAACAAAC | YRSKVNQESAAITEEVNQEQTAA |
| | | AGCTGCTAGATTTGTCGCGCGTGAAGCTCAGGAGGGGCAAAGAGTTGAGTTGAGGTTCCTCAATAAGT | RFVAREAQEGQRVELRFLNKLNP |
| | | TGAACCCTCTCGTTTTTACAAAGGAAAAAATTACAGCGGAGGATGGTACAACTATCAAAATTGCCATA | LVFTKEKITAEDGTTIKIAIVRD |
| | | GTTAGGGATAATCAGATAATTACATCTGGTCCACTTTCTTCTGCAAGAATTGAAATTTTGGCTCTCCA | NQIITSGPLSSARIEILALHGNF |
| | | TGGTAACTTTTATGATGTTGTGCCTGATAATTGGACCGAATCTGAGTTTGATCACCGCATAGTAAGTA | YDVVPDNWTESEFDHRIVSSSQG |
| | | GTTCACAAGGGCCCGCGTTAGGAGGAGTTTGTCAGGTCAAGCTGAAGAATGGGGAGGCCTCTCCTTCT | PALGGVCQVKLKNGEASPSDVFF |
| | | GATGTATTTTTCAATATACCATCTTCGAAGACTGAAAGTGGAAGGCTCATTCTAGCAGCAAAAGTTCA | NIPSSKTESGRLILAAKVHTSDN |
| | | TACAAGTGACAATGGTGGGTTTCGAATTAAGGAAGCTGTGATGATGAATCCTGTTGTGGTGCAGGTTT | GGFRIKEAVMMNPVVVQVYRNKL |
| | | ACCGAAACAAACTAAATAGAAGTAGTGACCGTCCGAAGCTAAAAGATGAAGTACATCGTTTAAAGGGA | NRSSDRPKLKDEVHRLKGISGKG |
| | | ATTTCAGGAAAGGGATGCCGCACCAAATGGCTTAAAGATAATCAAATCAACACTGTGGAAGAGTTCGT | CRTKWLKDNQINTVEEFVKALNK |
| | | AAAGGCTTTAAATAAGGATGAAGAAAAGATCCGCAATGAGTGTTTCAAGCTAAAGAAGGATAATAAGC | DEEKIRNECFKLKKDNKLWKDTI |
| | | TTTGGAAAGATACAATTAAACATGCTAAAGAGTGTGATCTTGAAGGGAATTGCAAGCTGAAATTATAT | KHAKECDLEGNCKLKLYRAEEQH |
| | | AGAGCTGAAGAGCAGCATGTTGTACTCTTCTTTAACTGCGTGCATGACCTCGTTGGTGCGAAATTTCG | VVLFFNCVHDLVGAKFRDHYVAK |
| | | TGATCACTATGTTGCAAAGGACAATTTCAGTTCAGATCAACAGGATGCAGTGAATCGTTTGAAGAAAC | DNFSSDQQDAVNRLKKQAYDELD |
| | | AAGCATATGATGAGCTGGATAGCATTGGCTTTGATCACGAAATGAAAAATAACTATCCGGTGATGACG | SIGFDHEMKNNYPVMTLSDDAYI |
| | | CTCAGTGATGATGCTTACATTCCCTTCACTGACACAGCACAAAATCCTCCAGATTTGCATGTTACATT | PFTDTAQNPPDLHVTFQVQGIAA |
| | | TCAAGTTCAAGGTATTGCAGCAGCTGAGATTTATCATGAACATGAACTGCCTCAAGCATTTCCTAACA | AEIYHEHELPQAFPNNNNDFGQD |
| | | ACAATAATGATTTTGGACAAGACTTTTTACATGGCTTTCAAGGTGCTCTGACTCAGATGGACCATGAC | FLHGFQGALTQMDHDYAQFGIAD |
| | | TATGCCCAATTTGGCATTGCTGATATGCAGTGTTATACTACACGGGCACCTGAAGGTACCTCTTATGG | MQCYTTRAPEGTSYGGNNMIGPA |
| | | AGGCAACAACATGATCGGACCAGCAAATGTACCGCAGAATGTTATTGGTGACGGCTCTATGGACATGT | NVPQNVIGDGSMDMFDCYAYIVP |
| | | TTGACTGTTATGCATACATCGTTCCGGACAATGAGAACCAGAATGAGAGGCCTCATAGCTCTGCATAT | DNENQNERPHSSAYPGPV* |
| | | CCAGGACCAGTTTAA SEQ ID NO: 279 | SEQ ID NO: 280 |
| 141 | Xa21_Os09g0425900 | ATGGCGTTCCGGCTGAGCAACAGCCTGCTCGGGATCCTGAACGCGGTGACGTTCCTCCTGTCGGTGCC | MAFRLSNSLLGILNAVTFLLSVP |
| | | CGTGCTGGGCGGCGGCATCTGGCTGGCGACGCGCGCCGACGGCACGGAGTGCGAGCGCTACTTCTCGG | VLGGGIWLATRADGTECERYFSA |
| | | CGCCGGTGATCGCGTTCGGGGTGTTCCTCCTCCTCGTCTCCCTGGCGGGCCTCGTCGGCGCCTGCTGC | PVIAFGVFLLLVSLAGLVGACCR |
| | | CGCGTCAACTGCCTCCTCTGGTTCTACCTCGTCGCCATGTTCGTCCTCATCGTCGTCCTCTTCTGCTT | VNCLLWFYLVAMFVLIVVLFCFT |
| | | CACCGTCTTCGCCTTCGTCGTCACCAACAAGGGCGCCGGCGAGGCCGTCTCCGGCAGAGGGTACAAGG | VFAFVVTNKGAGEAVSGRGYKEY |
| | | AGTACAGGCTCGGCGACTACTCCAACTGGCTGCAGAAGCGGATGGAGAACAGCAAGAACTGGAACAGG | RLGDYSNWLQKRMENSKNWNRIR |
| | | ATCAGGAGCTGCCTCCAGGACTCCAAGGTCTGCAAGAAACTGCAGGACAAGAACTGGGATCGGACCCA | SCLQDSKVCKKLQDKNWDRTQFF |
| | | GTTCTTCAAAGCCGACCTCTCCCCGCTCGAGTCCGGATGCTGCAAGCCACCCAGCAGCTGCAACTTCC | KADLSPLESGCCKPPSSCNFLYV |
| | | TCTACGTCAGCGGCACGAACTGGACGAAGGTGCCCACCAACTCGTCGGACCCGGACTGCAACACGTGG | SGTNWTKVPTNSSDPDCNTWVDD |
| | | GTCGACGACGGCACGCAGCTGTGCTACAACTGCCAGTCGTGCAAGGCCGGCGCGGTGGCGACCCTGAA | GTQLCYNCQSCKAGAVATLKRDW |
| | | GCGGGACTGGAAGCGCGTCGCCGTCGTCTGCATCGTCTTCCTCGTCTTCATCGTCATCGTCTACTCCC | KRVAVVCIVFLVFIVIVYSLGCC |
| | | TCGGCTGCTGCGCGTTCAGGAACAACCGGAGGGACAACCGCGGCGCCTACCGCGGTGCCGCGTGGAAG | AFRNNRRDNRGAYRGAAWKGGYA |
| | | GGCGGATACGCCTGA SEQ ID NO: 281 | * SEQ ID NO: 282 |
| 142 | Xa21_Os09g0456800 | ATGGCTGCTGCTGAGGCGGCGGCGGCGGCGGCGGTGGGGAAGCAGCAGCAGAAGGGCGGCGGGGGAAG | MAAAEAAAAAAVGKQQQKGGGGR |
| | | GGGAGGAGGCGGCGGCGGGCCGGCGCCGTTCCTGACGAAGACGCACCAGATGGTGGAGGAGAGCGCGA | GGGGGGPAPFLTKTHQMVEESAT |
| | | CGGACGAGGTGATCTCGTGGGGAAAGGAGGGGCGGTCGTTCGTGGTGTGGAAGCCGGTGGAGTTCGCC | DEVISWGKEGRSFVVWKPVEFAR |
| | | CGCGACCTCCTCCCGCTCCACTTCAAGCACTGCAACTTCTCCTCCTTCGTCCGCCAGCTCAACACCTA | DLLPLHFKHCNFSSFVRQLNTYG |
| | | CGGTTTCCGGAAGGTGGTGCCCGATCGGTGGGAGTTCGCGAACGGCAACTTCCGGCGAGGCGAGCAAG | FRKVVPDRWEFANGNFRRGEQGL |
| | | GCCTCCTCTCCGGCATCCGCCGCCGCAAGGCGACGACGCCTCAGTCCTCCAAATCCTGCGGCAGCGGC | LSGIRRRKATTPQSSKSCGSGVN |
| | | GTCAACGTCGCGTTCCCTCCGCCGCTGCCCCCTCTGCCCCCGGCGCCGTCGGCGACCACGTCCAGCGG | VAFPPPLPPLPPAPSATTSSGND |
| | | CAACGACCGCAGCTCCTCCTCCGCGTCCTCGCCGCCGCGGGCGGACATCACCAGCGAGAACGAGCAGC | RSSSSASSPPRADITSENEQLRK |
| | | TCAGGAAGGACAACCAAACGCTGACGACGGAGCTGGCGCGGGCTCGGCGGCACTGCGAGGAGCTTCTG | DNQTLTTELARARRHCEELLGFL |
| | | GGCTTCCTGTCGCGCTTCCTCGACGTCCGGCAGCTCGATCTCCGGCTGCTCATGCAGGAAGACATGCG | SRFLDVRQLDLRLLMQEDMRAAA |
| | | AGCGGCGGCCGCCGGAGGCGGCGGCGGCGAGCAGCGCGAGCAAGAGCACGCCCGGGAGGAGAAGTGCG | AGGGGGEQREQEHAREEKCVKLF |
| | | TGAAGCTGTTCGGCGTGCTCCTCGACGACACCCATGGCGCCGCCACGAGGAAGAGGGCGCGGTGCGAG | GVLLDDTHGAATRKRARCEEAAA |
| | | GAGGCAGCGGCCAGCGAGCGGCCGATCAAGATGATCAGGATCGGCGAGCCGTGGGTCAGCGTTCCGTC | SERPIKMIRIGEPWVSVPSSGPA |
| | | GTCGGGGCCGGCGCGGTGTGGCGGCGACAACTGA SEQ ID NO: 283 | RCGGDN* SEQ ID NO: 284 |
| 143 | Xa21_Os01g0949500 | ATGGTCAAGATCAAGATGCCCGCCCTGTTCCGCCGCCGGAGCGGCTCCAAGTCGCCGCCGCTGCCGCA | MVKIKMPALFRRRSGSKSPPLPQ |
| | | GGCCGACCCGGCGTCCGGCGGCGGGTCCCCGGCGCCGACGCCGGAGGAGGAGATGGAGCGGGTGTTCC | ADPASGGGSPAPTPEEEMERVFR |
| | | GCAAGTTCGACGCCAACGGCGACGGCCGGATCTCGCGGTCCGAGCTCGGCGCGCTGTTCGAGAGCCTC | KFDANGDGRISRSELGALFESLG |
| | | GGCCACGCGGCCACCGACGACGAGCTCGCCCGCATGATGGCCGAGGCGGACGCCGACGGCGACGGCTT | HAATDDELARMMAEADADGDGFI |
| | | CATCAGCCTCGACGAGTTCGCCGCCCTCAACGCCACCGCCAGCGGCGACGCGGCCGCCGTCGAGGAGG | SLDEFAALNATASGDAAAVEEDL |
| | | ACCTCCGCCACGCCTTCCGCGTCTTCGATGCCGACGGCAACGGCACCATCTCCGCCGCCGAGCTCGCC | RHAFRVFDADGNGTISAAELARV |
| | | CGCGTCCTCCACGGCCTCGGCGAGAAGGCCACCGTCCAGCAGTGCCGCCGCATGATCGAGGGCGTCGA | LHGLGEKATVQQCRRMIEGVDQN |
| | | CCAGAACGGCGACGGCCTCATCTCCTTCGAGGAATTCAAGGTCATGATGGCCGGAGGCGGCTCCTTCG | GDGLISFEEFKVMMAGGGSFAKI |
| | | CCAAGATCGCCTAG SEQ ID NO: 285 | A* SEQ ID NO: 286 |
| 144 | Xa21_Os05g0494600 | ATGAGTATGAGGCGGTTGAGCGGCAGCGGCAATGCCGGCGGCGGCGCGGCGGTGGCCGGAGAGTGGGA | MSMRRLSGSGNAGGGAAVAGEWD |
| | | TGGTGGTGGTGGTGGTGGCATTGCGGGGAGGATGAGGGGGGTGAACGCCGGGATCATGGACGAGAAGG | GGGGGGIAGRMRGVNAGIMDEKV |
| | | TGCTGGAGCTGGTGTTCCGGGCGCTGAACTGGGACCCGAGGGAGCTGTGCGTGGTGGCGAGGGTGAGC | LELVFRALNWDPRELCVVARVSR |
| | | CGGCGGCTGCGGGCGGTGGCGGAGCGGGTGCTGTGGCGGGAGCTGTGCGTGTCGAGGGCGCCGCGGAT | RLRAVAERVLWRELCVSRAPRMV |
| | | GGTGTCGGCGCTGTCGGGCCCGACCGCGGCGGTGGCGGCGGCGGCAGGGCGCATCGGGGGAGGCTGGC | SALSGPTAAVAAAAGRIGGGWPA |
| | | CGGCGATGGCGAAGCTGCTCTTCTTCTGCTGCGGCGCGGCGGGCGCCGCCGTGCCGGGCCACTTCGCG | MAKLLFFCCGAAGAAVPGHFAPV |
| | | CCCGTGTCGCGGTTCTCCAAGACGTCGGGCCGGAGCTTCCTGTCGCGGCGGTGCGCGGGGGACATGCT | SRFSKTSGRSFLSRRCAGDMLFV |
| | | GTTCGTGTCGGACCCGTGCGAGCACGCCGCCGCCGGCGCCGCGCCCGACGACGTCGTGGGCGCGTACC | SDPCEHAAAGAAPDDVVGAYRGV |
| | | GCGGCGTGTACCGCGGGTTCATGCGGTCGCGGACGCGCGCCTTCCTCGTCGGCCACCGCGCCCCGCTC | YRGFMRSRTRAFLVGHRAPLEPR |
| | | GAGCCGCGCGTCCGCTGCCCCTACTGCGGCGCCCGCGTGTGGAGCATGACCGCCGCGGGGCTCGCGCC | VRCPYCGARVWSMTAAGLAPRSA |
| | | GCGCAGCGCGAGCCGCCGCCTCGGCGCCAACGAGGGCCGCCTCGAGTACTTCGTCTGCGTCAGCGGCC | SRRLGANEGRLEYFVCVSGHLHG |
| | | ACCTCCACGGCAGCTGCTGGCTCGCCCGCCTCTCCTCCTCCTCCTCCTCCAGCGACGGCGAGCGCAGC | SCWLARLSSSSSSSDGERSADSD |
| | | GCCGACTCCGACTCCAACCACAGCGACGACGAGACGTTCGCCGCCGCCGACGTGAGCCTCCCGCTTCC | SNHSDDETFAAADVSLPLPPAGR |
| | | TCCAGCCGGCCGCGTGTCAGCCCGGCGCCTTCGAGGCAGGCCCGCCATGTGA | VSARRLRGRPAM* |
| | | SEQ ID NO: 287 | SEQ ID NO: 288 |
| 145 | Xa21_Os05g0560600 | ATGGAGGTGAGGGAGATGGCCGTGGCGGCGGCTGCGGCGGCGGCGGCGTCGTCAGGGGGAGGAGGTGG | MEVREMAVAAAAAAAASSGGGGG |
| | | GGGGTTGAGGATGCCGCCGCCCAACCCTAACCTGCCGTACAGGGAGGATTGCTGGAGCGAGGGCGAGA | GLRMPPPNPNLPYREDCWSEGET |
| | | CGGAGGCGCTGGTCCGCGCGTGGGGGAGCCGCTACGTCGAGCTCAACCGGGGGAACCTGCGGCAGAAG | EALVRAWGSRYVELNRGNLRQKQ |
| | | CAGTGGCAGGAGGTGGCCGACGCCGTCAACTCGCGGCGCGGGGCGGCCGCGCGCCGCCGCCCGCCGCG | WQEVADAVNSRRGAAARRRPPRT |
| | | CACCGACGTGCAGTGCAAGAACCGCGTCGACACCCTCAAGAAGAAGTACAAGGCGGAGCGCGCCCGCG | DVQCKNRVDTLKKKYKAERARVM |
| | | TCATGCCATCCACCTGGTCCTTCTTCCCCGAGCTCGACCGCCTCGTCGGGCCCACCCTCTCCGCCTCC | PSTWSFFPELDRLVGPTLSASAS |
| | | GCCTCCAAGAGGCCCTCGCCGTCGCCGTCGCCGGTGCCGCCGCCCCCTCACTTCGCCATGCCCATCCA | KRPSPSPSPVPPPPHFAMPIHPS |
| | | TCCTTCCGCGGTGAGGAAGCCCCCCTCGCCTTCGCCGTCGCCCTCTCCGCCGCCTCCCATGGCGTTGC | AVRKPPSPSPSPSPPPPMALPLP |
| | | CGCTGCCCAGCTACCGTCGCGGGTCTCCGCTCCCCGCCGCCGCTCTTATCCAGCAAGAGGCCGCGGCT | SYRRGSPLPAAALIQQEAAAAAA |
| | | GCAGCCGCTGCCGCGGTGTCTGATTCTGAGGACTCTGAAGGCCCTGGTGACAACAACAATCACAACGC | AAVSDSEDSEGPGDNNNHNAQRS |
| | | GCAGCGGTCACCGTCGCAATCTGTGTCATCGCGCTCCGGCAATAGCAACAAGCGCAGCCGCCAGGAAG | PSQSVSSRSGNSNKRSRQEVDGG |
| | | TTGATGGTGGTTTCAGGGAGCTGGCAAGGGCGATCGAGGCATTTGCGGAGATGTATGAGCGTGTGGAG | FRELARAIEAFAEMYERVESAKQ |
| | | AGTGCGAAGCAAAAGCAGGCGCTGGAGATTGAGCGGCAGCGAATAGACTTCCTGAAGCAGTTGGAGGT | KQALEIERQRIDFLKQLEVKRME |
| | | CAAGCGCATGGAGAACTTTGTGGATGCACATGTCAAGCTTGCCAGGGCAAAGCGCATCAAGAAGCATG | NFVDAHVKLARAKRIKKHAGTAP |
| | | CAGGGACTGCACCCGATGGTATTGGCGCAGCGGAATTGGTTTCCTCTGTCGCTGCGCTGCCTTTCCTC | DGIGAAELVSSVAALPFLSTSTY |
| | | TCCACCTCCACCTACATATGA SEQ ID NO: 289 | I* SEQ ID NO: 290 |
| 146 | Xa21_Os09g0526500 | ATGGCGTCGAGGACGATGACACTGGAGGTGACGGTGGTGTCCGCCGAGGAGGTCGTGCTGCCGCCGAC | MASRTMTLEVTVVSAEEVVLPPT |
| | | GAGGCGCCCGCTCGGCCGCGGCGCGTACGCGGTGGTGCGCACGGCGGCGTCGGCGTCGTCGCCGGCGG | RRPLGRGAYAVVRTAASASSPAA |
| | | CGGCGGTGTGCACGCGCGTCGACGAGGAATCGGGCGGCGACTGCAACGGGTACCCCTACTGGAAGGAG | AVCTRVDEESGGDCNGYPYWKEA |
| | | GCGCTGCGGGTGGCGTTGCCGGAGGGGGCGCGCTGGCTGGACGTCGAGATCTGCCGGAGGAGGCCGAA | LRVALPEGARWLDVEICRRRPNG |
| | | CGGCCAGGTGGAGGCGGTGGCGGCGGCGAGCGTCCCCGTCGGCGACTTCACCGTCGGCCCGCCGGGCC | QVEAVAAASVPVGDFTVGPPGHL |
| | | ACCTGCATTGCCTCAGCTACAGGCTGTTCGACGCCTCCGGGTGCAGGACGCGCCGCAACGGCATCGTC | HCLSYRLFDASGCRTRRNGIVNI |
| | | AACATCACCGTCAGGAGGACGGACGTCAAGTACACCGCGCCGCCTCCCCCGCCGCCGCCGGTGAAAGC | TVRRTDVKYTAPPPPPPPVKAPA |
| | | TCCGGCGTACTCCGGCGCGAGCGGGAGCGGCGGCTCTTGCTACGGCGTGCCGCCGGCGGGCGCGGCCA | YSGASGSGGSCYGVPPAGAAMGF |
| | | TGGGTTTCCCCGTCGGATTCACGGCCAATGGCAAGGCCTGCGCGTAG | PVGFTANGKACA* |
| | | SEQ ID NO: 291 | SEQ ID NO: 292 |
| 147 | Xa21_Os01g0373700 | ATGGGAGGCAAGAAGAGGAAATCCGGCTCCAGTTTGTGGTTGGCCGTGGCGTCTTGGTTCGCGCGATT | MGGKKRKSGSSLWLAVASWFARL |
| | | GGGCTCAAAGATGGTGGCGAGCTCTGGCTCCGCCGGTGGCCGCGCAGGGTATGATGCGGCTGGCGAGA | GSKMVASSGSAGGRAGYDAAGEM |
| | | TGGTCAGGGCGGCTAAACATTTCTCCAGCGCTCACAAGATAAACTTTGGTTGA | VRAAKHFSSAHKINFG* |
| | | SEQ ID NO: 293 | SEQ ID NO: 294 |
| 148 | Xa21_OsI_17083 | ATGACGCTTCTAGCATATTCTTCCGGAAATTTCTGGAGATCTCGTCCATCTCCCACAAGCCGAACGCA | MTLLAYSSGNFWRSRPSPTSRTQ |
| | | ATCTGCTCGTTCGTTCCTCGCTTCTGGGACTACGGAACTCGGATTCGAGGCATCCTGCGCAACTTACA | SARSFLASGTTELGFEASCATYK |
| | | AGTTACAAGTTACAAATCAAGGGATTCAAGATACAGGCATCGAATTACCATTACCAATGAAAGGAGCC | LQVTNQGIQDTGIELPLPMKGAA |
| | | GCAAGGTTTGTGGTGGAAAATGCAACCAGGCAAATCAAACCTGTCAATGGACTTCCGAGGTTGATGAC | RFVVENATRQIKPVNGLPRLMTI |
| | | AATTACTACTCCTCAGAAGCATGGCAAGGAAAATAATTCCAATGATTCAGTGCTCATAAAGGATGAAA | TTPQKHGKENNSNDSVLIKDENI |
| | | ACATCGAGCCACTTGTTGCTTTTAGCAGGCCACCTCCTTTGCCACCAGTTCTTGGCCCCTTGATCATG | EPLVAFSRPPPLPPVLGPLIMLS |
| | | CTCTCACTTTTCAATATGTCAAGCGGCGGTGATGAAAACAAAAAATAA | LFNMSSGGDENKK* |
| | | SEQ ID NO: 295 | SEQ ID NO: 296 |
| 149 | Xa21_OsI_07792 | ATGACCACCTGCCTGCCCCACTTCAAGAGCCTCCTCGCCGGCCTCAACCGCTCGCCCGGCGTCCCGCC | MTTCLPHFKSLLAGLNRSPGVPP |
| | (Os02g0577700) | GGTCACCTGCGTGGTGGCGGACGCCGGCCTCACCTTCGGCGTCGACGCCGCGGAGGCTCTCGGCGTGC | VTCVVADAGLTFGVDAAEALGVP |
| | | CGTGCGCGCTGCTGTGGACGGCCAGCGCCTGCGGCTCGCTCGGCTACCGCCATTACCGCCTCTTCATC | CALLWTASACGSLGYRHYRLFID |
| | | GACAAGGGCCTTGTCCCGCTCAAAGATGCAGAGCAGCTGACGAACGGGTTCCTCGACACGCCGGTGGA | KGLVPLKDAEQLTNGFLDTPVDW |
| | | CTGGGCGTTCGGGATGAGCAAGCACGCGCGGATCGGGGACTTCCCGAGCTTCCTCCGCACGACGGACC | AFGMSKHARIGDFPSFLRTTDRD |
| | | GCGACGACGCCATGCTCAACTACGTGCTGCACGAGACGGACCACATGGCGGACGCGGACGCCATCATC | DAMLNYVLHETDHMADADAIIYN |
| | | TACAACACCTTCGACGAGCTGGAGCAGCCGGCGCTCGACGCCCTGCGCGCCGCACTCCAGCCGGCGGC | TFDELEQPALDALRAALQPAAVY |
| | | CGTCTACACCGTCGGCCCGCTCAACCTGCTCGCCGAGAGCCTCGCGCCATCGGGCGGCGGCGGCGACC | TVGPLNLLAESLAPSGGGGDPLD |
| | | CGCTCGACGCGCTGGGCTCCAACCTCTGGAGAGAGGACGACGCCTGCCTCGTCTGGCTCGACGGCAGG | ALGSNLWREDDACLVWLDGRAPR |
| | | GCGCCCCGGTCCGTGGTGTACGTGAACTACGGGAGCATCGCCGTGATGAGCAACCAGCAGCTGGTGGA | SVVYVNYGSIAVMSNQQLVEFAW |
| | | GTTCGCGTGGGGGCTCGCCGGCAGCGGCTACGCCTTCCTGTGGGTGATCAGGCCAGACCTGGTGACCG | GLAGSGYAFLWVIRPDLVTGNDA |
| | | GCAACGACGCCTCCGCCGCGGCGCTGCCGCCGGAGTTCATGGAGGCGACGAGGGGCAGGGGCCTCCTG | SAAALPPEFMEATRGRGLLASWC |
| | | GCGAGCTGGTGCCCGCAGGAGGCGGTGCTGCGGCACGAGGCGGTGGCGCTGTTCCTGACGCACAGCGG | PQEAVLRHEAVALFLTHSGWNST |
| | | GTGGAACTCGACGCTGGAGAGCCTGTCCGGCGGGGTGCCGATGCTGTCGTGGCCGTTCTTCGCCGAGC | LESLSGGVPMLSWPFFAEQPTNS |
| | | AGCCGACCAACTCCCTGTACAAGCGCGCGGAGTGGGGCGTCGCGATGGACGTCGGCGGCGGCGGCGGC | LYKRAEWGVAMDVGGGGGVRREA |
| | | GTGCGGCGGGAGGCGGTGGAGGCGAGGATACGGGAGGCGATGGGAGGGGAGAAGGGGAGTGCGATGAG | VEARIREAMGGEKGSAMRKRAAE |
| | | GAAGAGGGCGGCGGAGTGGAGTGAGAGCGCCGCGCGAGCAACGCGACTCGGTGGGAGCTCGTTCGGCA | WSESAARATRLGGSSFGNLDSLI |
| | | ACCTGGACTCGCTGATCAAAGATGTGTTGCTCTCTGGCAGGAGGAGTTAG | KDVLLSGRRS* |
| | | SEQ ID NO: 297 | SEQ ID NO: 298 |
| 150 | Xa21_protkinase | ATGCTGCCCTTGCTTTTCTTTCCTGTATTCGTGTTTGCCACAACGCTTCATGGAGCCATCGGCCAAGA | MLPLLFFPVFVFATTLHGAIGQE |
| | | AGCTGGCTTTCTAAGCATCGACTGCGGCCTAGAGGACACCAGCGGCTACAAGGAAGACAAGACTGGCA | AGFLSIDCGLEDTSGYKEDKTGI |
| | | TCATCTACGTCTCCGACGGCTCCTACGTCGATGCCGGGGAGAACCACCGTGTGTCGGCGGAGGAAGAG | IYVSDGSYVDAGENHRVSAEEEN |
| | | AACGCGAAATTGCCACGTCCGTACGCGACGCTCCGGAGCTTCCCTTCCGGCATGCGGAATTGCTACAC | AKLPRPYATLRSFPSGMRNCYTL |
| | | GCTGCCCGCGGACGCTGGGGCCAAGTACCTTGTCCGGGTGGTGTCCTTCTACGGCAACTACGACGGGA | PADAGAKYLVRVVSFYGNYDGKN |
| | | AGAACAGCTCATCGTCGTTGCAGTTCGACCTGCACCTCGGGACAAACTACTGGGCCACTGTCCGCGCT | SSSSLQFDLHLGTNYWATVRADG |
| | | GACGGCAACAACGTGTACGAGGCGCTGTTCGTGGCCTGGGCGAGCTGGGTGCCGGTGTGTCTCGTGAA | NNVYEALFVAWASWVPVCLVNTG |
| | | CACCGGCCGTGGCACGCCGTTCGTGTCCGCGGTGGAGCTGAGGCAGCTCAGCGGCGAGCTCTACCCGG | RGTPFVSAVELRQLSGELYPGLN |
| | | GCCTCAATGCCAGCCAGTCCTTGTCCATGTATGGACGGCGCAACATGGGTTCAAACATTTCATATACA | ASQSLSMYGRRNMGSNISYTRYP |
| | | CGTTACCCCCACGATCGATATGACCGGTACTGGTGGCAGGTATGGTCTGATCCAATGTGGAAGAACAT | HDRYDRYWWQVWSDPMWKNISTP |
| | | TTCCACTCCATCAACCATCGAACAGAGCTCCATCTTCGCTTTGCCCCTCCCCGTTATCCAGACCGCAG | STIEQSSIFALPLPVIQTAVETV |
| | | TTGAGACGGTCAGCAATAAGACAATGCTCAACATTACATGGCAAGCGGAAACACCCCGAGGCTTTTTG | SNKTMLNITWQAETPRGFLVFLH |
| | | GTTTTCTTGCACTTCGCCGACTTCCAGAACAGTCAATTCCGACAGTTCAAGGTCGCTTTTAACAATGA | FADFQNSQFRQFKVAFNNDYRDN |
| | | CTATCGTGATAACTATAGCCCACCATACATGACCACAGATGCCTTGTTTGGCTTGACAAGGGCCACCA | YSPPYMTTDALFGLTRATSGMYS |
| | | GTGGCATGTACAGCATCAGTATTGGAGCCACCGATGCATCTAAGCTGCCGCCGATGCTAAACGCGATT | ISIGATDASKLPPMLNAIEAYTL |
| | | GAGGCATACACTCTCATGTCCCGTGACAACCCAATAACGTTCCCAAACGATTTTGACACTATCATGGC | MSRDNPITFPNDFDTIMAIKHEY |
| | | AATTAAACATGAATACGGGATAAAGAAGAATTGGATGGGTGATCCATGTTTCCCAACACAATTTGCAT | GIKKNWMGDPCFPTQFAWDGVKC |
| | | GGGATGGCGTGAAATGCAACAATGCAAGTGGTAACAATACGAGGATAATATATTTGGATCTCTCGAAC | NNASGNNTRIIYLDLSNNNLHGP |
| | | AACAACTTGCATGGGCCGATATCTAATAACTTTACATTGCTCACGGCACTCAAGTACCTGAATTTGTC | ISNNFTLLTALKYLNLSGNQLNA |
| | | GGGCAACCAGCTGAATGCACCCATTCCTAAGTCCCTCTGTGAAAACAATAATACAGGATCATTGATTC | PIPKSLCENNNTGSLILSFDSDR |
| | | TCAGTTTCGATTCTGACCGAGAAACGTGCACAATAATACCGCCTCCAACAATAAAAAAATTGAACAAA | ETCTIIPPPTIKKLNKAILAISV |
| | | GCAATCTTAGCTATTTCGGTAGTGGTTCCTGTGATGGCCATTGCTATTCTTGTTCTCGCTTATTTGAT | VVPVMAIAILVLAYLIWRQKRKP |
| | | CTGGAGACAGAAAAGAAAACCAAACATATCAATAGATGATACTCCGAGAGAACGACAAGCTGAAAATG | NISIDDTPRERQAENVPASRKNH |
| | | TTCCAGCAAGCAGAAAAAACCATGGGAATGCCTTGCTAATGGTTGAGAATCGCCAATTCACATACAAG | GNALLMVENRQFTYKELEKLTNK |
| | | GAGCTTGAGAAGTTAACTAATAAGTTTGAACGCTTCATTGGACAAGGAGGTTTTGGACTCGTGTACTA | FERFIGQGGFGLVYYGLIEDGIE |
| | | CGGACTTATAGAAGATGGTATTGAGGTTGCTGTCAAGATACGTTCTGAGTCATCGTGTCATGGACTCG | VAVKIRSESSCHGLDEFFAEVQS |
| | | ATGAATTTTTCGCTGAGGTTCAAAGTTTGACAAAGGTGCATCACAGAAATCTAGTTTCCTTGGTTGGT | LTKVHHRNLVSLVGYCWEKNHLA |
| | | TACTGCTGGGAGAAGAATCATTTAGCACTAGTTTATGAGTACATGGCTCGAGGCACTCTTTGTGATCA | LVYEYMARGTLCDHLRGNHGFSE |
| | | TCTGAGAGGTAATCATGGTTTTAGTGAAACCTTGAATTGGAAAACACGAGTCCGAGTTTTGGTTGAAG | TLNWKTRVRVLVEAAQGLDYLHK |
| | | CTGCACAAGGACTGGATTATCTGCATAAAGGTTGTAGTCCGCCAATAATTCACGGAGATGTGAAGACG | GCSPPIIHGDVKTSNILLDQNLQ |
| | | AGTAACATTCTGTTGGATCAAAATTTACAAGCTAAAATTGCAGATTTTGGTCTTTCCAAGACATATCT | AKIADFGLSKTYLSDTQTHISVN |
| | | TAGCGACACACAGACGCACATATCAGTCAATCCAGCTGGGTCAGCTGGTTATATGGACCCTGGGTACT | PAGSAGYMDPGYFHTGRLTESCD |
| | | TTCACACAGGCAGACTCACGGAGAGTTGTGATGTTTATAGTTTTGGAATTGTTCTGTTAGAGATAGCC | VYSFGIVLLEIATGESPILPGQD |
| | | ACCGGCGAGTCTCCCATACTACCAGGTCAAGACCACATTATTCAGCGTGTGAATAAGAAGATTGCTAC | HIIQRVNKKIATGNISLVADARL |
| | | CGGTAATATCAGCTTGGTTGCTGATGCACGACTCAGAGGTGCCTATGATATCAGCTCAATGTGGAAGG | RGAYDISSMWKVVDIGLLCTTDT |
| | | TGGTGGACATCGGACTGTTATGCACTACTGACACTGGTGCAAAAAGGCCGACAATGGCTGCTGTGGTT | GAKRPTMAAVVVRLNESLALEEA |
| | | GTGCGACTCAATGAGAGCCTTGCATTGGAGGAAGCTCATTGCGAGAGTGGCTTCAGGGGAAGCACAGG | HCESGFRGSTGTSCDTTVSTSTF |
| | | CACATCGTGTGATACCACAGTTTCAACATCTACGTTTGGGCCTTCAGCAAGATGA | GPSAR* |
| | | SEQ ID NO: 299 | SEQ ID NO: 300 |

### Example 2

### Estimation of nifH Transcript Abundance

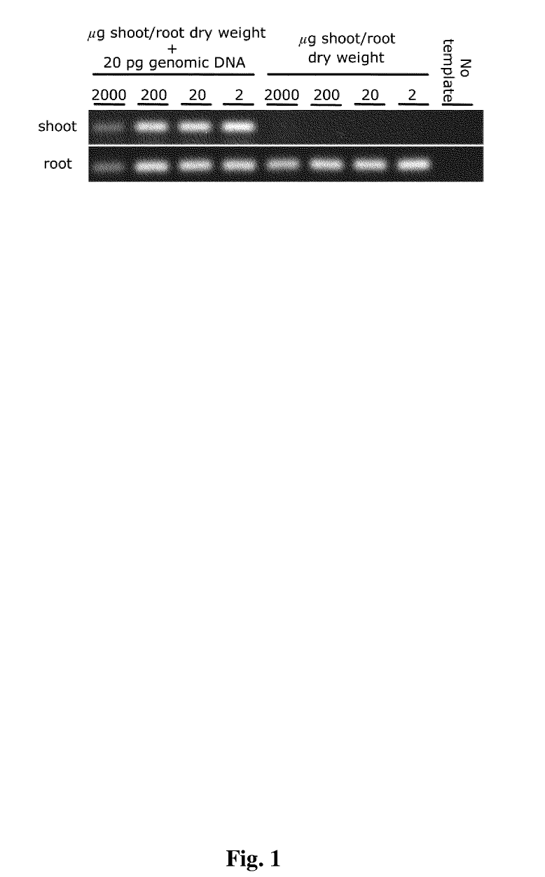

NifH transcript levels associated with plant roots were assessed from total RNA extracts from plant roots as previously published (Hurek et al., 2002) with improvements (Burbano et al., 2011, supra). In FIG. 1, with similar primers DNA-amplification of nifH fragments was done as in Hurek et al., 2002. For FIG. 2, the amplification of nifH fragments was obtained by universal primer sets in a nested reverse transcription (RT)-PCR approach (Burbano et al., 2011, supra): a nested RT-PCR protocol was used. The RT step was done at 42° C. using the primer nifH3. The PCR amplification was carried out with annealing 55° C. with primers nifH3 and nifH4. The second round of the nested PCR amplification was performed with 1 μl of the first-round product with the primers ZehrF and ZehrR. For this second round, quantitative real-time PCR was applied for quantification (Bio-Rad CFX 96). The actin gene was used to normalize the quantification of nifH expression.

For the production of in vitro transcribed nifH mRNA as control, a 1172-bp PvuII-EcoRI fragment from plasmid pBGvN322 corresponding to base 262 to 1433 of the Mo-nitrogenase operon of Azoarcus sp. BH72 (GenBank accession AF200742) was subcloned into pBluescript KS (Stratagene) to yield plasmid pBTH2. A 1.2 kb sense transcript was transcribed from the T3 promoter, after truncation with EcoRI. Transcription reactions were carried out as described in B. Reinhold-Hurek, D. A. Shub, Nature 357, 173 (1992).

In FIG. 2, levels of nifH transcripts in roots are shown. Asian cultivated rice (Oryza sativa) roots showed significantly lower transcript levels than wild rice roots (O. longistaminata). In interspecific hybids (F1) high transcript levels were retained (multiple t tests with multiple comparison post test; *, P<0.05). Values are means with SD from at least 3 replicates. All values from the rice cultivars were compared to those from wild rice. NUN transcripts were quantified by real-time RT-PCR. The actin gene was used to normalize the quantification of nifH expression.

### Example 3

### Determination of Nitrogen Incorporation by ¹⁵N Measurement

Oryza longistaminata and other plants were grown in a growth chamber as described in L. Miché, F. Battistoni, S. Gemmer, M. Belghazi, B. Reinhold-Hurek, Mol. Plant Microbe Interact. 19, 502 (2006), at 30° C. with a 16 h day/8 h night cycle in pots in water-saturated soil from the Carmague, France, which has been not treated with N fertilizer for 10 years. For ¹⁵N₂ tracer experiments, pots were placed into a gas-tight chamber in a Conviron phytotron, with controlled light, humidity and CO₂ concentration. Immediately before use, ¹⁵N₂ gas (99.30 atom % ¹⁵N; Euriso-top, Gif-Sur-Yvette, France) was successively treated with alkaline permanganate and diluted sulfuric acid to remove possible impurities such as nitrogen oxides and NH₃ as described in R. H. Burris, in: A. Quispel (Ed), The biology of nitrogen fixation, (North Holland Publishing Company, Amsterdam, 1974), pp. 9-23. ¹⁵N % abundance in headspace of the chamber was measured with a Finnigan MAT 8200 mass spectrometer (Finnigan MAT GmbH) at the beginning and at the end of incubation with ¹⁵N-labeled N₂.

Plants were sampled after the 2^{nd} consecutive cut, after growth under a headspace of 0.41±0.15 atom % ¹⁵N excess. The incorporation of ¹⁵N₂ tracer into re-grown shoots of a wild rice species, F1-hybrids and backcrosses with Oryza sativa was tested. In contrast to the O. sativa cultivar and the backcross tested, two different F1-hybrids retained high N₂-gain potential similar to wild rice (one-way ANOVA and Bonferroni's multiple comparison post test *, P<0.05).

1. A method of increasing yield, biomass, growth rate, vigor, nitrogen gain derived from biological nitrogen fixation, nitrogen use efficiency, abiotic stress tolerance and/or preferably of increasing tolerance to nutrient deficiency of a plant, comprising introducing isolated genetic material of Oryza longistaminata to a target plant or plant cell.

2. The method of claim 1, wherein the isolated genetic material is selected from the group consisting of the nucleic acid sequences set forth in Table 1 or nucleic acid sequences encoding a polypeptide at least 60% identical to an amino acid sequence encoded by the nucleic acid sequences set forth in Table 1.

3. The method of claim 1, wherein the target plant or plant cell is selected from the group of plants or plant cells, respectively, of
a) family Poaceae,
b) subfamily Ehrhartoidae, Pooideae, Panicoideae or Chloridoideae,
c) tribum Oryzeae, Triticeae, Paniceae or Andropogoneae,
d) genus Oryza, Hordeum, Secale, Triticum, Triticosecale, Saccharum, Eleusine, Sorghum, Pennisetum or Zea; and
e) Oryza sativa, Oryza glaberrima (cultivated rice), Hordeum vulgare (barley), Secale cereale (rye), Triticum aestivum (bread wheat), Triticum monococcum, Saccharum officinarum, Saccharum robustum, Saccharum sinense, Saccharum barberi, Saccharum edule, Saccharum spontaneum (sugar cane), Eleusine coracana, Sorghum bicolor (Sorghum), Pennisetum glaucum (millet), Zea mays (maize), or hybrids thereof.

4. The method of claim 1, wherein the genetic material is introduced by amphiploidization, single chromosome addition/substitution, centric translocation and/or homologous recombination.

5. The method of claim 1, wherein the genetic material of Oryza longistaminata is introduced to the target plant or plant cell by
a) wide hybridization,
b) chemical methods,
c) microinjection,
d) electroporation,
e) particle acceleration and/or particle bombardment,
f) one or more viral vectors or bacterial vectors, preferably Agrobacterium,
g) receptor-mediated mechanisms,
h) injection of genetic material into a reproductive organ of the target plant,
i) protoplast transformation, and/or
j) injection into an immature embryo of the target plant, or
k) two or more of (a)-(j).

6. The method of claim 1, further comprising the steps of:
(i) growing a root and/or shoot of the plant or plant cell obtained by the method of claim 1 inoculated with a nitrogen fixing microorganism or not inoculated in soil;
(ii) determining expression levels of nitrogenase mRNA in the plant roots;
(iii) selecting a plant having higher root-associated expression levels of nitrogenase mRNA than the parental cultivated plant; and
(iv) determining the level of plant nitrogen gained by nitrogen fixation by isotopic, ¹⁵N-based methods.

7. The method of claim 1, wherein the nitrogen fixing microorganism is a bacterium, preferably of family Rhodocyclaceae.

8. The method of claim 1, wherein the introduction of the isolated genetic material to the target plant or plant cell imparts, increases or modifies root association of the plant with a nitrogen fixing microorganism, and/or reduces nitrogen fertilization demand of the plant or the plant cell.

9. A polynucleotide comprising the isolated genetic material of claim 1.

10. A vector comprising the polynucleotide of claim 9.

11. The vector of claim 10, wherein the genetic material is selected from the group consisting of the nucleic acid sequences set forth in Table 1 or encode a polypeptide at least 60% identical to a polypeptide encoded by such nucleic acid sequence and is operably linked to heterologous control sequences capable of directing transcription and preferably expression of the nucleic acid sequences in a host plant cell.

12. A cultivated plant, plant cell or seed thereof comprising the genetic material of claim 1.

13. (canceled)

14. A method for imparting, increasing or modifying root association of a plant with a nitrogen fixing microorganism, reducing fertilization demand of a plant and/or improving nitrogen sustainability of a cereal plant which comprises introducing into the plant the genetic material of claim 1.

15. The method of claim 2, wherein the target plant or plant cell is selected from the group of plants or plant cells, respectively, of
a) family Poaceae,
b) subfamily Ehrhartoidae, Pooideae, Panicoideae or Chloridoideae,
c) tribum Oryzeae, Triticeae, Paniceae or Andropogoneae,
d) genus Oryza, Hordeum, Secale, Triticum, Triticosecale, Saccharum, Eleusine, Sorghum, Pennisetum or Zea; and
e) Oryza sativa, Oryza glaberrima (cultivated rice), Hordeum vulgare (barley), Secale cereale (rye), Triticum aestivum (bread wheat), Triticum monococcum, Saccharum officinarum, Saccharum robustum, Saccharum sinense, Saccharum barberi, Saccharum edule, Saccharum spontaneum (sugar cane), Eleusine coracana, Sorghum bicolor (Sorghum), Pennisetum glaucum (millet), Zea mays (maize), or hybrids thereof.

16. The method of claim 2, wherein the genetic material is introduced by amphiploidization, single chromosome addition/substitution, centric translocation and/or homologous recombination.

17. The method of claim 3, wherein the genetic material is introduced by amphiploidization, single chromosome addition/substitution, centric translocation and/or homologous recombination.

18. The method of claim 3, wherein the genetic material of Oryza longistaminata is introduced to the target plant or plant cell by
a) wide hybridization,
b) chemical methods,
c) microinjection,
d) electroporation,
e) particle acceleration and/or particle bombardment,
f) one or more viral vectors or bacterial vectors, preferably Agrobacterium,
g) receptor-mediated mechanisms,
h) injection of genetic material into a reproductive organ of the target plant,
i) protoplast transformation, and/or
j) injection into an immature embryo of the target plant, or
k) two or more of (a)-(j).

19. The method of claim 3, further comprising the steps of:
(i) growing a root and/or shoot of the plant or plant cell obtained by the method of claim 1 inoculated with a nitrogen fixing microorganism or not inoculated in soil;
(ii) determining expression levels of nitrogenase mRNA in the plant roots;
(iii) selecting a plant having higher root-associated expression levels of nitrogenase mRNA than the parental cultivated plant; and
(iv) determining the level of plant nitrogen gained by nitrogen fixation by isotopic, ¹⁵N-based methods.

20. The method of claim 5, further comprising the steps of:
(i) growing a root and/or shoot of the plant or plant cell obtained by the method of claim 1 inoculated with a nitrogen fixing microorganism or not inoculated in soil;
(ii) determining expression levels of nitrogenase mRNA in the plant roots;
(iii) selecting a plant having higher root-associated expression levels of nitrogenase mRNA than the parental cultivated plant; and
(iv) determining the level of plant nitrogen gained by nitrogen fixation by isotopic, ¹⁵N-based methods.

21. The method of claim 3, wherein the nitrogen fixing, microorganism is a bacterium, preferably of family Rhodocyclaceae.
